(19) United States
(12) Patent Application Publication
(10) Pub. No.: US 2026/0364011 A1
(43) Pub. Date: Nov. 19, 2026

(54) LIPID COMPOSITION

(71) Applicant: STEMIRNA THERAPEUTICS CO., LTD., Pudong New Area, Shanghai (CN)

(72) Inventors: Yu HANG, Shanghai (CN); Lei HUANG, Shanghai (CN); Haifa SHEN, Shanghai (CN); Hangwen LI, Shanghai (CN)

(21) Appl. No.: 19/123,047

(22) PCT Filed: Nov. 6, 2023
(86) PCT No.: PCT/CN2023/129883

§ 371 Date: Apr. 21, 2025

(30) Foreign Application Priority Data
Nov. 6, 2022 (CN) 202211381081.5

(51) Int. Cl. A61K 9/1272; A61K 9/00; A61K 9/51; A61K 31/7105

(52) U.S. Cl. CPC A61K 9/1272; A61K 9/0043; A61K 9/5123; A61K 31/7105

(57) ABSTRACT

The present invention relates to mucosal drug delivery systems, and specifically to a lipid composition; the lipid composition containing a therapeutic agent and/or prophylactic agent such as RNA can be used for mucosal delivery of the therapeutic agent and/or prophylactic agent to treat or prevent disease such as infectious disease.

### CROSS-REFERENCES

This application claims priority from Chinese Patent Application No. 202211381081.5 filed on Nov. 6, 2022, the entirety of which is incorporated herein by reference.

### TECHNICAL FIELD

The present invention relates to drug delivery systems, and in particular to lipid compositions for mucosal administration as well as related products and uses in the treatment or prevention of infectious diseases.

### BACKGROUND

Lipid-containing nanoparticle compositions, liposomes and lipoplexes serve as transport vehicles that can effectively deliver bioactive substances such as small molecule drugs, proteins and nucleic acids to cells and/or intracellular compartments. In the room. These lipid compositions generally include cationic lipids, structural lipids, helper lipids and/or surfactants.

Existing lipid-based drug delivery systems, such as liposomes, lipid nanoparticle (LNP) drug delivery systems, etc., have been widely used in recent years. However, during actual use, it was found that when these drug delivery systems were used for mucosal administration of biologically active substances such as nucleic acids, there were problems such as low expression of mRNA in the mucosa and weak immunogenicity.

At present, some researchers have found that mucosal administration, such as nasal administration, using disclosed polymers, positively charged liposomes, viral vectors or some commercial transfection reagents will lead to a large amount of protein expression in the lungs. Some researchers also compared three-component LNP drug delivery systems containing different lipid combinations such as cationic lipids, auxiliary phospholipids, and PEG, and found that the drugs delivered through different administration routes (intramuscular route, intradermal route, intranasal administration) The immunogenicity and expression of the same nucleic acid in the body are different. The immune effect of nasal administration is significantly weaker than that of intramuscular and subcutaneous administration. Moreover, after nasal administration, the delivered nucleic acid will be distributed in large amounts in the throat and stomach of mice. Department (G. Anderluzzi, et al. The role of nanoparticle format and route of administration on self-amplifying mRNA vaccine potency. Journal of Controlled Release 342 (2022) 388-399.).

This field needs a drug delivery system that can be highly expressed only in the mucosa after administration to the mucosa to stimulate mucosal immunity and better protect the body.

### SUMMARY OF THIS INVENTION

In one aspect, the present invention provides a lipid composition for mucosal administration, comprising a therapeutic or prophylactic agent and a lipid encapsulating the therapeutic or prophylactic agent, wherein the therapeutic or prophylactic agent is encapsulated in a lipid. Lipids include cationic lipids, phospholipids, steroids, and polyethylene glycol modified lipids: the lipid composition further includes a cationic polymer, wherein the cationic polymer is associated with the therapeutic or preventive agent as a complex substance, co-encapsulated in lipids to form lipid multimeric complexes. In one embodiment, the lipid composition contains 2.5-20 mole % of polyethylene glycol modified lipids, based on the total amount of all lipids in the lipid composition.

In one embodiment, the therapeutic or prophylactic agent is a nucleic acid, such as RNA, especially mRNA.

In one embodiment, the cationic lipid comprises a lipid compound of formula (I), (II), (III), (IV), or a pharmaceutically acceptable salt thereof, as defined herein. Preferably, the cationic lipid is M5, MC3, ALC-0315,
SM-102, SW-II-121, SW-II-138-1, SW-II-139-1, SW-II-140-1 or SW-II-140-2.

In one embodiment, the cationic lipid does not include T5′.

In one embodiment, the lipid composition contains 30-60 mol % cationic lipids, 5-40 mol % phospholipids, 10-70 mol % steroids, and 2.5-20 mol % polyethylene glycol modification of lipids.

In one embodiment, the lipid composition contains 30-60 mol % cationic lipids, 5-40 mol % phospholipids, 10-60 mol % steroids, and 2.5-20 mol % polyethylene glycol modification of lipids.

In one embodiment, the lipid composition contains 35-50 mol % cationic lipids, 10-35 mol % phospholipids, 15-50 mol % steroids, and 2.5-20 mol % polyethylene glycol modification of lipids.

In one embodiment, the lipid composition contains 37.5-42.5 mol % cationic lipids, 25-35 mol % phospholipids, 15-30 mol % steroids, and 2.5-20 mol % polyethylene glycol modification of lipids.

In a preferred embodiment, the lipid composition contains 37.5-42.5 mol % cationic lipids, 25-35 mol % DOPE, 15-30 mol % cholesterol, and 2.5-20 mol % DMG-PEG.

In a preferred embodiment, the lipid composition contains 37.5-42.5 mol % cationic lipids, 25-35 mol % DOPE, 15-30 mol % cholesterol, and 3.75-10 mol % DMG-PEG.

In a preferred embodiment, the lipid composition contains 37.5-42.5 mol % cationic lipids, 25-35 mol % DOPE, 15-30 mol % cholesterol, and 3.75-5 mol % DMG-PEG.

In a particularly preferred embodiment, the lipid composition contains 42.5 mol % cationic lipids, 35 mol % DOPE, 18.75 mol % cholesterol and 3.75 mol % DMG-PEG.

In one aspect, the present invention also provides a pharmaceutical composition comprising the lipid composition of the present invention, and optional pharmaceutically acceptable excipients.

On the other hand, the lipid composition or pharmaceutical composition of the present invention is used for nasal, oral, conjunctival, rectal, and vaginal mucosa administration.

In a preferred embodiment, the lipid composition or pharmaceutical composition is used for nasal administration.

In a preferred embodiment, the nasal administration includes nasal instillation, nasal spray administration or nasal inhalation.

In one embodiment, the nasal spray administration is performed via an aerosol administration device.

In another aspect, the invention also provides a method for treating or preventing a disease or condition, comprising administering to a subject a therapeutic or prophylactic agent in a multiple dose regimen, wherein at least one dose is administered by a mucosal route The lipid composition of the present invention or the pharmaceutical composition of the present invention.

In another aspect, the present invention also provides a nasal drop or nasal spray, which contains the lipid composition of the present invention and a pharmaceutically acceptable excipient.

On the other hand, the present invention also provides the use of the lipid composition of the present invention, the pharmaceutical composition of the present invention or the nasal drops or nasal spray of the present invention in the preparation of medicaments for the treatment or prevention of diseases. The disease or condition of the subject in need.

### DESCRIPTION OF DRAWINGS

FIGS. 1A and 1B show the in vivo expression of LPP preparations prepared through different recipes in the third round of screening. FIG. 1A is a mouse in vivo imaging diagram; FIG. 1B is a statistical diagram of luciferase expression, expressed by the area under the curve (AUC).

FIGS. 2A-2D illustrate the immunization of LPP formulations containing COVID-19 mRNA by different immunization protocols. FIG. 2A shows the immunization scheme; FIG. 2B shows the ELISpot detection results of spleen cells in each group of mice immunized with different immunization schemes; FIG. 2C shows the ELISpot detection results of lung cells in each group of mice; FIG. 2D shows the ELISA detection Results of IgA antibody levels in nasal lavage fluid of mice in each group.

FIGS. 3A-3E show the detection results of the immunogenicity of LPP preparations containing mRNA encoding influenza virus antigens applied through different immunization protocols. FIG. 3A shows the immunization scheme; FIG. 3B shows the ELISpot detection results of spleen cells in each group of mice immunized with different immunization schemes; FIG. 3C shows the ELISpot detection results of lung cells in each group of mice; FIG. 3D shows the detection by ELISA The results of the IgA antibody levels in the nasal lavage fluid of mice in each group; FIG. 3E shows the results of detecting the IgA antibody levels in the lung lavage fluid of mice in each group by ELISA.

FIGS. 4A and 4B show the cell transfection efficiency of LPP formulations prepared with different cations for mucosal administration. FIG. 4A shows the results of measuring the cell transfection efficiency in A549 cells; FIG. 4B shows the results of measuring the cell transfection efficiency in DC2.4 cells.

FIGS. 5A and 5B show the in vivo expression of LPP formulations prepared with different cations for mucosal administration. FIG. 5A is a mouse in vivo imaging diagram; FIG. 5B is a statistical diagram of luciferase expression, expressed by total flux.

FIGS. 6A-6C show the cell transfection efficiency of LPP formulations before and after nebulization. FIG. 6A is a schematic diagram of the aerosol drug delivery device; FIG. 6B is the luciferase expression of the B11/Luc LPP preparation containing luciferase mRNA in DC2.4 cells before and after atomization; FIG. 6C is The expression of eGFP in A549 cells by the B11/eGFP LPP preparation containing eGFP mRNA before nebulization, before nebulization, during nebulization and after nebulization.

### DETAIL DESCRIPTION

### General Definitions

All patents, patent applications, scientific publications, manufacturer's instructions and instructions, etc. cited herein, whether supra or infra, are hereby incorporated by reference in their entirety. Nothing contained herein should be construed as an admission that this disclosure is not entitled to antedate such disclosure.

Unless otherwise stated, scientific and technical terms used herein have the meaning commonly understood by those skilled in the art. Moreover, the terms related to protein and nucleic acid chemistry, molecular biology, cell and tissue culture, and microbiology used in this article are all terms widely used in the corresponding fields. Meanwhile, in order to better understand the present invention, definitions and explanations of relevant terms are provided below.

As used herein, the expressions “comprises,” “comprising,” “containing,” and “having” are open-ended and mean the inclusion of recited elements, steps or components but not the exclusion of other unrecited elements, steps or components. The expression “consisting of” does not include any element, step or component not specified. The expression “consisting essentially of” means that the scope is limited to the specified elements, steps or components plus the optional presence of elements, steps or components that do not materially affect the basic and novel properties of the claimed subject matter. It will be understood that the expressions “consisting essentially of” and “consisting of” are encompassed within the meaning of the expression “comprising”.

As used herein, the singular forms “a,” “an,” or “the” include plural referents unless the context dictates otherwise. The term “one or more” or “at least one” encompasses 1, 2, 3, 4, 5, 6, 7, 8, 9 or more.

The recitation of ranges of values herein is intended only as a shorthand method of individually referring to each distinct value that falls within the stated range. Unless otherwise stated herein, each individual value is incorporated into this specification as if it were individually recited herein. Unless expressly stated to the contrary, numerical values or ranges shown herein are modified by “about” to indicate the recited or claimed value or range ±20%, ±10%, ±5% or ±3%.

Unless otherwise stated, all methods described herein can be performed in any suitable order.

In the lipid compositions of the present invention, the amounts of each lipid are calculated in mole percent (mol %) unless otherwise specified. The percentages can be calculated based on the total amount of all lipids in the lipid composition. Those skilled in the art will understand that the content of each lipid can be appropriately selected so that the total amount is 100%.

As used herein, “nucleotide” includes deoxyribonucleotides and ribonucleotides and their derivatives. As used herein, “ribonucleotide” is the constituent material of ribonucleic acid (RNA), which is composed of one molecule of base, one molecule of five-carbon sugar, and one molecule of phosphate, which refers to the sugar in β-D-ribofuranose (β-D-ribofuranosyl) A nucleotide with a hydroxyl group at the 2′ position. “Deoxyribonucleotide” is the constituent material of deoxyribonucleic acid (DNA). It is also composed of one base molecule, one five-carbon sugar molecule, and one phosphate molecule. It refers to the sugar in β-D-ribofuranose (β-D-ribofuranosyl) is a nucleotide in which the hydroxyl group at the 2′ position is replaced by hydrogen. It is the main chemical component of chromosomes. “Nucleotide” is usually referred to by a single letter that represents the base within it: “A(a)” refers to deoxyadenosine or adenylate containing adenine, and “C(c)” refers to deoxyadenosine containing cytosine. Cytidylic acid or cytidylic acid, “G(g)” refers to deoxyguanylic acid or guanylic acid containing guanine. “U(u)” refers to uridylic acid containing uracil, “T(t)” refers to Deoxythymidylate containing thymine.

As used herein, the terms “polynucleotide” and “nucleic acid” are used interchangeably to refer to a polymer of deoxyribonucleotides (DNA) or a polymer of ribonucleotides (ribonucleic acid, RNA). “Polynucleotide sequence,” “nucleic acid sequence,” and “nucleotide sequence” are used interchangeably to refer to the ordering of nucleotides in a polynucleotide. Those in the art should understand that the DNA coding strand (sense strand) and the RNA it codes for can be regarded as having the same nucleotide sequence, and the deoxythymidylate in the DNA coding strand sequence corresponds to the uridylic acid in the RNA sequence it codes for.

As used herein, “modified” refers to non-natural. For example, the RNA can be modified RNA. That is, RNA may include one or more non-naturally occurring nucleobases, nucleosides, nucleotides, or linking groups. “Modified” groups may also be referred to herein as “altered” groups. Groups may be modified or altered chemically, structurally, or functionally. For example, a modified nucleobase may include one or more non-naturally occurring substitutions.

As used herein, the term “expression” includes the transcription and/or translation of a nucleotide sequence. Thus, expression may involve the production of transcripts and/or polypeptides. The term “transcription” refers to the process of transcribing the genetic code in a DNA sequence into RNA (transcript). The term “in vitro transcription” refers to the in vitro synthesis of RNA, in particular mRNA, in a cell-free system, for example in a suitable cell extract. Vectors that can be used to produce transcripts are also called “transcription vectors” and contain the regulatory sequences required for transcription. The term “transcription” encompasses “in vitro transcription”.

As used herein, the term “host cell” refers to a cell used to receive, maintain, replicate, or express a polynucleotide or vector.

As used herein, an “aliphatic” group is a non-aromatic group in which carbon atoms are linked into a chain, and may be saturated or unsaturated.

As used herein, the term “alkyl” refers to an optionally substituted straight or branched chain saturated hydrocarbon including one or more carbon atoms. The term “C₁-C₁₂ Alkyl” or “C₁₋₁₂ “Alkyl” refers to an optionally substituted straight or branched chain saturated hydrocarbon containing 1 to 12 carbon atoms. As used herein, the term “alkoxy” refers to an alkyl group as described herein, which is attached to the molecule through an oxygen atom The remainder of the term “alkylene” refers to the bivalent group formed by the loss of one hydrogen atom of the corresponding alkyl group. C₁-C₁₂ Alkylene” or “C₁₋₁₂ “Alkylene” refers to an optionally substituted straight or branched chain alkylene group containing 1 to 12 carbon atoms.

As used herein, the term “alkenyl” refers to an optionally substituted straight or branched hydrocarbon chain including two or more carbon atoms and at least one double bond. The term “C₂-C₁₂ Alkenyl” or “C₂₋₁₂ “Alkenyl” refers to a group containing 2 to 12 carbon atoms and at least one carbon-straight or branched chain hydrocarbons with optionally substituted carbon double bonds. Alkenyl groups may include one, two, three, four or more carbon-carbon double bonds.

As used herein, the term “halogen” refers to fluorine, chlorine, bromine and iodine.

As used herein, the term “carbocycle” refers to a monocyclic or polycyclic non-aromatic system that includes one or more rings composed of carbon atoms. The term “C₃₋₈ “Carbocycle” means a carbocycle including 3 to 8 carbon atoms. A carbocycle may include one or more carbon-carbon double or triple bonds. Examples of carbocycles include, but are not limited to, cyclopropyl, cyclopentyl, cyclopentyl, Hexyl, etc. As used herein, when a carbocycle is saturated (i.e., contains no unsaturated bonds), the corresponding cycloalkyl group may also be referred to. Unless otherwise specified, the carbocycle referred to herein refers to unsubstituted, and substituted, i.e., optionally substituted carbocyclic rings.

As used herein, the term “heterocycle” refers to a monocyclic or polycyclic ring system that includes one or more rings and includes at least one heteroatom. Heteroatoms may be, for example, nitrogen, oxygen, phosphorus or sulfur atoms. Heterocycles may include one or more double or triple bonds and may be nonaromatic. Examples of heterocycles include, but are not limited to, imidazolidinyl, oxazolidinyl, thiazolidinyl, pyrazolidinyl, isoxazolidinyl, isothiazolidinyl, morpholinyl, pyrrolidinyl, tetrahydrofuranyl, and piperidine base. Heterocycles may contain, for example, 3-10 atoms (non-hydrogen), i.e., 3-10 membered heterocycles (e.g., 3, 4, 5, 6, 7, 8, 9, or 10 members), one or more of which are heterocycles. Atom (such as N, O, S or P). When the heterocycle is saturated (ie, contains no unsaturated bonds), the corresponding heterocycloalkyl group may also be referred to. Unless otherwise specifically stated, heterocycle as used herein refers to both unsubstituted and substituted heterocyclic groups, optionally substituted heterocycles.

As used herein, the term “aryl” refers to an all-carbon monocyclic or fused polycyclic aromatic ring group having a conjugated pi electron system. For example, C₆-C₁₀ Alkylaryl groups may have 6 to 10 carbon atoms, such as 6, 7, 8, 9, 10 carbon atoms. Examples of aryl groups include, but are not limited to, phenyl, naphthyl, and the like.

As used herein, the term “heteroaryl” refers to a monocyclic or fused polycyclic ring system containing at least one ring atom selected from N, O, S, the remaining ring atoms being C. and having at least one aromatic ring. Heteroaryl groups may have 5 to 10 ring atoms (5-10 membered heteroaryl groups), including 5, 6, 7, 8, 9 or 10 membered heteroaryl groups, especially 5 or 6 membered heteroaryl groups. Examples of heteroaryl groups include, but are not limited to, pyrrolyl, furyl, thienyl, imidazolyl, oxazolyl, pyrazolyl, pyridyl, pyrimidinyl, pyrazinyl, quinolyl, isoquinolyl, tetrazole base, triazolyl, triazinyl, benzofuranyl, benzothienyl, indolyl, isoindolyl, etc.

As used herein, the term “interrupted by one or more groups” means that the one or more groups are present on the carbon chain, and the remainder of the carbon chain is connected to both ends of the one or more groups.

Unless otherwise specified, the groups described herein (e.g., R₁-R₇ any of them, such as alkyl, alkylene, alkenyl, aryl, amino, etc.) may be optionally substituted. Optional substituents may be selected from the following, but are not limited to: halogen atoms (such as chlorine, bromo, fluoro or iodine), carboxylic acids (such as —C(O)OH), alcohols (such as hydroxyl, —OH), ester (such as —C(O)OR or —OC(O)R), aldehyde (such as —C(O)H), carbonyl group (such as —C(O)R, or represented by C═O), acid halide (e.g. —C(O)X, where (e.g. —C(OR)₂R″″, where each OR is the same or different alkoxy and R″″ is alkyl or alkenyl), phosphate (e.g. P(O)₄³⁻), thiols (e.g. —SH), sulfoxides (e.g. —S(O)R), sulfinic acids (e.g. —S(O)OH), sulfonic acids (e.g. —S(O)₂ OH), sulfide (e.g. —C(S)H), sulfate (e.g. S(O)₄²⁻) sulfonyl group (e.g. —S(O)₂—), amides (e.g. —C(O)NR₂ or —N(R)C(O)R), azido (such as —N₃), nitro (for example —NO₂), cyano group (such as —CN), isocyanate group (such as —NC), acyloxy group (such as —OC(O)R), amino group (such as —NR₂, NRH or —NH₂), carbamoyl (e.g. —OC(O)NR₂, —OC(O)NRH or —OC(O)NH₂), sulfonamide (e.g. —S(O)₂NR₂, —S(O)₂NRH, —S(O)₂NH₂, —N(R)S(O)₂R, —N(H)S(O)₂R, —N(R)S(O)₂H. —N(H)S(O)₂H), C₁-C₁₂

Alkyl, C₂-C₁₂ Alkenyl, C₆-C₁₀ Aryl, 5-10 membered heteroaryl or 3-10 membered heterocycle. In any of the foregoing, R can each independently be a substituent as defined herein, such as alkyl, alkoxy, alkylene, halogen, carbocycle, heterocycle, aryl, heteroaryl, alkenyl. In some embodiments, the substituents themselves may be further substituted by, for example, one, two, three, four, five or six substituents as defined herein. For example, an alkyl group may be further substituted with one, two, three, four, five or six substituents as described herein.

As used herein, the term “compound” is intended to include isotopic compounds of the depicted structures. “Isotopes” are atoms that have the same atomic number but different mass numbers due to the number of neutrons in the nucleus, such as deuterium isotopes. For example, isotopes of hydrogen include tritium and deuterium. Additionally, the compounds, salts or complexes of the present invention can be prepared in combination with solvents or water molecules to form solvates and hydrates by conventional methods.

The term “optionally” or “optionally” (e.g., substituted optionally) means that the subsequently described event may or may not occur, and that the description includes instances where the event or circumstance occurs as well as where the event or circumstance occurs. Instances where the situation does not occur. For example, “optionally substituted alkyl” means that the alkyl group may or may not be substituted, and this description includes both substituted and unsubstituted alkyl radicals.

It will be understood that when chemical groups are written in a specific order, the reverse order is also contemplated unless otherwise stated. For example, in M₁ defined as the general formula —(R) of —C(O)NH-ᵢ-(M1)ₖ—(R)ₘ— (i.e. —(R)ᵢ—C(O)—NH—(R)ₘ—), unless otherwise stated, also covers M₁ Compounds that are —NHC(O)— (i.e., —(R)ᵢ—NHC(O)—(R)ₘ—).

As used herein, the term “delivery” refers to providing an entity to a target. For example, delivering a therapeutic or prophylactic agent to a subject may involve administering to the subject a composition comprising the therapeutic or prophylactic agent.

As used herein, the term “subject” describes an organism to which use of the compositions of the present invention may be provided. Subjects to whom these compositions are intended to be administered include, but are not limited to, humans, other primates, and other mammals, such as cattle, pigs, horses, sheep, cats, dogs, mice, or rats. Preferably, the subject may be a mammal, especially a human.

As used herein, “encapsulation efficiency” refers to the ratio of the amount of therapeutic or prophylactic agent that becomes part of the composition to the initial total amount of therapeutic or prophylactic agent used to prepare the composition. For example, if 97 mg of therapeutic or prophylactic agent are encapsulated in the composition out of a total of 100 mg of therapeutic or prophylactic agent initially provided to the composition, it may be concluded that the encapsulation efficiency is 97%. As used herein, “encapsulating” may mean completely, substantially or partially enclosing, sealing, surrounding or packaging.

As used herein, a “lipid component” is a component of a composition that includes one or more lipids. For example, the lipid component may include one or more cationic lipids, pegylated lipids, structural lipids, or helper lipids.

The phrase “pharmaceutically acceptable” is used herein to mean, within the scope of reasonable medical judgment, suitable for use in contact with human and animal tissue without undue toxicity, irritation, allergic reaction, or other problems or complications, and with reasonable compounds, salts, materials, compositions and/or dosage forms that have a benefit/risk ratio.

As used herein, “pharmaceutically acceptable salts” refer to derivatives of the disclosed compounds in which the parent compound is converted into its salt form by converting an existing acid or base moiety (e.g., by combining the free basic group with a suitable organic acid reaction). Examples of pharmaceutically acceptable salts include, but are not limited to, inorganic or organic acid salts of basic residues such as amines; alkali metal or organic salts of acidic residues such as carboxylic acids, and the like. Representative acid addition salts include, but are not limited to, acetate, adipate, alginate, ascorbate, aspartate, benzenesulfonate, benzoate, bisulfate, borate, Butyrate, camphorate, camphorsulfonate, citrate, cyclopentane propionate, digluconate, lauryl sulfate, ethane sulfonate, fumarate, Glucoheptonate, glycerophosphate, hemisulfate, enanthate, caproate, hydrobromide, hydrochloride, hydroiodide, 2-hydroxy-ethanesulfonate, lactouronate, lactate, laurate, lauryl sulfate

Salt, malate, maleate, malonate, methane sulfonate, 2-naphthalene sulfonate, nicotinate, nitrate, oleate, oxalate, palmitate, Pamoate, pectate, persulfate, 3-phenylpropionate, phosphate, picrate, pivalate, propionate, stearate, succinate, sulfate, tartrate, thiocyanate, tosylate, undecanoate, valerate, etc. Representative alkali metal or alkaline earth metal salts include, but are not limited to, sodium, lithium, potassium, calcium, magnesium salts, etc.; and non-toxic ammonium, quaternary ammonium, and amine cations, including, but are not limited to, ammonium, tetramethylammonium, tetraethylammonium, methylamine, dimethylamine, trimethylamine, triethylamine, ethylamine, etc. Pharmaceutically acceptable salts of the present invention include, for example, conventional nontoxic salts of the parent compounds formed from nontoxic inorganic or organic acids. Pharmaceutically acceptable salts of the present invention can be synthesized by conventional chemical methods from parent compounds containing basic or acidic moieties. In general, these salts can be prepared by reacting the free acid or base form of these compounds with a stoichiometric amount of the appropriate base or acid in water or in an organic solvent, or in a mixture of the two; generally preferred Non-aqueous media such as diethyl ether, ethyl acetate, ethanol, isopropyl alcohol or acetonitrile.

As used herein, “polydispersity index” or “PDI” is a ratio that describes the homogeneity of the particle size distribution of a system. Smaller values, such as less than 0.3, indicate a narrower particle size distribution.

As used herein, “zeta potential” refers to, for example, the electrokinetic potential of a lipid in a lipid composition and is an important indicator of the stability of a dispersion.

As used herein, “size” or “average size” in the context of a composition refers to the average diameter of the composition.

As used herein, the term “treatment” means to partially or completely alleviate, ameliorate, ameliorate, alleviate, delay the onset of, inhibit the progression of, or reduce the severity of one or more symptoms or characteristics of a particular infection, disease, disorder or condition, degree or reduce its occurrence. “Prevention” means guarding against an underlying disease or preventing the worsening of symptoms or progression of a disease.

The term “therapeutically or prophylactically effective amount” refers to an agent (e.g., nucleic acid, drug, composition, therapeutic agent, diagnostic agent, preventive agent, etc.). The therapeutically or prophylactically effective amount is affected by factors including, but not limited to, the speed and severity of the development of the disease or symptoms, the age, gender, weight and physiological condition of the subject, the duration of treatment and the specific route of administration. A therapeutically or prophylactically effective amount may be administered in one or more doses. Therapeutically or prophylactically effective amounts can be achieved by continuous or intermittent administration.

### Lipid Composition

Provided herein is a lipid composition for mucosal administration. The lipid composition is a lipid delivery carrier, and the lipid can encapsulate nucleotides to form nanoparticles, thereby delivering them into the living body.

As used herein, the term “lipid” refers to an organic compound that contains a hydrophobic portion and, optionally, a hydrophilic portion. Lipids are generally poorly soluble in water but soluble in many organic solvents. Generally, amphipathic lipids containing hydrophobic and hydrophilic parts can be organized into lipid bilayer structures in an aqueous environment, for example in the form of vesicles. Lipids may include, but are not limited to: fatty acids, glycerides, phospholipids, sphingolipids, glycolipids, steroids, cholesterol esters, etc.

As used herein, “lipid nanoparticle” or “LNP” refers to a lipid vesicle with a uniform lipid core, which is a particle formed of lipids whose components undergo intermolecular interactions to form nanostructures entity. Nucleic acids (such as mRNA) are encapsulated in lipids.

A particularly preferred lipid composition may be, for example, a lipid polyplex (LPP) as described herein. Methods for preparing such compositions can be as described herein. LPP is a particle with a core-shell structure, in which nucleic acids are contained in a multimeric complex, and the multimeric complex itself is encapsulated in a biocompatible lipid bilayer shell to constitute the lipid nanoparticle of the present invention.

In some embodiments, the lipid composition of the invention is a lipid polyplex (LPP). In some embodiments, the lipid composition of the invention is a lipid polyplex (LPP) containing RNA.

In some embodiments, the lipid encapsulating the polynucleotide is selected from one or more of the following lipids: cationic lipids, phospholipids, steroids and/or polyethylene glycol modified lipids. In a preferred embodiment, the cationic lipid is an ionizable cationic lipid.

The lipid composition of the present invention can be used for administration through the mucosa and contains a therapeutic or prophylactic agent and a lipid encapsulating the therapeutic or prophylactic agent. The lipids encapsulating therapeutic or prophylactic agents include cationic lipids, phospholipids, steroids and polyethylene glycol modified lipids.

In one embodiment, the lipid composition of the invention comprises a cationic lipid, wherein the cationic lipid comprises DOTMA, DOTAP, DDAB, DOSPA, DODAC, DODAP, DC-Chol, DMRIE, DMOBA, DLinDMA, DLenDMA, CLinDMA, DMORIE, DLDMA, DMDMA, DOGS, N4-cholesteryl-spermine, DLin-KC2-DMA, DLin-MC3-DMA, compounds of formula (I), (II), (III) or (IV) as described herein or combination thereof. In a preferred embodiment, the cationic lipids include M5, MC3, ALC-0315, SM-102. In a preferred embodiment, the cationic lipid comprises SW-II-121, SW-II-138-1, SW-II-139-1, SW-II-140-1 or SW-II-140-2. In a preferred embodiment, the cationic lipids include M5, MC3, ALC-0315, SM-102, SW-II-121, SW-II-138-1, SW-II-139-1, SW-II-140-1 or SW-II-140-2.

In one embodiment, the lipid composition of the invention comprises phospholipids and/or steroids. In one embodiment, the lipid composition of the invention comprises a phospholipid as described herein, wherein the phospholipid comprises 1,2-dilinoleoyl-sn-glycero-3-phosphocholine (DLPC), 1,2-Dimyristoyl-sn-glycero-phosphocholine (DMPC), 1,2-dioleoyl-sn-glycero-3-phosphocholine (DOPC), 1,2-dipalmitoyl-sn-glycerol-3-Phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesteryl hemisuccinyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-bisdocosahexa Enoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0PE), 1,2-distearoyl-sn-glycero-3-phosphoethanolamine, 1,2-dilinoleoyl-sn-glycero-3-phosphoethanolamine, 1,2-dilinolenoyl-sn-glycerol-3-phosphoethanolamine, 1,2-diarachidonoyl-sn-glycerol-3-phosphoethanolanine, 1,2-bidocosahexaenoyl-sn-glycerol-3-phosphoethanolamine, 1,2-Dioleoyl-sn-glycerol-3-phosphate-rac-(1-glycerol) sodium salt (DOPG), dipalmitoylphosphatidylglycerol (DPPG), palmitoyloleoylphosphatidylethanolamine (POPE), Distearoyl-phosphatidyl-ethanolamine (DSPE), dipalmitoylphosphatidylethanolamine (DPPE), dimyristoylphosphatidyl-ethanolamine (DMPE), 1-stearoyl-2-oleoyl-stearoylethanolamine (SOPE)), 1-stearoyl-2-oleoyl-phosphatidylcholine (SOPC), sphingomyelin, phosphatidylcholine, phosphatidylethanolanine, phosphatidylserine, phosphatidylinositol, phosphatidic acid, palmitoyloleoyl phospholipid Acylcholine, lysophosphatidylcholine, lysophosphatidylethanolamine (LPE) or combinations thereof. In one embodiment, the lipid composition of the invention comprises a steroid as described herein, wherein the steroid comprises cholesterol, coprosterol, sitosterol, ergosterol, campesterol, stigmasterol, brassicasterol, tomatine, ursolic acid, α-tocopherol and its derivatives. In one embodiment, the lipid composition of the invention comprises a phospholipid and a steroid as described herein. In one embodiment, the lipid composition includes DOPE. In one embodiment, the lipid composition of the invention comprises DSPC. In one embodiment, the lipid composition of the invention includes cholesterol. In one embodiment, the lipid group of the present invention

The compound contains DOPE and cholesterol. In one embodiment, the lipid composition of the invention comprises DSPC and cholesterol.

In one embodiment, the lipid composition of the present invention includes cationic lipids M5, MC3, ALC-0315, SM-102, SW-II-121, SW-II-138-1, SW-II-139-1, SW-II-140-1 or SW-II-140-2, phospholipid DOPE and cholesterol. In one embodiment, the lipid composition of the present invention includes cationic lipids M5, MC3, ALC-0315, SM-102, SW-II-121, SW-II-138-1, SW-II-139-1, SW-II-140-1 or SW-II-140-2, phospholipid DSPC and cholesterol.

In some embodiments, the polynucleotide-encapsulating lipid further comprises a polyethylene glycol-modified lipid. In one embodiment, the polyethylene glycol modified lipid comprises DMG-PEG (eg, DMG-PEG 2000), DOG-PEG, DSPE-PEG, ALC-0159, or a combination thereof. In one embodiment, the polyethylene glycol modified lipid is ALC-0159. In one embodiment, the polyethylene glycol modified lipid is DSPE-PEG. In one embodiment, the polyethylene glycol modified lipid is DMG-PEG (eg, DMG-PEG 2000).

In one embodiment, the lipid composition of the present invention includes cationic lipids, DOPE, cholesterol and DSPE-PEG.

In one embodiment, the lipid composition of the invention comprises cationic lipids. DSPC, cholesterol and DSPE-PEG.

In one embodiment, the lipid composition of the present invention includes cationic lipids, DSPC, cholesterol and DMG-PEG.

In a preferred embodiment, the lipid composition of the present invention contains cationic lipids, DOPE, cholesterol and DMG-PEG.

In a preferred embodiment, the lipid composition of the present invention includes cationic lipids M5, MC3, ALC-0315, SM-102, SW-II-121, SW-II-138-1, SW-II-139-1, SW-II-140-1 or SW-II-140-2, DOPE, cholesterol and DMG-PEG.

In some embodiments, the lipid composition of the invention further comprises a cationic polymer associated with the polynucleotide as a complex and co-encapsulated in the lipid.

In one embodiment, the cationic polymer includes poly-L-lysine, protamine, polyethylenimine (PEI), or combinations thereof. In one embodiment, the cationic polymer is protamine. In one embodiment, the cationic polymer is polyethyleneimine.

In one embodiment, the amount of lipid in the lipid composition of the invention is calculated as mole percent (mol %), which is determined based on the total moles of all lipids in the lipid composition. Unless otherwise specified, the sum of the amounts (mol %) of each lipid in the composition is 100 mol %, that is, the sum of the amounts (mol %) of cationic lipids, phospholipids, steroids and polyethylene glycol modified lipids is 100 mol %.

In one embodiment, the amount of cationic lipids in the lipid composition of the invention is from about 30 to about 6) mole percent, based on the total amount of all lipids in the lipid composition. In some embodiments, the amount of cationic lipids in the lipid composition of the invention is about 35 to about 60 mol %, about 30 to about 50 mol %, about 35 to about 50 mol %, about 35 to about 45 mol % %, about 35 to about 42.5 mol %, about 37.5 to about 45 mol %, about 37.5% to about 42.5 mol %, about 40 to about 45 mol %, or about 40 to about 50 mol %. For example, the amount of cationic lipid may be about 30, 32.5, 35, 37.5, 40, 42.5, 45, 47.5, 50, 52.5, 55, 57.5, or 60 mole percent.

In one embodiment, the amount of phospholipids in the lipid composition of the invention is from about 5 to about 40 mole %, based on lipids.

The total amount of all lipids in the composition. In one embodiment, the amount of phospholipids in the lipid composition of the present invention is about 5 to about 35 mol %, about 5 to about 30 mol %, about 5 to about 25 mol %, about 5 to about 20 mol %, About 10 to about 35 mol %, about 10 to about 30 mol %, about 15 to about 35 mol %, about 20 to about 35 mol %, about 25 to about 35 mol %, about 10 to about 25 mol %, or about 15-about 25 mol %. For example, the amount of phospholipid may be about 5, 10, 15, 20, 25, 30, 35, or 40 mole percent.

In one embodiment, the amount of cholesterol in the lipid composition of the invention is from about 10 to about 70 mole percent, based on the total amount of all lipids in the lipid composition. In one embodiment, the amount of cholesterol in the lipid composition of the present invention is about 10 to about 65 mol %, about 10 to about 60 mol %, about 15 to about 50 mol %, about 15 to about 30 mol %, About 20 to about 60 mol %, about 30 to about 50 mol %, about 35 to about 40 mol %, about 35 to about 45 mol %, about 40 to about 45 mol %, or about 45 to about 50 mol %. For example, the amount of cholesterol may be about 10, 15, 17.5, 18.75, 20, 22.5, 25, 27.5, 28.75, 30, 32.5, 33.75, 35, 40, 42.5, 45, 46.25, 47.5, 48.75, 50, 52.5, 53.75, 55, 60, 62.5, 63.75, 65 or 70 mol %.

In one embodiment, the amount of polyethylene glycol-modified lipids in the lipid composition of the invention is from about 2.5 to about 20 mole %, based on the total amount of all lipids in the lipid composition. In one embodiment, the amount of polyethylene glycol-modified lipids in the lipid composition of the present invention is about 2.5 to about 10 mol %, about 3 to about 10 mol %, about 3.5 to about 10 mol %, about 3.75-about 10 mol %, about 3.75-about 7.5 mol %, about 3.75-about 5 mol %, about 4-about 10 mol %, about 5-about 10 mol %, about 7.5-about 10 mol %, about 5-About 7.5 mol %, about 10 to about 20 mol %, about 10 to about 15 mol %, or about 15 to about 20 mol %. For example, the amount of polyethylene glycol modified lipid can be about 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 15 or 20 mol %.

In one embodiment, the lipid composition of the present invention contains 30-60 mol % cationic lipids, 5-40 mol % phospholipids, 10-70 mol % steroids and 2.5-20 mol % polyethylene glycol Modified lipids. In a preferred embodiment, the lipid composition of the present invention contains 30-60 mol % cationic lipids, 5-40 mol % phospholipids, 10-60 mol % steroids and 2.5-20 mol % polyethylene glycol. Alcohol-modified lipids. In a preferred embodiment, the lipid composition of the present invention contains 35-50 mol % cationic lipids, 10-35 mol % phospholipids, 15-50 mol % steroids and 2.5-20 mol % polyethylene glycol. Alcohol-modified lipids. In a preferred embodiment, the lipid composition of the present invention contains 37.5-42.5 mol % cationic lipids, 25-35 mol % phospholipids, 15-30 mol % steroids and 2.5-20 mol % polyethylene glycol. Alcohol-modified lipids. In a preferred embodiment, the lipid composition of the present invention contains 37.5-42.5 mol % cationic lipids, 25-35 mol % phospholipids, 15-30 mol % steroids and 2.5-10 mol % polyethylene glycol. Alcohol-modified lipids. In a preferred embodiment, the lipid composition of the present invention contains 37.5-42.5 mol % cationic lipids, 25-35 mol % phospholipids, 15-30 mol % steroids and 3.75-10 mol % polyethylene glycol. Alcohol-modified lipids. In a preferred embodiment, the lipid composition of the present invention contains 37.5-42.5 mol % cationic lipids, 25-35 mol % phospholipids, 15-30 mol % steroids and 3.75-7.5 mol % polyethylene glycol. Alcohol-modified lipids. In a preferred embodiment, the lipid composition of the present invention contains 37.5-42.5 mol % cationic lipids, 25-35 mol % phospholipids, 15-30 mol % steroids and 3.75-5 mol % polyethylene glycol. Alcohol-modified lipids.

For the application purposes of the lipid composition of the present invention, the above-mentioned contents and their ranges are advantageous.

In one embodiment, the LPP comprises a therapeutic or prophylactic agent and a lipid encapsulating the therapeutic or prophylactic agent, wherein the lipid encapsulating the therapeutic or prophylactic agent comprises a cationic lipid, a phospholipid, a steroid, and a polyethylene glycol, alcohol-modified lipids:

The LPP also includes a cationic polymer, wherein the cationic polymer is associated with the therapeutic or prophylactic agent as a complex. In one embodiment, the LPP contains 2.5-20 mole % polyethylene glycol modified lipids, based on the total amount of all lipids in the lipid composition.

In one embodiment, the lipid composition of the present invention includes a therapeutic or preventive agent and a lipid encapsulating the therapeutic or preventive agent, wherein the lipid encapsulating the therapeutic or preventive agent comprises a cationic lipid, phospholipids, steroids and polyethylene glycol modified lipids; the lipid composition further includes a cationic polymer, wherein the cationic polymer is associated with the therapeutic or preventive agent into a complex, and is co-encapsulated in Lipid multimeric complexes are formed in the lipids. In one embodiment, the lipid composition contains 2.5-20 mole % of polyethylene glycol modified lipids, based on the total amount of all lipids in the lipid composition.

In one embodiment, the phospholipid is selected from 1,2-dioleoyl-sn-glycero-3-phosphoethanolamine (DOPE), distearoylphosphatidylcholine (DSPC), or a combination thereof. In one embodiment, the steroid is cholesterol. In one embodiment, the cationic polymer is protamine. In one embodiment, the polyethylene glycol-modified lipid is selected from the group consisting of 2-[(polyethylene glycol)-2000]—N,N-tetracosane (ALC-0159), 1,2-ditetracosane Myristoyl-rac-glycerol-3-methoxypolyethylene glycol (DMG-PEG), 1,2-distearoyl-sn-glycerol-3-phosphoethanolamine-poly(ethylene glycol) (DSPE-PEG) or combinations thereof. In one embodiment, the cationic lipid is selected from M5, MC3, ALC-0315, SM-102, SW-II-121, SW-II-138-1, SW-II-139-1, SW-II-140-1 or SW-II-140-2.

In a preferred embodiment, the lipid composition contains 30-60 mol % cationic lipids, 5-40 mol % DOPE, 10-70 mol % cholesterol and 2.5-20 mol % DMG-PEG.

In a preferred embodiment, the lipid composition contains 30-60 mol % cationic lipids, 5-40 mol % DOPE, 10-60 mol % cholesterol and 2.5-20 mol % DMG-PEG.

In a preferred embodiment, the lipid composition contains 35-50 mol % cationic lipids, 10-35 mol % DOPE, 15-50 mol % cholesterol and 2.5-20 mol % DMG-PEG.

In a preferred embodiment, the lipid composition contains 37.5-42.5 mol % cationic lipids, 25-35 mol % DOPE, 15-30 mol % cholesterol and 2.5-20 mol % DMG-PEG.

In a preferred embodiment, the lipid composition contains 37.5-42.5 mol % cationic lipids, 25-35 mol % DOPE, 15-30 mol % cholesterol, and 2.5-10 mol % DMG-PEG.

In a preferred embodiment, the lipid composition contains 37.5-42.5 mol % cationic lipids, 25-35 mol % DOPE, 15-30 mol % cholesterol, and 3.75-10 mol % DMG-PEG.

In a preferred embodiment, the lipid composition contains 37.5-42.5 mol % cationic lipids, 25-35 mol % DOPE, 15-30 mol % cholesterol and 3.75-5 mol % DMG-PEG.

In a preferred embodiment, the lipid composition contains 42.5 mol % cationic lipids, 35 mol % DOPE, 18.75 mol % cholesterol, and 3.75 mol % DMG-PEG.

In a particularly preferred embodiment, the lipids of the encapsulated complex comprise 42.5 mol % of M5, MC3, ALC-0315, SM-102, SW-II-121, SW-II-138-1, SW-II-139-1, SW-II-140-1 or SW-II-140-2, 35 mol % DOPE, 18.75 mol % cholesterol and 3.75 mol % DMG-PEG.

### Cationic Lipids

Cationic lipids are lipids that can carry a net positive charge at a specified pH. Lipids with a net positive charge can associate with nucleic acids through electrostatic interactions.

Examples of cationic lipids include, but are not limited to, 1,2-di-O-octadecenyl-3-trimethylammonium propane (1,2-di-O-octadecenyl-3-trimethylammonium-propane, DOTMA), 1,2-dioleoyl-3-trimethylammonium-propane (1,2-dioleoyl-3-trimethylammonium-propane, DOTAP), disdecyldimethyl Didecyldimethylammonium bromide (DDAB), 2,3-dioleoyloxy-N-[2 (sperminecarboxamide)ethyl]-N,N-dimethyl-1-propylamine trifluoroacetic acid Salt (2,3-dioleoyloxy-N-[2 (spermine carboxamide)ethyl]-N,N-dimethyl-1-propanamium trifluoroacetate, DOSPA), dioctadecyldimethyl ammonium chloride (DODAC)), 1,2-dioleoyl-3-dimethylammonium-propane (DODAP), 3-(N—(N′,N′-dimethylamino Ethane)-carbamoyl)cholesterol (3-(N—(N′,N′-dimethylaminoethane)-carbamoyl)cholesterol, DC-Chol), 2,3-di(tetradecyloxy)propyl-(2-hydroxyethyl)-dimethylazanium (2,3-di(tetradecoxy)propyl-(2-hydroxyethyl)-dimethylazanium, DMRIE), N,N-dimethyl-3,4-dioleyl Oxybenzylamine (N,N-dimethyl-3,4-dioleyloxybenzylamine, DMOBA), 1,2-dilinoleyloxy-N,N-dimethylaminopropane (1,2-dilinoleyloxy-N,N-dimethylaminopropane, DLinDMA), 1,2-dilinoleyloxy-N,N-dimethylaminopropane (1,2-dilinolenyloxy-N,N-dimethylaminopropane, DLenDMA), 3-dimethylamino-2-(cholest-5-ene-3-p-oxybutane-4-oxy)-1-(cis,cis-9,12-octadecadienyloxy)propane(3-dimethylamino-2-(cholest-5-en-3-beta-oxybutan-4-oxy)-1-(cis,cis-9,12-oc-tadecadienoxy)propane, CLinDMA), N-(2-aminoethyl N-(2-aminoethyl)-N,N-dimethyl-2,3-bis(tetradecyloxy)propane-1-amine bromide-bis(tetradecyloxy)propan-1-aminium bromide, DMORIE), N,N-dimethyl-2,3-bis(dodecyloxy)propan-1-amine (N,N-dimethyl-2, 3-bis(dodecyloxy)propan-1-amine, DLDMA), N,N-dimethyl-2,3-bis(tetradecyloxy)propan-1-amine (N,N-dimethyl-2, 3-bis (tetradecyloxy)propan-1-amine (DMDMA), dioctadecylamidoglycyl spermine (DOGS), N4-cholesteryl-spermine (N4-cholesteryl-spermine), 2,2-dilinoleyl-4-(2-dimethylaminoethyl)-[1,3]-dioxolane(2,2-dilinoleyl-4-(2-dimethylaminoethyl)-[1,3]-dioxolane, DLin-KC2-DMA), heptatriaconta-6,9,28,31-tetraen-19-yl-4-(dimethylamino)butyrate (heptatriaconta-6,9,28,31-tetraen-19-yl-4-(dimethylamino)butanoate, DLin-MC3-DMA), a compound of formula (I), (II), (III) or (IV) as described herein, or a combination thereof.

In some embodiments, the cationic lipid is preferably an ionizable cationic lipid. Ionizable cationic lipids have a net positive charge at, for example, acidic pH and are neutral at higher pH (eg, physiological pH). Examples of ionizable cationic lipids include, but are not limited to: dioctadecylamidoglycyl spermine (DOGS), N4-cholesteryl-spermine, 2,2-Dilinoleyl-4-(2-dimethylaminoethyl)-[1,3]-dioxolane(2,2-dilinoleyl-4-(2-dimethylaminoethyl)-[1,3]-dioxolane, DLin-KC2-DMA), heptatriaconta-6,9,28,31-tetraen-19-yl-4-(dimethylamino)butyrate (heptatriaconta-6,9,28,31-tetraen-19-yl-4-(dimethylamino)butanoate, DLin-MC3-DMA), a compound of formula (I), (II), (III) or (IV) as described herein, or a combination thereof in one embodiment, the cationic lipid comprises a compound of formula (I) or a pharmaceutically acceptable salt thereof:

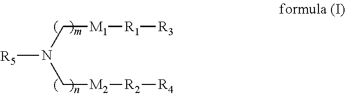

wherein,
R₁ and R₂ each independently selected from key, C₁-C₁₂ Alkyl and C₂-C₁₂ Alkenyl;
R₃ and R₄ each is independently selected from C₁-C₁₂ Alkyl, C₂-C₁₂ Alkenyl, C₆-C₁₀ Aryl and 5-10 membered heteroaryl; and R₃ and R₄ each independently selects t R₆ Instead, t is an integer selected from 1-5;
R₆ each is independently selected from C₁-C₁₂ Alkyl and C₂-C₁₂ Alkenyl;
M₁ and M₂ each independently selected from bond, H, —O—, —S—, —C(O)—, —OC(O)—, —C(O)O—, —OC(O)O—, —SC(S)—, —C(S)S—, 3-10 membered heterocycle. —NR₇—, or
R₅ with M₁ and M₂ One of them together with the connected N atom forms a 3-10 membered heterocyclic ring, and the corresponding R₁/R₃ or
R₂/R₄ is not present, the heterocycle is optionally replaced by R₇ replace:
R₅ Selected from C₃₋₈ Carbocyclic ring, —C₁₋₁₂ Alkylene-Q, Q is selected from H, —OR₇, —SR₇, —OC(O)R₇, —C(O)OR₇, —N(R₇)C(O)R₇, —N(R₇)S(O)₂R₇, —N(R₇)C(S)R₇, —N(R₇)₂, cyano group, C₃₋₈ Carbocyclic ring, 3-10 membered heterocyclic ring, C₆-C₁₀ Aryl, each of the above groups is optionally substituted by one or more C₁-C₁₂ Alkyl, C₂-C₁₂ Alkenyl, C₁-C₁₂ Alkoxy, C₆-C₁₀ to Aryl, 5-10 membered heteroaryl, 3-10 membered heterocycle, halogen, hydroxyl, oxo (═O) substitution;
m and n are each independently an integer selected from 0-12;
The alkyl, alkenyl and alkylene groups are each optionally independently interrupted by one or more groups selected from: —O—, —S—, —NR₇—, —C(O)—, —OC(O)—, —C(O)O—, —SC(S)—, —C(S)S—, C₃₋₈ carbocyclic ring, and each of the alkyl, alkenyl and alkylene groups is optionally replaced by one or more R₇ replace;
R₇ each is independently selected from H, C₁-C₁₂ Alkyl, C₂-C₁₂ Alkenyl, C₁-C₁₂ Alkoxy, carboxylic acid, sulfinic acid, sulfonic acid, sulfonyl, nitro, cyano, amino, carbamoyl, sulfonamide, C₆-C₁₀ Aryl, 5-10 membered heteroaryl, 3-10 membered heterocycle, halogen, C₃₋₈ Carbocyclic ring, each of the above groups is optionally replaced by one or more C₁-C₁₂ Alkyl, C₂-C₁₂ Alkenyl, C₁-C₁₂ Alkoxy, C₆-C₁₀ Aryl, 5-10 membered heteroaryl, 3-10 membered heterocycle, halogen, hydroxyl, oxo (═O) substitution.

In one embodiment, R₁ and R₂ each is independently selected from C₁-C₁₂ Alkyl and C₂-C₁₂ Alkenyl, such as C₁-C₁₂ alkyl. In yet another embodiment, R₁ and R₂ One is a key and the other is independently selected from C₁-C₁₂ Alkyl and C₂-C₁₂ Alkenyl, such as C₁-C₁₂ alkyl.

In one embodiment, R₃ and R₄ each is independently selected from C₁-C₁₂ Alkyl, C₂-C₁₂ Alkenyl, C₆-C₁₀ to Aryl and 5-10 membered heteroaryl. In yet another embodiment, R₃ and R₄ each is independently selected from C₁-C₁₂ Alkyl and C₂-C₁₂ alkenyl.

R₃ and R₄ Can be independently selected by t R₆ Instead, t is 1, 2, 3, 4, 5. In one embodiment, R₆ each is independently selected from C₁-C₁₂ alkyl.

In yet another embodiment, R₃ and R₄ At least one of them is C₆-C₁₀ to Aryl or 5-10 membered heteroaryl, such as C₆-C₁₀ to Aryl.

In one embodiment, R₅ Selected from C₃₋₈ Carbocyclic ring, —C₁₋₁₂ Alkylene-Q. Q can be selected from H, —OR₇, —SR₇, —OC(O)R₇, —C(O)OR₇, —N(R₇)C(O)R₇, —N(R₇)S(O)₂R₇, —N(R₇)C(S)R₇, —N(R₇)₂, cyano group, C₃₋₈ Carbocyclic ring, 3-10 membered heterocyclic ring, C₆-C₁₀ Aryl. The above groups, including groups covering options for Q, where appropriate, may each optionally be replaced by one or more C₁-C₁₂ Alkyl, C₂-C₁₂ Alkenyl, C₁-C₁₂ Alkoxy, C₆-C₁₀ to Aryl, 5-10 membered heteroaryl, 3-10 membered heterocycle, halogen, hydroxyl, oxo (═O) substitution.

In yet another embodiment, R₅ Selected from C₃₋₈ Carbocyclic ring, —C₁₋₁₂ Alkylene-Q, Q is selected from H, —OR₇, —SR₇, —OC(O)R₇, —C(O)OR₇, —N(R₇)C(O)R₇, —N(R₇)S(O)₂R₇, —N(R₇)C(S)R₇, —N(R₇)₂, cyano group, C₃₋₈ Carbocyclic ring, 3-10 membered heterocyclic ring, C₆-C₁₀ Aryl. The above groups, including groups encompassing the options of Q, may each be optionally replaced by one or more C, where appropriate, 1-C₁₂ Alkyl, C₂-C₁₂ Alkenyl, C₁-C₁₂ Alkoxy, C₆-C₁₀ Aryl, 5-10 membered heteroaryl, 3-10 membered heterocycle, halogen, hydroxyl, oxo (═O) substitution.

In compounds of formula (I), R₇ Can be selected independently from H, C₁-C₁₂ Alkyl, C₂-C₁₂ Alkenyl, C₁-C₁₂ Alkoxy, carboxylic acid, sulfinic acid, sulfonic acid, sulfonyl, nitro, cyano, amino, carbamoyl, sulfonamide, C₆-C₁₀ Aryl, 5-10 membered heteroaryl, 3-10 membered heterocycle, halogen, C₃₋₈ Carbocyclic ring, preferably selected from H, C₁-C₁₂ Alkyl, C₂-C₁₂ Alkenyl, C₁-C₁₂ Alkoxy, carboxylic acid, sulfinic acid, sulfonic acid, sulfonyl, nitro, cyano, amino, carbamoyl, sulfonamide. C₆-C₁₀ Aryl and 5-10 membered heteroaryl. The above groups (when appropriate, e.g. H, C₁-C₁₂ Alkyl, C₂-C₁₂ Alkenyl, C₁-C₁₂ Alkoxy, carboxylic acid, sulfinic acid, sulfonic acid, sulfonyl, amino, carbamoyl, sulfonamide, C₆-C₁₀ Aryl, 5-10 membered heteroaryl, 3-10 membered heterocycle, C₃₋₈ carbocyclic rings) are each optionally replaced by one or more C₁-C₁₂ Alkyl, C₂-C₁₂ Alkenyl, C₁-C₁₂ Alkoxy, C₆-C₁₀ to Aryl, 5-10 membered heteroaryl, 3-10 membered heterocycle, halogen, hydroxyl, oxo (═O) substitution.

In one embodiment, each group in the above description, such as C₃₋₈ Carbocyclic ring, —C₁₋₁₂ Alkylene -Q, including —OR covering Q options 7, —SR₇, —OC(O)R₇, —C(O)OR₇, —N(R₇)C(O)R₇, —N(R₇)S(O)₂R₇, —N(R₇)C(S)R₇, —N(R₇)₂, C₃₋₈ Carbocyclic ring, 3-10 membered heterocyclic ring, C₆-C₁₀ Aryl, C₁-C₁₂ Alkyl, C₂-C₁₂ Alkenyl, C₁-C₁₂ Alkoxy, carboxylic acid, sulfinic acid, sulfonic acid, sulfonyl, amino, carbamoyl, sulfonamide, C₆-C₁₀ to Aryl, 5-10 membered heteroaryl, 3-10 membered heterocycle, halogen. C₃₋₈ Carbocycles, etc. may each be optionally replaced by one or more C₁-C₁₂ Alkyl, C₂-C₁₂ Alkenyl, C₁-C₁₂ Alkoxy, C₆-C₁₀ Aryl, 5-10 membered heteroaryl, 3-10 membered heterocycle, halogen, hydroxyl, oxo (═O) substitution.

In one embodiment, in the compound of formula (I) (e.g. R₁-R₇ The alkyl, alkenyl and alkylene groups mentioned in) may each optionally be independently interrupted by one or more groups selected from: —O—, —S—, —NR₇—, —C(O)—, —OC(O)—, —C(O)O—, —SC(S)—, —C(S)S—, C₃₋₈ carbocyclic ring, and each of the alkyl, alkenyl and alkylene groups is optionally replaced by one or more R₇ replace. That is, the chains (linear or branched) of the alkyl, alkenyl and alkylene groups may each optionally contain one or more groups selected from the following: —O—, —S—, —NR₇—, —C(O)—, —OC(O)—, —C(O)O—, —SC(S)—, —C(S)S—, C₃₋₈ Carbon ring.

R₇ each is independently selected from H, C₁-C₁₂ Alkyl, C₂-C₁₂ Alkenyl, C₁-C₁₂ Alkoxy, carboxylic acid, sulfinic acid, sulfonic acid, sulfonyl, nitro, cyano, amino, carbamoyl, sulfonamide, C₆-C₁₀ to Aryl, 5-10 membered heteroaryl, 3-10 membered heterocycle, halogen, C₃₋₈ Carbocycle; preferably, R₇ Independently selected from H, C₁-C₁₂ Alkyl, C₂-C₁₂ Alkenyl, C₁-C₁₂ Alkoxy, carboxylic acid, sulfinic acid, sulfonic acid, sulfonyl, nitro, cyano, amino, carbamoyl, sulfonamide, C₆-C₁₀ Aryl and 5-10 membered heteroaryl. The above groups (when appropriate, e.g. H, C₁-C₁₂ Alkyl, C₂-C₁₂ Alkenyl, C₁-C₁₂ Alkoxy, carboxylic acid, sulfinic acid, sulfonic acid, sulfonyl, amino, carbamoyl, sulfonamide, C₆-C₁₀ Aryl, 5-10 membered heteroaryl, 3-10 membered heterocycle, C₃-8 carbocyclic rings) are each optionally replaced by one or more C₁-C₁₂ Alkyl, C₂-C₁₂ Alkenyl, C₁-C₁₂ Alkoxy, C₆-C₁₀ Aryl, 5-10 membered heteroaryl, 3-10 membered heterocycle, halogen, hydroxyl, oxo (═O) substitution.

In the compound of formula (I), m and n can each be independently an integer selected from 0-12, for example, 0, 1, 2, 3, 4, 5, 6, 7, 8, 9, 10, 11, 12. When taken as 0, it means that the corresponding group does not exist.

In one embodiment, M₁ or M₂ is a key, the corresponding m or n is not 0, M₁ or M₂ The previous carbon chain corresponds to

R₁ or R₂ connect.

In one embodiment, m or n is 0, corresponding to M₁ or M₂Not a bond, N atom is directly connected to M₁ or M₂ connected.

In one embodiment M₁ or M₂ is a bond, the corresponding m or n is 0, and the N atom is directly connected to the corresponding R₁ or R₂ connected.

In one embodiment, M₁ and M₂ each is independently selected from —C(O)—, —OC(O)—, and —C(O)O—. In yet another embodiment, M₁ and M₂ each independently selected from —NR₇—, R₇ As mentioned above.

In another embodiment, R₅ with M₁ and M₂ One of them together with the connected N atom forms a 3-10 membered heterocyclic ring, and the corresponding R₁/R₃ or R₂/R₄ is not present, the heterocycle is optionally replaced by R₇ Replacement, R₇ As mentioned above.

In one embodiment, R₅ selected from —C₁₋₁₂ Alkylene-Q, Q is selected from H, —OR₇, —OC(O)R₇, —C(O)OR₇, —N(R₇)C(O)R₇, —N(R₇)₂, cyano group, R₇ As mentioned above.

In a preferred embodiment, R₁ and R₂ each is independently selected from C₁-C₁₂ Alkyl and C₂-C₁₂ Alkenyl;
where R₃ and R₄ each is independently selected from C₁-C₁₂ Alkyl and C₂-C₁₂ Alkenyl; and R₃ and R₄ each independently selects t R₆ Replace, t is an integer selected from 1-5; R₆ each is independently selected from C₁-C₁₂ Alkyl and C₂-C₁₂ alkenyl.

M₁ and M₂ each is independently selected from —OC(O)—, —C(O)O—, —OC(O)O—, —SC(S)— and —C(S)S—;
R₅ selected from —C₁₋₁₂ Alkylene-Q, Q is selected from —OR₇ and —SR₇R₇ Independently selected from H, C₁-C₁₂ Alkyl, C₂-C₁₂ Alkenyl, C₁-C₁₂ Alkoxy, carboxylic acid, sulfinic acid, sulfonic acid, sulfonyl, nitro, cyano, amino, carbamoyl, sulfonamide, C₆-C₁₀ Aryl and 5-10 membered heteroaryl:
m and n are each independently an integer selected from 1-12.
   In a preferred embodiment, the cationic lipid comprises a lipid compound having the structure shown below or a pharmaceutically acceptable salt thereof:

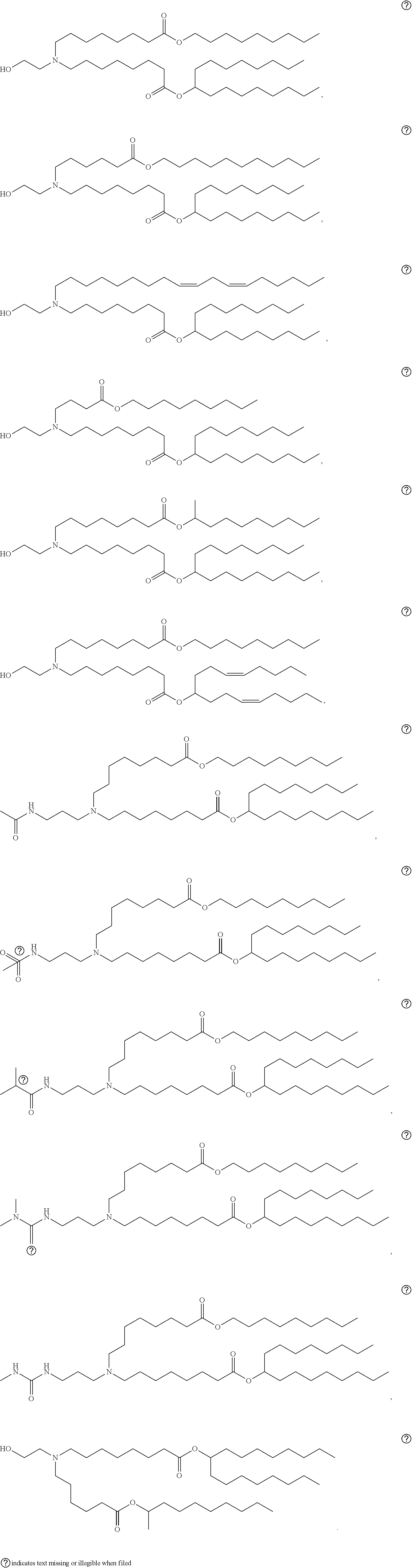

In a preferred embodiment, the cationic lipid contains M5 or SM-102.

In a preferred embodiment, the cationic lipid comprises a lipid compound having the structure shown below or a pharmaceutically acceptable salt thereof:

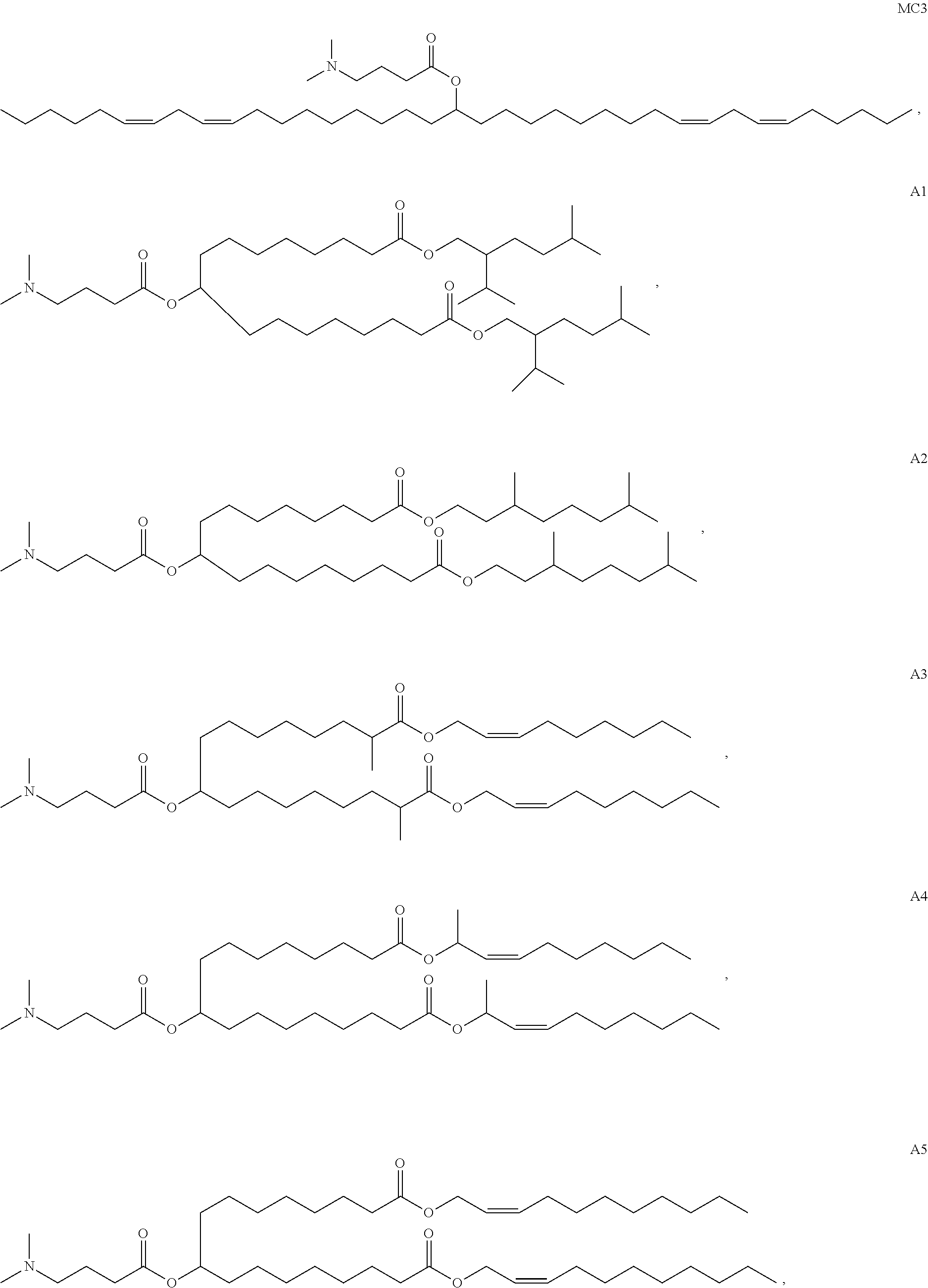

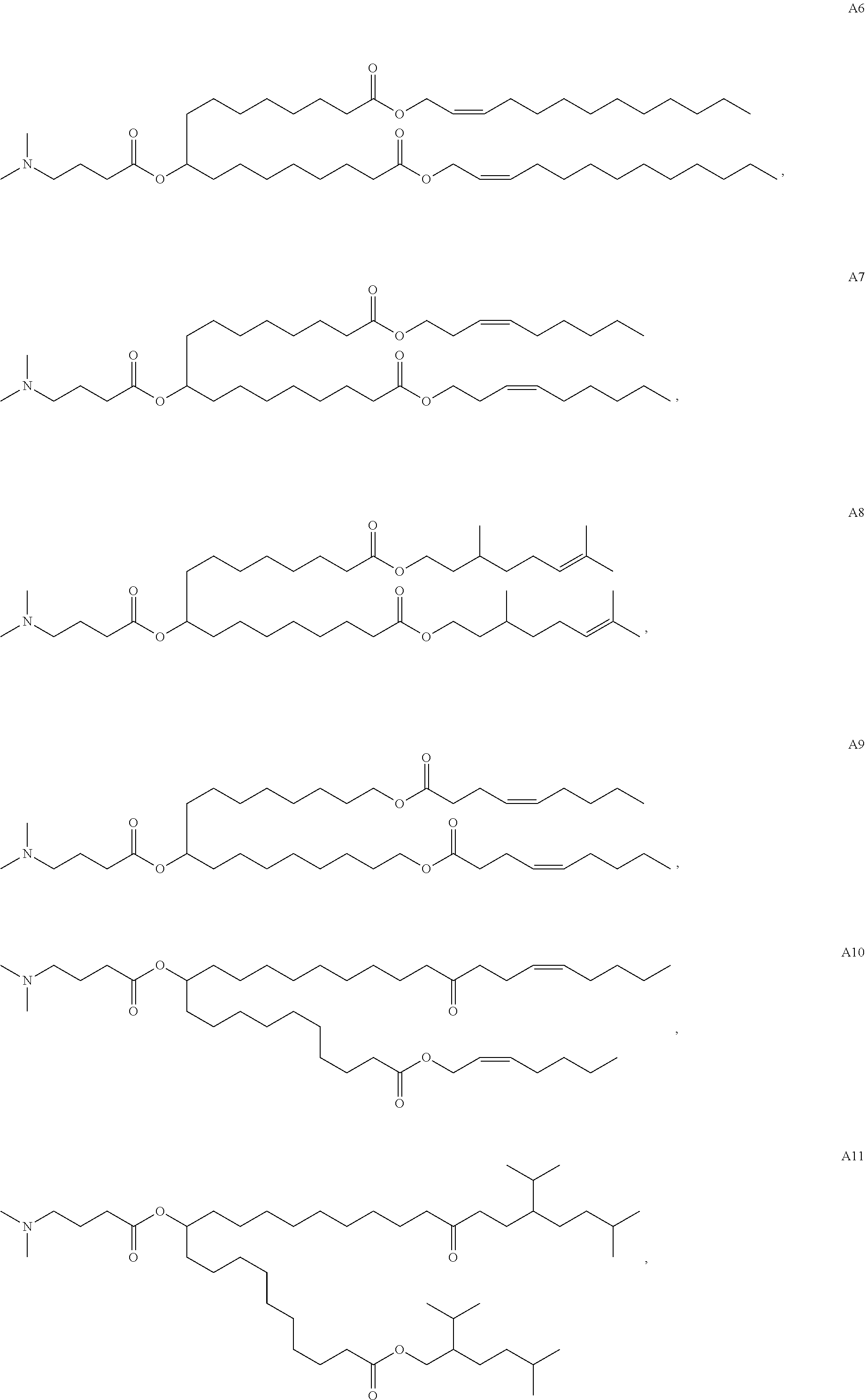

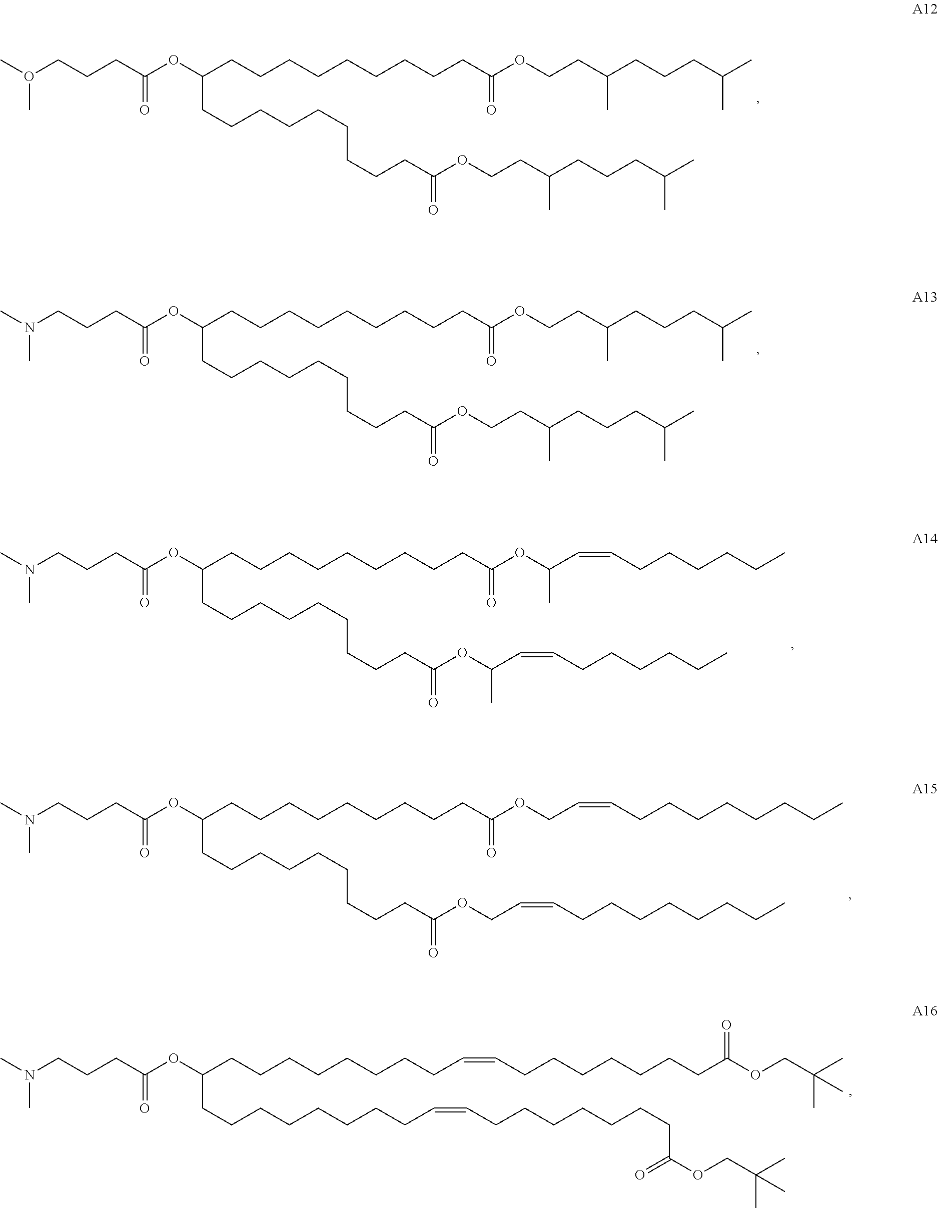

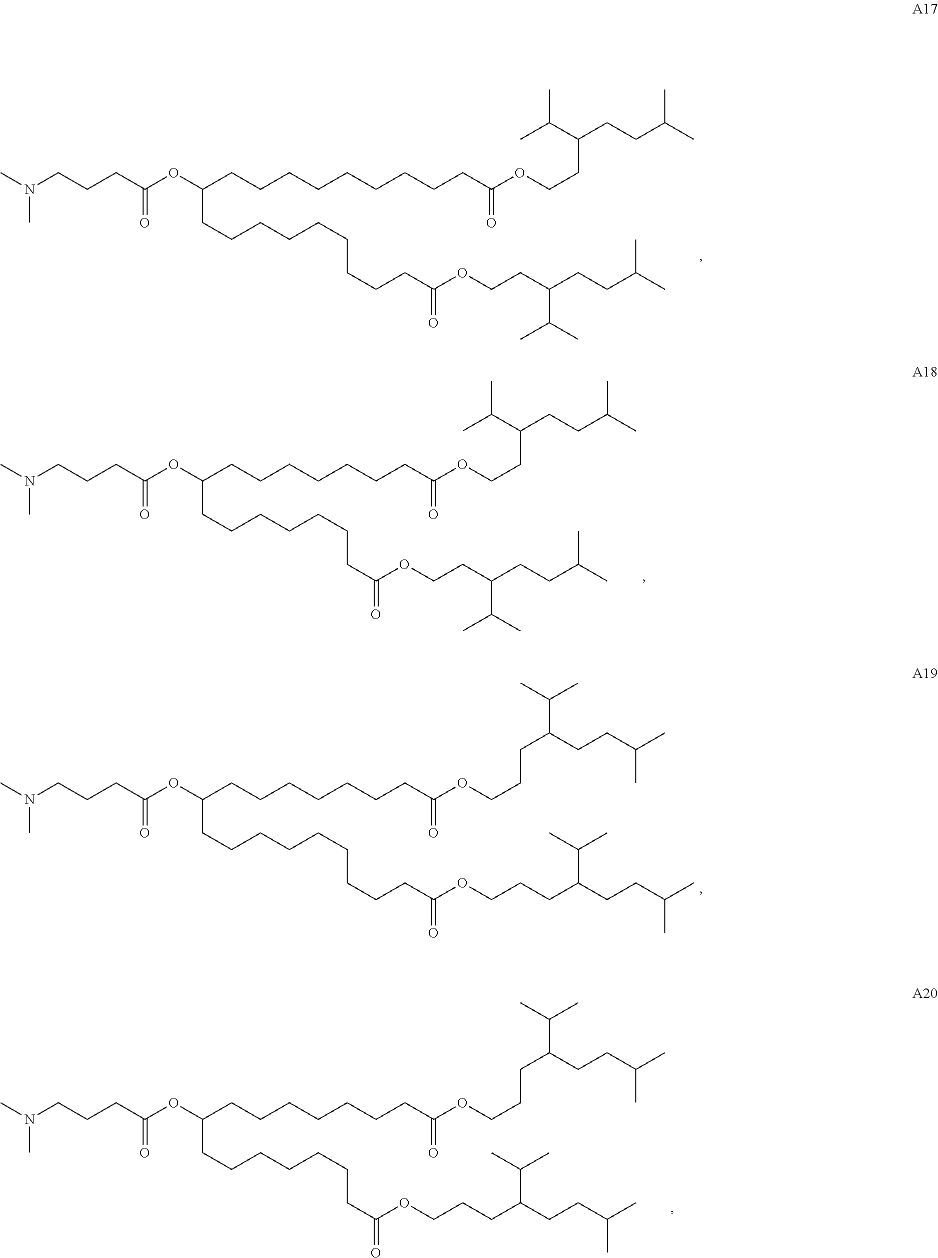

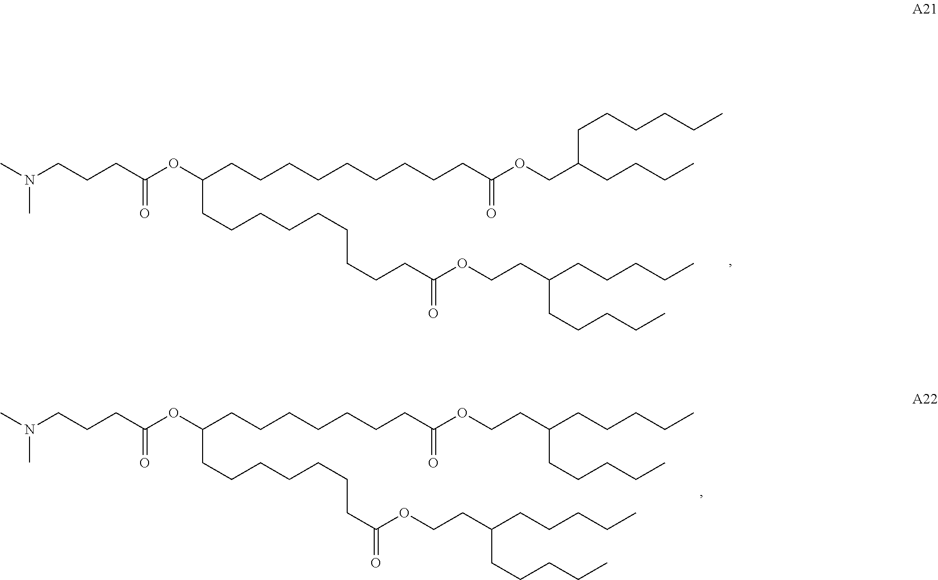

In a preferred embodiment, the cationic lipid comprises MC3.

In a preferred embodiment, cationic lipid comprises a lipid compound having the structure shown below or a pharmaceutically acceptable salt thereof:

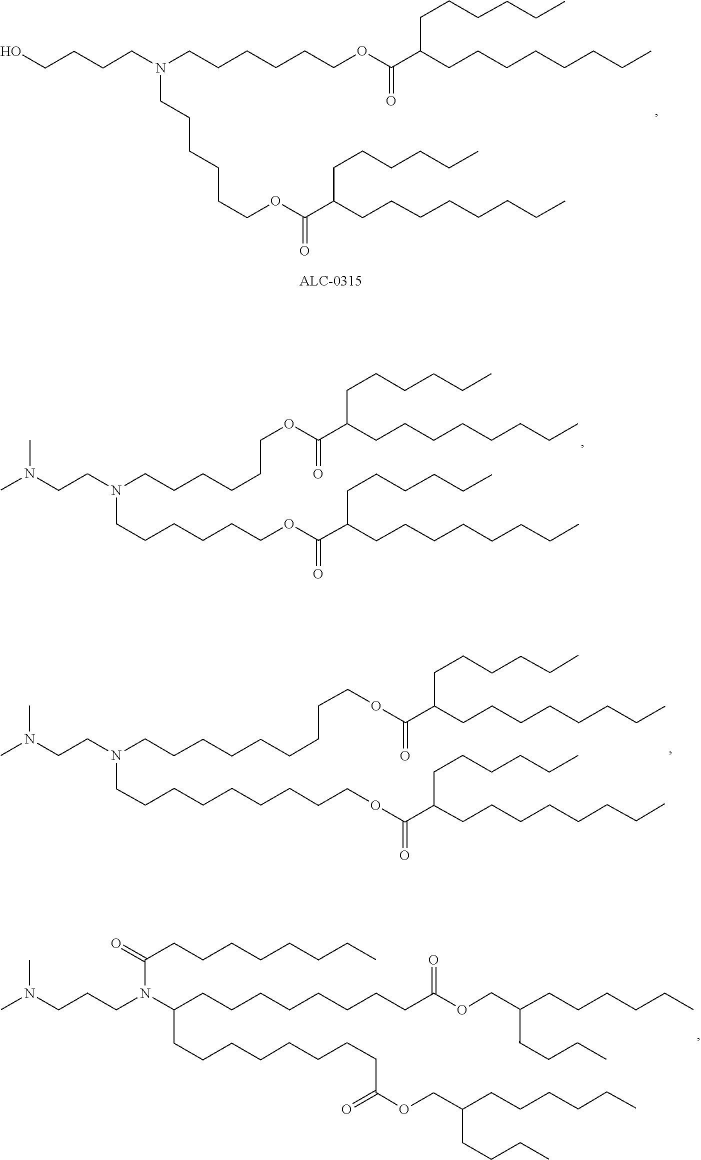

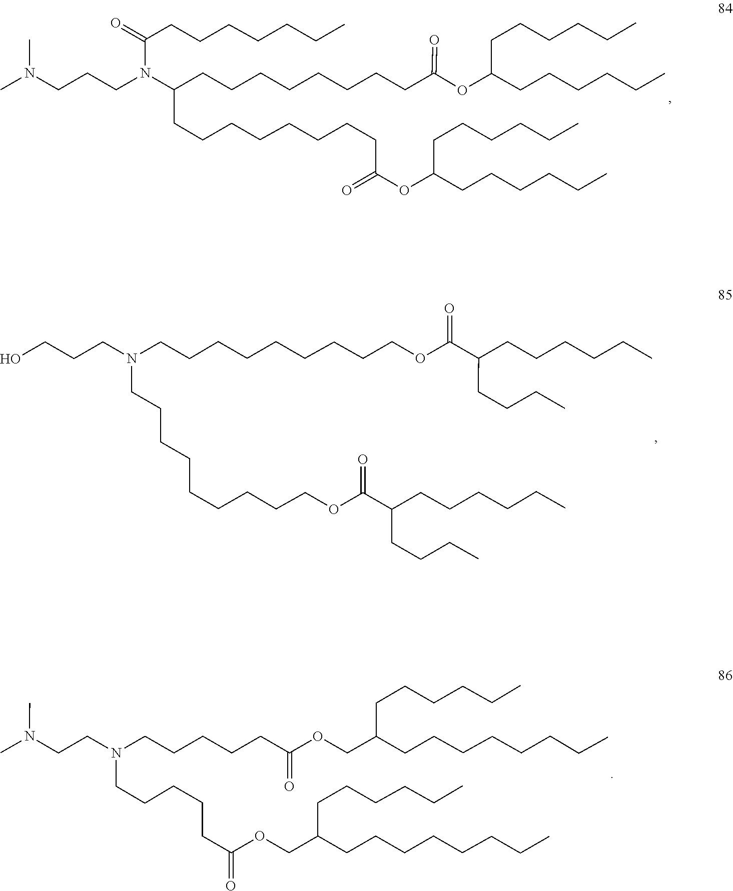

In a preferred embodiment, the cationic lipid contains ALC-0315.

In one embodiment, the cationic lipid comprises a compound of formula (I) or a pharmaceutically acceptable salt thereof:

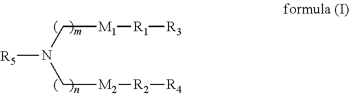

wherein
R₁ and R₂ each is independently selected from C₁-C₁₂ Alkyl and C₂-C₁₂ Alkenyl;
R₃ and R₄ each is independently selected from C₁-C₁₂ Alkyl, C₂-C₁₂ Alkenyl, C₆-C₁₀ Aryl and 5-10 membered heteroaryl;

The condition is R₃ and R₄ At least one of them is C₆-C₁₀ Aryl or 5-10 membered heteroaryl, and R₃ and R₄ each independently selects t R₆ Replace, t is an integer selected from 1-5; R₆ each is independently selected from C₁-C₁₂ Alkyl and C₂-C₁₂ Alkenyl;
M₁ and M₂ each is independently selected from —OC(O)—, —C(O)O—, —OC(O)O—. —SC(S)— and —C(S)S—;
R₅ selected from —C₁₋₁₂ Alkylene-Q, Q is selected from —OR₇ and —SR₇, R₇ Independently selected from H, C₁-C₁₂ Alkyl, C₂-C₁₂ ALKENYL, C₁-C₁₂ Alkyl, carboxylic acid, sulfinic acid, sulfonic acid, sulfonyl, nitro, cyano, amino, carbamoyl, sulfonamide. C₆-C₁₀ Aryl and 5-10 membered heteroaryl;
m and n are each independently an integer selected from 1-12.

In one embodiment, R₂ Selected from C₁-C₁₂ alkyl. In another embodiment, R₂ Selected from C₁-C₆ alkyl.

In one embodiment, R₃ and R₄ One is C₆-C₁₀ Aryl or 5-10 membered heteroaryl, the other is C₁-C₁₂ Alkyl or C₂-C₁₂ alkenyl.

In a specific embodiment, R₃ and R₄ each is independently selected from C₁-C₁₂ Alkyl and phenyl, provided R₃ and R₄ At least one of them is phenyl. In another embodiment, R₃ and R₄ One is phenyl and the other is C₁-C₁₂ alkyl.

In yet another embodiment. R₃ and R₄ each independently by t R₆ Instead, t is an integer selected from 1-5; for example, 1, 2, 3, 4 or 5. Preferably, t is an integer from 1 to 3, such as 1, 2 or 3, especially 1 or 2.

In one embodiment, R₆ each is independently selected from C₁-C₁₂ Alkyl, such as C₁-C₁₀ alkyl.

In one embodiment, t is 1 and R₆ Substituted on the benzene ring relative to R₁ or R₂ of metaposition or counterposition.

In another embodiment, t is 2. R₆ Substituted on the benzene ring relative to R₁ or R₂ Metaposition and counterposition.

In one embodiment, R₄ Replaced by R₂ The 1st or last digit. The 1 bit refers to R₂ Medium and M₂ The position of the directly connected C atom. The last bit refers to R₂ Medium and M₂ The position of the farthest C atom. In a specific embodiment, R₄ Selected from C₁-C₁₂ Alkyl, R₃ is phenyl.

In one embodiment, R₃ Replaced by R₁ The 1st or last digit. The 1 bit refers to R₁ Medium and M The position of the directly connected C atom. The last bit refers to R₁ Medium and M₁ The position of the farthest C atom. In a specific embodiment, R₃ Selected from C₁-C₁₂ Alkyl, R₄ is phenyl.

In one embodiment, M₁ and M₂ each is independently selected from —OC(O)—, —C(O)O— and —OC(O)O—.

In one embodiment, R₅ selected from —C₁₋₅ Alkylene-Q, e.g. C₁, C₂, C₃, C₄ or C₅ Alkylene-Q. In an exemplary embodiment. R₅ selected from —C₁₋₃ Alkylene-Q, e.g. C₁, C₂ or C₃ Alkylene-Q.

In another embodiment, Q is selected from —OH and —SH, especially —OH.

In some embodiments, m and n are each independently an integer selected from 2-9, such as 2, 3, 4, 5, 6, 7, 8, or 9. Preferably, m and n are each independently an integer selected from 2-7, such as 2, 3, 4, 5, 6 or 7. More preferably, m and n are each independently an integer selected from 5-7, such as 5, 6 or 7.

In certain embodiments, compounds of Formula (I) include compounds of Formula (II):

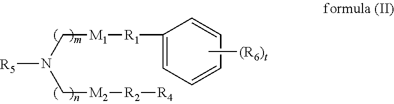

or a pharmaceutically acceptable salt thereof, wherein each group is as defined herein.

In one embodiment,
R₁ Selected from C₁-C₆ alkyl;
R₂ Selected from C₁-C₁₀ alkyl;
R₄ Selected from C₁-C₁₀ alkyl;
M₁ and M₂ each is independently selected from —OC(O)—, —C(O)O— and —OC(O)O—;
R₅ selected from —C₁₋₅ Alkylene-Q, Q is selected from —OR₇ and —SR₇, R₇ Independently selected from H, C₁-C₁₂ Alkyl and C₂-C₁₂ Alkenyl;
R₆ each is independently selected from C₁-C₁₂ Alkyl and C₂-C₁₂ Alkenyl, especially C₁-C₁₂ alkyl;
m and n are each independently an integer selected from 2-9, such as 2, 3, 4, 5, 6, 7, 8 or 9;
t is an integer selected from 1-3.

In one embodiment, R₅ selected from —C₁₋₃ Alkylene -Q, Q is selected from —OH and —SH, especially —OH.

In one embodiment, m and n are each independently an integer selected from 2-7, such as 2, 3, 4, 5, 6 or 7.

In some embodiments, t is 1 or 2.

In one embodiment, R₄ Replaced by R₂ The 1st or last digit. The 1 bit refers to R₂ Medium and M₂ The position of the directly connected C atom. The last bit refers to R₂ Medium and M₂ The position of the farthest C atom.

In one embodiment, t is 1 and R₆ Substituted on the benzene ring relative to R₁ of metaposition or counterposition.

In another embodiment, t is 2, R₆ Substituted on the benzene ring relative to R₁ Metaposition and counterposition.

In certain embodiments, compounds of Formula (I) include compounds of Formula (III):

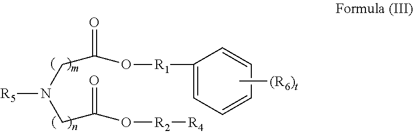

or a pharmaceutically acceptable salt thereof, wherein each group is as defined herein.

In one embodiment,
R₁ Selected from C₁-C₆ alkyl;
R₂ Selected from C₁-C₁₀ alkyl;
R₄ Selected from C₁-C₁₀ alkyl:
R₅ selected from —C₁₋₃ Alkylene -Q, Q is selected from —OH and —SH, especially —OH,
t is 1 or 2;
R₆ Selected from C₁-C₁₂ Alkyl and C₂-C₁₂ Alkenyl, especially C₁-C₁₂ alkyl;
m and n are each independently an integer selected from 2-7, such as 2, 3, 4, 5, 6 or 7.

In one embodiment, R₄ Replaced by R₂ The 1st or last digit. The 1 bit refers to R₂Neutralize

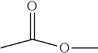

The position of some directly connected C atoms. The last bit refers to R₂Neutralize

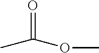

The position of the most distant C atom.

In one embodiment, t is 1 and R₆ Substituted on the benzene ring relative to R₁ of metaposition or counterposition.

In another embodiment, t is 2, R₆ Substituted on the benzene ring relative to R₁ Metaposition and counterposition.

In certain embodiments, compounds of Formula (I) include compounds of Formula (IV):

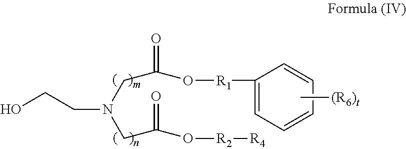

or a pharmaceutically acceptable salt thereof, wherein each group is as defined herein.

In one embodiment,
R₁ Selected from C₁-C₆ alkyl;
R₂ Selected from C₁-C₁₀ to alkyl;
R₄ Selected from C₁-C₁₀ alkyl;
t is 1 or 2;
R₆ each is independently selected from C₁-C₁₂ Alkyl and C₂-C₁₂ Alkenyl, especially C₁-C₁₂ alkyl;
m and n are each independently an integer selected from 2-7, such as 2, 3, 4, 5, 6 or 7.

In one embodiment, R₄ Replaced by R₂ The 1st or last digit. The 1 bit refers to R₂Neutralize

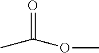

The position of some directly connected C atoms. The last bit refers to R₂Neutralize

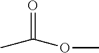

The position of the most distant C atom.

In one embodiment, t is 1 and R₆ (Substituted on the benzene ring relative to R₁ of metaposition or counterposition.

In another embodiment, t is 2, R₆ Substituted on the benzene ring relative to R₁ Metaposition and counterposition.

In a specific embodiment, the substituents in the lipid compounds of the invention (e.g., R₁-R₇) does not contain an alkenyl group.

In a preferred embodiment, the cationic lipid comprises a lipid compound having the structure shown below or a pharmaceutically acceptable salt thereof:

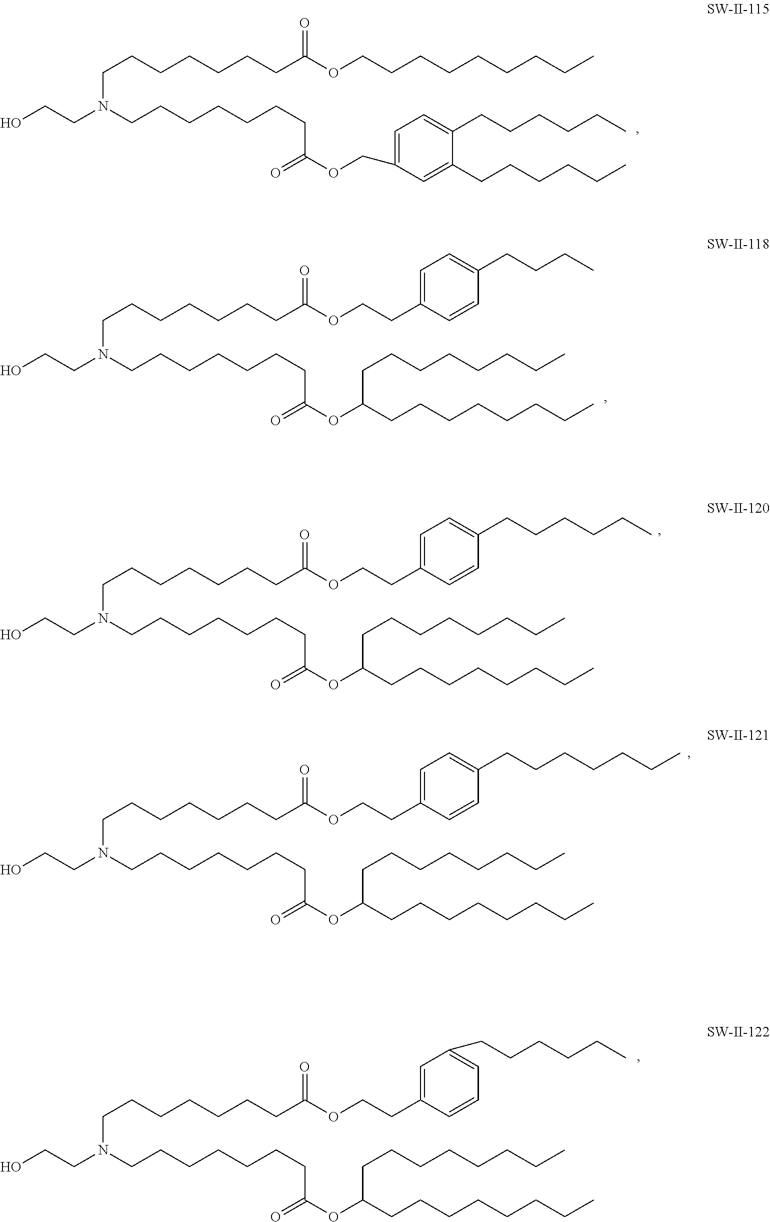 [SMILES: CCCCCCCCCOC(=O)CCCCCCCN(CCO)CCCCCCCC(=O)OCc1ccc(CCCCCC)c(CCCCCC)c1 ; CCCCCCCCC(CCCCCCCC)OC(=O)CCCCCCCN(CCO)CCCCCCCC(=O)OCCc1ccc(CCCC)cc1 ; CCCCCCCCC(CCCCCCCC)OC(=O)CCCCCCCN(CCO)CCCCCCCC(=O)OCCc1ccc(CCCCCC)cc1 ; CCCCCCCCC(CCCCCCCC)OC(=O)CCCCCCCN(CCO)CCCCCCCC(=O)OCCc1ccc(CCCCCCC)cc1 ; CCCCCCCCC(CCCCCCCC)OC(=O)CCCCCCCN(CCO)CCCCCCCC(=O)OCCc1cccc(CCCCCC)c1]

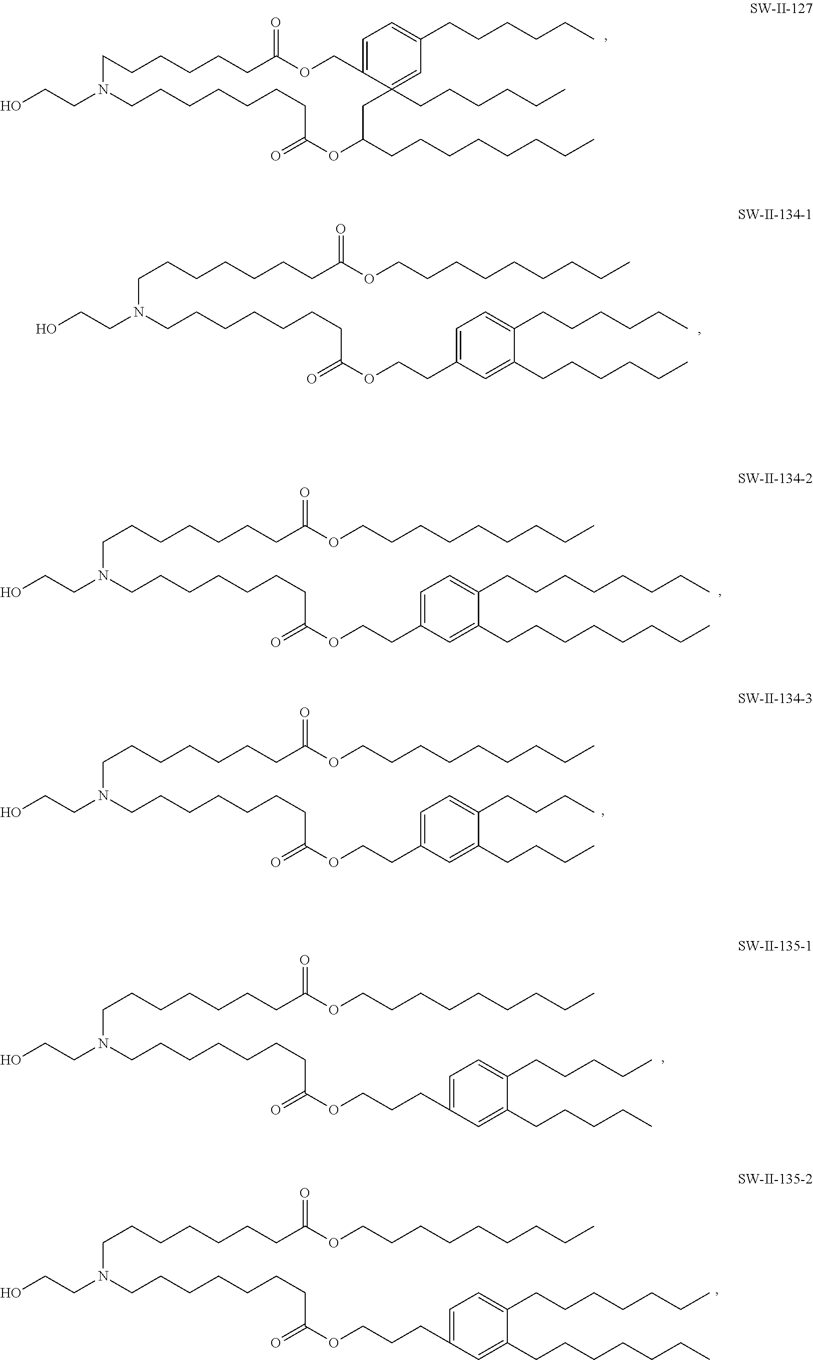 [SMILES: CCCCCCCCC(CCCCCCCC)OC(=O)CCCCCCCN(CCO)CCCCCCC(=O)OCc1ccc(CCCCCC)cc1 ; CCCCCCCCCOC(=O)CCCCCCCN(CCO)CCCCCCCC(=O)OCCc1ccc(CCCCCC)c(CCCCCC)c1 ; CCCCCCCCCOC(=O)CCCCCCCN(CCO)CCCCCCCC(=O)OCCc1ccc(CCCCCCCC)c(CCCCCCCC)c1 ; CCCCCCCCCOC(=O)CCCCCCCN(CCO)CCCCCCCC(=O)OCCc1ccc(CCCC)c(CCCC)c1 ; CCCCCCCCCOC(=O)CCCCCCCN(CCO)CCCCCCCC(=O)OCCCc1ccc(CCCCC)c(CCCCC)c1 ; CCCCCCCCCOC(=O)CCCCCCCN(CCO)CCCCCCCC(=O)OCCCc1ccc(CCCCCCC)c(CCCCCCC)c1]

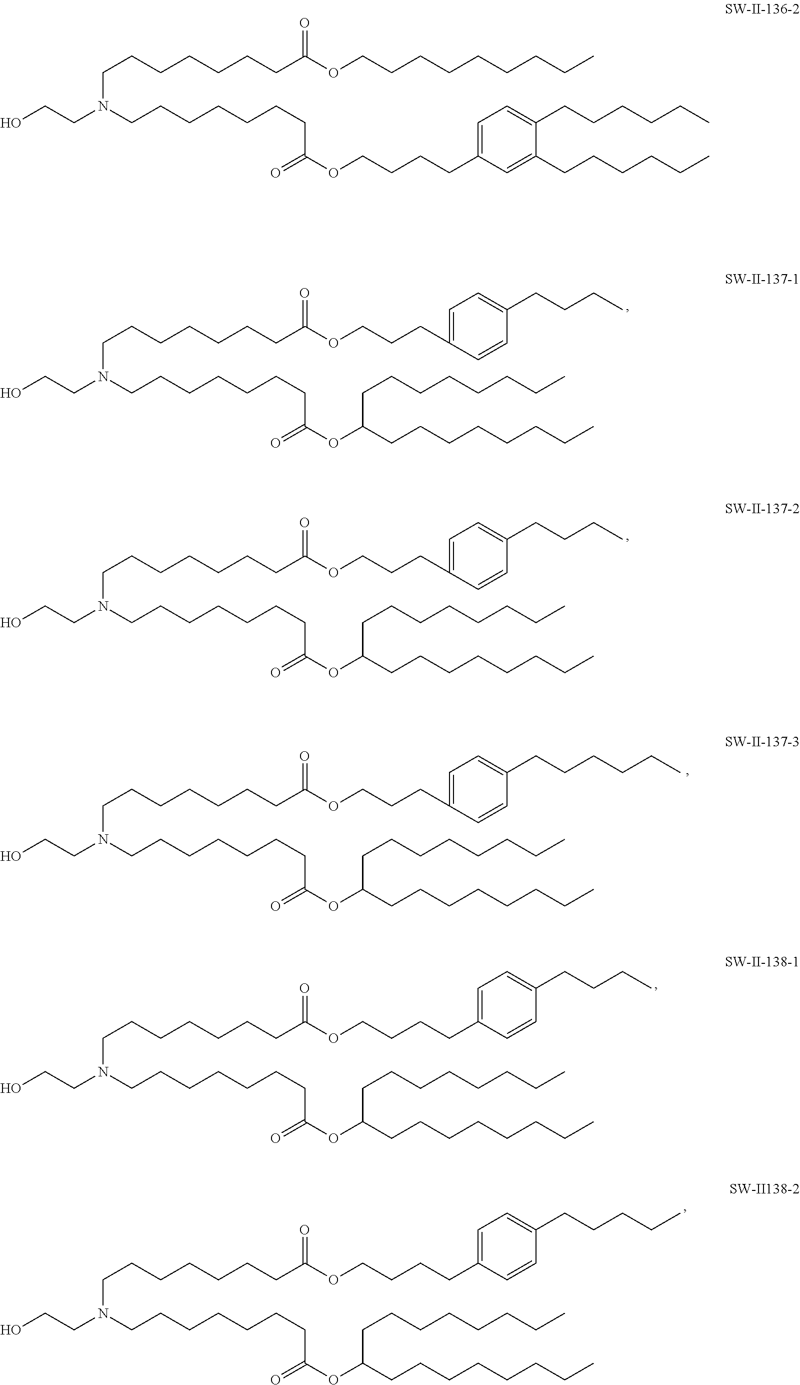 [SMILES: CCCCCCCCCOC(=O)CCCCCCCN(CCO)CCCCCCCC(=O)OCCCCc1ccc(CCCCCC)c(CCCCCC)c1 ; CCCCCCCCC(CCCCCCCC)OC(=O)CCCCCCCN(CCO)CCCCCCCC(=O)OCCCc1ccc(CCCC)cc1 ; CCCCCCCCC(CCCCCCCC)OC(=O)CCCCCCCN(CCO)CCCCCCCC(=O)OCCCc1ccc(CCCC)cc1 ; CCCCCCCCC(CCCCCCCC)OC(=O)CCCCCCCN(CCO)CCCCCCCC(=O)OCCCc1ccc(CCCCCC)cc1 ; CCCCCCCCC(CCCCCCCC)OC(=O)CCCCCCCN(CCO)CCCCCCCC(=O)OCCCCc1ccc(CCCC)cc1 ; CCCCCCCCC(CCCCCCCC)OC(=O)CCCCCCCN(CCO)CCCCCCCC(=O)OCCCCc1ccc(CCCCC)cc1]

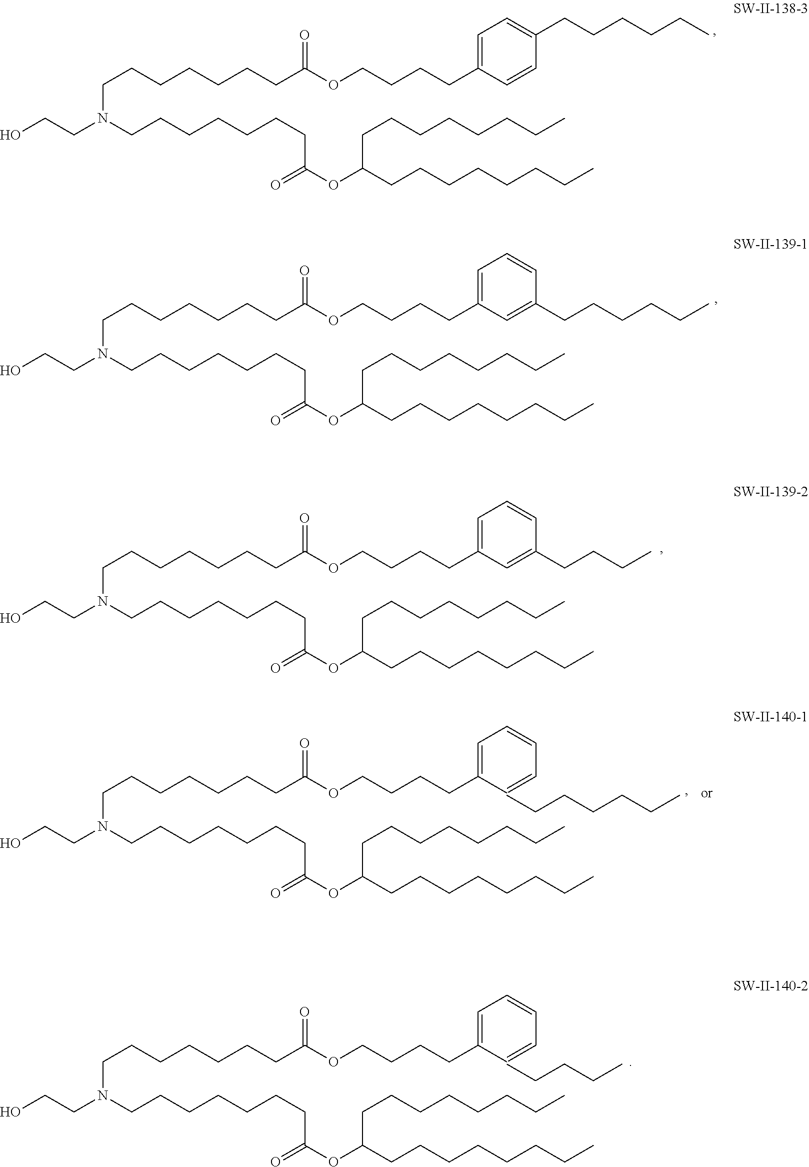

In a preferred embodiment, the cationic lipids comprise the following lipid compounds SW-II-121, SW-II-138-1, SW-II-139-1, SW-II-140-1 or SW-II-140-2.

In a preferred embodiment, the cationic lipid does not contain T5′.

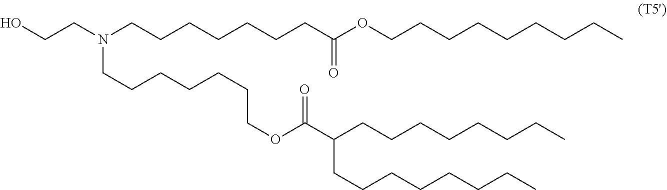 [SMILES: CCCCCCCCCOC(=O)CCCCCCCN(CCO)CCCCCCCOC(=O)C(CCCCCCCC)CCCCCCCC]

In a preferred embodiment, the cationic lipids include the following lipid compounds: M5, MC3, ALC-0315, SM-102, SW-II-121, SW-II-138-1, SW-II-139-1, SW-II-140-1 or SW-II-140-2.

### Lipid

The lipid composition of the present invention contains phospholipids, which can assist cell penetration of the lipid composition.

Examples of phospholipids include, but are not limited to: 1,2-dilinoleoyl-sn-glycero-3-phosphocholine (DLPC), 1,2-dimyristoyl-sn-glycero-phosphocholine (DMPC), 1,2-Dioleoyl-sn-glycero-3-phosphocholine (DOPC), 1,2-dioleoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycerol-3-Phosphocholine (POPC), 1,2-di-O-octadecenyl-sn-glycero-3-phosphocholine (18:0Diether PC), 1-oleoyl-2-cholesteryl hemisuccinyl-sn-glycero-3-phosphocholine (OChemsPC), 1-hexadecyl-sn-glycero-3-phosphocholine (C16 Lyso PC), 1,2-dilinolenoyl-sn-glycerol-3-Phosphocholine, 1,2-diarachidonoyl-sn-glycero-3-phosphocholine, 1,2-bidocosahexaenoyl-sn-glycero-3-phosphocholine, 1,2-Dioleoyl-sn-glycero-3-phosphoethanolamine (DOPE), 1,2-diphytanoyl-sn-glycero-3-phosphoethanolamine (ME 16.0 PE), 1,2-distearoyl-sn-Glycerol-3-phosphoethanolamine, 1,2-dilinoleoyl-sn-glycero-3-phosphoethanolamine, 1,2-dilinoleoyl-sn-glycerol-3-phosphoethanolamine, 1,2-diarachitetradine Enoyl-sn-glycerol-3-phosphoethanolamine, 1,2-bidocosahexaenoyl-sn-glycerol-3-phosphoethanolamine, 1,2-dioleoyl-sn-glycerol-3-phosphate-rac-(1-glycerol) sodium salt (DOPG), dipalmitoylphosphatidylglycerol (DPPG), palmitoyloleoylphosphatidylethanolamine (POPE), distearoyl-phosphatidyl-ethanolamine (DSPE), dipalmitoyl Phosphatidylethanolamine (DPPE), dimyristoylphosphoethanolamine (DMPE), 1-stearoyl-2-oleoyl-stearoylethanolamine (SOPE), 1-stearoyl-2-oleoyl-phosphatidylchol Base (SOPC), sphingomyelin, phosphatidylcholine, phosphatidylethanolamine, phosphatidylserine, phosphatidylinositol, phosphatidic acid, palmitoyloleoylphosphatidylcholine, lysophosphatidylcholine, lysophosphatidylethanolamine (LPE)) or a combination thereof.

### Steroid

Steroids are included in the lipid compositions of the present invention and can serve as structural components of the lipid compositions.

Examples of steroids include, but are not limited to, cholesterol, coprosterol, sitosterol, ergosterol, campesterol, stigmasterol, campesterol, tomatine, ursolic acid, alpha-tocopherol, and derivatives thereof.

### Polyethylene Glycol Modified Lipids

As used herein, the term “polyethylene glycol modified lipid” or “PEG modified lipid” or “PEG lipid” refers to a molecule containing a polyethylene glycol moiety and a lipid moiety that is modified with polyethylene glycol. Alcohol-modified lipids. The PEG lipid may be selected from the non-limiting group consisting of: PEG-modified phosphatidylethanolamine, PEG-modified phosphatidic acid, PEG-modified ceramide (PEG-CER). PEG-modified dialkylamine, PEG-modified diacylglycerol (PEG-DEG). PEG-modified dialkylglycerol, or combinations thereof. For example, examples of polyethylene glycol-modified lipids include, but are not limited to: 2-[(polyethylene glycol)-2000]—N,N-tetracosane (2-[(polyethylene glycol)-2000]-N,N-ditetradecylacetamide, also known as ALC-0159), 1,2-dimyristoyl-rac-glycero-3-methoxypolyethyleneglycol (DMG-PEG), 1, 2-Dioleoyl-rac-glycerol, methoxypolyethylene glycol (1,2-Dioleoyl-rac-glycerol, methoxy polyethylene Glycol, DOGPEG) and 1,2-distearoyl-sn-glycerol-3-DSPE-PEG.

In one embodiment, the polyethylene glycol modified lipid is DMG-PEG, such as DMG-PEG 2000. In one embodiment, DMG-PEG 2000 has the following structure:

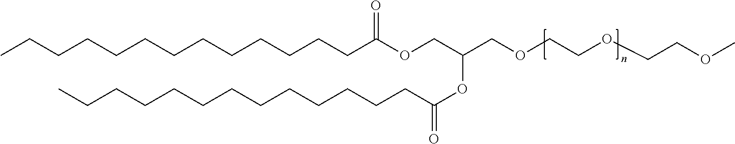

where the average value of n is 44.

### Cationic Polymer

As used herein, the term “cationic polymer” refers to any ionic polymer capable of carrying a net positive charge at a specified pH to electrostatically bind to nucleic acids. Examples of cationic polymers include, but are not limited to: poly-L-lysine, protamine, polyethylenimine (PEI), or combinations thereof. The polyethyleneimine may be linear or branched polyethyleneimine.

The term “protamine” refers to an arginine-rich low molecular weight basic protein that is present in the sperm cells of various animals (especially fish) and binds to DNA instead of histones. In a preferred embodiment, the cationic polymer is protamine (eg protamine sulfate).

### Physical and Chemical Properties

The physicochemical properties of a lipid composition may depend on its components. For example, a composition including cholesterol as a structural lipid may have different physicochemical properties than a composition including a different structural lipid. Similarly, the physicochemical properties of a composition may depend on the absolute or relative amounts of its components. For example, a composition including a higher mole fraction of phospholipids may have different physicochemical properties than a composition including a lower mole fraction of phospholipids. Physicochemical properties may also vary depending on the method and conditions used to prepare the composition.

The physicochemical properties of lipid compositions can be characterized by a variety of methods. For example, the morphology and size distribution of the composition can be examined using microscopy, such as transmission electron microscopy or scanning electron microscopy. Dynamic light scattering or potentiometric methods (eg potentiometric titration) can be used to measure zeta potential. Dynamic light scattering can also be used to determine particle size. Instruments such as the Zetasizer Nano ZS (Malvern Instruments Ltd, Malvern, Worcestershire, UK) can also be used to measure various characteristics of the composition, such as particle size, polydispersity index and zeta potential

The average size of the composition can be between tens and hundreds of nanometers, as measured, for example, by dynamic light scattering (DLS). For example, the average size may be about 40 nm to about 250 nm, such as about 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm or 300 nm. In some embodiments, the average size of the composition can be about 50 nm to about 300 nm, about 50 nm to about 290 nm, about 50 nm to about 280 nm, about 50 nm to about 270 nm, about 50 nm to about 260 nm, about 60 nm to about 300 nm, about 60 nm to about 290 nm, about 60 nm to about 280 nm, about 60 nm to about 270 nm.

About 70 nm to about 300 nm, about 70 nm to about 290 nm, about 70 nm to about 280 nm, about 70 nm to about 270 nm, about 70 nm to about 260 nm, about 80 nm to about 280 nm, about 80 nm to about 270 nm, about 80 nm to about 260 nm, about 80 nm to about 250 nm, about 90 nm to about 280 nm, about 90 nm to about 270 nm, or about 90 nm to about 260 nm. In certain embodiments, the average size of the lipid composition can be from about 90 nm to about 290 nm or from about 100 nm to about 250 nm. In a specific embodiment, the average size may be about 100 nm. In other embodiments, the average size may be about 150 nm. In other embodiments, the average size may be about 200 nm.

The lipid composition can be relatively homogeneous. The polydispersity index can be used to indicate the homogeneity of a lipid composition, such as the particle size distribution of the lipid composition. A smaller (eg, less than 0.3) polydispersity index generally indicates a narrower particle size distribution. The polydispersity index of the composition can be from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24 or 0.25. In some embodiments, the polydispersity index of the lipid composition can be from about 0.10 to about 0.20.

The zeta potential of a composition can be used to indicate the zeta potential of the composition. For example, zeta potential can describe the surface charge of a composition. Compositions that have a relatively low charge, ie, either positively or negatively charged, are generally desirable because more highly charged compositions may have undesirable interactions with cells, tissues, and other elements in the body. In some embodiments, the zeta potential of the composition can be from about −10 mV to about +20 mV, from about −10 mV to about +15 mV, from about −10 mV to about +10 mV, from about −10 mV to about +5 mV, from about −10 mV to about 0 mV, about −10 mV to about −5 mV, about −5 mV to about +20 mV, about −5 mV to about +15 mV, about −5 mV to about +10 mV, about −5 mV to about +5 mV, about −5 mV to about 0 mV, about 0 mV to approximately +20 mV, approximately 0 mV to approximately +15 mV, approximately 0 mV to approximately +10 mV, approximately 0 mV to approximately +5 mV, approximately +5 mV to approximately +20 mV, approximately +5 mV to approximately +15 mV, or approximately +5 mV to approximately +10 mV.

The encapsulation efficiency of a therapeutic or prophylactic agent describes the ratio of the amount of therapeutic or prophylactic agent that is encapsulated in or otherwise associated with a composition after preparation relative to the initial amount provided. Higher encapsulation efficiencies are desirable (eg, close to 100%). Encapsulation efficiency can be measured, for example, by comparing the amount of therapeutic or prophylactic agent in a solution containing the composition before and after splitting the composition with one or more organic solvents or detergents. Fluorescence can be used to measure the amount of therapeutic or prophylactic agents (eg, RNA) in solution. For compositions described herein, the encapsulation efficiency of the therapeutic or prophylactic agent can be at least 50%, such as 50%, 55%, 60%, 65%, 70%, 75%, 80%, 85%, 90%, 91%, 92%, 93%, 94%, 95%, 96%, 97%, 98%, 99% or 100%. In some embodiments, encapsulation efficiency can be at least 80%. In certain embodiments, encapsulation efficiency can be at least 90%.

### Pharmaceutical Composition

The present invention also provides a pharmaceutical composition, which contains the lipid composition of the present invention and a pharmaceutically acceptable carrier.

Pharmaceutically acceptable carriers may include, but are not limited to: propellants, diluents, binders and adhesives, lubricants, disintegrants, preservatives, vehicles, dispersants, glidants, sweeteners, packaging Coatings, excipients, preservatives, antioxidants (such as ascorbic acid, cysteine hydrochloride, sodium bisulfate, sodium metabisulfite, sodium sulfite, ascorbyl palmitate, butylated hydroxyanisole (BHA), butylated hydroxytoluene (BHT), Lecithin, propyl gallate, alpha-tocopherol, citric acid, ethylenediaminetetraacetic acid (EDTA), sorbitol, tartaric acid, phosphoric acid, etc.), solubilizer, gelling agent, softener, solvent (e.g., water, alcohol, acetic acid, and syrup), buffers (e.g., phosphate buffers, histidine buffers, and acetate buffers), surfactants (e.g., nonionic surfactants such as polysorbate ester 80, polysorbate 20, poloxamer or polyethylene glycol), antibacterial agents, antifungal agents, isotonic agents (e.g. trehalose, sucrose, mannitol, sorbitol, lactose, glucose), delayed absorption agents, chelating agents and emulsifiers. For pharmaceutical compositions, suitable carriers may be selected from buffers (eg, citrate buffer, acetate buffer, phosphate buffer, histidine buffer, histidine salt buffer), etc. Osmotic agents (such as trehalose, sucrose, mannitol, sorbitol, lactose, glucose), nonionic surfactants (such as polysorbate 80, polysorbate 20, poloxamer) or combinations thereof.

Pharmaceutical compositions provided herein may be in a variety of dosage forms, including, but not limited to, solid, semi-solid, liquid, powder, or lyophilized forms. For pharmaceutical compositions, preferred dosage forms may generally be, for example, solutions and lyophilized powders. Pharmaceutical compositions can be prepared in a variety of forms suitable for a variety of routes and methods of administration. For example, pharmaceutical compositions can be prepared in liquid dosage forms (such as emulsions, microemulsions, nanoemulsions, solutions, suspensions, syrups and elixirs), injectable forms, solid dosage forms (such as capsules, tablets, pills, powders and granules formulations), dosage forms for topical and/or transdermal administration (such as ointments, pastes, creams, lotions, gels, powders, solutions, sprays, inhalants and patches), suspensions, powders and others form.

On the other hand, the lipid composition or pharmaceutical composition of the present invention is used for nasal, oral, conjunctival, rectal, and vaginal mucosa administration.

In a preferred embodiment, the lipid composition or pharmaceutical composition is used for nasal administration.

Nasal drug delivery system refers to a type of preparation that is administered through the nasal cavity and exerts local or systemic therapeutic or preventive effects. It is especially suitable for drugs that require high local expression in the nasal cavity or drugs that avoid the first-pass effect of the liver. The advantage of nasal administration is that the nasal mucosa has a large area and rich blood vessels, so the drug is absorbed quickly and takes effect quickly after administration. Moreover, it has low first-pass effect in the liver, low systemic toxicity, and high bioavailability.

Common dosage forms for nasal administration include nasal drops, nasal sprays, powders, gel preparations, and emulsions. Among them, nasal spray drugs have a wider dispersion degree and dispersion area in the nasal mucosa. Currently, the commonly used atomization devices include quantitative and constant pressure inhalers (MDI), dry powder inhalers (DPI) and nebulizers. After nasal spraying, the drug is deposited in the front of the nasal cavity, and only a small part is slowly cleared into the throat. This prolongs the residence time of the drug in the nasal cavity, which is beneficial to absorption and improves bioavailability.

The pharmaceutical composition of the present invention can be nasal drops or nasal spray, which contains the lipid composition of the present invention and pharmaceutically acceptable excipients. Therefore, the present invention also provides a nasal drop or nasal spray, which contains the lipid composition of the present invention and a pharmaceutically acceptable excipient.

The excipients may include propellants, such as trichloromonofluoromethane, dichlorodifluoromethane, dichlorotetrafluoroethane, 1,1,1,2-tetrafluoroethane, etc. Excipients may also include one or more of water, sugar solution, electrolyte solution, and amino acid solution. For example, it may include one of water, Ringer's solution, glucose solution, glucose sodium chloride solution, isotonic sodium chloride solution, fructose solution, dextran, amino acid solution, heparin solution, mannitol solution, sodium bicarbonate solution, or Various.

In some embodiments, the nasal administration includes nasal instillation, nasal spray administration, or nasal inhalation.

In a preferred embodiment, the nasal administration includes nasal drip or nasal spray administration.

In one embodiment, the nasal spray administration is performed via an aerosol administration device.

In one embodiment, the aerosol delivery device includes a syringe, a plastic needle, a nasal spray device, and a dose limiter.

In one embodiment, the atomized drug delivery device can convert liquid medicine into mist particles (jet atomization). Use umbrella spray.

As used in this article, “jet atomization” is based on the Venturi injection principle, using compressed air or high-flow medical oxygen to form a high-speed airflow through a small nozzle, and the generated negative pressure drives liquid or other fluids to be sprayed onto the barrier., splashing around under high-speed impact, causing the liquid droplets to turn into mist particles and spray out from the air outlet.

In one embodiment, the liquid pharmaceutical agent is atomized into fine mist particles with a size of 10-70 μm and sprayed on the surface of human body surface tissues (or organs).

In one embodiment, the atomized drug delivery device has a built-in self-destruction design, and the push rod self-destructs after use, ensuring one-time use.

### Therapeutic/Preventive Agents

Lipid compositions may contain one or more therapeutic or prophylactic agents. The present invention provides methods of delivering a therapeutic or prophylactic agent to a mammalian mucosa, producing a polypeptide of interest in the mammalian mucosa, and treating a disease or condition in a mammal in need thereof, which methods comprise administering to the mammalian mucosa a therapeutic or prophylactic agent, lipid composition or pharmaceutical composition.

In some embodiments, the disease or disorder is selected from the group consisting of rare diseases, infectious diseases, cancer and proliferative diseases, genetic diseases, autoimmune diseases, neurodegenerative diseases, cardiovascular and renovascular diseases, and metabolic diseases.

In some embodiments, the disease is an infectious disease.

In some embodiments, the infectious disease includes novel coronavirus pneumonia, influenza, acute upper respiratory tract infection, pneumococcal disease, haemophilic influenza meningitis, epidemic cerebrospinal meningitis, diphtheria, pertussis, Measles, human papillomavirus (HPV), rabies, tetanus, plague, hepatitis and tuberculosis.

In some embodiments, the therapeutic or prophylactic agent is a vaccine or compound that elicits an immune response. Vaccines include vaccines that can provide protection against infectious diseases such as new coronavirus pneumonia, influenza, acute upper respiratory tract infection, pneumococcal disease, haemophilic influenza meningitis, epidemic cerebrospinal meningitis, diphtheria, whooping cough, measles, Compounds and preparations immunizing against one or more conditions associated with human papillomavirus (HPV), rabies, tetanus, plague, hepatitis and tuberculosis and may include mRNA encoding infectious disease-derived antigens and/or epitopes. In some embodiments, vaccines and/or compounds capable of eliciting an immune response are administered mucosally via a lipid composition comprising a compound according to Formula (I), (II), (III) or (IV).

### Polynucleotide

In some embodiments, the therapeutic or prophylactic agent is a polynucleotide or nucleic acid (eg, ribonucleic acid or deoxyribonucleic acid).

In some embodiments, the therapeutic or prophylactic agent of the invention is RNA. As used herein, the definition of “RNA” encompasses single-stranded, double-stranded, linear and circular RNA. The RNA of the present invention can be chemically synthesized, recombinantly produced, or in vitro transcribed RNA. In one embodiment, the RNA of the invention is used to express polypeptides in host cells.

In one embodiment, the therapeutic or prophylactic agent of the invention is single-stranded RNA. In one embodiment, the RNA of the invention is in vitro transcribed RNA (IVT-RNA). IVT-RNA can be obtained by in vitro transcription using RNA polymerase using a DNA template.

In some embodiments, the therapeutic or prophylactic agent of the invention is messenger RNA (mRNA). Generally speaking, the mRNA may include a 5′-UTR sequence, a polypeptide coding sequence, a 3′-UTR sequence and an optional poly(A) sequence.

List. The mRNA can be produced, for example, by in vitro transcription or chemical synthesis. In one embodiment, the mRNA of the invention comprises (1) 5′-UTR, (2) coding sequence, (3) 3′-UTR, and (4) optionally a poly(A) sequence. In one embodiment, the mRNA of the invention is a nucleoside-modified mRNA. In one embodiment, the mRNA of the invention contains an optional 5′ cap.

As used herein, the term “untranslated region (UTR)” generally refers to a region in RNA (eg, mRNA) that is not translated into an amino acid sequence (non-coding region), or the corresponding region in DNA. Generally, the UTR located at the 5′ end (upstream) of the open reading frame (start codon) can be called the 5′ untranslated region 5′-UTR; the UTR located at the 3′ end (downstream) of the open reading frame (stop codon) The UTR can be called 3′-UTR. In the presence of a 5′ cap, the 5′-UTR is located downstream of the 5′ cap, e.g., directly adjacent to the 5′ cap. In certain embodiments, an optimized “Kozak sequence” may be included in the 5′-UTR, for example, adjacent to the start codon, to increase translation efficiency. In the presence of a poly(A) sequence, the 3′-UTR is located upstream of, e.g., directly adjacent to, the poly(A) sequence.

As used herein, the term “poly(A) sequence” or “poly(A) tail” refers to a nucleotide sequence containing contiguous or discontinuous adenosine nucleotides. The poly(A) sequence is usually located at the 3′ end of the RNA, such as the 3′ end (downstream) of the 3′-UTR. In some embodiments, the poly(A) sequence contains no nucleotides other than adenylate at its 3′ end. The poly(A) sequence can be transcribed by DNA-dependent RNA polymerase according to the coding sequence of the DNA template during the preparation of IVT-RNA, or linked to the IVT by a DNA-independent RNA polymerase (poly(A) polymerase)—The free 3′ end of the RNA, for example the 3′ end of the 3′-UTR

As used herein, the term “5′ cap” generally refers to an N7-methylguanosine structure (also known as “m7G cap”, “m7Gppp-”) linked to the 5′ end of an mRNA via a 5′ to 5′ triphosphate bond. The 5′ cap can be co-transcriptionally added to the RNA during in vitro transcription (e.g. using the anti-reverse cap analog “ARCA”) or can be ligated to the RNA post-transcriptionally using a capping enzyme.

In some embodiments, the therapeutic or prophylactic agent of the invention is DNA. Such DNA may be, for example, a DNA template for in vitro transcription of the RNA of the invention or a DNA vaccine for expression of a polypeptide antigen in a host cell. DNA can be double-stranded, single-stranded, linear, and circular DNA.

The DNA template can be provided in a suitable transcription vector. In general, a DNA template can be a double-stranded complex comprising a nucleotide sequence identical to a coding sequence described herein (coding strand) and a nucleotide sequence complementary to a coding sequence described herein (template strand). As known to those skilled in the art, the DNA template may comprise a promoter, 5′-UTR, coding sequence, 3′-UTR and optionally a poly(A) sequence. The promoter may be one known to those skilled in the art to be usable by suitable RNA polymerases (especially DNA-dependent RNA polymerases), including but not limited to promoters for SP6, T3 and T7 RNA polymerases. The 5′-UTR, coding sequence, 3′-UTR and poly(A) sequences in the DNA template are the corresponding sequences contained in the RNA described herein or are complementary to them. Polynucleotides that are DNA vaccines can be provided in plasmid vectors (eg, circular plasmid vectors).

In a preferred embodiment, the therapeutic or preventive agent of the present invention is an mRNA encoding a SARS-CoV-2 spike protein variant, the exemplary coding sequence of which can be found in SEQ ID NO: 2. In another preferred embodiment, the therapeutic or preventive agent of the present invention is mRNA encoding an influenza virus antigen such as NP protein, and its exemplary coding sequence can be found in SEQ ID NO: 3.

### Modified Nucleotides

In some ways, the mRNA herein includes modified nucleotides, wherein the modified nucleotides are selected from one or several of the following nucleotides: 2-aminoadenosine, 2-thiothymidine, inosine, pyrrole Pyrimidine, 3-methyladenosine, 5-Methylcytidine, C-5 propynyl-cytidine, C-5 propynyl-uridine, 2-aminoadenosine, C5-bromouridine, C5-fluorouridine, C5-iodouridine, C5-Propamyl-uridine, C5-propynyl-cytidine, C5-methylcytidine, 2-aminoadenosine, 7-deazaadenosine, 7-deazaguanosine, 8-oxadenosine, 8-oxoguanine, O(6)-methylguanine, pseudouridine, N-1-methyl-pseudouridine, 2-thiouridine, and 2-thiocytidine; methylation Bases; inserted bases; 2′-fluoribose, ribose, 2′-deoxyribose, arabinose and hexose; phosphorothioate and 5′-N-phosphoramidite bonds. And modified with the modified nucleotides described in PCT/CN2020/074825 and PCT/CN2020/106696.

### Use of Lipid Compositions and Pharmaceutical Compositions

The lipid compositions, pharmaceutical compositions, nasal drops and nasal sprays of the present invention can be used to treat diseases, disorders or conditions. Specifically, these lipid compositions, pharmaceutical compositions, nasal drops and nasal sprays may be used to treat diseases, disorders or conditions characterized by missing or abnormal protein or polypeptide activity. For example, lipid compositions and pharmaceutical compositions containing mRNA encoding a missing or aberrant polypeptide can be administered or delivered to the mucosa. Subsequent translation of the mRNA can produce the polypeptide, thereby reducing or eliminating problems caused by the absence or abnormal activity of the polypeptide.

There are a variety of diseases, disorders or conditions that can be characterized by a loss of protein activity (or a substantial reduction such that appropriate protein function cannot occur). These proteins may not be present, or they may be essentially non-functional. The invention provides a method of treating such a disease, disorder or condition in a subject by administering a lipid composition, pharmaceutical composition, nasal drop or nasal spray of the invention, said lipid composition comprising RNA and lipid components, the lipid components include cationic lipids, phospholipids, PEG lipids and structural lipids, wherein the RNA may encode an activity that antagonizes or otherwise overcomes the abnormal protein activity present in the cells of the subject Peptide mRNA.

The present invention provides methods involving the administration of lipid compositions, pharmaceutical compositions, nasal drops or nasal sprays containing one or more therapeutic or prophylactic agents. For features and embodiments of the invention, the terms therapeutic agent and prophylactic agent may be used interchangeably herein. Lipid compositions and pharmaceutical compositions may be administered to a subject in any reasonable amount and by any route of administration effective to prevent, treat, diagnose, or for any other purpose the disease, disorder, or condition. The specific amount administered to a given subject may vary depending on the species, age, and general condition of the subject; the purpose of administration: the specific composition; the mode of administration, and the like.

Diseases, disorders or conditions characterized by dysfunction or abnormal protein or polypeptide activity to which lipid compositions, pharmaceutical compositions, nasal drops and nasal sprays may be administered include, but are not limited to, rare diseases, infectious diseases (in the form of vaccines) and therapeutic agent forms), cancer and proliferative diseases, genetic diseases (such as cystic fibrosis), autoimmune diseases, neurodegenerative diseases, cardiovascular and renovascular diseases, and metabolic diseases. The cancers include, for example, non-small cell lung cancer, small cell lung cancer, lung adenocarcinoma, nasopharyngeal cancer, laryngeal cancer, pharyngeal cancer, tracheal cancer, melanoma, thyroid cancer, gastrointestinal cancer, gastric cancer, pancreatic cancer, neuroendocrine cancer, Glioblastoma, cervical cancer, ovarian cancer, bladder cancer, brain cancer, colon cancer, colorectal cancer, endometrial cancer, uterine cancer, salivary gland cancer, leukemia, lymphoma, myeloma, acute myeloid leukemia, chronic Myeloid leukemia, acute lymphoblastic leukemia, chronic lymphocytic leukemia, hairy cell leukemia, Hodgkin lymphoma, non-Hodgkin lymphoma, or multiple myeloma. Such autoimmune diseases include, for example, systemic lupus erythematosus, rheumatoid arthritis, ankylosing spondylitis or type I diabetes. Such neurodegenerative diseases include, for example. Parkinson's disease, Alzheimer's disease, spinal cord injury, retinal degenerative diseases, stroke, Huntington's disease or amyotrophic lateral sclerosis. Such metabolic diseases include, for example, type II diabetes, scurvy, hypoglycemia, hyperlipidemia or osteoporosis.

In some embodiments, the disease is an infectious disease.

In some embodiments, the infectious disease includes novel coronavirus pneumonia, influenza, acute upper respiratory tract infection, pneumococcal disease, haemophilic influenza meningitis, epidemic cerebrospinal meningitis, diphtheria, pertussis, Measles, human papillomavirus (HPV), rabies, tetanus, plague, hepatitis and tuberculosis.

In one aspect, the invention also provides the use of the lipid composition, pharmaceutical composition, nasal drops or nasal spray of the invention in the preparation of a medicament for treating or preventing diseases in a subject in need thereof or illness. The disease or disorder is as described above.

In one embodiment, the disease or disorder is characterized by dysfunctional or abnormal protein or polypeptide activity. In one embodiment, the disease or disorder is selected from the group consisting of rare diseases, infectious diseases, cancer and proliferative diseases, genetic diseases, autoimmune diseases, neurodegenerative diseases, cardiovascular and renovascular diseases, and metabolic diseases. In a preferred embodiment, the disease is an infectious disease.

In another aspect, the invention also provides a method for treating or preventing a disease or condition, comprising administering to a subject a therapeutic or prophylactic agent in a multiple dose regimen, wherein at least one dose is administered by a mucosal route The lipid composition of the present invention or the pharmaceutical composition of the present invention. The disease or disorder is as described above.

In one embodiment, at least one additional dose of the multiple dose regimen is administered by a route selected from: intramuscular, intratumoral, transdermal, intravenous, intradermal, subcutaneous, intraperitoneal, intraventricular, intracranial, or its combination.

In a preferred embodiment, said at least one additional dose is administered by the intramuscular route.

As mentioned above, a therapeutic or prophylactic agent as described herein may be administered by two or more modes of administration, in other words, the therapeutic or prophylactic agent may be administered in a multiple dose regimen. At least one dose is administered via a mucosal route, such as the lipid composition or pharmaceutical composition of the present invention, and at least one dose is administered via a different administration route than the mucosal route. In a particularly preferred embodiment, the therapeutic or prophylactic agent administered by different routes of administration is the same. In a preferred embodiment, the therapeutic or preventive agent is an mRNA encoding a SARS-CoV-2 spike protein variant, the exemplary coding sequence of which can be found in SEQ ID NO: 2. In another preferred embodiment, the therapeutic or preventive agent is mRNA encoding an influenza virus antigen such as NP protein, an exemplary coding sequence of which can be found in SEQ ID NO: 3.

Different administration methods can be performed sequentially or simultaneously. Where appropriate, multiple-dose regimens can achieve better therapeutic effects, such as eliciting better immune responses.

In one embodiment, at least one dose of a therapeutic or prophylactic agent as described herein is administered by the intramuscular route, followed by at least one dose of the lipid composition or pharmaceutical composition of the invention by the mucosal route.

In one embodiment, a dose of a therapeutic or prophylactic agent as described herein is administered by the intramuscular route, followed by a dose of the lipid composition or pharmaceutical composition of the invention by the mucosal route.

In one embodiment, at least two doses of a therapeutic or prophylactic agent as described herein are administered by the intramuscular route, followed by at least one dose of the lipid composition or pharmaceutical composition of the invention by the mucosal route.

In one embodiment, two doses of a therapeutic or prophylactic agent as described herein are administered by the intramuscular route, followed by one dose of the lipid composition or pharmaceutical composition of the invention by the mucosal route.

The invention accordingly also relates to the use of a lipid composition of the invention or a pharmaceutical composition of the invention in the preparation of a treatment or prevention of a disease or condition, wherein the therapeutic or prophylactic agent is administered to a subject in a multi-dose regimen, wherein at least one dose is administered through the mucosal route of the lipid composition of the present invention or the pharmaceutical composition of the present invention. The multiple dose regimen is as described above. The disease or disorder is as described above.

### Beneficial Effects

The lipid composition, pharmaceutical composition, nasal drops or nasal spray provided by the present invention can exhibit excellent effects, such as having at least one of the following beneficial effects: (1) increasing the concentration of the contained mRNA in the nasal cavity Expression efficiency; (2) Good targeting and low systemic toxicity; (3) No significant changes in physical and chemical properties and expression efficiency before and after atomization; (4) Can cause mucosal immune response; (5) Can be combined with Other routes, such as intramuscular combined administration in the form of “primary+boost” (systemic immunity+mucosal immunity), can induce a higher level of humoral immunity and cellular immunity, and can also induce mucosal immune responses.

### EXAMPLE

The invention is further described with reference to the following examples. It should be understood that these embodiments are only examples and do not limit the invention. The following materials and instruments are either commercially available or prepared according to methods well known in the art. The following experiments were performed according to the manufacturer's instructions or according to methods and procedures known in the art.

Unless otherwise specified, the percentages in the following examples are molar percentages (mol %).

### Experimental Materials

The cationic lipid according to formula (I) is synthesized by microorganisms or prepared by reference, such as CN110520409A, WO2018081480A1 or U.S. Pat. No. 11,246,933B1; phospholipid (DOPE) is purchased from CordenPharma; cholesterol is purchased from Sigma-Aldrich; mPEG2000-DMG (i.e. DMG-PEG 2000) was purchased from Avanti Polar Lipids, Inc.; PBS was purchased from Invitrogen; protamine sulfate was purchased from Beijing Silian Pharmaceutical Co., Ltd.; mPEG2000-DSPE was purchased from lipoid GmbH; DSPC was purchased from Avanti Polar Lipids, Inc.

### Example 1 Synthesis of Compounds According to Formula (I)

### General Considerations

Unless otherwise stated, all solvents and reagents used were commercially available and used as received. ¹H NMR spectra were obtained at 300K using a Bruker Ultrashield 300 MHz instrument at CDCl₃ record. Chemical shifts are about 1H is reported in parts per million (ppm) relative to TMS (0.00). Silica gel chromatography was performed on an ISCO CombiFlash Rf+Lumen instrument using an ISCO RediSep Rf Gold column (particle size: 20-40 μm).

The procedure described below can be used to synthesize compounds SW-II-115 to SW-II-140-2.

This article uses the following abbreviations:
THF: Tetrahydrofuran
MeCN: acetonitrile
LAH: lithium aluminum hydride
DCM: dichloromethane
DMAP: 4-dimethylaminopyridine
LDA: lithium diisopropylamide
rt: room temperature
DME: 1,2-Dimethoxyethane
n-BuLi: n-butyllithium
CPME: cyclopentyl methyl ether
EDCI: N-(3-dimethylaminopropyl)-N′-ethylcarbodiimide
DIEA: N,N-diisopropylethylamine
PE: Petroleum ether
EA: Ethyl acetate

### A. Compound SW-II-115

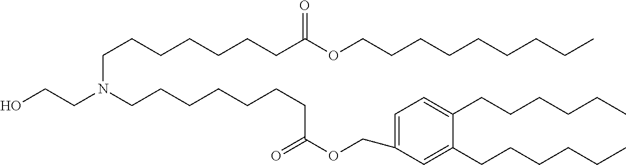 [SMILES: CCCCCCCCCOC(=O)CCCCCCCN(CCO)CCCCCCCC(=O)OCc1ccc(CCCCCC)c(CCCCCC)c1]

### 1. Synthesis of Intermediate 3

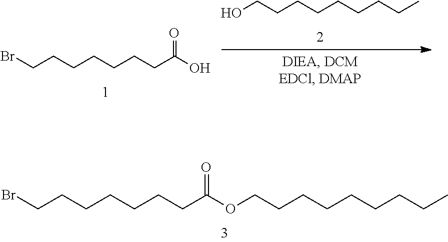

To the DCM solution (100 mL) containing compound 1 (10 g, 45 mmol, 1 eq.) and compound 2 (7.8 g, 54 mmol, 1.2 eq.), EDCI (17.3 g, 90 mmol, 2 eq.) and DMAP (2.2 g, 18 mmol, 0.4 eq.), then add DIEA (23.2 g, 180 mmol, 4 eq.). The reaction mixture was brought to room temperature in N₂ Stir under protection for 16 hours. TLC (petroleum ether:ethyl acetate=30:1) showed that compound 1 was consumed and the desired product was formed. The reaction mixture was diluted with DCM (20 mL) and washed with H₂O (40 mL), washed with anhydrous Na₂SO₄ Dry, filter and concentrate under reduced pressure. The residue was purified by silica gel column chromatography, eluting with petroleum ether:ethyl acetate (1:0-20:1), to obtain compound 3 (4.365 g, 28%) as a colorless oil.

### 2. Synthesis of Intermediate 5

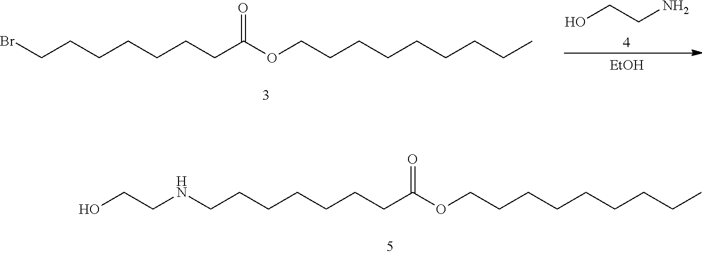

The EtOH solution of compound 3 (500 mg, 1.437 mmol, 1 eq.) and compound 4 (2.63 g, 43.103 mmol, 30 eq.) was dissolved in N₂ Stir at 60 °C for 16 hours under protection. TLC (DCM:MeOH=10:1) showed that compound 3 was consumed and TLC (DCM/MeOH=10/1) showed that anew major spot was observed. The reaction mixture was concentrated under reduced pressure. The residue was diluted with EtOAc (50 mL) and washed with H₂ Wash with O (3×50 mL). Anhydrous Na was used for the organic layer₂SO₄ Dry, filter and concentrate under reduced pressure. The residue was purified by silica gel column chromatography, eluting with DCM/MeOH (1:0-10:1, v/v), to obtain compound 5 (264 mg, 56%) as a yellow oil.

### 3. Synthesis of Intermediate 8

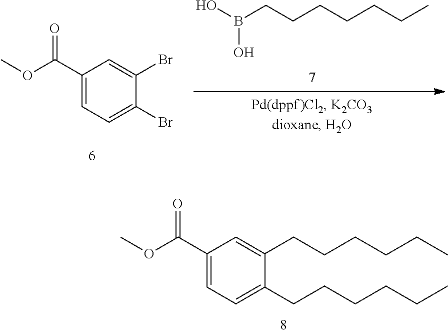

Pd(dppf)Cl was added to compound 6 (500 mg, 1.712 mmol, 1 eq.) and compound (1.113 g, 8.562 mmol, 5 eq.) in dioxane/water (5 mL/0.5 mL) mixed solvent.₂ (112 mg, 0.171 mmol, 0.1 eq.) and potassium carbonate (709 mg, 5.136 mmol, 3 eq.). Place the mixture in N₂ Stir overnight at 100 °C. TLC (PE:EA=15:1) showed the reaction was complete and a new major spot was observed. The mixture was extracted with EA and washed with water, and the organic layer was washed with anhydrous Na₂SO₄ Dry, filter and concentrate in vacuo. The residue was purified by silica gel column chromatography, eluting with PE:EA (1:0-10:1), to give compound 8 as a colorless oil (455 mg, 88%).

### 4. Synthesis of Intermediate 9

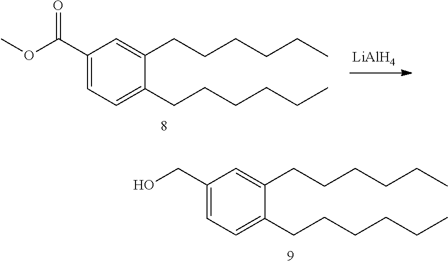

at 0 °C and N₂ Under protection, LiAlH was added to a solution of compound 8 (455 mg, 1.497 mmol, 1 eq.) in THF (5 mL), 4 (1.5 mL, 1.497 mmol, IM in THF, 1 eq.). Bring the mixture to room temperature in N₂ Stir for 2 hours. TLC (PE:EtOAc=5:1) showed the reaction was complete and a new major spot was observed. The mixture was quenched with water (1.5 mL) and treated with 2N HCl to adjust the pH between 6 and 7, extracted with EA and washed with brine. Anhydrous Na was used for the organic layer₂SO₄ Dry, filter and concentrate in vacuo to afford crude compound 9 (419 mg, >100%) as a colorless oil without further purification.

### 5. Synthesis of Intermediate 10

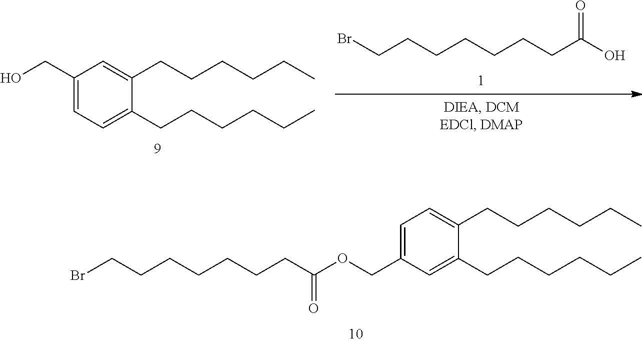 [SMILES: O=C(O)CCCCCCCBr ; CCCCCCc1ccc(CO)cc1CCCCCC ; CCCCCCc1ccc(COC(=O)CCCCCCCBr)cc1CCCCCC]

To the DCM (4 mL) solution containing compound 1 (339 mg, 1.518 mmol, 1 eq.) and compound 9 (419 mg, 1.518 mmol, 1 eq.), EDCI (583 mg, 3.036 mmol, 2 eq.) and DMAP (74 mg, 0.607 mmol, 0.4 eq.), then DIEA (783 mg, 6.072 mmol, 4 eq.) was added. The reaction mixture was brought to room temperature in N₂ Stir under protection for 16 hours. TLC (petroleum ether:ethyl acetate=10:1) showed the desired product was formed. The reaction mixture was extracted with EA and washed with water. Anhdrous Na was used for the organic Na₂SO₄ Dry, filter and concentrate in vacuo. The residue was purified by silica gel column chromatography, eluting with petroleum ether:ethyl acetate (1:0-10:1), to afford compound 10 (443 mg, 60.7%) as a colorless oil.

### 6. Synthesis of Final Product SW-II-115

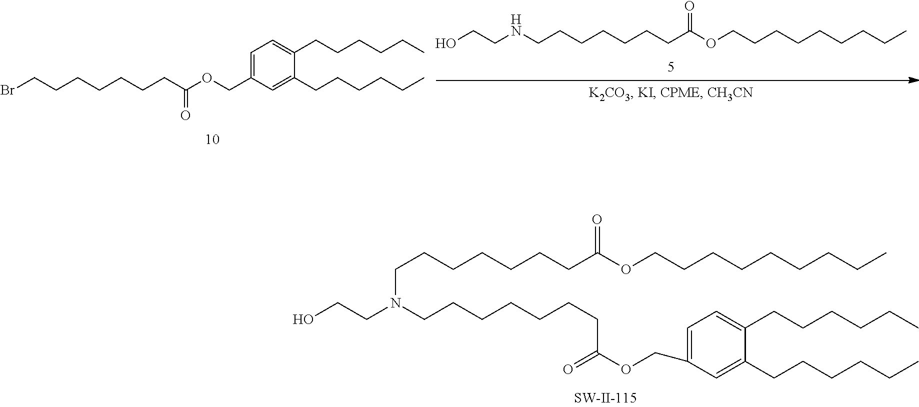

To a mixture containing compound 10 (307 mg, 0.64 mmol, 1 eq.) and compound 5 (210 mg, 0.64 mmol, 1 eq.)

Solvent CPME/CH₃ Add K to CN (3 mL/3 mL)₂ CO₃ (530 mg, 3.84 mmol, 6 eq.) and KI (212 mg, 1.28 mmol, 2 eq.). After the addition is complete, bring the mixture to N₂ Stir overnight at 90 °C. TLC (DCM:MeOH=10:1) showed the reaction was complete and a new major spot was observed. The mixture was extracted with EA and washed with water. Anhydrous Na was used for the organic Na₂SO₄ Dry, filter and concentrate under reduced pressure. The residue was purified by silica gel column chromatography, eluting with DCM:MeOH (1:0-10:1, v/v), to obtain compound SW-II-115 (266 mg, 57%) as a yellow oil.

LCMS: Rt: 1.293 min; MS m/z (ELSD): 730.5 [M+H]⁺;

HPLC: 99.472% purity, ELSD: RT=4.895 min.

¹H NMR (400 MHz, CDCl₃) δ 7.21-6.99 (m, 3H), 5.05 (s, 2H), 4.05 (t, J=6.8 Hz, 2H), 3.58 (t, J=5.3 Hz, 2H), 2.69-2.46 (m, 10H), 2.31 (dt, J=20.0, 7.5 Hz, 4H), 1.69-1.18 (m, 51H), 0.89 (dt, J=12.4, 6.3 Hz, 9H).

¹³C NMR (101 MHz, CDCl₃) δ 173.90 (s), 173.68 (s), 140.80 (d, J=13.0 Hz), 133.31 (s), 129.25 (d, J=16.2 Hz), 128.30 (s), 125.75 (s), 77.30 (d, J=11.5 Hz), 77.04 (s), 76.72 (s), 66.22 (s), 64.43 (s), 58.12 (s), 55.72 (s), 53.90 (s), 34.32 (d, J=1.9 Hz), 32.69 (s), 32.48 (s), 31.81 (d, J=11.2 Hz), 31.25 (s), 29.59-28.91 (m), 28.66 (s), 27.17 (s), 26.64 (s), 25.94 (s), 24.91 (d, J=5.1 Hz), 22.65 (d, J=3.3 Hz), 14.10 (s).

### B. Compound SW-II-118

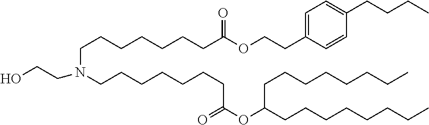 [SMILES: CCCCCCCCC(CCCCCCCC)OC(=O)CCCCCCCN(CCO)CCCCCCCC(=O)OCCc1ccc(CCCC)cc1]

### 1. Synthesis of Intermediate 3

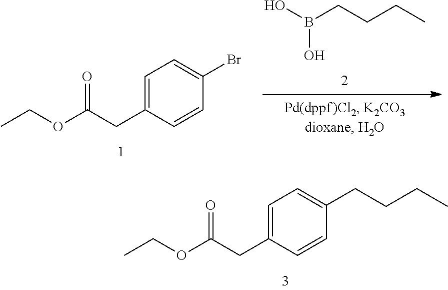

Compound 1 (1.22 g, 5.0 mmol, 1.0 eq.) and compound 2 (765 mg, 7.5 mmol, 1.5 eq.), Pd(PPh₃)₄ (Tetrakis triphenylphosphine palladium, 289 mg, 0.25 mmol, 0.05 eq.) and K₂CO₃ (1.38 g, 10.0 mmol, 2.0 eq.) of toluene (10 ml) and H₂O (1 ml) solution at 110 °C, N₂ Stir protected for 1 hour. TLC (petroleum ether:ethyl acetate=19:1) showed that compound 1 was consumed and a new spot was observed. The reaction mixture was diluted with DCM (50 mL) and washed with H₂O (40 mL), washed with anhydrous Na₂SO₄ Dry, filter and concentrate under reduced pressure. The residue was purified by silica gel column chromatography, eluting with petroleum ether:ethyl acetate (1:0-10:1), to obtain compound 3 (0.5 g, 45%) as a colorless oil.

¹H NMR (400 MHz, CDCl₃) δ 7.16 (dd, J=23.5, 8.1 Hz, 4H), 4.14 (q, J=7.1 Hz, 2H), 3.57 (s, 2H), 2.64-2.48 (m, 2H), 1.66-1.51 (m, 2H), 1.35 (dd, J=15.0, 7.4 Hz, 2H), 1.25 (t, J=7.1 Hz, 3H), 0.92 (t, J=7.3 Hz, 3H).

### 2. Synthesis of Intermediate 4

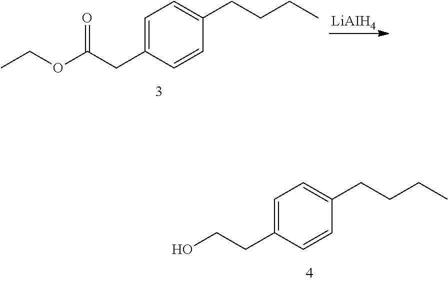

LiAlH at −78 °C₄ (193 mg, 5.09 mmol, 4.0 eq.) was added to a solution containing compound 3 (280 mg, 1.27 mmol, 1.0 eq.) in THF (10 mL), and the reaction was carried out at 10 °C for 3 hours. TLC showed that the reaction was very good, the reaction was concentrated and treated with Na₂SO₄ (20 mL) was diluted and extracted with EA (30 mL×2), and the organic phase was treated with anhydrous Na₂SO₄ Dry, filter and concentrate under reduced pressure to obtain compound 4 (3.12 g, crude product) as yellow oil.

### 3. Synthesis of Intermediate 6

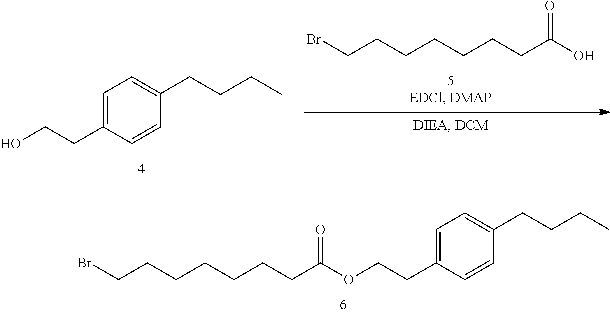

Contains compound 4 (215 mg, 1.2 mmol, 1.0 eq.), compound 5 (404 mg, 1.8 mmol, 1.5 eq.), EDCI (1.15 g, 6.0 mmol, 5.0 eq.), DMAP (732 mg, 1.8 eq.), DIEA (1.29 g, 12.0 mmol, 10.0 eq.) and DIEA (1.29 g, 12.0 mmol, 10.0 eq.) in DCM (5 mL) in N₂ Under protection, stir at 10 °C for 16h. TLC (DCM:MeOH=10:1) showed the reaction was complete and anew major spot was observed. The mixture was concentrated under reduced pressure, and the residue was purified by silica gel column chromatography, eluting with PE:EA (1:0-10:1, v/v) to obtain compound 6 (145 mg, 31%) as a colorless oil.

¹H NMR (400 MHz, CDCl₃) δ 7.12 (s, 4H), 4.27 (t, J=7.1 Hz, 2H), 3.52 (t, J=6.7 Hz, 1H), 3.40 (t, J=6.8 Hz, 1H), 2.90 (t, J=7.1 Hz, 2H), 2.65-2.50 (m, 2H), 2.28 (t, J=7.5 Hz, 2H), 1.93-1.70 (m, 2H), 1.64-1.56 (m, 4H), 1.44-1.27 (m, 8H), 0.92 (t, J=7.3 Hz, 3H).

### 4. Synthesis of Final Product SW-II-118

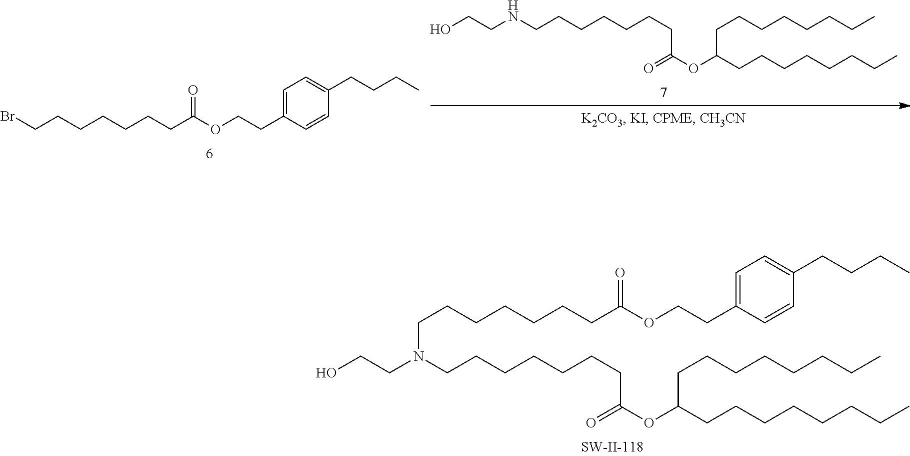 [SMILES: CCCCCCCCC(CCCCCCCC)OC(=O)CCCCCCCNCCO ; CCCCc1ccc(CCOC(=O)CCCCCCCBr)cc1 ; CCCCCCCCC(CCCCCCCC)OC(=O)CCCCCCCN(CCO)CCCCCCCC(=O)OCCc1ccc(CCCC)cc1]

Contains compound 6 (140 mg, 0.37 mmol, 1.0 eq.), compound 7 (243 mg, 0.55 mmol, 1.5 eq.), K₂ CO₃ A mixture of (153 mg, 1.11 mmol, 3.0 eq.) and KI (123 mg, 0.74 mmol, 2.0 eq.) was dissolved in CPME (1 mL) and CH₃CN (1 mL) mixed solvent in N₂ and stirred at 90 °C for 16 hours. The reaction mixture was concentrated under reduced pressure, and the residue was diluted with EtOAc (50 mL) and washed with NaHCO₃ (30 mL) for washing. Anhydrous Na was used for the organic Na₂SO₄ Dry, filter and concentrate under reduced pressure. The residue was purified by silica gel column chromatography, eluting with DCM:MeOH (1:0-10:1, v/v), to give SW-II-118 as a yellow oil (105 mg, 61%).

LCMS: Rt: 1.946 min; MS m/z (ELSD): 744.4 [M+H]⁺;

HPLC: 99.64% purity, ELSD; RT=5.875 min.

¹H NMR (400 MHz, CDCl₃) δ 7.11 (s, 4H), 4.91-4.79 (m, 1H), 4.26 (t, J=7.2 Hz, 2H), 3.80-3.68 (m, 2H), 2.90 (t, J=7.1 Hz, 4H), 2.81-2.67 (m, 4H), 2.62-2.52 (m, 2H), 2.28 (td, J=7.5, 2.6 Hz, 4H), 1.64-1.51 (m, 1H), 1.38-1.17 (m, 42H), 0.93-0.82 (m, 9H).

¹³C NMR (101 MHz, CDCl₃) δ 173.61 (d, J=11.7 Hz), 141.11 (s), 134.90 (s), 128.74 (s), 128.51 (s), 77.40 (s), 77.08 (s), 76. 77 (s), 74.17 (s), 64.90 (s), 57.48 (s), 56.24 (s), 53.98 (s), 35.25 (s), 34.66 (d, J=14.4 Hz), 34.16 (d, J=5.1 Hz), 33.67 (s), 31.86 (s), 29.52 (d, J=2.4 Hz), 29.24 (s), 29.21-28.74 (m), 26.9 (Nd, J=4.9 Hz), 25.42-24.92 (m), 24.92-24.88 (m), 24.74 (s), 22.67 (s), 22.37 (s), 14.04 (d, J=15.7 Hz).

### C. Compound SW-II-120

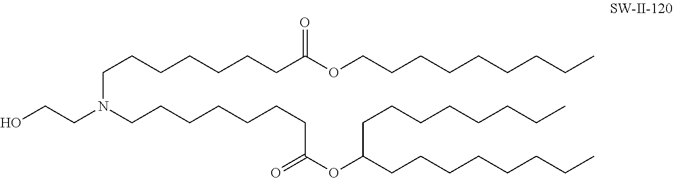 [SMILES: CCCCCCCCCOC(=O)CCCCCCCN(CCO)CCCCCCCC(=O)OC(CCCCCCCC)CCCCCCCC]

### 1. Synthesis of Intermediate 3

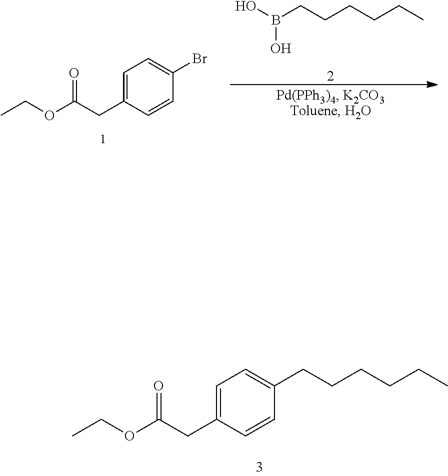

Contains compound 1 (1.22 g, 5.0 mmol, 1.0 eq.), compound 2 (1.30 mg, 10.0 mmol, 2.0 eq.), Pd(PPh₃)₄ (289 mg, 0.25 mmol, 0.05 eq.) and K₂CO₃ (1.38 g, 10.0 mmol, 2.0 eq.) in toluene (10 ml) and H₂ In a mixed solution of O (1 ml) at 110 °C. N₂ Stir protected for 1 hour. TLC (petroleum ether: ethyl acetate=19:1) showed that compound 1 was consumed and a new spot was observed. The reaction mixture was diluted with DCM (50 mL) and washed with H₂O (40 mL), washed with anhydrous Na₂SO₄ Dry, filter and concentrate under reduced pressure. The residue was purified by silica gel column chromatography, eluting with petroleum ether:ethyl acetate (1:0-10:1), to obtain compound 3 (0.78 g, 62%) as a colorless oil.

¹H NMR (400 MHz, CDCl₃) δ 7.19 (d, J=8.1 Hz, 2H), 7.13 (d, J=8.1 Hz, 2H), 4.14 (q, J=7.1 Hz, 2H), 3.57 (s, 2H), 2.62-2.51 (m, 2H), 1.58 (d, J=11.1 Hz, 2H), 1.35-1.21 (m, 9H), 0.88 (t, J=6.7 Hz, 3H).

### 2. Synthesis of Intermediate 4

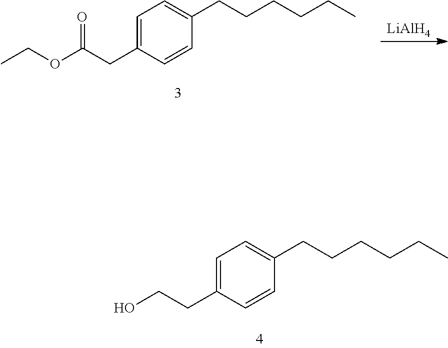 [SMILES: CCCCCCc1ccc(CC(=O)OCC)cc1 ; "LiAlH4" ; CCCCCCc1ccc(CCO)cc1]

LiAlH at −78 °C₄ (477 mg, 12.56 mmol, 4.0 eq.) was added to a solution of compound 3 (780 mg, 3.14 mmol, 1.0 eq.) in THF (10 mL), and the reaction was stirred at 10 °C for 3 hours. Thin layer chromatography showed the reaction was going well. The reaction was concentrated and washed with Na₂SO₄ (20 mL) was diluted and extracted with EA (30 mL*2), and the organic phase was used with anhydrous Na₂SO₄ Dry, filter and concentrate under reduced pressure to obtain compound 4 (640 mg, crude product) as colorless oil.

### 3. Synthesis of Intermediate 6

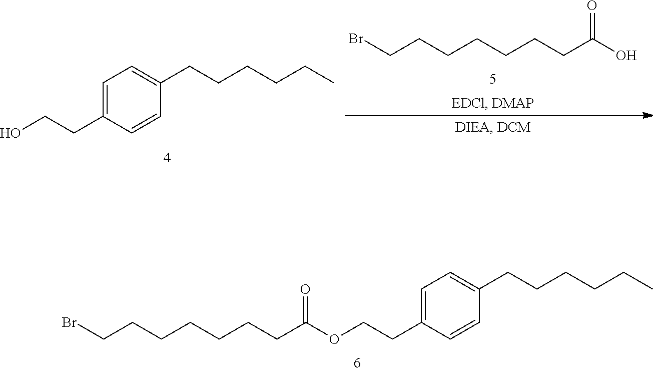 [SMILES: O=C(O)CCCCCCCBr ; CCCCCCc1ccc(CCO)cc1 ; CCCCCCc1ccc(CCOC(=O)CCCCCCCBr)cc1]

Contains compound 4 (640 mg, 3.10 mmol, 1.0 eq.), compound 5 (1.06 g, 4.70 mmol, 1.5 eq.), EDCI

(2.98 g, 15.5 mmol, 5.0 eq.), DMAP (1.85 g, 15.0 eq.) and DIEA (4.0 g, 31.0 mmol, 10.0 eq.) in DCM (10 mL) in N₂ Under protection, stir at 10 °C for 16h. TLC (DCM:MeOH=10:1) showed the reaction was complete and anew major spot was observed. The mixture was concentrated under reduced pressure, and the residue was purified by silica gel column chromatography, eluting with PE:EA (1:0-10:1, v/v), to obtain compound 6 (465 mg, 36%) as a colorless oil.

### 4. Synthesis of Final Product SW-II-120

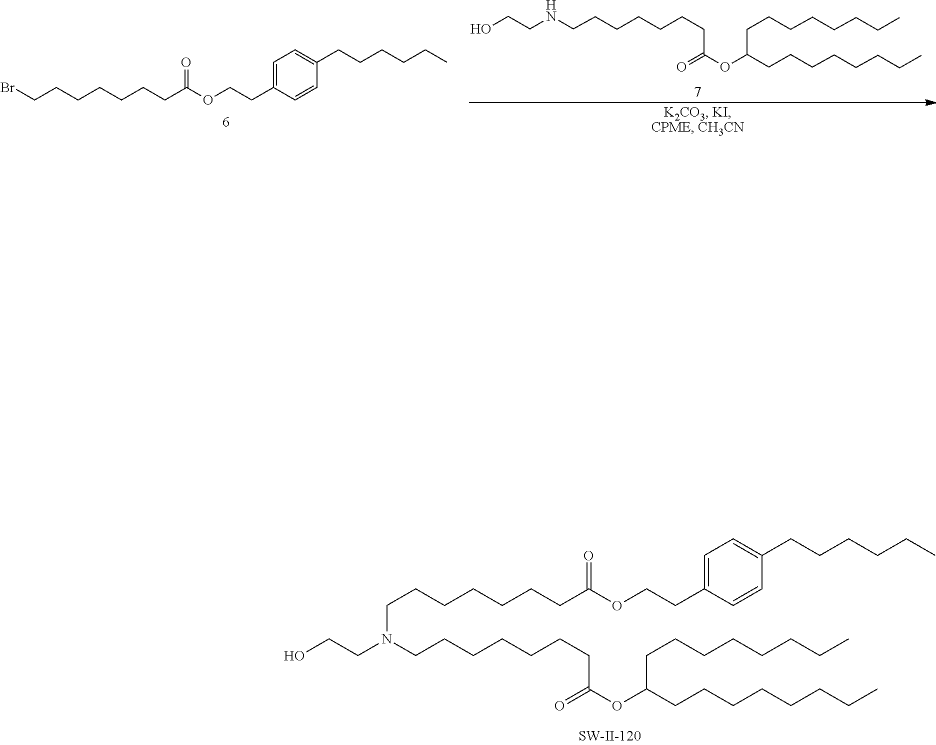

Contains compound 6 (100 mg, 0.25 mmol, 1.0 eq) compound 7 (161 mg, 0.36 mmol, 1.5 eq.), K₂CO₃ A mixture of (104 mg, 0.75 mmol, 3.0 eq.) and KI (83 mg, 0.50 mmol, 2.0 eq.) was dissolved in CPME (1 mL) and CH₃ CN (1 mL) in N₂ and stirred at 90 °C for 16 hours. The reaction mixture was concentrated under reduced pressure, and the residue was diluted with EtOAc (50 mL) and washed with NaHCO₃ (30 mL) for washing. Anhydrous Na was used for the organic Na₂SO₄ Dry, filter and concentrate under reduced pressure. The residue was purified by silica gel column chromatography, eluting with DCM:MeOH (1:0-10:1, v/v), to give SW-II-120 (100 mg, 52%) as a yellow oil.

LCMS: Rt: 2.500 min; MS m/z (ELSD): 772.4 [M+H]⁺;

HPLC: 99.70% purity, ELSD; RT=8.675 min.

¹H NMR (400 MHz, CDCl₃) δ 7.07 (d, J=8.9 Hz, 4H), 4.89-4.73 (m, 1H), 4.23 (t, J=7.2 Hz, 2H), 3.83-3.65 (m, 2H), 2.87 (t, J=7.2 Hz, 4H), 2.82-2.67 (m, 4H), 2.61-2.45 (m, 2H), 2.25 (td, J=7.5, 2.5 Hz, 4H), 1.65-1.44 (m, 15H), 1.27 (dd, J=13.2, 11.3 Hz, 42H), 0.85 (t, J=6.8 Hz, 9H).

¹³C NMR (101 MHz, CDCl₃δ 173.57 (d, J=11.5 Hz), 141.13 (s), 134.88 (s), 128.73 (s), 128.48 (s), 77.45 (s), 77.13 (s), 76.81 (s), 74.14 (s), 64.89 (s), 57.34 (s), 56.17 (s), 53.92 (s), 35.57 (s), 34.64 (d, J=16.1 Hz), 34.14 (d, J=3.3 Hz), 31.79 (d, J=13.4 Hz), 31.49 (s), 29.50 (d, J=2.2 Hz), 29.23 (s), 29.10-28.71 (m), 26.85 (d, J=5.0 Hz), 25.49-25.38 (m), 25.13 (d, J=35.4 Hz), 24.72 (s), 22.63 (d, J=5.8 Hz), 14.11 (s).

### D. Compound SW-II-121

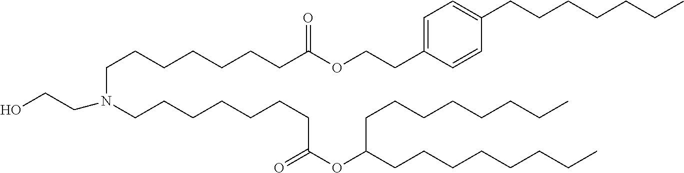 [SMILES: CCCCCCCCC(CCCCCCCC)OC(=O)CCCCCCCN(CCO)CCCCCCCC(=O)OCCc1ccc(CCCCCCC)cc1]

### 1. Synthesis of Intermediate 3

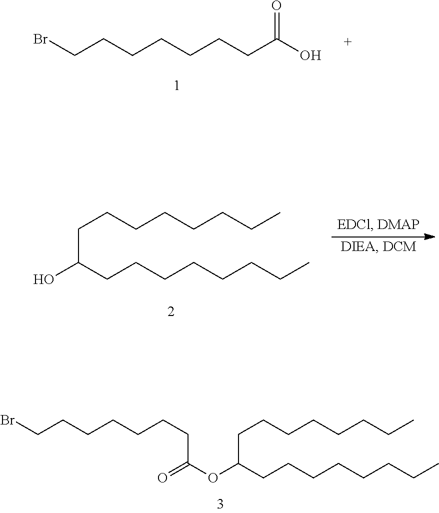 [SMILES: O=C(O)CCCCCCCBr ; CCCCCCCCC(O)CCCCCCCC ; "DIEA, DCM" ; "EDCl, DMAP" ; CCCCCCCCC(CCCCCCCC)OC(=O)CCCCCCCBr]

To the DCM (20 mL) solution containing compound 1 (1.3 g, 5.86 mmol, 1.5 eq.) and compound 2 (1 g, 3.9 mmol, 1.0 eq.), EDCI (1.495 g, 7.8 mmol, 2.0 eq.) and DMAP were added (0.19 g, 1.56 mmol, 0.4 eq.) and DIEA (2.57 mL, 15.6 mmol, 4.0 eq.). The reaction mixture was brought to room temperature in N₂ Stir for 16 hours. TLC(stone Oil ether:ethyl acetate=19:1) showed that compound 2 was consumed and the desired product was formed. The reaction mixture was diluted with DCM (20 mL) and washed with H₂O (40 mL), washed with anhydrous Na₂SO₄ Dry, filter and concentrate under reduced pressure. The residue was purified by silica gel column chromatography, eluting with petroleum ether:ethyl acetate (1:0-10:1), to obtain compound 3 (1.2 g, 66.9%) as a yellow oil.

¹H NMR (400 MHz, CDCl₃) δ 4.92-4.82 (m, 1H), 3.42 (t, J=6.8 Hz, 2H), 2.31 (t, J=7.5 Hz, 2H), 1.95-1.82 (m, 2H), 1.70-1.19 (m, 36H), 0.90 (t, J=6.8 Hz, 6H).

### 2. Synthesis of Intermediate 5

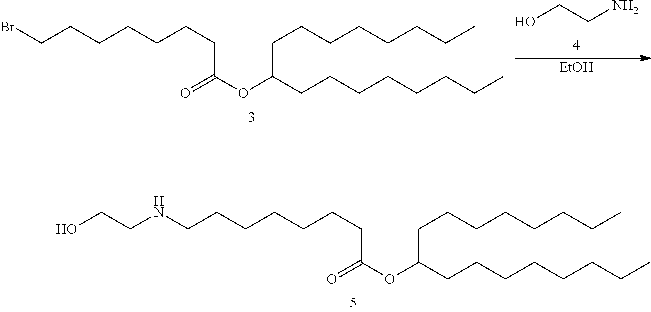

A solution of compound 3 (5.2 g, 11.30 mmol, 1.0 eq.) and compound 4 (20.6 g, 339 mmol, 30 eq.) in EtOH (5 mL) was dissolved in N₂ Stir at 60 °C for 16 hours under protection. TLC (petroleum ether:ethyl acetate=19:1) showed that compound 3 was consumed and TLC (DCM/MeOH=10/1) showed that a new major spot was observed. The reaction mixture was concentrated under reduced pressure, and the residue was diluted with EtOAc (50 mL) and washed with H₂ Wash with O (3-50 mL). Anhydrous Na was used for the organic Na₂SO₄ After drying, filtration and concentration under reduced pressure, the residue was purified by silica gel column chromatography, eluting with DCM:MeOH (1:0-10:1, v/v) to obtain compound 5 (3 g, 60%) as a yellow oil.

### 3. Synthesis of Intermediate 8

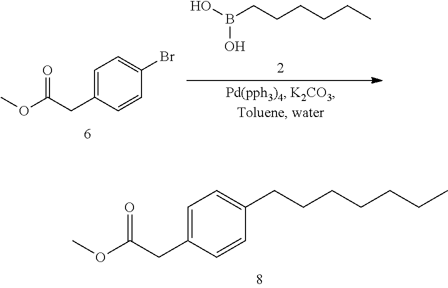

Pd(pph₃)₄ (238 mg, 0.206 mmol, 0.05 eq.), K₂CO₃ (1.7 g, 12.35 mmol, 3 eq). Place the mixture in N₂ and stirred at 110 °C for 2 hours. TLC (PE:EA=10:1) showed the reaction was complete and a new major spot was observed. The mixture was extracted with EA and washed with water. Anhydrous Na was used for the organic Na₂SO₄ Dry, filter and concentrate in vacuo. The residue was purified by silica gel column chromatography, eluting with PE:EA (1:0-10:1), to give compound 8 as a colorless oil (714 mg, 66%).

### 4. Synthesis of Intermediate 9

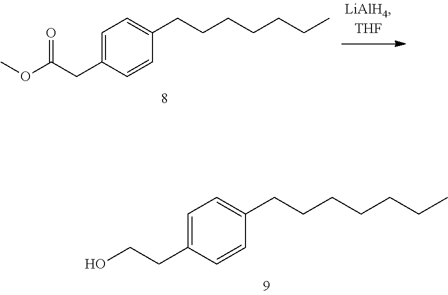 [SMILES: CCCCCCCc1ccc(CC(=O)OC)cc1 ; CCCCCCCc1ccc(CCO)cc1]

N₂ To a mixture of compound 8 (714 mg, 2.725 mmol, 1 eq.) in THF (7 mL) was added LiAlH at 0 °C under protection, 4 (2.7 mL, 2.725 mmol, 1 M in THF, 1 eq.) and the mixture was stirred at room temperature for 2 hours. TLC (PE:EtOAc=10:1) showed the reaction was complete and a new major spot was observed. The mixture was quenched with water (2.7 mL) and treated with 2N HCl to adjust the pH between 6 and 7, extracted with EA and washed with brine. Anhydrous Na was used for the organic Na₂SO₄ Dry, filter and concentrate in vacuo. The residue was purified by silica gel column chromatography, eluting with PE:EA (1:0-10:1), to give compound 9 as a colorless oil (103 mg, 63%).

### 5. Synthesis of Intermediate 11

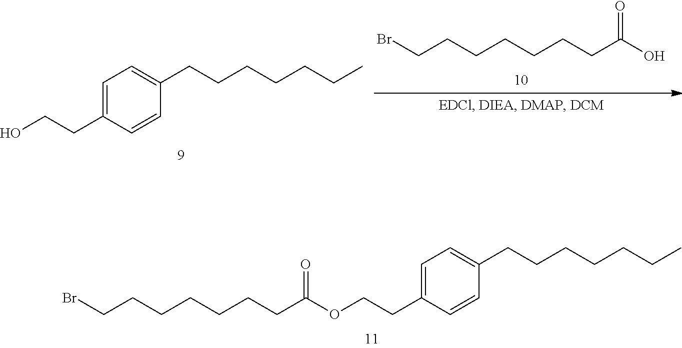

To DCM (3 mL) containing compound 9 (300 mg, 1.364 mmol, 1 eq.) and compound 10 (363 mg, 1.64 mmol, 1.2 eq.), EDCI (524 mg, 2.728 mmol, 2 eq.) and DMAP (67 mg, 0.546 mmol, 0.4 eq.), and DIEA (704 mg, 5.456 mmol, 4 eq.). The reaction mixture was brought to room temperature in N₂ Stir for 16 hours. TLC (petroleum ether:ethyl acetate=10:1) showed the desired product was formed. The reaction mixture was extracted with EA and washed with water. Anhydrous Na was used for the organic Na₂SO₄ Dry, filter and concentrate in vacuo. The residue was purified by silica gel column chromatography, eluting with petroleum ether:ethyl acetate (1:0-10:1), to obtain compound 11 as a colorless oil (169 mg, 29%).

### 6. Synthesis of the Final Product SW-II-121

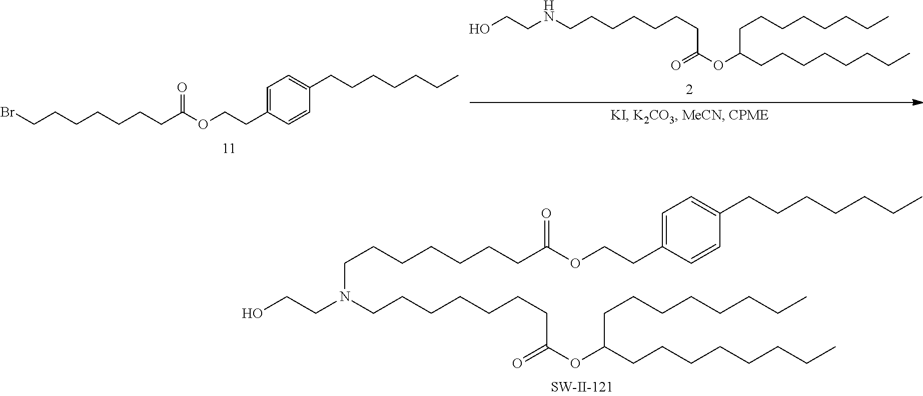 [SMILES: CCCCCCCCC(CCCCCCCC)OC(=O)CCCCCCCNCCO ; CCCCCCCc1ccc(CCOC(=O)CCCCCCCBr)cc1 ; CCCCCCCCC(CCCCCCCC)OC(=O)CCCCCCCN(CCO)CCCCCCCC(=O)OCCc1ccc(CCCCCCC)cc1]

To CPME/CH containing compound 11 (169 mg, 0.399 mmol, 1 eq) and compound 5 (176 mg, 0.399 mmol, 1 eq.) 3 Add K to CN (2 mL/2 mL) mixed solvent₂ CO₃ (330 mg, 2.394 mmol, 6 eq.) and KI (132 mg, 0.798 mmol, 2 eq.). After the addition is complete, bring the mixture to N₂ Stir overnight at 90 °C. TLC (DCM:MeOH=10:1) showed the reaction was complete and a new major spot was observed. The mixture was extracted with EA and washed with water, and the organic layer was washed with anhydrous Na₂SO₄ Dry, filter and concentrate under reduced pressure. The residue was purified by silica gel column chromatography, eluting with DCM:MeOH (1:0-10:1, v/v), to obtain compound SW-II-121 (145 mg, 46%) as a yellow oil.

LCMS: Rt: 1.493 min; MS m/z (ELSD): 786.5 [M+H]⁺;

HPLC: 99.869% purity, ELSD; RT=10.655 min.

¹H NMR (400 MHz, CDCl₃) δ 7.11 (s, 4H), 4.92-4.80 (m, 1H), 4.26 (t, J=7.2 Hz, 2H), 3.80 (s, 2H), 2.87 (dd, J=26.6, 19.4 Hz, 7H), 2.62-2.51 (m, 2H), 2.28 (td, J=7.2, 3.6 Hz, 4H), 1.75-1.45 (m, 14H), 1.42-1.09 (m, 45H), 0.88 (t, J=6.8 Hz, 9H).

¹³C NMR (101 MHz, CDCl₃) δ 173.61 (d, J=12.3 Hz), 141.20 (s), 134.90 (s), 128.75 (s), 128.51 (s), 77.35 (s), 77.03 (s), 76.72 (s), 74.21 (s), 64.93 (s), 54.15 (s), 35.59 (s), 34.66 (d, J=16.6 Hz), 34.16 (d, J=3.0 Hz), 31.85 (d, J=4.4 Hz), 31.55 (s), 29.64-29.15 (m), 29.15-28.78 (m), 26.85 (d, J=4.5 Hz), 25.33 (s), 24.95 (s), 24.72 (s), 22.68 (s), 14.12 (s)).

### E. Compound SW-II-122

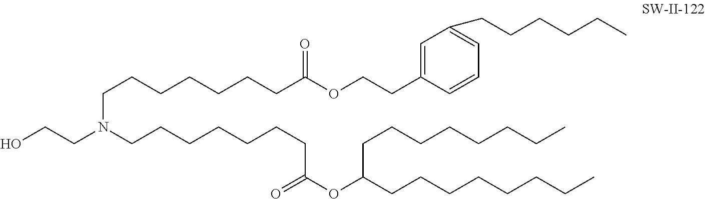 [SMILES: CCCCCCCCC(CCCCCCCC)OC(=O)CCCCCCCN(CCO)CCCCCCCC(=O)OCCc1cccc(CCCCCC)c1]

### 1. Synthesis of Compound 3

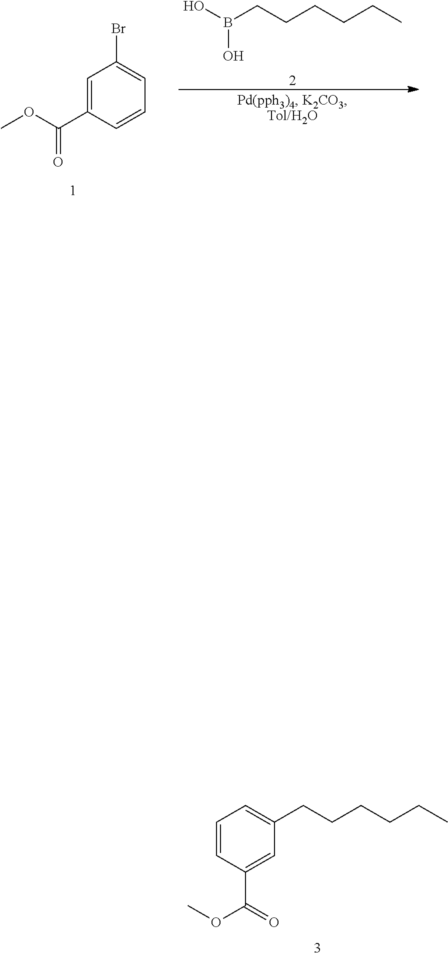

Compound 1 (1 g, 4.65 mmol, 1 eq.) and compound 2 (726 mg, 5.58 mmol, 1.2 eq.) were dissolved in toluene/water (10/1, 20 mL), and then K was added to the mixture₂ CO₃ (1.92 g, 13.9 mmol, 3 eq.) and Pd(pph₃)₄ (269 mg, 0.23 mmol, 0.05 eq). Place the reaction mixture in N₂ Heat to 110 °C and stir for 2 hours. TLC (petroleum ether/ethyl acetate=19/1) showed that compound 1 was consumed and a new major spot was observed. The reaction mixture was reacted with H₂ Quenched with O (80 mL) and extracted with ethyl acetate (60 mL×3), the organic layer was dried over anhydrous sodium sulfate, filtered and concentrated under reduced pressure. The residue was purified by silica gel column chromatography, eluting with petroleum ether/ethyl acetate (1/0-10/1), to obtain compound 3 (800 mg, 78%) as a yellow oil.

### 2. Synthesis of Compound 4

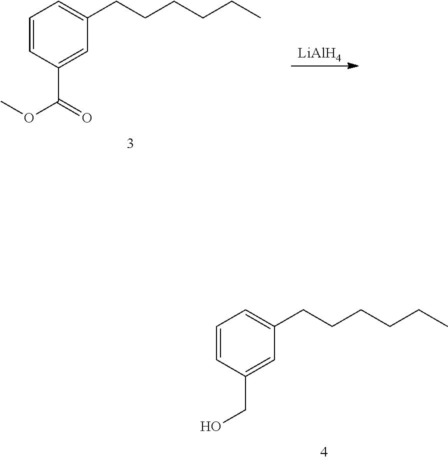 [SMILES: CCCCCCc1cccc(C(=O)OC)c1 ; "LiAlH4" ; CCCCCCc1cccc(CO)c1]

LiAlH was added to compound 3 (700 mg, 3.18 mmol, 1.0 eq.) dissolved in THF (14 mL) under nitrogen protection at 0 °C.₄ (3.2 mL, 3.18 mmol, 1 eq). The reaction mixture was brought to room temperature and stirred under nitrogen protection for 2 hours. TLC (PE/EtOAc=10/1) showed the reaction was complete and new major spots were observed. The mixture was quenched with water (3.2 mL) and IM HCl (3.2 mL) respectively. Water (6 mL) was added to the mixture, and the mixture was extracted with ethyl acetate (60 mL×3). The organic layer was washed with brine (30 mL×2), dried over anhydrous sodium sulfate, filtered, and concentrated under reduced pressure. The residue was purified by silica gel column chromatography, eluting with ethyl acetate/petroleum ether=1/10, to obtain compound 4 (600 mg, 98%) as a yellow oil.

### 3. Synthesis of Compound 6

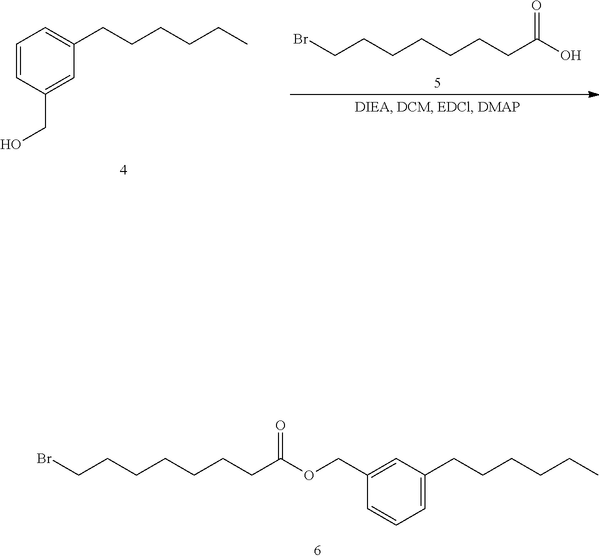

Compound 4 (680 mg, 3.5 mmol, 1.0 eq.) and compound 5 (1.13 g, 5.1 mmol, 1.5 eq.) were dissolved in DCM (10 mL), and EDCI (1.20 g, 6.25 mmol, 2.0 eq.) was added to the mixture.), DMAP (166 mg, 1.36 mmol, 0.4 eq.) and DIEA (1.78 g, 13.8 mmol, 4.0 eq.). After the addition, the reaction mixture was stirred at room temperature overnight under nitrogen protection. TLC (DCM/MeOH=30/1) showed consumption of starting material and formation of a new spot. The mixture was quenched with water (70 mL) and extracted with DCM (80 mL×3). The combined organic layers were washed with brine (2×20 mL), dried over anhydrous sodium sulfate, filtered and concentrated under reduced pressure. The residue was purified by silica gel column chromatography, eluting with a solution of ethyl acetate/petroleum ether=3/97, to obtain compound 6 (680 mg, 48.5%) as a yellow oil.

### 4. Synthesis of SW-II-122

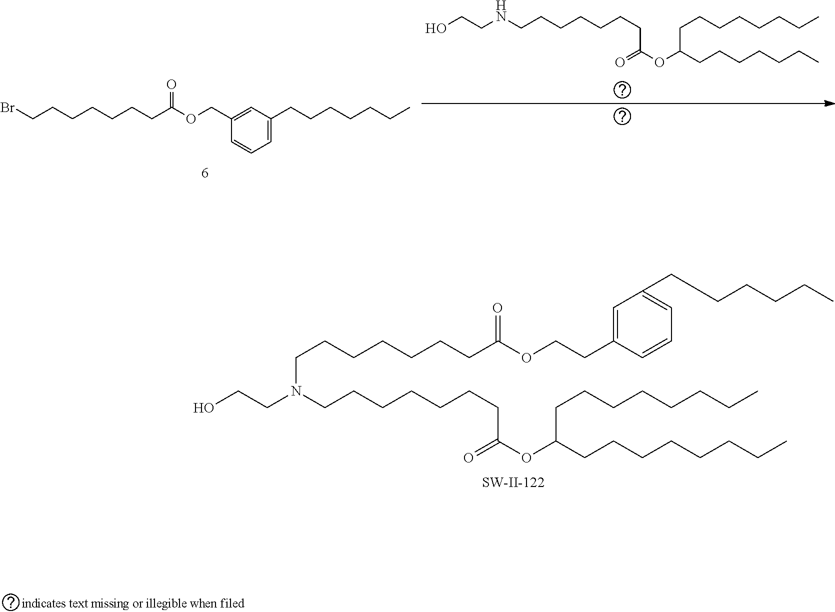 [SMILES: CCCCCCCCC(CCCCCCC)OC(=O)CCCCCCCNCCO ; CCCCCCCc1cccc(COC(=O)CCCCCCCBr)c1 ; CCCCCCCCC(CCCCCCCC)OC(=O)CCCCCCCN(CCO)CCCCCCCC(=O)OCCc1cccc(CCCCCC)c1]

Compound 6 (108 mg, 0.27 mmol, 1.2 eq.) and compound 7 (100 mg, 0.23 mmol, 1 eq.) were dissolved in CPME (2 mL) and CH₃ CN (2 mL), to the mixture were added potassium carbonate (157 mg, 1.14 mmol, 5.0 eq) and potassium iodide (75 mg, 0.45 mmol, 2.0 eq). After the addition, the reaction mixture was stirred at 90 °C for 16 hours under nitrogen protection. TLC (DCM/MeOH=10/1) showed the reaction was complete. The reaction mixture was concentrated under reduced pressure. The residue was purified by silica gel column chromatography, eluting with DCM/MeOH (1/0-10:1, v/v), to give SW-II-122 (68 mg, 40%) as a colorless oil.

LCMS: Rt: 1.487 min; MS m/z (ELSD): 758.5 [M+H]⁺;

HPLC: 97.3% purity, ELSD; RT=7.622 min.

¹H NMR (400 MHz, CDCl₃) δ 7.32 (d, J=26.4 Hz, 1H), 7.17 (dd, J=27.2, 21.1 Hz, 3H), 5.09 (s, 2H), 4.91-4.79 (m, 1H), 3.85 (s, 2H), 2.98 (s, 2H), 2.87 (s, 4H), 2.65-2.54 (m, 2H), 2.35 (t, J=7.6 Hz, 2H), 2.28 (t, J=7.6 Hz, 2H), 1.74-1.57 (m, 9H), 1.50 (d, J=5.6 Hz, 4H), 1.37-1.15 (m, 43H), 0.94-0.80 (m, 9H).

¹³C NMR (101 MHz, CDCl₃) δ 173.55 (d, J=2.4 Hz), 143.35 (s), 135.92 (s), 128.67-128.19 (m), 125.47 (s), 77.36 (s), 77.04 (s), 76.73 (s), 74.22 (s), 66.27 (s), 57.15 (s), 56.74 (s), 54.14 (s), 35.88 (s), 34.55 (s), 34.15 (d, J=3.6 Hz), 31.79 (d, J=15.2 Hz), 31.43 (s), 29.52 (d, J=2.8 Hz), 29.25 (s), 28.92 (dd, J=14.2, 5.8 Hz), 26.77 (d, J=4.8 Hz), 25.33 (s), 24.92 (s), 24.71 (s), 24.48 (s), 22.64 (d, J=6.8 Hz), 14.12 (s).

### F. Compound SW-II-127

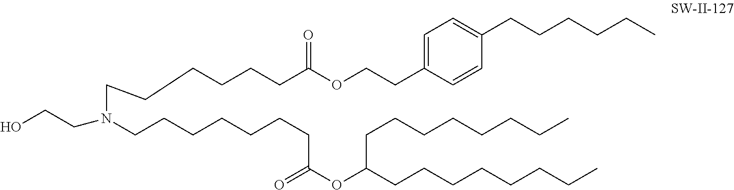 [SMILES: CCCCCCCCC(CCCCCCCC)OC(=O)CCCCCCCN(CCO)CCCCCCCC(=O)OCCc1ccc(CCCCCC)cc1]

### 1. Synthesis of Compound 3

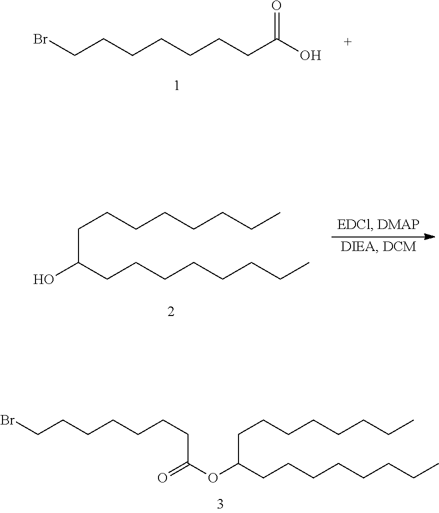 [SMILES: O=C(O)CCCCCCCBr ; CCCCCCCCC(O)CCCCCCCC ; "DIEA, DCM" ; "EDCl, DMAP" ; CCCCCCCCC(CCCCCCCC)OC(=O)CCCCCCCBr]

Compound 1 (1.3 g, 5.86 mmol, 1.5 eq) and compound 2 (1 g, 3.9 mmol, 1.0 eq.) were dissolved in DCM (20 mL), and EDCI (1.495 g, 7.8 mmol, 2.0 eq.) was added to the mixture.) and DMAP (0.19 g, 1.56 mmol, 0.4 eq.), then DIEA (2.57 mL, 15.6 mmol, 4.0 eq.) was added. The reaction mixture was stirred at room temperature under nitrogen protection for 16 hours. TLC (petroleum ether/ethyl acetate=19/1) showed consumption of compound 2 and formation of the desired product. The reaction mixture was diluted with DCM (20 mL) and washed with H₂O (40 mL), washed with anhydrous Na₂SO. Dry, filter and concentrate under reduced pressure. The residue was purified by silica gel column chromatography, eluting with petroleum ether/ethyl acetate (1/0-10/1), to obtain compound 3 (1.2 g, 66.9%) as a yellow oil.

¹H NMR (400 MHz, CDCl₃) δ 4.92-4.82 (m, 1H), 3.42 (t, J=6.8 Hz, 2H), 2.31 (t, J=7.5 Hz, 2H), 1.95-1.82 (m, 2H), 1.70-1.19 (m, 36H), 0.90 (t, J=6.8 Hz, 6H).

### 2. Synthesis of Compound 5

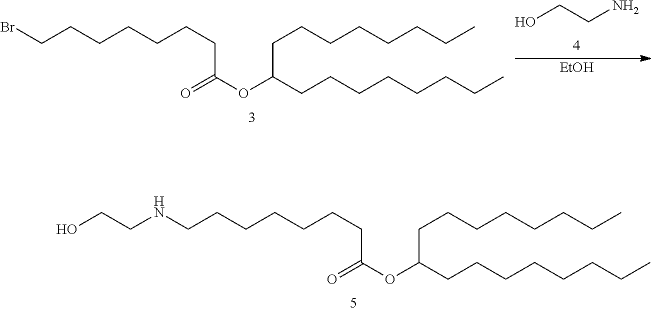

Compound 3 (5.2 g, 11.30 mmol, 1.0 eq.) and compound 4 (20.6 g, 339 mmol, 30 eq.) were added to EtOH (5 mL), and then the mixture was stirred at 60 °C for 16 hours under nitrogen protection. TLC (petroleum ether/ethyl acetate=19/1) showed that compound 3 was consumed and TLC (DCM/MeOH=10/1) showed that a new major spot was observed. The reaction mixture was concentrated under reduced pressure. The residue was diluted with EtOAc (50 mL) and washed with H₂ Wash with O (3×50 mL). The organic layer was dried over anhydrous sodium sulfate, filtered and concentrated under reduced pressure. The residue was purified by silica gel column chromatography, eluting with DCM/MeOH (1/0-10:1, v/v), to obtain compound 5 as a yellow oil (3 g, 60%).

¹H NMR (400 MHz, CDCl₃) δ 4.95-4.75 (m, 1H), 3.74-3.58 (m, 2H), 2.87-2.74 (m, 2H), 2.69-2.56 (m, 2H), 2.36 (s, 2H), 2.28 (t, J=7.5 Hz, 2H), 1.65-1.42 (m, 8H), 1.38-1.17 (m, 30H), 0.88 (t, J=6.8 Hz, 6H).

### 3. Synthesis of Compound 8

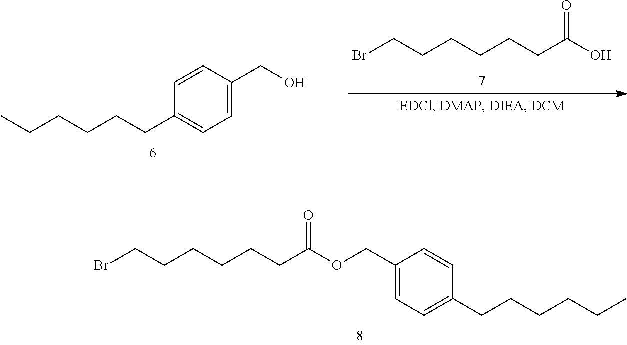 [SMILES: O=C(O)CCCCCCBr ; CCCCCCc1ccc(CO)cc1 ; CCCCCCc1ccc(COC(=O)CCCCCCBr)cc1]

Compound 7 (522 mg, 2.5 mmol, 1.2 eq.) and compound 6 (400 mg, 2.083 mmol, 1 eq.) were dissolved in DCM (4 mL), and EDCI (800 mg, 4.166 mmol, 2 eq.), DMAP (102 mg, 0.833 mmol, 0.4 eq.) and DIEA (1.075 mg, 8.332 mmol, 4 eq.). After the addition, the reaction mixture was stirred at room temperature overnight under nitrogen protection. TLC (PE:EA=10:1) showed that the starting material was consumed and a new spot formed. The reaction mixture was concentrated under reduced pressure. The residue was purified by silica gel column chromatography, eluting with PE/EA (1/0-10/1), to obtain compound 8 (454 mg, 57%) as a colorless oil.

### 4. Synthesis of SW-II-127

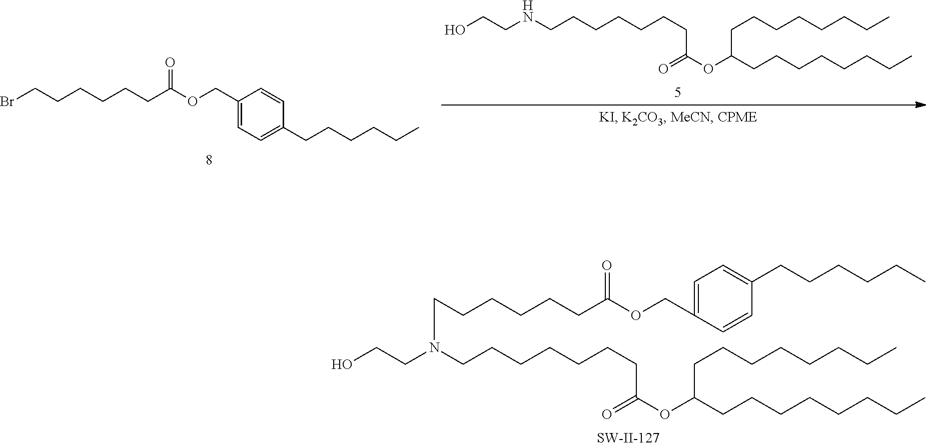 [SMILES: CCCCCCc1ccc(COC(=O)CCCCCCBr)cc1 ; CCCCCCCCC(CCCCCCCC)OC(=O)CCCCCCCNCCO ; CCCCCCCCC(CCCCCCCC)OC(=O)CCCCCCCN(CCO)CCCCCCC(=O)OCc1ccc(CCCCCC)cc1]

Compound 8 (100 mg, 0.262 mmol, 1 eq.) and compound 5 (139 mg, 0.314 mmol, 1.2 eq.) were dissolved in CPME/CH3CN (1 mL/1 mL), and potassium carbonate (217 mg, 1.572 mmol, 6 eq.) was added to the mixture.) and potassium iodide (87 mg, 0.524 mmol, 2 eq.). After the addition, the reaction mixture was stirred at 90 °C overnight under nitrogen protection. TLC (DCM/MeOH=10/1) showed that the reaction was complete and the desired product was formed. The mixture was extracted with ethyl acetate and washed with water. The organic layer was dried over anhydrous sodium sulfate, filtered and concentrated under reduced pressure. The residue was purified by silica gel column chromatography, eluting with DCM/MeOH (1/0-10:1, v/v), to obtain compound SW-II-127 (42.49 mg, 22%) as a yellow oil.

LCMS: Rt: 1.323 min; MS m/z (ELSD): 744.5[M+H]⁺;

HPLC: 99.742% purity. ELSD: RT=7.339 min.

¹H NMR (400 MHz, CDCl₃) δ 7.25 (s, 2H), 7.17 (d, J=8.0 Hz, 2H), 5.07 (s, 2H), 4.91-4.82 (m, 1H), 3.83 (s, 2H), 2.90 (d, J=44.8 Hz, 5H), 2.64-2.55 (m, 2H), 2.35 (t, J=7.4 Hz, 2H), 2.28 (t, J=7.5 Hz, 2H), 1.76-1.46 (m, 14H), 1.42-1.19 (m, 41H), 0.88 (t, J=6.8 Hz, 9H).

¹³C NMR (101 MHz, CDCl₃δ 173.50 (d, J=8.5 Hz), 133.17 (s), 128.61 (s), 128.34 (s), 77.29 (d, J=11.4 Hz), 77.03 (s), 76.7 I(s), 74.23 (s), 66.19 (s), 54.20 (s), 35.71 (s), 34.56 (s), 34.10 (d, J=8.8 Hz), 31.80 (d, J=15.4 Hz), 31.43 (s), 29.53 (d, J=2.5 Hz), 29.25 (s), 28.95 (d, J=1 0.5 Hz), 28.63 (s), 26.71 (d, J=18.2 Hz), 25.33 (s), 24.93 (s), 24.62 (s), 22.65 (d, J=6.6 Hz), 14.13 (s).

### G. Compound SW-II-134-1

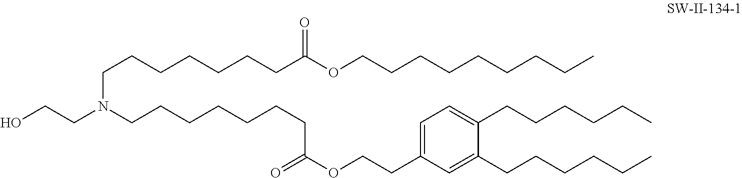 [SMILES: CCCCCCCCCOC(=O)CCCCCCCN(CCO)CCCCCCCC(=O)OCCc1ccc(CCCCCC)c(CCCCCC)c1]

### 1. Synthesis of Compound 3

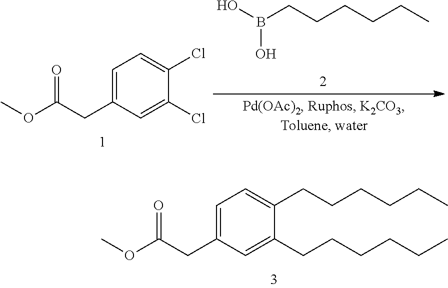

To a mixture of compound 1 (500 mg, 2.283 mmol, 1 eq.) and compound 2 (890 mg, 6.849 mmol, 3 eq.) in toluene/water (5 mL/1 mL) was added palladium acetate (51 mg, 0.228 mmol, 0.1 eq.), Ruphos (213 mg, 0.457 mmol, 0.2 eq.) and potassium carbonate (945 mg, 6.849 mmol, 3 eq.). The mixture was stirred at 110 °C overnight under nitrogen. TLC (PE/EA=20/1) showed the reaction was complete and new major spots were observed. The mixture was extracted with ethyl acetate and washed with water. The organic layer was dried over anhydrous sodium sulfate, filtered and concentrated in vacuo. The residue was purified by silica gel column chromatography, eluting with PE/EA (1/0-20/1), to obtain compound 3 (723 mg, 99.6%) as a colorless oil.

### 2. Synthesis of Compound 4

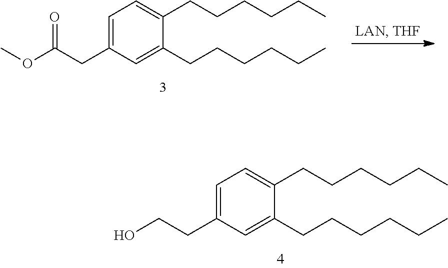 [SMILES: CCCCCCc1ccc(CC(=O)OC)cc1CCCCCC ; CCCCCCc1ccc(CCO)cc1CCCCCC]

To a mixture of compound 3 (723 mg, 2.27 mmol, 1 eq.) in THF (8 mL) was added lithium aluminum hydride (2.3 mL, 2.27 mmol, 1 M, 1 eq. in THF) at 0 °C under nitrogen. The mixture was stirred at room temperature for 3 hours. TLC (PE/EA=5/1) indicated that the reaction was complete and new major spots were observed. The mixture was quenched with water (2.3 mL) and treated with 2N hydrochloric acid to adjust the pH between 6 and 7, extracted with ethyl acetate and washed with brine. The organic layer was dried over anhydrous sodium sulfate, filtered and concentrated in vacuo to afford compound 4 (381 mg, 58%) as a colorless oil without further purification.

### 3. Synthesis of Compound 6

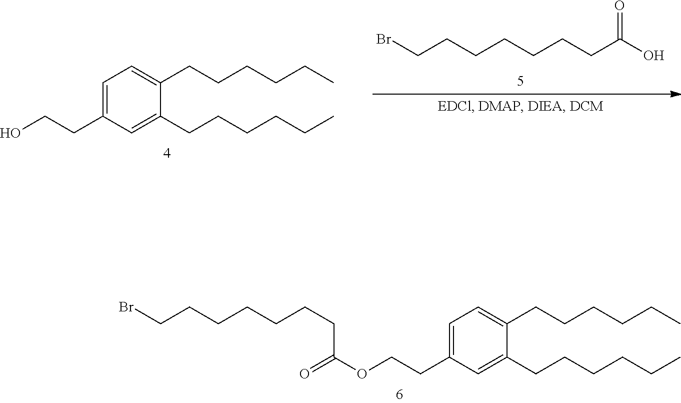 [SMILES: O=C(O)CCCCCCCBr ; CCCCCCc1ccc(CCO)cc1CCCCCC ; CCCCCCc1ccc(CCOC(=O)CCCCCCCBr)cc1CCCCCC]

To a mixture of compound 4 (381 mg, 1.3 mmol, 1 eq.) and compound 5 (352 mg, 1.6 mmol, 1.2 eq.) in DCM (4 mL) was added EDCI (499 mg, 2.6 mmol, 2 eq.) and DMAP (63 mg, 0.52 mmol, 0.4 eq.), then DIEA (671 mg, 5.2 mmol, 4 eq.) was added. The reaction mixture was stirred at room temperature under nitrogen for 16 hours. TLC (petroleum ether/ethyl acetate=20/1) showed the formation of the desired product. The reaction mixture was extracted with ethyl acetate and washed with water. The organic layer was dried over anhydrous sodium sulfate, filtered and concentrated in vacuo. The residue was purified by silica gel column chromatography using stone Elution with oil ether/ethyl acetate (1/0-20/1) gave compound 6 (272 mg, 44%) as a colorless oil.

### 4. Synthesis of SW-II-134-1

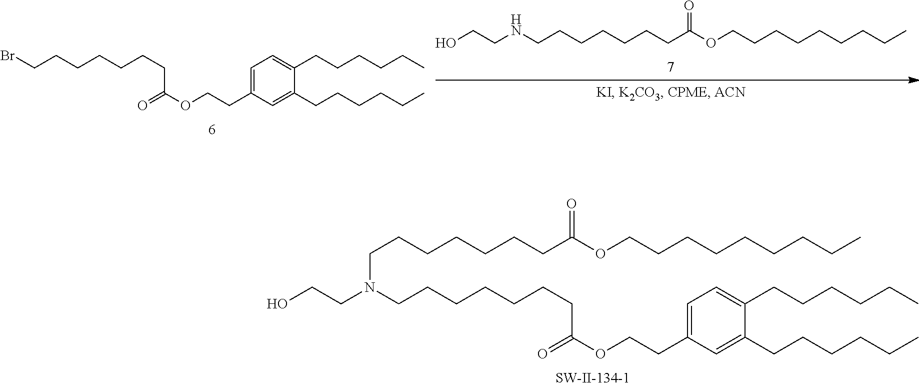

To compound 6 (150 mg, 0.303 mmol, 1 eq.) and compound 7 (110 mg, 0.333 mmol, 1.1 eq.) in CPME/CH₃Potassium carbonate (251 mg, 1.818 mmol, 6 eq.) and potassium iodide (101 mg, 0.61 mmol, 2 eq.) were added to the mixture in CN (2 mL/2 mL). After addition, the mixture was stirred at 90 °C overnight under nitrogen. TLC (DCM/MeOH=15/1) showed the reaction was complete and new major spots were observed. The mixture was extracted with ethyl acetate and washed with water. The organic layer was dried over anhydrous sodium sulfate, filtered and concentrated under reduced pressure. The residue was purified by silica gel column chromatography, eluting with DCM/MeOH (1/0-10:1, v/v), to obtain compound SW-II-134-1 (168 mg, 75%) as a yellow oil.

LCMS: Rt: 1.276 min; MS m/z (ELSD): 744.4[M+H]⁺;

HPLC: 98.481% purity, ELSD: RT=10.724 min.

¹H NMR (400 MHz, CDCl₃) δ 7.06 (d, J=7.6 Hz, 1H), 7.01-6.93 (m, 2H), 4.25 (t, J=7.3 Hz, 2H), 4.05 (t, J=6.8 Hz, 2H), 3.85-3.72 (m, 2H), 2.98-2.69 (m, 8H), 2.62-2.48 (m, 4H), 2.29 (t, J=7.5 Hz, 4H), 1.72-1.48 (m, 14H), 1.45-1.17 (m, 36H), 0.89 (dt, J=11.9, 6.0 Hz, 9H).

¹³CNMR (101 MHz, CDCl₃) δ 173.78 (d, J=16.7 Hz), 140.72 (s), 138.81 (s), 134.91 (s), 129.70 (s), 129.22 (s), 126.19 (s), 7 7.30 (d, J=11.4 Hz), 77.03 (s), 76.72 (s), 65.02 (s), 64.49 (s), 57.42 (s), 56.36 (s), 54.08 (s), 34.76 (s), 34.22 (d, J=4.2 Hz), 32.74 (s), 32.36 (s), 31.81 (d, J=9.1 Hz), 31.35 (d, J=5.3 Hz), 29.49 (d, J=2.8 Hz), 29.2 4 (d, J=2.2 Hz), 28.92 (s), 28.66 (s), 26.86 (s), 25.93 (s), 25.04 (s), 24.78 (d, J=6.6 Hz), 22.65 (d, J=2.6H z), 14.10 (s).

### H. Compound SW-II-134-2

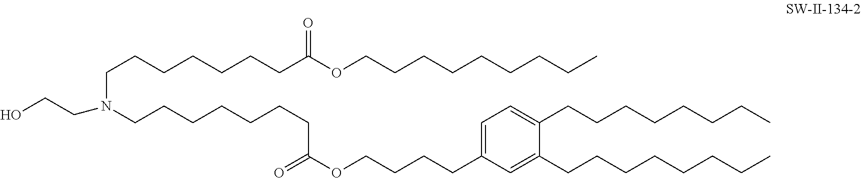 [SMILES: CCCCCCCCCOC(=O)CCCCCCCN(CCO)CCCCCCCC(=O)OCCCCc1ccc(CCCCCCCC)c(CCCCCCCC)c1]

### 1. Synthesis of Compound 3

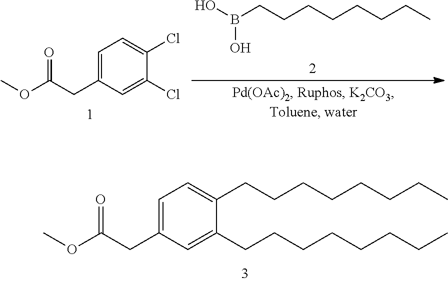 [SMILES: COC(=O)Cc1ccc(Cl)c(Cl)c1 ; CCCCCCCCB(O)O ; CCCCCCCCc1ccc(CC(=O)OC)cc1CCCCCCCC]

To a mixture of compound 1 (500 mg, 2.283 mmol, 1 eq.) and compound 2 (1.08 g, 6.849 mmol, 3 eq.) in toluene/water (5 mL/1 mL) was added palladium acetate (51 mg, 0.228 mmol, 0.1 eq.), Ruphos (213 mg, 0.457 mmol, 0.2 eq.) and potassium carbonate (945 mg, 6.849 mmol, 3 eq.). The mixture was stirred at 110 °C overnight under nitrogen. TLC (PE/EA=20/1) showed the reaction was complete and new major spots were observed. The mixture was extracted with ethyl acetate and wash with water The organic layer was dried over anhydrous sodium sulfate, filtered and concentrated in vacuo. The residue was purified by silica gel column chromatography, eluting with PE/EA (1/0-20/1), to obtain compound 3 (854 mg, 100%) as a colorless oil.

### 2. Synthesis of Compound 4

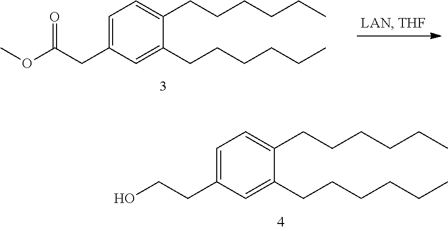 [SMILES: CCCCCCc1ccc(CC(=O)OC)cc1CCCCCC ; CCCCCCc1ccc(CCO)cc1CCCCCC]

To a mixture of compound 3 (854 mg, 2.28 mmol, 1 eq.) in THF (9 mL) was added lithium aluminum hydride (2.3 mL, 2.28 mmol, 1 M, 1 eq. in THF) at 0 °C under nitrogen. The mixture was stirred at room temperature for 3 hours. TLC (PE/EA=5/1) indicated that the reaction was complete and new major spots were observed. The mixture was quenched with water (2.3 mL) and treated with 2N hydrochloric acid to adjust the pH between 6 and 7, extracted with ethyl acetate and washed with brine. The organic layer was dried over anhydrous sodium sulfate, filtered and concentrated in vacuo to afford compound 4 (724 mg, 92%) as a colorless oil without further purification.

### 3. Synthesis of Compound 6

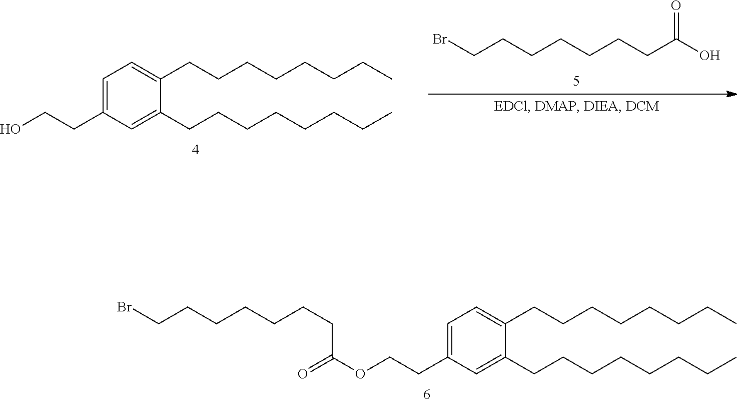 [SMILES: O=C(O)CCCCCCCBr ; CCCCCCCCc1ccc(CCO)cc1CCCCCCCC ; CCCCCCCCc1ccc(CCOC(=O)CCCCCCCBr)cc1CCCCCCCC]

To a mixture of compound 4 (724 mg, 2.09 mmol, 1 eq.) and compound 5 (560 mg, 2.51 mmol, 1.2 eq.) in DCM (8 mL) was added EDCI (803 mg, 4.18 mmol, 2 eq.) and DMAP (102 mg, 0.84 mmol, 0.4 eq.), then DIEA (1.078 g, 8.36 mmol, 4 eq.) was added. The reaction mixture was stirred at room temperature under nitrogen for 16 hours. TLC (petroleum ether/ethyl acetate=20/1) showed the formation of the desired product. The reaction mixture was extracted with ethyl acetate and washed with water. The organic layer was dried over anhydrous sodium sulfate, filtered and concentrated in vacuo. The residue was purified by silica gel column chromatography, eluting with petroleum ether/ethyl acetate (1/0-20/1), to obtain compound 6 (473 mg, 41%) as a colorless oil.

### 4. Synthesis of SW-II-134-2

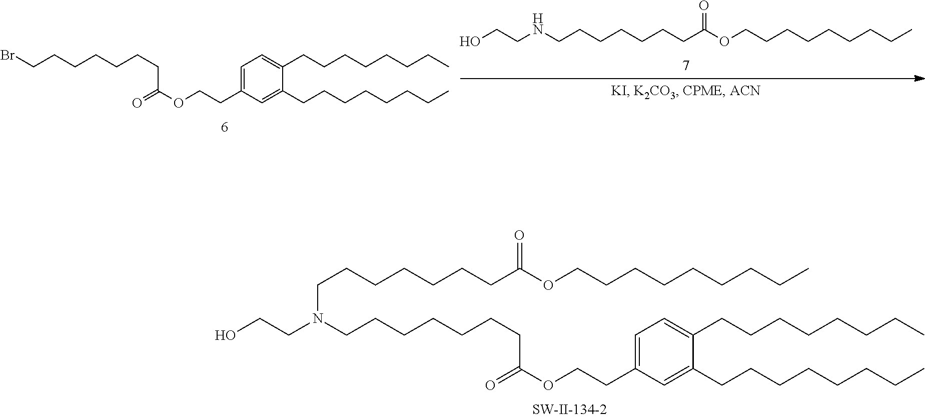

To compound 6 (150 mg, 0.27 mmol, 1 eq.) and compound 7 (108 mg, 0.33 mmol, 1.1 eq.) in CPME/CH₃ Potassium carbonate (225 mg, 1.63 mmol, 6 eq.) and potassium iodide (90 mg, 0.54 mmol, 2 eq.) were added to the mixture in CN (2 mL/2 mL). After addition, the mixture was stirred at 90 °C overnight under nitrogen. TLC (DCM/MeOH=15/1) showed the reaction was complete and new major spots were observed. The mixture was extracted with ethyl acetate and washed with water. The organic layer was dried over anhydrous sodium sulfate, filtered and concentrated under reduced pressure. The residue was purified by silica gel column chromatography, eluting with DCM/MeOH (1/0-10:1, v/v), to obtain compound SW-II-134-2 (71.77 mg, 33%) as a yellow oil.

LCMS: Rt: 1.527 min; MS m/z (ELSD): 800.4 [M+H]⁺;

HPLC: 97.311% purity, ELSD: RT=9.025 min.

¹H NMR (400 MHz, CDCl₃) δ 7.06 (d, J=7.6 Hz, 1H), 6.96 (d, J=9.6 Hz, 2H), 4.25 (t, J=7.3 Hz, 2H), 4.05 (t, J=6.8 Hz, 2H), 3.80-3.66 (m, 2H), 2.86 (dd, J=12.8, 5.6 Hz, 4H), 2.78-2.67 (m, 4H), 2.60-2.52 (m, 4H), 2.29 (t, J=7.5 Hz, 4H), 1.57 (dt, J=15.8, 7.3 Hz, 14H), 1.30 (d, J=20.3 Hz, 45H), 0.88 (t, J=6.7 Hz, 9H).

¹³C NMR (101 MHz, CDCl₃) δ 173.82 (d, J=16.9 Hz), 140.73 (s), 138.82 (s), 134.91 (s), 129.71 (s), 129.23 (s), 126.19 (s), 7 7.36 (s), 77.14 (d, J=20.4 Hz), 76.72 (s), 65.03 (s), 64.49 (s), 57.57 (s), 56.13 (s), 54.02 (s), 34.76 (s), 34.25 (d, J=4.2 Hz), 32.76 (s), 32.37 (s), 31.89 (d, J=5.3 Hz), 31.40 (d, J=6.0 Hz), 29.84 (d, J=3.7 Hz), 29.63-29.14 (m), 28.97 (s), 28.65 (s), 26.93 (s), 25.66 (d, J=54.4 Hz), 24.80 (d, J=6.6 Hz), 22.68 (d, J=1.8 Hz), 14.12 (s).

### I. Compound SW-II-134-3

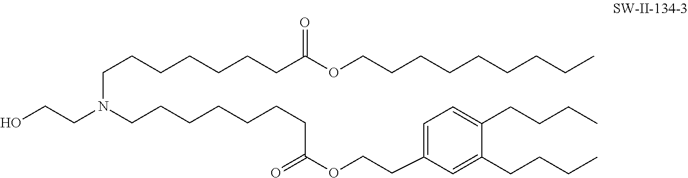 [SMILES: CCCCCCCCCOC(=O)CCCCCCCN(CCO)CCCCCCCC(=O)OCCc1ccc(CCCC)c(CCCC)c1]

### 1. Synthesis of Compound 3

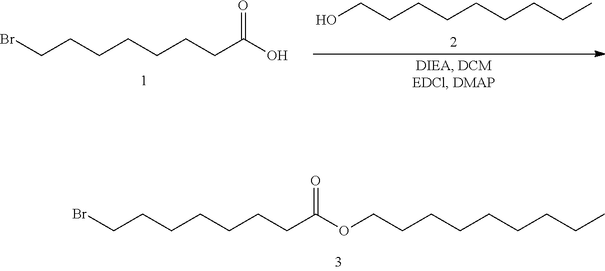

To a mixture of compound 1 (10 g, 45 mmol, 1 eq.) and compound 2 (7.8 g, 54 mmol, 1.2 eq.) in DCM (100 mL) was added EDCI (17.3 g, 90 mmol, 2 eq.) and DMAP (2.2 g, 18 mmol, 0.4 eq.), then DIEA (23.2 g, 180 mmol, 4 eq.) was added. The reaction mixture was stirred at room temperature under nitrogen for 16 hours. TLC (petroleum ether/ethyl acetate=30/1) showed consumption of compound 1 and formation of the desired product. The reaction mixture was extracted with ethyl acetate (20 mL) and washed with water (40 mL×3), dried over anhydrous sodium sulfate, filtered and concentrated under reduced pressure. The residue was purified by silica gel column chromatography, eluting with petroleum ether/ethyl acetate (1/0-20/1), to obtain compound 3 (4.365 g, 28%) as a colorless oil.

### 2. Synthesis of Compound 5

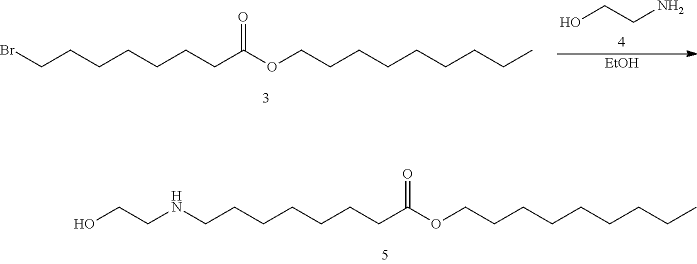

A mixture of compound 3 (5 g, 14.38 mmol, 1 eq.) and compound 4 (8.8 g, 143.7 mmol, 10 eq.) in ethanol (2 mL) was stirred at 55 °C under nitrogen for 16 hours. TLC (DCM/MeOH=10/1) showed that new major spots were observed. The reaction mixture was extracted with ethyl acetate (50 mL) and washed with water (3×50 mL). The organic layer was dried over anhydrous sodium sulfate, filtered and concentrated under reduced pressure. The residue was purified by silica gel column chromatography, eluting with DCM/MeOH (1/0-10:1, v/v), to give compound 5 (1.008 g, 21%) as a yellow oil.

### 3. Synthesis of Compound 8

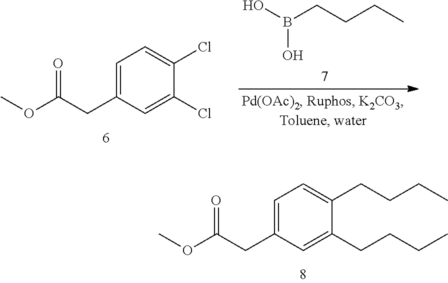

To compound 6 (500 mg, 2.283 mmol, 1 eq.) and compound 7 (699 mg, 6.849 mmol, 3 eq.) in toluene/water

To the mixture in (5 mL/1 mL) were added palladium acetate (51 mg, 0.228 mmol, 0.1 eq.), Ruphos (213 mg, 0.457 mmol, 0.2 eq.) and potassium carbonate (945 mg, 6.849 mmol, 3 eq.). The mixture was stirred at 110 °C overnight under nitrogen. TLC (PE/EA=20/1) showed the reaction was complete and new major spots were observed. The mixture was extracted with ethyl acetate and washed with water. The organic layer was dried over anhydrous sodium sulfate, filtered and concentrated in vacuo. The residue was purified by silica gel column chromatography, eluting with PE/EA (1/0-20/1), to obtain compound 8 (507 mg, 85%) as a colorless oil.

### 4. Synthesis of Compound 9

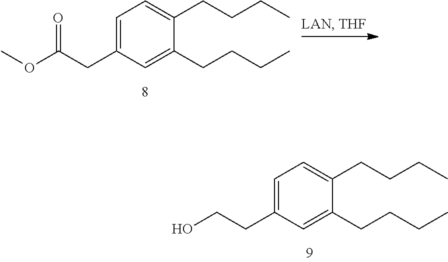 [SMILES: CCCCc1ccc(CC(=O)OC)cc1CCCC ; CCCCc1ccc(CCO)cc1CCCC]

To a mixture of compound 8 (507 mg, 1.935 mmol, 1 eq.) in THF (5 mL) was added lithium aluminum hydride (2 mL, 1.935 mmol, 1 M, 1 eq. in THF) at 0 °C under nitrogen. The mixture was stirred at room temperature for 3 hours. TLC (PE/EA=5/1) indicated that the reaction was complete and new major spots were observed. The mixture was quenched with water (2 mL) and treated with 2N hydrochloric acid to adjust the pH between 6 and 7, extracted with ethyl acetate and washed with brine. The organic layer was dried over anhydrous sodium sulfate, filtered and concentrated in vacuo to afford compound 9 (492 mg, >100%) as a colorless oil without further purification.

### 5. Synthesis of Compound 10

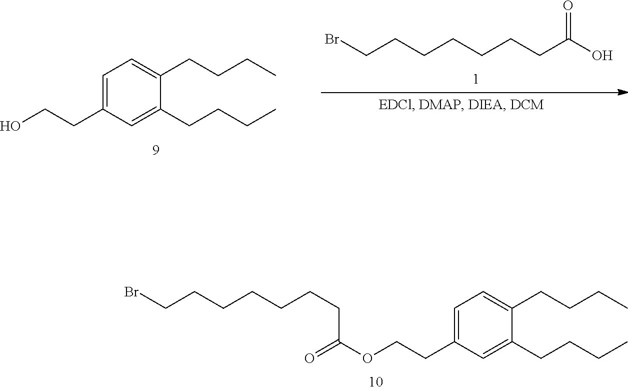 [SMILES: O=C(O)CCCCCCCBr ; CCCCc1ccc(CCO)cc1CCCC ; CCCCc1ccc(CCOC(=O)CCCCCCCBr)cc1CCCC]

To a mixture of compound 9 (492 mg, 2.103 mmol, 1 eq.) and compound 1 (563 mg, 2.523 mmol, 1.2 eq.) in DCM (5 mL) was added EDCI (808 mg, 4.206 mmol, 2 eq.) and DMAP (103 mg, 0.84 mmol, 0.4 eq.), then DIEA (1.085 g, 8.412 mmol, 4 eq.) was added. The reaction mixture was stirred at room temperature under nitrogen for 16 hours. TLC (petroleum ether/ethyl acetate=15/1) showed the formation of the desired product. The reaction mixture was extracted with ethyl acetate and washed with water. The organic layer was dried over anhydrous sodium sulfate, filtered and concentrated in vacuo. The residue was purified by silica gel column chromatography, eluting with petroleum ether/ethyl acetate (1/0-10/1), to obtain compound 10 (329 mg, 36%) as a colorless oil.

### 6. Synthesis of SW-II-134-3

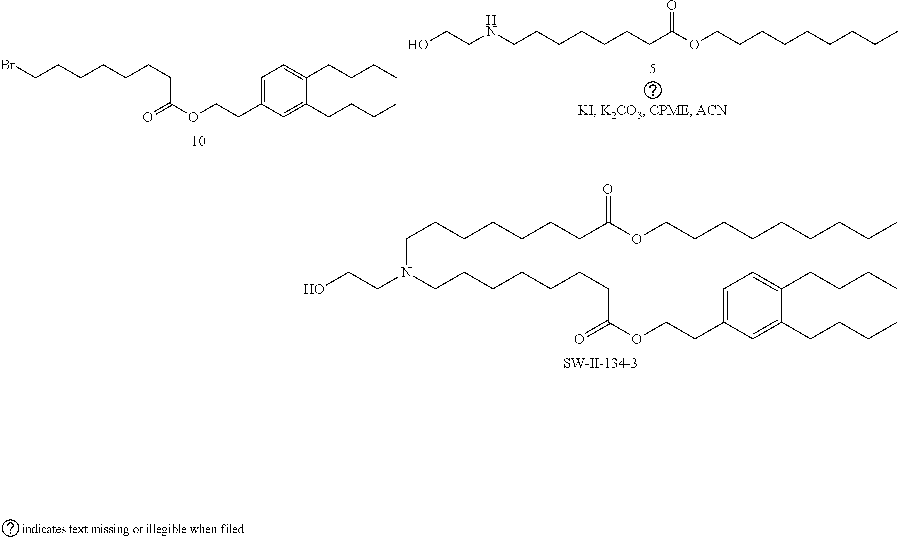 [SMILES: CCCCCCCCCOC(=O)CCCCCCCNCCO ; CCCCc1ccc(CCOC(=O)CCCCCCCBr)cc1CCCC ; CCCCCCCCCOC(=O)CCCCCCCN(CCO)CCCCCCCC(=O)OCCc1ccc(CCCC)c(CCCC)c1]

To compound 10 (150 mg, 0.34 mmol, 1 eq.) and compound 5 (134 mg, 0.41 mmol, 1.2 eq.) in CPME/CH₃ To the mixture in CN (2 mL/2 mL) were added potassium carbonate (282 mg, 2.04 mmol, 6 eq.) and potassium iodide (113 mg, 0.68 mmol, 2 eq.). After addition, the mixture was stirred at 90 °C overnight under nitrogen. TLC (DCM/MeOH=10/1) showed the reaction was complete and new major spots were observed. The mixture was extracted with ethyl acetate and washed with water. The organic layer was dried over anhydrous sodium sulfate, filtered and concentrated under reduced pressure. The residue was purified by silica gel column chromatography, eluting with DCM/MeOH (1/0-10:1, v/v), to obtain compound SW-II-134-3 (63.59 mg, 25%) as a yellow oil.

LCMS: Rt: 1.247 min; MS m/z (ELSD): 688.3[M+H]⁺;

HPLC: 95.945% purity, ELSD; RT=6.186 min.

¹H NMR (400 MHz, CDCl₃) δ 7.07 (d, J=7.6 Hz, 1H), 6.97 (dd, J=9.9, 2.2 Hz, 2H), 4.26 (t, J=7.2 Hz, 2H), 4.05 (t, J=6.8 Hz, 2H), 2.88 (dd, J=14.8, 7.6 Hz, 4H), 2.78-2.74 (m, 2H), 2.67-2.54 (m, 8H), 2.29 (t, J=7.5 Hz, 4H), 1.68-1.47 (m, 15H), 1.37-1.22 (m, 27H), 0.98-0.86 (m, 9H).

¹³C NMR (101 MHz, CDCl₃) δ 173.86 (d, J=17.1 Hz), 140.66 (s), 138.76 (s), 134.93 (s), 129.74 (s), 129.24 (s), 126.19 (s), 7 7.36 (s), 77.04 (s), 76.72 (s), 65.01 (s), 64.48 (s), 57.73 (s), 55.73 (s), 53.93 (s), 34.76 (s), 34.28 (d, J=3.9 Hz), 33.54 (d, J=4.5 Hz), 32.41 (s), 31.95 (d, J=16.5 Hz), 29.49 (s), 29.15 (dd, J=21.1, 2.4 Hz), 28.66 (s), 27.04 (s), 25.95 (d, J=3.3 Hz), 24.85 (d, J=6.6 Hz), 22.98-22.58 (m), 14.08 (d, J=7.5 Hz).

### J. SW-II-135-1

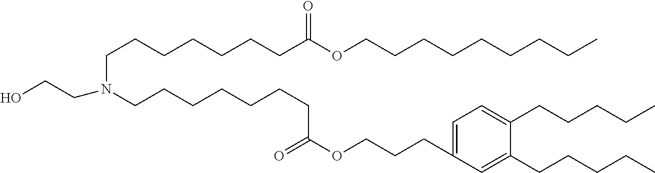 [SMILES: CCCCCCCCCOC(=O)CCCCCCCN(CCO)CCCCCCCC(=O)OCCCc1ccc(CCCCC)c(CCCCC)c1]

### 1. Synthesis of Compound 3

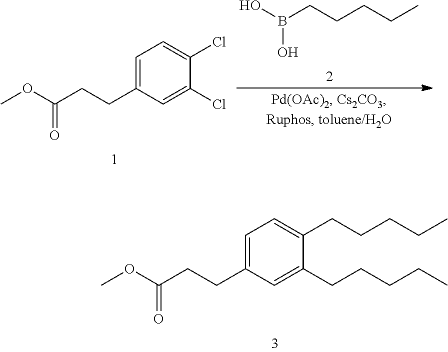

Compound 1 (500 mg, 2.16 mmol, 1.0 eq.) and compound 2 (750 mg, 6.46 mmol, 3.0 eq.) were dissolved in toluene/H₂O (5 mL/1 mL), add Ruphos (201 mg, 0.43 mmol, 0.2 eq), Pd(OAc) to the mixture₂ (48.5 mg, 0.22 mmol, 0.1 eq) and Cs₂ CO₃ (2.10 g, 6.46 mmol, 3.0 eq.). The reaction mixture was heated to reflux at 110 °C for 16 hours under nitrogen protection. TLC (petroleum ether/ethyl acetate=10/1) showed that the reaction was complete and the desired product was formed. The reaction mixture was reacted with H₂ Wash with O (40 mL) and extract 3 times with EA (50 mL). The resulting organic phase is washed twice with brine (20 mL) and washed with anhydrous Na₂SO₄ Dry, filter and concentrate under reduced pressure. The residue was purified by silica gel column chromatography, eluting with petroleum ether/ethyl acetate (1/0-30/1), to obtain compound 3 (540 mg, 82.44%) as a yellow oil.

### 2. Synthesis of Compound 4

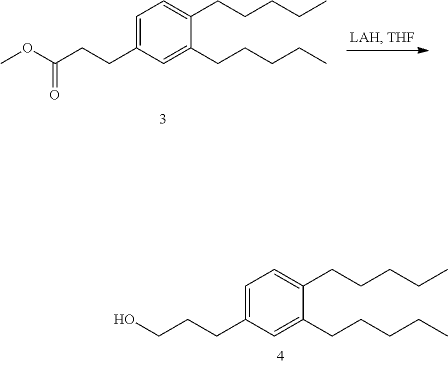 [SMILES: CCCCCc1ccc(CCC(=O)OC)cc1CCCCC ; CCCCCc1ccc(CCCO)cc1CCCCC]

LiAlH was added to compound 3 (540 mg, 1.78 mmol, 1.0 eq.) dissolved in THF (5 mL) under nitrogen protection at 0 °C, 4 (3.55 mL, 3.55 mmol, 1M THF, 2 eq.). The reaction mixture was raised to room temperature and stirred under nitrogen protection for 2 hours. TLC (PE/EtOAc=10/1) showed the reaction was complete and new major spots were observed. The mixture was quenched with water (10 mL), then adjusted to pH=6-7 with 1M hydrochloric acid, and extracted three times with ethyl acetate (50 mL). The organic layer was washed with brine, dried over anhydrous sodium sulfate, filtered, and concentrated under reduced pressure. The residue was purified by silica gel column chromatography, eluting with PE/EA (1/0-10/1), to obtain compound 4 (442 mg, 90.2%) as a colorless oil.

### 3. Synthesis of Compound 6

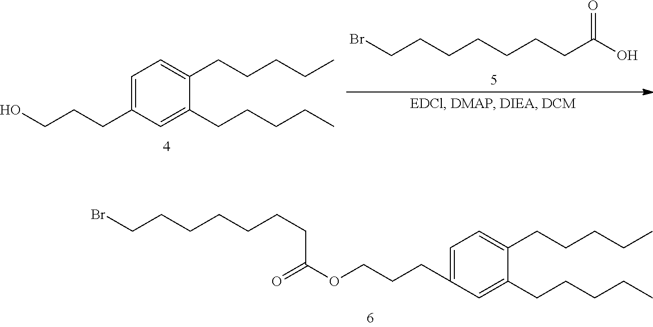

Compound 4 (442 mg, 1.60 mmol, 1.0 eq.) and compound 5 (428.5 mg, 1.92 mmol, 1.2 eq.) were dissolved in DCM (5 mL), and EDCI (612 mg, 3.2 mmol, 2.0 eq.) was added to the mixture.) and DMAP (78.2 mg, 0.64 mmol, 0.4 eq.), then DIEA (826 mg, 6.4 mmol, 4.0 eq.) was added. The reaction mixture was stirred at room temperature under nitrogen protection for 16 hours. TLC (petroleum ether/ethyl acetate=10/1) showed that compound 4 was consumed and the desired product formed. The reaction mixture was reacted with H₂ Wash with O (40 mL) and extract 3 times with EA (50 mL). The obtained organic phase is washed twice with brine (20 mL) and washed with anhydrous Na₂SO₄ Dry, filter and concentrate under reduced pressure. The residue was purified by silica gel column chromatography, eluting with petroleum ether/ethyl acetate (1/0-10/1), to obtain compound 3 (342 mg, 44.5%) as a yellow oil.

### 4. Synthesis of SW-II-135-1

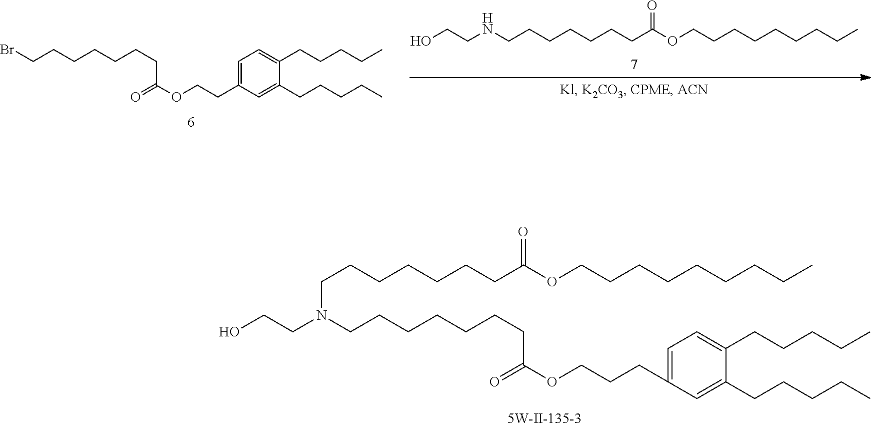

Compound 6 (175 mg, 0.365 mmol, 1.2 eq.) and compound 7 (100 mg, 0.304 mmol, 1.0 eq.) were dissolved in CPME/CH₃CN (1 mL/1 mL), to the mixture were added potassium carbonate (210 mg, 1.52 mmol, 5.0 eq) and potassium iodide (101 mg, 0.61 mmol, 2.0 eq). After the addition, the reaction mixture was stirred at 90 °C overnight under nitrogen protection. TLC (DCM/MeOH=10/1) showed that the reaction was complete and the desired product was formed. The mixture was extracted with ethyl acetate and washed with water. The organic layer was dried over anhydrous sodium sulfate, filtered and concentrated under reduced pressure. The residue was purified by silica gel column chromatography, eluting with DCM/MeOH (1/0-10:1, v/v), to obtain compound SW-II-135-1 (83.89 mg, 55.6%) as a yellow oil.

LCMS: Rt: 1.356 min; MS m/z (ELSD): 730.5[M+H]⁺;

HPLC: 100% purity at ELSD; RT=12.614 min.

¹H NMR (400 MHz, CDCl₃) δ 6.97 (d, J=7.6 Hz, 1H), 6.91-6.74 (m, 2H), 4.76 (s, 1H), 3.99 (dt, J=13.6, 6.4 Hz, 4H), 3.72-3.58 (m, 2H), 2.85-2.73 (m, 2H), 2.72-2.61 (m, 4H), 2.59-2.41 (m, 6H), 2.22 (dd, J=13.2, 7.2 Hz, 4H), 1.93-1.79 (m, 2H), 1.62-1.41 (m, 14H), 1.23 (d, J=24.4 Hz, 32H), 0.82 (ddd, J=13.6, 8.0, 5.6 Hz, 9H).

¹³C NMR (101 MHz, CDCl3) δ 172.81 (d, J=6.4 Hz), 139.55 (s), 137.38 (s), 137.14 (s), 128.16 (d, J=2.4 Hz), 124.69 (s), 76.51 (s), 76.19 (s), 75.88 (s), 63.43 (s), 62.78 (s), 56.53 (s), 54.90 (s), 52.84 (s), 33.23 (d, J=2.4 Hz), 31. 73 (s), 31.28 (s), 30.91 (dd, J=20.0, 6.4 Hz), 30.10 (d, J=3.2 Hz), 29.29 (s), 28.36 (d, J=22.8 Hz), 28.23 (s), 27.97 (s), 27.64 (s), 25.92 (s), 24.92 (s), 24.34 (s), 23.84 (s), 21.62 (d, J=7.6 Hz), 13.08 (d, J=4.7 Hz)

### K. Compound SW-II-135-2

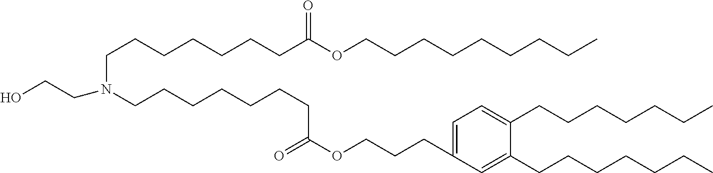 [SMILES: CCCCCCCCCOC(=O)CCCCCCCN(CCO)CCCCCCCC(=O)OCCCc1ccc(CCCCCCC)c(CCCCCCC)c1]

### 1. Synthesis of Compound 3

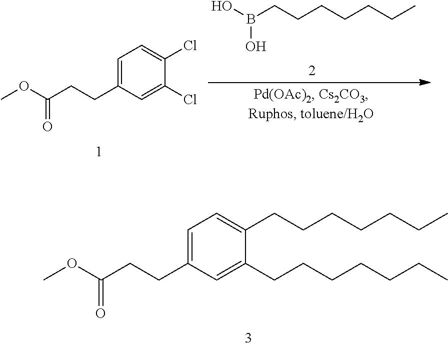

Compound 1 (500 mg, 2.16 mmol, 1.0 eq.) and compound 2 (931 mg, 6.46 mmol, 3.0 eq.) were dissolved in toluene/H₂O (5 mL/1 mL), add Ruphos (201 mg, 0.43 mmol, 0.2 eq), Pd(OAc) to the mixture₂ (48.5 mg, 0.22 mmol, 0.1 eq) and Cs₂ CO₃ (2.10 g, 6.46 mmol, 3.0 eq.). The reaction mixture was heated to reflux at 110 °C for 16 hours under nitrogen protection. TLC (petroleum ether/ethyl acetate=10/1) showed that the reaction was complete and the desired product was formed. The reaction mixture was reacted with H₂ Wash with O (40 mL) and extract 3 times with EA (50 mL). The resulting organic phase is washed twice with brine (20 mL) and washed with anhydrous Na₂SO₄ Dry, filter and concentrate under reduced pressure. The residue was purified by silica gel column chromatography, eluting with petroleum ether/ethyl acetate (1/0-30/1), to obtain compound 3 (651 mg, 84%) as a yellow oil.

### 2. Synthesis of Compound 4

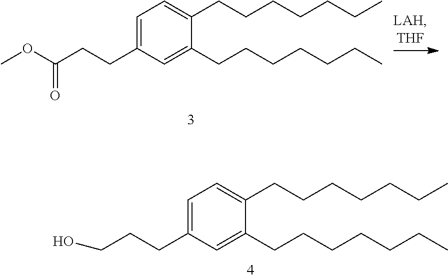 [SMILES: CCCCCCCc1ccc(CCC(=O)OC)cc1CCCCCCC ; CCCCCCCc1ccc(CCCO)cc1CCCCCCC]

LiAlH was added to compound 3 (651 mg, 1.81 mmol, 1.0 eq.) dissolved in THF (7 mL) under nitrogen protection at 0 °C, 4 (3.62 mL, 3.62 mmol, 1M in THF, 2 eq.). The reaction mixture was raised to room temperature and stirred under nitrogen protection for 2 hours. TLC (PE/EtOAc=10/1) showed the reaction was complete and new major spots were observed. The mixture was quenched with water (10 mL), then adjusted to pH=6-7 with IM hydrochloric acid, and extracted three times with ethyl acetate (50 mL). The organic layer was washed with brine, dried over anhydrous sodium sulfate, filtered, and concentrated under reduced pressure. The residue was purified by silica gel column chromatography, eluting with PE/EA (1/0-10/1), to obtain compound 4 (571 mg, 95.2%) as a colorless oil.

### 3. Synthesis of Compound 6

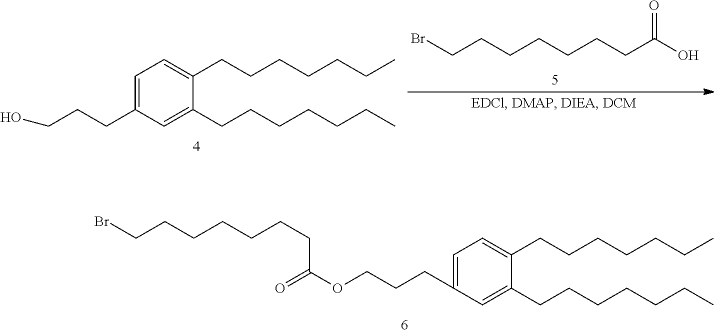

Compound 4 (571 mg, 1.72 mmol, 1.0 eq.) and compound 5 (459 mg, 2.06 mmol, 1.2 eq.) were dissolved in DCM (6 mL), and EDCI (657 mg, 3.44 mmol, 2.0 eq.) was added to the mixture, and DMAP (84 mg, 0.68 mmol, 0.4 eq.), followed by DIEA (887.5 mg, 6.88 mmol, 4.0 eq.). The reaction mixture was stirred at room temperature under nitrogen protection for 16 hours. TLC (petroleum ether/ethyl acetate=10/1) showed that compound 4 was consumed and the desired product formed. The reaction mixture was reacted with H₂ Wash with O (50 mL) and extract 3 times with EA (60 mL). The resulting organic phase is washed twice with brine (25 mL) and washed with anhydrous Na₂SO a Dry, filter and concentrate under reduced pressure. The residue was purified by silica gel column chromatography, eluting with petroleum ether/ethyl acetate (1/0-10/1), to obtain compound 3 (245 mg, 26.5%) as a yellow oil.

### 4. Synthesis of SW-II-135-2

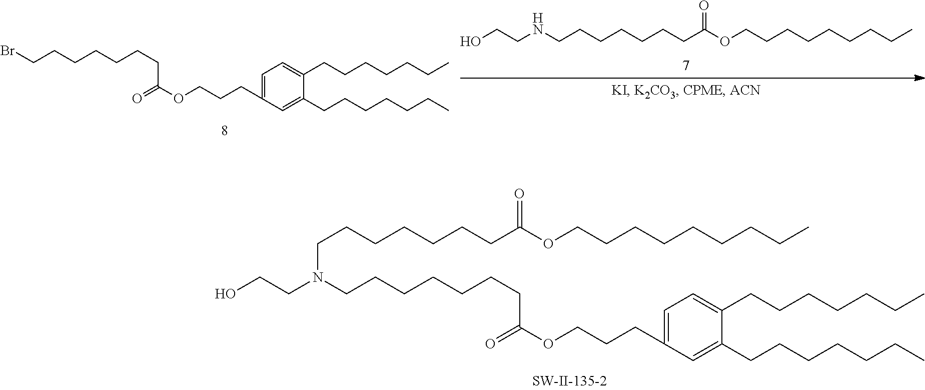

Compound 6 (245 mg, 0.456 mmol, 1.5 eq.) and compound 7 (100 mg, 0.3 mmol, 1.0 eq.) were dissolved in CPME/CH₃ CN (1 mL/1 mL), to the mixture were added potassium carbonate (210 mg, 1.52 mmol, 5.0 eq) and potassium iodide (101 mg, 0.61 mmol, 2.0 eq). After the addition, the reaction mixture was stirred at 90 °C overnight under nitrogen protection.

TLC (DCM/MeOH=10/1) showed that the reaction was complete and the desired product was formed. The mixture was extracted with ethyl acetate and washed with water. The organic layer was dried over anhydrous sodium sulfate, filtered and concentrated under reduced pressure. The residue was purified by silica gel column chromatography, eluting with DCM/MeOH (1/0-10:1, v/v), to obtain compound SW-II-135-2 (31.41 mg, 21.9%) as a yellow oil.

LCMS: Rt: 1.608 min; MS m/z (ELSD): 786.4[M+H]⁺;

HPLC: 95.16% purity, ELSD; RT=7.919 min.

¹H NMR (400 MHz, CDCl₃) δ 6.98 (d, J=7.6 Hz, 1H), 6.87 (d, J=2.4 Hz, 2H), 4.28-4.13 (m, 1H), 4.04-3.95 (m, 4H), 3.94-3.84 (m, 2H), 3.14-2.89 (m, 6H), 2.59-2.43 (m, 6H), 2.23 (dd, J=13.8, 7.2 Hz, 4H), 1.88-1.82 (m, 2H), 1.70 (s, 4H), 1.57-1.46 (m, 10H), 1.33-1.16 (m, 40H), 0.90-0.72 (m, 9H).

¹³C NMR (100 MHz, CDCl₃) δ 172.82 (d, J=6.8 Hz), 139.61 (s), 137.29 (d, J=16.4 Hz), 128.15 (s), 124.67 (s), 76.41 (s), 76.09 (s), 75.77 (s), 63.50 (s), 62.87 (s), 55.49 (s), 54.92 (s), 52.98 (s), 33.16 (d, J=2.4 Hz), 31.77 (s), 31.33 (s), 30.80 (d, J=6.5 Hz), 30.42 (d, J=3.6 Hz), 29.29 (s), 28.99-28.66 (m), 28.47 (s), 28.23 (d, J=2.8 Hz), 28.06-7.45 (m), 25.58 (s), 24.91 (s), 23.71 (s), 22.79 (s), 21.66 (s), 13.10 (s).

### L. Compound SW-II-136-2

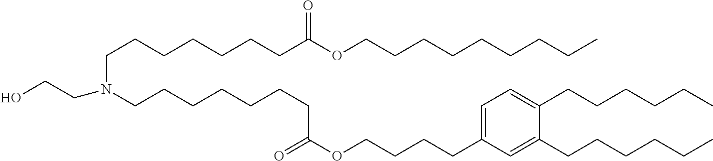 [SMILES: CCCCCCCCCOC(=O)CCCCCCCN(CCO)CCCCCCCC(=O)OCCCCc1ccc(CCCCCC)c(CCCCCC)c1]

### 1. Synthesis of Compound 3

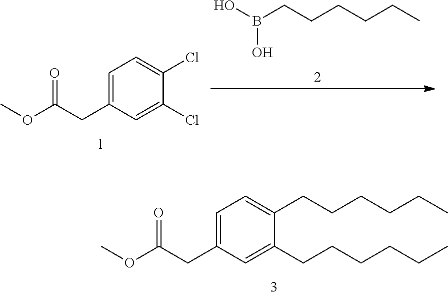

Compound 1 (3 g, 13.70 mmol, 1.0 eq.) and compound 2 (5.34 g, 41.09 mmol, 3.0 eq.) were dissolved in toluene/H₂O (30 mL/3 mL), add Ruphos (1.28 g, 2.74 mmol, 0.2 eq), Pd(OAc) to the mixture 2 (308.3 mg, 1.37 mmol, 0.1 eq) and K₂CO₃ (5.67 g, 41.10 mmol, 3.0 eq.). The reaction mixture was heated to reflux at 110 °C for 16 hours under nitrogen protection. TLC (PE/EA=10/1) showed the reaction was complete and the desired product was formed. The reaction mixture was reacted with H₂ Wash with O (90 mL) and extract 3 times with EA (110 mL). The obtained organic phase is washed twice with brine (40 mL) and washed with anhydrous Na₂SO₄ Dry, filter and concentrate under reduced pressure. The residue was purified by silica gel column chromatography, eluting with PE/EA (1/0-30/1), to obtain compound 3 (1.98 g, 45.5%) as a yellow oil.

### 2. Synthesis of Compound 4

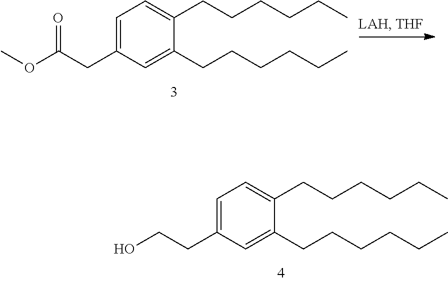 [SMILES: CCCCCCc1ccc(CC(=O)OC)cc1CCCCCC ; CCCCCCc1ccc(CCO)cc1CCCCCC]

LiAlH was added to compound 3 (1.98 g, 6.23 mmol, 1.0 eq.) dissolved in THF (20 mL) under nitrogen protection at 0 °C.₄ (1 M, 12.45 mL, 2.0 eq). The reaction mixture was raised to room temperature and stirred under nitrogen protection for 2 hours. TLC (PE/EtOAc=10/1) showed the reaction was complete and new major spots were observed. Mixture with H₂O (70 mL) quenched,

Then adjust pH=6-7 with IM hydrochloric acid, and extract 3 times with EA (80 mL). The organic layer was washed with brine and washed with anhydrous Na₂SO₄ Dry, filter, and concentrate under reduced pressure. The residue was purified by silica gel column chromatography, eluting with PE/EA (1/0-10/1), to obtain compound 4 (1.28 g, 71.1%) as a colorless oil.

### 3. Synthesis of Compound 7

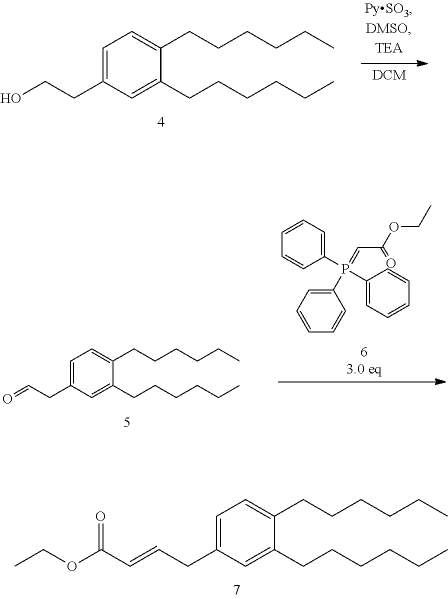

Under nitrogen protection at 0 °C, DMSO (3.63 g, 51.72 mmol, 15 eq) and TEA (1.25 g, 12.4 mmol, 4.0 eq.) were added to compound 4 (900 g, 3.1 mmol, 1.0 eq.) dissolved in DCM (9 mL).) and PySO₃ (1.27 g, 7.97 mmol, 2.57 eq). The mixture was stirred at 0 °C for 30 minutes, then raised to room temperature and stirred under nitrogen protection for 90 minutes. Then compound 6 (4.74 g, 13.62 mmol, 3.0 eq.) was added to the mixture, and the reaction mixture was reacted at 25 °C for 2 hours under nitrogen protection. TLC (PE/EA=10/1) showed the reaction was complete and the desired product was formed. The reaction mixture was reacted with H₂ Wash with O (60 mL) and extract 3 times with EA (70 mL). The obtained organic phase is washed twice with brine (40 mL) and washed with anhydrous Na₂SO₄ Dry, filter and concentrate under reduced pressure. The residue was purified by silica gel column chromatography, eluting with PE/EA (1/0-10/1), to obtain compound 7 (345 mg, 27.9%) as a yellow oil.

### 4. Synthesis of Compound 8

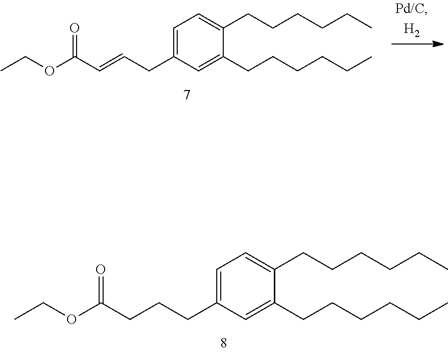

Compound 7 (340 mg, 0.95 mmol, 1.0 eq.) and Pd/C (100 mg) were added to MeOH (4 ml), and the reaction mixture was stirred under hydrogen protection at room temperature for 16 h. TLC (PE/EA=10/1) showed complete consumption of starting material and formation of desired product. The reaction mixture was filtered through celite and washed with MeOH (40 mL×2), and washed with anhydrous Na₂SO₄ The filtrate was dried and concentrated under reduced pressure to obtain compound 8 (298 mg, 88.2%) as a light yellow oil.

### 5. Synthesis of Compound 9

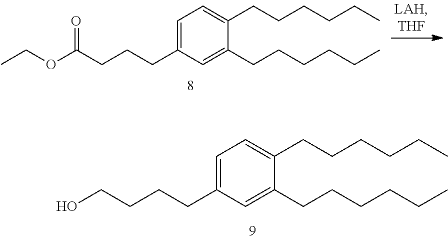 [SMILES: CCCCCCc1ccc(CCCC(=O)OCC)cc1CCCCCC ; CCCCCCc1ccc(CCCCO)cc1CCCCCC]

LiAlH was added to compound 8 (298 mg, 0.83 mmol, 1.0 eq.) dissolved in THF (3 mL) under nitrogen protection at 0 °C.₄ (1M, 1.66 mL, 2.0 eq). The reaction mixture was raised to room temperature and stirred under nitrogen protection for 2 hours. TLC (PE/EtOAc=10/1) showed the reaction was complete and new major spots were observed. Mixture with H₂ Quench with O (20 mL), then adjust pH=6-7 with IM hydrochloric acid, and extract 3 times with EA (30 mL). The organic laver was washed with brine and washed with anhydrous Na₂SO₄ Dry, filter, and concentrate under reduced pressure. The residue was purified by silica gel column chromatography, eluting with PE/EA (1/0-10/1), to obtain compound 9 (254 mg, 98.3%) as a colorless oil.

### 6. Synthesis of Compound 11

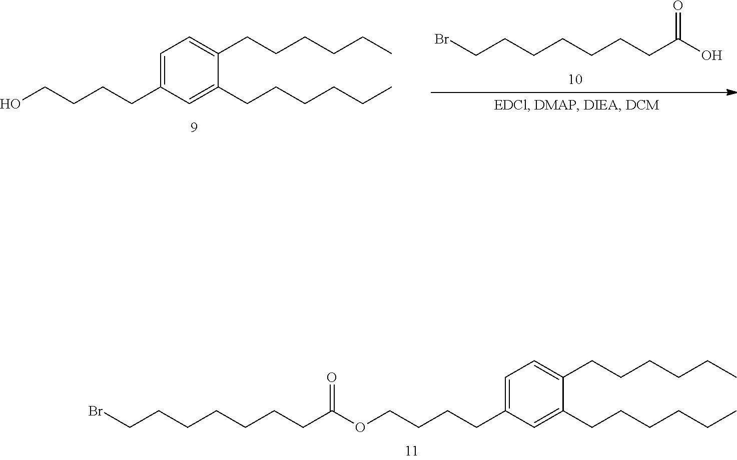 [SMILES: CCCCCCc1ccc(CCCCO)cc1CCCCCC ; O=C(O)CCCCCCCBr ; CCCCCCc1ccc(CCCCOC(=O)CCCCCCCBr)cc1CCCCCC]

Dissolve compound 9 (254 mg, 0.80 mmol, 1.0 eq.) and compound 10 (214 mg, 0.96 mmol, 1.2 eq.)

In DCM (3 mL), to the mixture were added EDCI (305.6 mg, 1.6 mmol, 2.0 eq.) and DMAP (39 mg, 0.32 mmol, 0.4 eq.), followed by DIEA (412.8 mg, 3.2 mmol, 4.0 eq.). The reaction mixture was stirred at room temperature under nitrogen protection for 16 hours. TLC (PE/EA=10/1) showed that compound 9 was consumed and the desired product formed. The reaction mixture was adjusted to pH=4-6 with IM hydrochloric acid, and extracted three times with EA (30 mL). The resulting organic phase was washed twice with brine (15 mL), and washed with anhydrous Na₂SO₄ Dry, filter and concentrate under reduced pressure. The residue was purified by silica gel column chromatography, eluting with PE/EA (1/0-7/1), to obtain compound 11 (210 mg, 50.5%) as a yellow oil.

### 7. Synthesis of SW-II-136-2

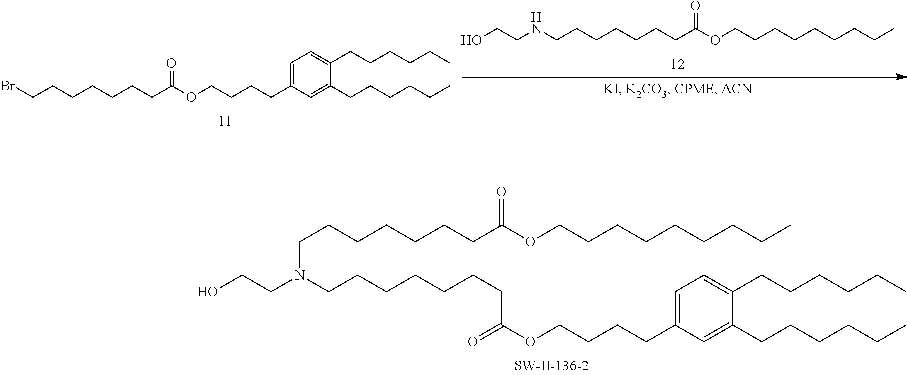

Compound 11 (200 mg, 0.38 mmol, 1.2 eq.) and compound 12 (105 mg, 0.32 mmol, 1.0 eq.) were dissolved in CPME/CH₃ CN (1.5 mL/1.5 mL), add K to this mixture₂ CO₃ (220.2 mg, 1.60 mmol, 5.0 eq) and KI (106 mg, 0.64 mmol, 2.0 eq). After the addition, the reaction mixture was stirred at 90 °C overnight under nitrogen protection. TLC (DCM/MeOH=10/1) showed that the reaction was complete and the desired product was formed. The mixture was extracted with EA and washed with water. Anhydrous Na was used for the organic Na₂SO₄ Dry, filter and concentrate under reduced pressure. The residue was purified by silica gel column chromatography, eluting with DCM/MeOH (1/0-10:1, v/v), to obtain compound SW-II-136-2 (208 mg, 90.4%) as a yellow oil.

LCMS: Rt: 2.146 min; MS m/z (ELSD): 773.3[M+H]⁺;

HPLC: 99.49% purity, ELSD: RT=8.055 min.

¹H NMR (400 MHz, CDCl₃) δ 7.04 (d, J=7.6 Hz, 1H), 6.92 (d, J=9.6 Hz, 2H), 4.45 (s, 1H), 4.06 (dd, J=12.0, 5.2 Hz, 4H), 3.6 4 (t, J=5.2 Hz, 2H), 2.72 (t, J=5.2 Hz, 2H), 2.65-2.50 (m, 10H), 2.29 (t, J=7.6 Hz, 4H), 1.69-1.48 (m, 18H), 1.41-1.24 (m, 36H), 0.95-0.78 (m, 9H).

¹³C NMR (101 MHz, CDCl₃) δ 173.86 (d, J=2.8 Hz), 140.48 (s), 139.24 (s), 138.01 (s), 129.13 (d, J=14.8 Hz), 125.67 (s), 7 7.37 (s), 77.05 (s), 76.73 (s), 64.45 (s), 64.23 (s), 57.88 (s), 55.91 (s), 53.94 (s), 35.07 (s), 34.29 (d, J=3.2 Hz), 32.79 (s), 32.35 (s), 31.82 (d, J=8.4 Hz), 31.38 (s), 29.50 (d, J=2.4 Hz), 29.16 (dd, J=18.0, 2.0 Hz), 28.66 (s), 28.35 (s), 27.78 (s), 27.08 (s), 26.02 (d, J=17.2 Hz), 24.89 (d, J=1.6 Hz), 22.65 (s), 14.10 (s).

### M. Compound SW-II-137-1

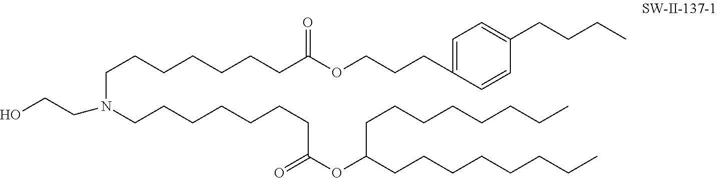 [SMILES: CCCCCCCCC(CCCCCCCC)OC(=O)CCCCCCCN(CCO)CCCCCCCC(=O)OCCCc1ccc(CCCC)cc1]

### 1. Synthesis of Compound 3

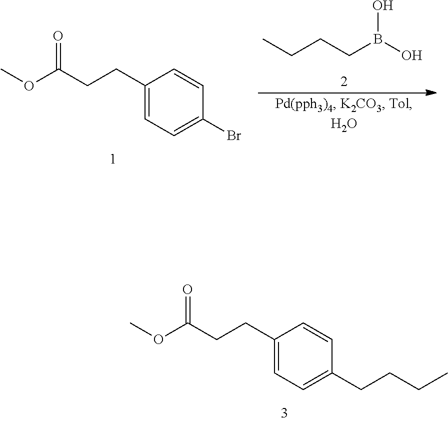

Compound 1 (500 mg, 1.95 mmol, 1.0 eq.) was dissolved in toluene (5.0 mL), then compound 2 (239 mg, 2.34 mmol, 1.2 eq.), Pd(PPh) were added₃)₄ (225 mg, 0.19 mmol, 0.1 eq), water (1 mL) and K₂ CO₃ (808 g, 5.85 mmol, 3.0 eq.). React at 110 °C for 3 hours under nitrogen protection. TLC (PE/EA=5/1) showed that the starting material had reacted completely and the desired product was formed. Add H to the reaction₂ O (70 mL), EA (80 mL×3) extraction. Combine the organic phases and wash with saturated brine (2×30 mL), anhydrous Na₂SO₄ Dry, filter and spin dry under reduced pressure. The residue was purified with a silica gel column and eluted with PE/EA (1/0-5:1, v/v) to obtain a colorless oily compound (320 mg, 70%).

### 2. Synthesis of Compound 4

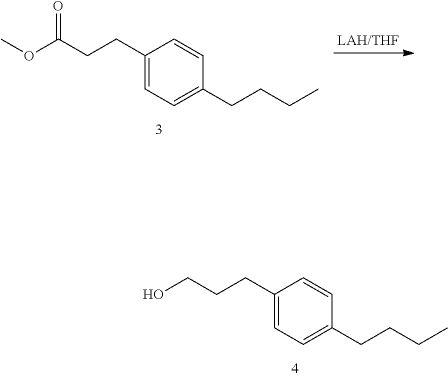 [SMILES: CCCCc1ccc(CCC(=O)OC)cc1 ; CCCCc1ccc(CCCO)cc1]

Compound 3 (3×) mg, 1.28 mmol, 1.0 eq.) was dissolved in THF (4.0 mL), and LAH (97 mg, 2.56 mmol, 2.0 eq.) was added at 0 °C under nitrogen protection. Then react at room temperature for 2 hours. TLC (PE/EA=10/1) showed that the starting material reacted completely and the desired product was formed. Add HCl (1M, 4 mL) solution and H₂ Quench with O (10 mL) and extract with EA (50 mL×3). The organic phase was washed with saturated brine (2×30 mL) and anhydrous Na₂SO₄ Dry, filter and spin dry under reduced pressure. The residue was purified with a silica gel column and eluted with PE/EA (1/0-10/1, v/v) to obtain compound 4 (224 mg, 84.8%) as a yellow oil.

### 3. Synthesis of Compound 6

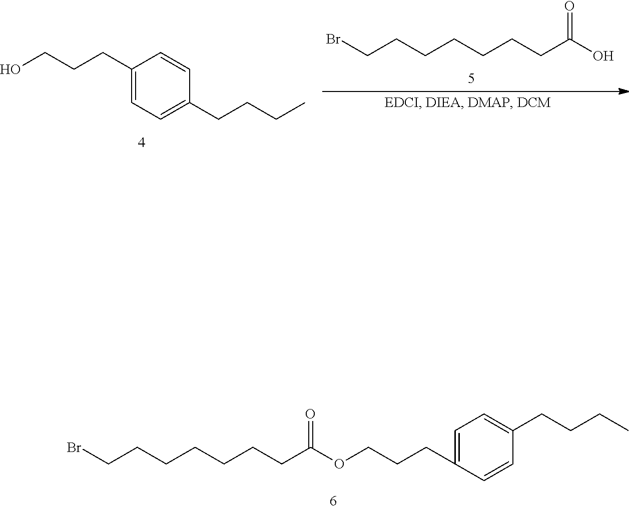 [SMILES: CCCCc1ccc(CCCO)cc1 ; O=C(O)CCCCCCCBr ; CCCCc1ccc(CCCOC(=O)CCCCCCCBr)cc1]

Compound 4 (90 mg, 0.47 mmol, 1.0 eq.) was dissolved in DCM (3.0 mL), and compound 5 (127 mg, 0.56 mmol, 1.2 eq.), EDCI (180 mg, 0.94 mmol, 2.0 eq.), DIEA (242 mg) were added, 1.88 mmol, 4.0 eq.) and DMAP (23 mg, 0.18 mmol, 0.4 eq.). Then, the reaction was carried out at room temperature overnight under nitrogen protection. TLC (PE/EA=20/1) showed that the starting material had been reacted and the desired product had been formed. Quench the reaction with HCl (1M) solution, adjust pH=4-6, and extract with EA (40 mL×3). Combine the organic phases and wash with saturated brine (2×30 mL), anhydrous Na₂SO₄ Dry, filter and spin dry under reduced pressure. The residue was purified using a silica gel column and eluted with PE/EA (1/0-20/1, v/v) to obtain compound 6 (90 mg, 48.6%) as a colorless oil.

### 4. Synthesis of SW-II-137-1

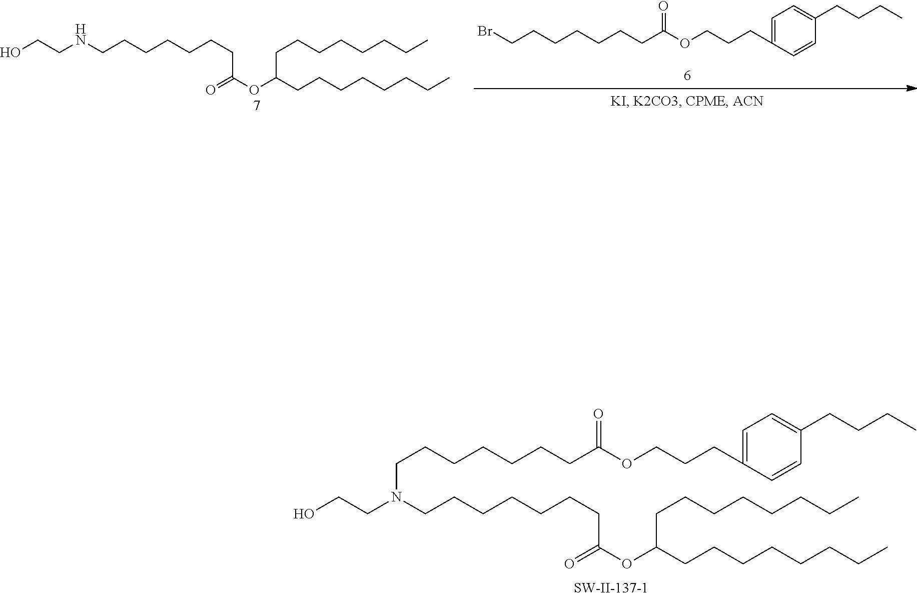 [SMILES: CCCCCCCCC(CCCCCCCC)OC(=O)CCCCCCCNCCO ; CCCCc1ccc(CCCOC(=O)CCCCCCCBr)cc1 ; CCCCCCCCC(CCCCCCCC)OC(=O)CCCCCCCN(CCO)CCCCCCCC(=O)OCCCc1ccc(CCCC)cc1]

Compound 6 (90 mg, 0.25 mmol, 1.0 eq.) was dissolved in MeCN (2 mL) and compound 7 (110 mg, 0.25 mmol, 1.0 eq.), KI (76 mg, 0.50 mmol, 2.0 eq.), CPME (2 mL) and K were added.₂ CO₃ (157 mg, 1.25 mmol, 5.0 eq). The reaction was carried out overnight at 90 °C under nitrogen protection. TLC (DCM/MeOH=10/1) showed complete reaction of the starting material and formation of the desired product. Quench with water (50 mL) and extract with EA (40 mL×3). Combine the organic phases and wash with saturated brine (2×30 mL), anhydrous Na₂SO₄ Dry, filter and spin dry under reduced pressure. The residue was purified by silica gel column and eluted with DCM/MeOH (1/0-10/1, v/v) to obtain a yellow oily compound (98 mg, 52.12%, SW-II-137-1).

LCMS: Rt: 1.596 min; MS m/z (ELSD): 758.4[M+H]⁺;

HPLC: 98.02% purity, ELSD; RT=5.993 min.

¹H NMR (400 MHz, CDCl₃) δ 7.02 (d, J=8.8 Hz, 4H), 4.92-4.71 (m, 1H), 4.01 (t, J=6.4 Hz, 2H), 3.78 (s, 1H), 3.55 (t, J=5.2 Hz, 2H), 2.76-2.40 (m, 10H), 2.21 (dd, J=15.6, 7.7 Hz, 4H), 1.95-1.80 (m, 2H), 1.49 (ddd, J=24.4, 15.8, 6.2 Hz, 15H), 1.34-1.13 (m, 37H), 0.82 (dt, J=13.6, 7.2 Hz, 9H).

¹³C NMR (101 MHz, CDCl₃) δ 173.79 (s), 173.57 (s), 140.49 (s), 138.3 (s), 128.43 (s), 128.22 (s), 77.43 (s), 77.11 (s), 76.7 9 (s), 74.11 (s), 63.66 (s), 57.96 (s), 55.75 (s), 53.90 (s), 35.22 (s), 34.63 (s), 34.20 (d, J=11.6 Hz), 33.70 (s), 31.80 (d, J=11.2 Hz), 30.30 (s), 29.51 (d, J=2.8 Hz), 29.13 (dd, J=9.6, 6.8 Hz), 27.12 (d, J=2.8 Hz), 26.29 (s), 25.31 (s), 24.97 (d, J=15.6 Hz), 22.66 (s), 22.37 (s), 14.02 (d, J=15.2 Hz).

### N. Compound SW-II-137-2

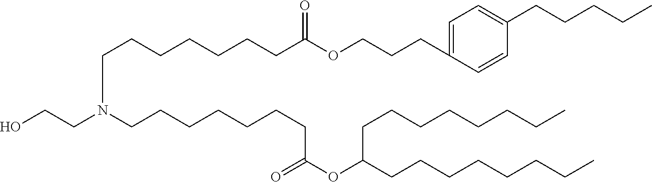 [SMILES: CCCCCCCCC(CCCCCCCC)OC(=O)CCCCCCCN(CCO)CCCCCCCC(=O)OCCCc1ccc(CCCCC)cc1]

### 1. Synthesis of Compound 3

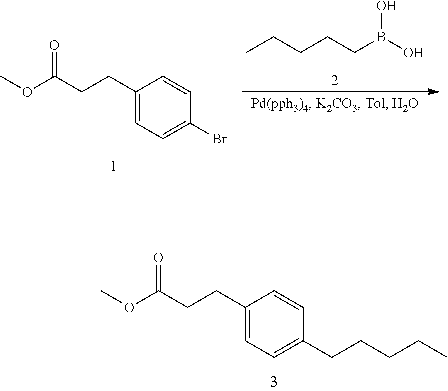

Compound 1 (500 mg, 2.06 mmol, 1.0 eq.), Compound 2 (286 mg, 2.47 mmol, 1.2 eq.), Pd(PPh₃)₄ (119 mg, 0.1 mmol, 0.1 eq) and K₂ CO₃ (851 mg, 6.21 mmol, 3.0 eq.) was dissolved in toluene (5.0 mL), and water (0.5 mL) was added. Then, the reaction was carried out at 110 °C for 3 hours under nitrogen protection. TLC (PE/EA=5/1) showed that the starting material reacted completely and the desired compound was formed. Use H₂ The reaction was quenched with O (70 mL) and extracted with EA (80 mL×3). Wash the organic phase with saturated brine (2-30 mL), anhydrous Na₂SO₄ Dry, filter and spin dry under reduced pressure. The residue was purified with a silica gel column and eluted with (PE/EA=5/1, v/v) to obtain compound 3 (420 mg, 87.5%) as a colorless oil.

### 2. Synthesis of Compound 4

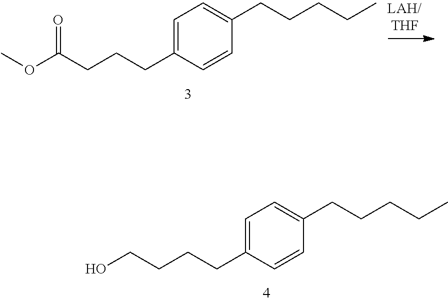 [SMILES: CCCCCc1ccc(CCCC(=O)OC)cc1 ; CCCCCc1ccc(CCCCO)cc1]

Compound 3 (420 mg, 1.78 mmol, 1.0 eq.) was dissolved in THF (3.0 mL), and LAH (1 M, 7 mL, 2.0 eq.) was added dropwise at 0 °C under nitrogen protection. Then, the reaction was carried out at room temperature for 2 hours. TLC (PE/EA=5/1) showed that the starting material was completely reacted and the desired product was formed. Use HCl (1M, 4 mL) solution and H₂ Quench with O (10 mL) and extract with EA (50 mL×3). The organic phase was washed with saturated brine (2×30 mL) and anhydrous Na₂SO₄ Dry, filter and spin dry under reduced pressure. The residue was purified using a silica gel column and eluted with (PE/EA=5/1, v/v) to obtain compound 4 (320 mg, 94%) as a colorless oil.

### 3. Synthesis of Compound 6

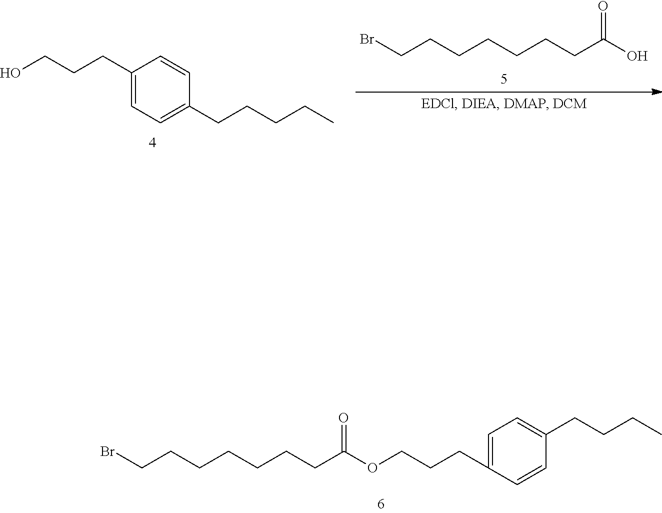 [SMILES: O=C(O)CCCCCCCBr ; CCCCCc1ccc(CCCO)cc1 ; CCCCc1ccc(CCCOC(=O)CCCCCCCBr)cc1]

Compound 4 (320 mg, 1.55 mmol, 1.0 eq.) was dissolved in DCM (4.0 mL), and compound 5 (416 mg, 1.86 mmol, 1.2 eq.), EDCI (594 mg, 3.11 mmol, 2.0 eq.), DIEA (802 mg, 6.21 mmol, 4.0 eq.) and DMAP (76 mg, 0.62 mmol, 0.4 eq.). Then, the reaction was carried out at room temperature overnight under nitrogen protection. TLC (PE/EA=20/1) showed that the starting material was completely reacted and the desired product was formed. The reaction was quenched with HCl (IM) solution and adjusted to pH=4-6, and extracted with DCM (60 mL×3). The organic phase was washed with saturated brine (2×35 mL) and anhydrous Na₂SO₄ Dry, filter and spin dry under reduced pressure. The residue was purified with a silica gel column and eluted with (PE/EA=5/1, v/v) to obtain compound 6 (300 mg, 47.17%) as a colorless oil.

### 4. Synthesis of SW-II-137-2

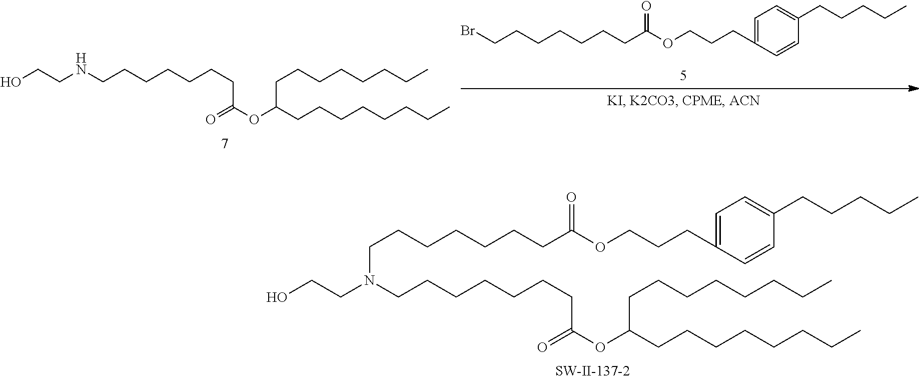 [SMILES: CCCCCc1ccc(CCCOC(=O)CCCCCCCBr)cc1 ; CCCCCCCCC(CCCCCCCC)OC(=O)CCCCCCCNCCO ; CCCCCCCCC(CCCCCCCC)OC(=O)CCCCCCCN(CCO)CCCCCCCC(=O)OCCCc1ccc(CCCCC)cc1]

Compound 6 (167 mg, 0.41 mmol, 1.2 eq.), compound 7 (150 mg, 0.34 mmol, 1.0 eq.), KI (113 mg, 0.68 mmol, 2.0 eq.) and CPME (2 mL) were dissolved in MeCN (2 mL), and K was added₂ CO₃ (235 mg, 1.70 mmol, 5.0 eq). The reaction was carried out overnight at 90 °C under nitrogen protection. TLC (DCM/MeOH=10/1) showed that the starting material reacted completely and the desired product was formed. The reaction was quenched with water (50 mL) and extracted with EA (60 ml×3). Use anhydrous Na for the organic phase₂SO₄ Dry, filter and spin dry under reduced pressure. The residue was purified with a silica gel column and eluted with DCM/MeOH (1/0-10/1, v/v) to obtain a light yellow oily compound (105 mg, 40.3%, SW-II-137-2).

LCMS: Rt: 1.660 min; MS m/z (ELSD): 772.4[M+H]⁺;

HPLC: 98.38% purity, ELSD; RT=8.743 min.

¹H NMR (400 MHz, CDCl₃) δ 7.10 (d, J=8.8 Hz, 4H), 5.04-4.74 (m, 1H), 4.08 (t, J=6.4 Hz, 2H), 3.58 (t, J=5.2 Hz, 2H), 2.65 (dd, J=9.6, 5.6 Hz, 4H), 2.60-2.44 (m, 6H), 2.29 (dd, J=16.4, 7.6 Hz, 4H), 2.01-1.88 (m, 2H), 1.59 (dt, J=9.2, 7.2 Hz, 6H), 1.54-1.42 (m, 8H), 1.39-1.11 (m, 41H), 0.88 (dt, J=11.8, 6.0 Hz, 9H).

¹³C NMR (101 MHz, CDCl₃) δ 173.86 (s), 173.63 (s), 140.59 (s), 138.34 (s), 128.45 (s), 128.24 (s), 77.36 (s), 77.04 (s), 76.7 2 (s), 74.14 (s), 63.69 (s), 58.11 (s), 55.71 (s), 53.90 (s), 35.53 (s), 34.68 (s), 34.23 (d, J=14.8 Hz), 31.82 (d, J=11.6 Hz), 31.56 (s), 31.26 (s), 30.32 (s), 29.53 (d, J=2.8 Hz), 29.19 (dd, J=8.0, 4.4 Hz), 27.20 (d, J=2.4 Hz), 26.64 (s), 25.33 (s), 25.02 (d, J=15.6 Hz), 22.62 (d, J=11.6 Hz), 14.08 (d, J=8.0 Hz).

### O. Compound SW-II-137-3

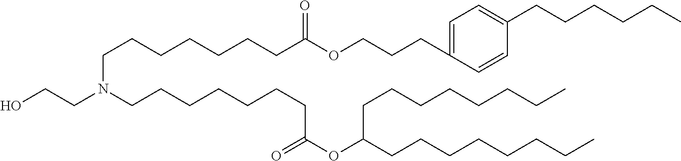 [SMILES: CCCCCCCCC(CCCCCCCC)OC(=O)CCCCCCCN(CCO)CCCCCCCC(=O)OCCCc1ccc(CCCCCC)cc1]

### 1. Synthesis of Compound 3

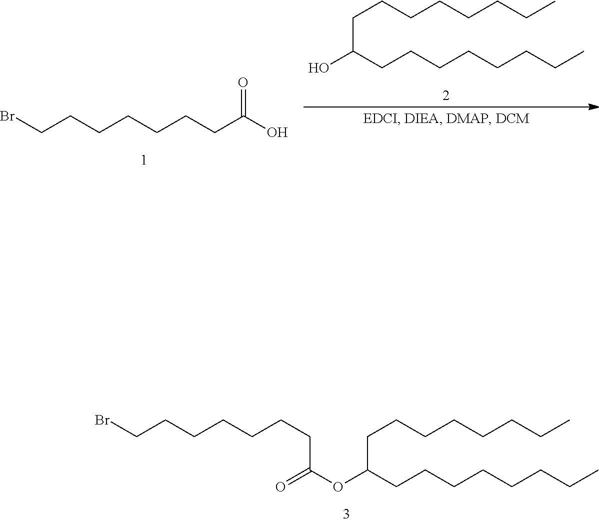 [SMILES: CCCCCCCCC(O)CCCCCCCC ; O=C(O)CCCCCCCBr ; CCCCCCCCC(CCCCCCCC)OC(=O)CCCCCCCBr]

To a mixture of compound 1 (11.8 g, 53 mmol, 1.2 eq.) and compound 2 (11.2 g, 44 mmol, 1 eq.) in DCM (10 mL) was added EDCI (16.9 g, 88 mmol, 2 eq.) and DMAP (2.1 g, 18 mmol, 0.4 eq.), then add DIEA (22.7 g, 176 mmol, 4 eq.). The reaction mixture was stirred at room temperature under nitrogen for 16 hours. TLC (petroleum ether/ethyl acetate=30/1) showed consumption of compound 1 and formation of the desired product. The reaction mixture was extracted with ethyl acetate (200 mL) and washed with water (200 mL×3), dried over anhydrous sodium sulfate, filtered and concentrated under reduced pressure. The residue was purified by silica gel column chromatography, eluting with petroleum ether/ethyl acetate (1/0-20/1), to obtain compound 3 (7.391 g, 37%) as a colorless oil.

### 2. Synthesis of Compound 5

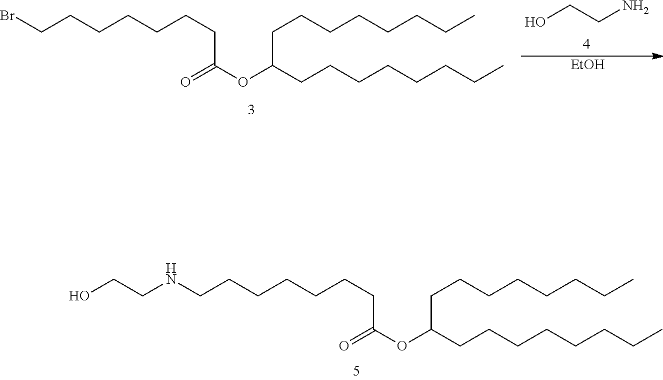

A mixture of compound 3 (7.391 mg, 16.07 mmol, 1 eq.) and compound 4 (29.4 g 482.02 mmol, 30 eq.) in ethanol (2 mL) was stirred at 55 °C under nitrogen for 16 hours. TLC (DCM/MeOH=10/1) showed that new major spots were observed. The reaction mixture was extracted with ethyl acetate (100 mL) and washed with water (3×100 mL). The organic layer was dried over anhydrous sodium sulfate, filtered and concentrated under reduced pressure. The residue was purified by silica gel column chromatography, eluting with DCM/MeOH (1/0-10:1, v/v), to obtain compound 5 (3.695 g, 52%) as a yellow oil.

### 3. Synthesis of Compound 8

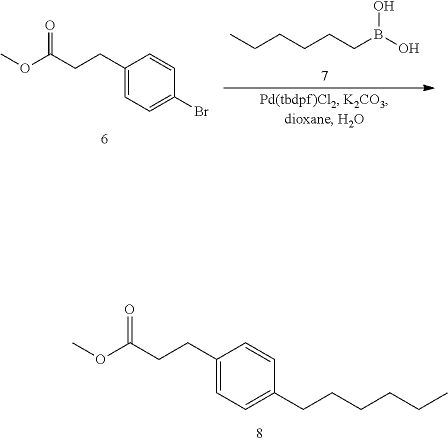

To a mixture of compound 6 (1 g, 4.12 mmol, 1 eq.) and compound 7 (803 g, 6.17 mmol, 1.5 eq.) in 1,4-dioxane/water (10 mL/1 mL) was added Pd(dtbpf)Cl₂ (269 mg, 0.41 mmol, 0.1 eq.) and potassium carbonate (1.7 g, 12.36 mmol, 3 eq.). The mixture was stirred at 100 °C overnight under nitrogen. TLC (PE/EA=20/1) showed the reaction was complete and new major spots were observed. The mixture was extracted with ethyl acetate and washed with water. The organic layer was dried over anhydrous sodium sulfate, filtered and concentrated in vacuo. The residue was purified by silica gel column chromatography, eluting with PE/EA (1/0-20/1), to obtain compound 8 (568 mg, 56%) as a colorless oil.

### 4. Synthesis of Compound 9

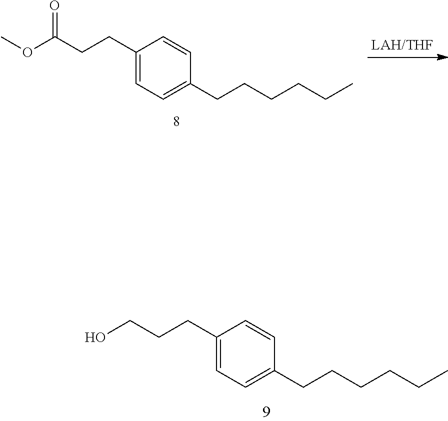 [SMILES: CCCCCCc1ccc(CCC(=O)OC)cc1 ; CCCCCCc1ccc(CCCO)cc1]

To a mixture of compound 8 (568 mg, 2.29 mmol, 1 eq.) in THF (6 mL) was added lithium aluminum hydride (2.3 mL, 2.29 mmol, 1 M, 1 eq. in THF) at 0 °C under nitrogen. The mixture was stirred at room temperature for 3 hours. TLC (PE/EA=5/1) indicated that the reaction was complete and new major spots were observed. The mixture was quenched with water (2.3 mL) and treated with 2N hydrochloric acid to adjust the pH between 6 and 7, extracted with ethyl acetate and washed with brine. The organic laver was dried over anhydrous sodium sulfate, filtered and concentrated in vacuo to afford compound 9 as a colorless oil (541 mg, >100%) without further purification.

### 5. Synthesis of Compound 10

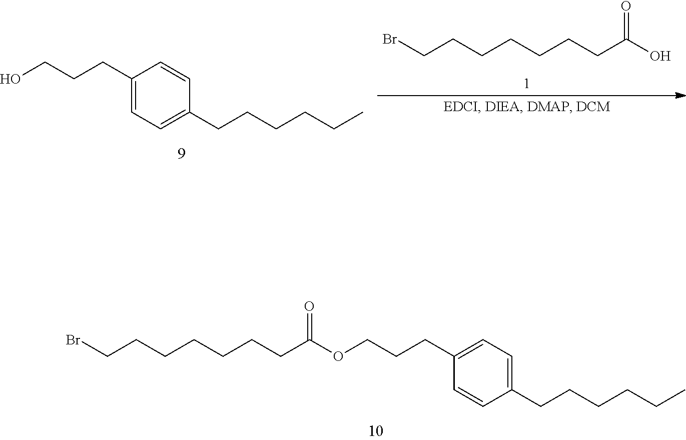 [SMILES: O=C(O)CCCCCCCBr ; CCCCCCc1ccc(CCCO)cc1 ; CCCCCCc1ccc(CCCOC(=O)CCCCCCCBr)cc1]

To a mixture of compound 9 (441 mg, 2 mmol, 1 eq.) and compound 1 (536 mg, 2.4 mmol, 1.2 eq.) in DCM (5 mL) was added EDCI (768 mg, 4 mmol, 2 eq.) and DMAP (98 mg, 0.8 mmol, 0.4 eq.), then add DIEA (1.032 g, 8 mmol, 4 eq.). The reaction mixture was stirred at room temperature under nitrogen for 16 hours. TLC (petroleum ether/ethyl acetate=10/1) showed the formation of the desired product. The reaction mixture was extracted with ethyl acetate and washed with water. The organic layer was dried over anhydrous sodium sulfate, filtered and concentrated in vacuo. The residue was purified by silica gel column chromatography, eluting with petroleum ether/ethyl acetate (1/0-10/1), to obtain compound 10 (372 mg, 44%) as a colorless oil.

### 6. Synthesis of SW-II-137-3

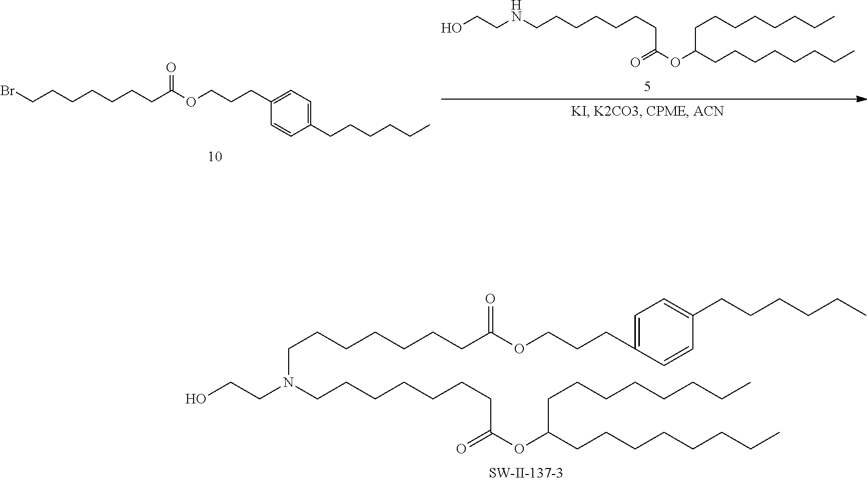 [SMILES: CCCCCCCCC(CCCCCCCC)OC(=O)CCCCCCCNCCO ; CCCCCCc1ccc(CCCOC(=O)CCCCCCCBr)cc1 ; CCCCCCCCC(CCCCCCCC)OC(=O)CCCCCCCN(CCO)CCCCCCCC(=O)OCCCc1ccc(CCCCCC)cc1]

To compound 10 (150 mg, 0.353 mmol, 1 eq.) and compound 5 (156 mg, 0.353 mmol, 1 eq.) in CPME/CH₃ To the mixture in CN (2 mL/2 mL) were added potassium carbonate (244 mg, 1.765 mmol, 6 eq.) and potassium iodide (117 mg, 0.706 mmol, 2 eq.). After addition, the mixture was stirred at 90 °C overnight under nitrogen. TLC (DCM/MeOH=10/1) showed the reaction was complete and new major spots were observed. The mixture was extracted with ethyl acetate and washed with water. The organic layer was dried over anhydrous sodium sulfate, filtered and concentrated under reduced pressure. The residue was purified by silica gel column chromatography, eluting with DCM/MeOH (1/0-10:1, v/v), to obtain compound SW-II-137-3 (56.17 mg, 20%) as a yellow oil.

LCMS: Rt: 1.550 min; MS m/z (ELSD): 786.4[M+H]⁺;

HPLC: 98.597% purity, ELSD: RT=13.153 min.

¹H NMR (400 MHz, CDCl₃) δ 7.09 (s, 4H), 4.92-4.78 (m, 1H), 4.08 (t, J=6.6 Hz, 2H), 3.62 (t, J=5.2 Hz, 2H), 2.78-2.50 (m, 10H), 2.35-2.22 (m, 4H), 2.00-1.88 (m, 2H), 1.57 (ddd, J=28.9, 13.5, 4.5 Hz, 14H), 1.38-1.20 (m, 42H), 0.88 (t, J=6.8 Hz, 9H).

¹³C NMR (101 MHz, CDCl₃) δ 173.83 (s), 173.60 (s), 140.60 (s), 138.33 (s), 128.44 (s), 128.24 (s), 77.36 (s), 77.04 (s), 76.7 2 (s), 74.15 (s), 63.69 (s), 57.95 (s), 55.83 (s), 53.95 (s), 35.56 (s), 34.65 (s), 34.21 (d, J=13.0 Hz), 31.81 (d, J=12.3 Hz), 31.54 (s), 30.32 (s), 29.52 (d, J=3.1 Hz), 29.34-28.94 (m), 27.13 (d, J=2.5 Hz), 26.31 (s), 25.33 (s), 24.99 (d, J=15.7 Hz), 22.64 (d, J=5.7 Hz), 14.11 (s).

### P. Compound SW-II-138-1

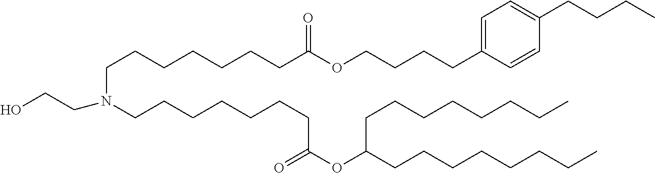 [SMILES: CCCCCCCCC(CCCCCCCC)OC(=O)CCCCCCCN(CCO)CCCCCCCC(=O)OCCCCc1ccc(CCCC)cc1]

### 1. Synthesis of Compound 2

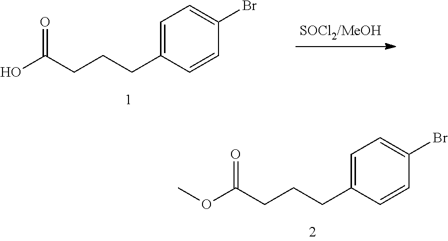

Compound 1 (4 g, 16.46 mmol, 1.0 eq.) was dissolved in MeOH (40 mL), cooled to 0 °C, and SOCl was added dropwise, 2 (3.9 g, 32.92 mmol, 2.0 eq). Then react at room temperature for 1 hour. TLC (PE/EA=5/1) showed consumption of starting material and formation of desired product. The system was directly spin-dried under reduced pressure, and NaHCO was added to the residue.₃ (70 mL) solution, extracted with EA (80 mL×3). Combine the organic phases and wash with saturated brine (2×30 mL), anhydrous Na₂SO₄ Dry, filter and spin dry under reduced pressure. The residue was purified with a silica gel column and eluted with PE/EA (1/0-5:1, v/v) to obtain compound 2 (4.1 mg, 95%) as a yellow oil.

### 2. Synthesis of Compound 4

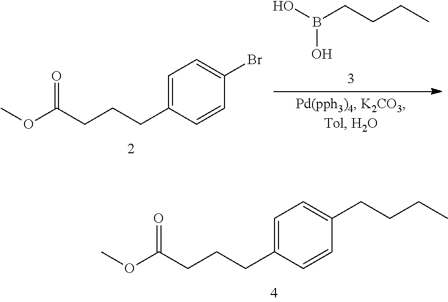

Compound 2 (500 mg, 1.95 mmol, 1.0 eq.), Compound 3 (239 mg, 2.34 mmol, 1.2 eq.), Pd(PPh₃)₄ (225 mg, 0.19 mmol, 0.1 eq) and K₂ CO₃ (808 g, 5.85 mmol, 3.0 eq.) was dissolved in toluene (5.0 mL) and water (1 mL) was added. Then react at 110 °C for 3 hours under nitrogen protection. TLC (PE/EA=5/1) showed consumption of starting material and formation of desired product. Add H to the reaction 2 Quench with O (70 mL) and extract with EA (80 mL×3). Combine the organic phases and wash with saturated brine (2×30 mL), anhydrous Na₂SO₄ Dry, filter and spin dry under reduced pressure. The residue was purified using a silica gel column and eluted with PE/EA (1/0-5:1, v/v) to obtain compound 4 (320 mg, 70%) as a yellow oil.

### 3. Synthesis of Compound 5

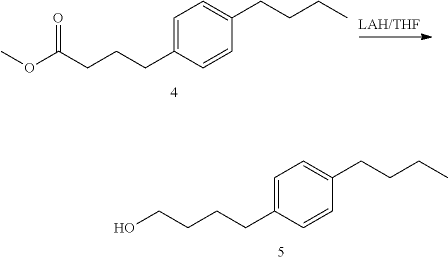 [SMILES: CCCCc1ccc(CCCC(=O)OC)cc1 ; CCCCc1ccc(CCCCO)cc1]

Compound 4 (300 mg, 1.28 mmol, 1.0 eq.) was dissolved in THF (4.0 mL), and LAH (97 mg, 2.56 mmol, 2.0 eq.) was added at 0 °C. Then react at room temperature for 2 hours under nitrogen protection. TLC (PE/EA=5/1) showed consumption of starting material and formation of desired product. Add HCl (1M, 4 mL) solution and H₂O (10 mL) quenched the reaction and extracted with EA (50 mL×3). The organic phase was washed with saturated brine (2×30 mL) and anhydrous Na₂SO₄ Dry, filter and spin dry under reduced pressure. The residue was purified with a silica gel column and eluted with PE/EA (1/0-5:1, v/v) to obtain compound 5 (224 mg, 84.8%) as a yellow oil.

### 4. Synthesis of Compound 7

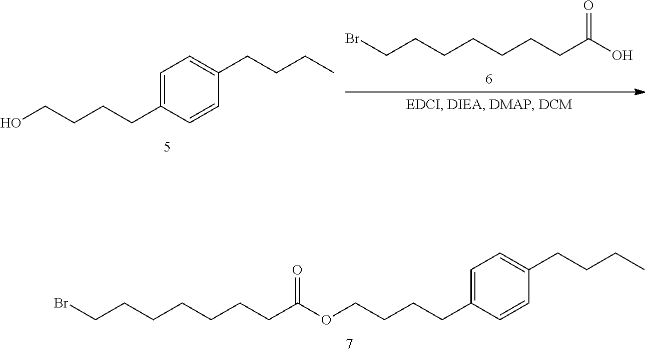

Compound 7 (224 mg, 1.09 mmol, 1.0 eq.) was dissolved in DCM (3.0 mL), and compound 6 (290 mg, 1.30 mmol, 1.2 eq.), EDCI (415 mg, 2.17 mmol, 2.0 eq.), DIEA (561 mg) were added, 4.35 mmol, 4.0 eq.) and DMAP (53 mg, 0.43 mmol, 0.4 eq.). Then, the reaction was carried out at room temperature overnight under nitrogen protection. TLC (PE/EA=30/1) showed consumption of starting material and formation of desired product. The reaction was quenched with HCl (1M) solution and adjusted to pH=4-6, and extracted with DCM (80 mL×3). The combined organic phases were washed with saturated brine (2×30 mL), anhydrous Na₂SO₄ Dry, filter and spin dry under reduced pressure. The residue was purified with a silica gel column and eluted with PE/EA (1/0-30:1, v/v) to obtain compound 7 (208 mg, 46.7%) as a colorless oil.

### 5. Synthesis of SW-II-138-1

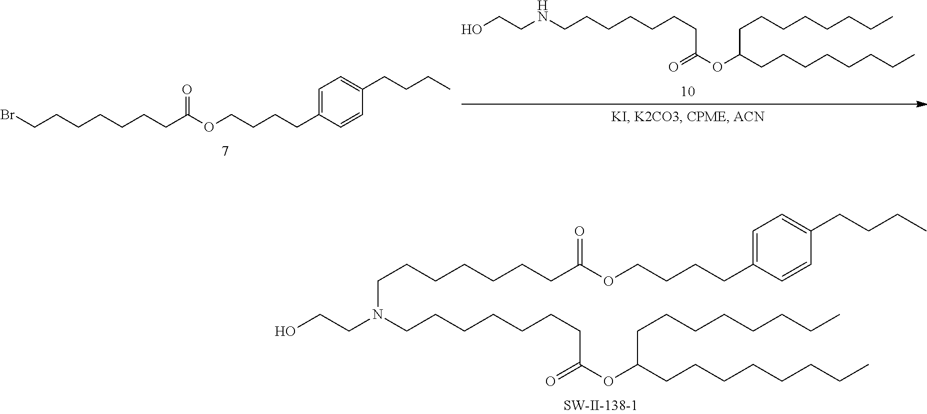 [SMILES: CCCCCCCCC(CCCCCCCC)OC(=O)CCCCCCCNCCO ; CCCCc1ccc(CCCCOC(=O)CCCCCCCBr)cc1 ; CCCCCCCCC(CCCCCCCC)OC(=O)CCCCCCCN(CCO)CCCCCCCC(=O)OCCCCc1ccc(CCCC)cc1]

Compound 10 (110 mg, 0.25 mmol, 1 eq.), compound 7 (153 mg, 0.37 mmol, 1.5 eq.), KI (83 mg, 0.50 mmol, 2.0 eq.) and CPME (2 mL) were dissolved in MeCN (2 mL) and K was added₂ CO₃ (172 mg, 1.25 mmol, 5.0 eq). The reaction was carried out overnight at 90 °C under nitrogen protection. TLC (DCM/MeOH=10/1) showed consumption of starting material and formation of the desired product. The reaction was directly rotated to dryness under reduced pressure. The residue was purified by silica gel column and eluted with DCM/MeOH (1/0-10:1, v/v) to obtain a light yellow oily compound (65 mg, 32%, SW-II-138-1).

LCMS: Rt: 1.684 min; MS m/z (ELSD): 772.4[M+H]⁺;

HPLC: 96.56% purity, ELSD; RT=6.346 min.

¹H NMR (400 MHz, CDCl₃) δ 7.09 (s, 4H), 4.86 (s, 1H), 4.09 (d, J=6.0 Hz, 2H), 3.97 (s, 2H), 3.07 (d, J=38.8 Hz, 6H), 2.69-2.51 (m, 4H), 2.28 (td, J=7.3, 3.6 Hz, 4H), 1.79 (s, 4H), 1.70-1.46 (m, 16H), 1.42-1.17 (m, 37H), 0.90 (dt, J=13.6, 7.2 Hz, 9H).

¹³C NMR (101 MHz, CDCl₃) δ 173.80 (s), 173.53 (s), 140.32 (s), 139.13 (s), 128.28 (d, J=13.6 Hz), 77.43 (s), 77.11 (s), 76.80 (s), 74.21 (s), 64.22 (s), 56.85 (s), 55.98 (s), 53.93 (s), 35.22 (s), 35.01 (s), 34.54 (s), 34.14 (d, J=5.6 Hz), 33.71 (s), 31.85 (s), 29.50 (d, J=2.8 Hz), 29.22 (s), 29.12-28.60 (m), 28.26 (s), 27.78 (s), 26.70 (d, J=4.4 Hz), 25.31 (s), 24.82 (d, J=17.6 Hz), 24.28 (s), 22.65 (s), 22.37 (s), 14.03 (d, J=15.2 Hz).

### Q. SW-II-138-2

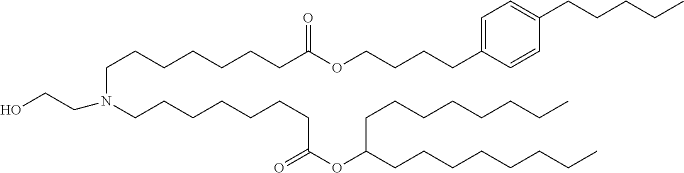 [SMILES: CCCCCCCCC(CCCCCCCC)OC(=O)CCCCCCCN(CCO)CCCCCCCC(=O)OCCCCc1ccc(CCCCC)cc1]

### 1. Synthesis of Compound 3

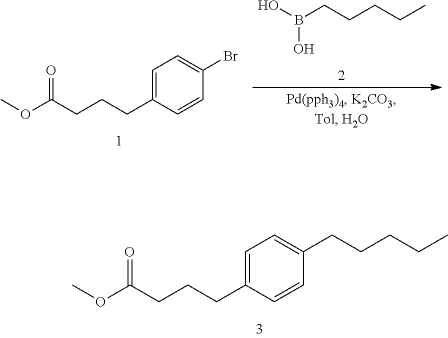

Compound 1 (500 mg, 1.95 mmol, 1.0 eq.), Compound 2 (271 mg, 2.34 mmol, 1.2 eq.), Pd(PPh₃)₄ (225 mg, 0.20 mmol, 0.1 eq) and K₂ CO₃ (809 g, 5.86 mmol, 3.0 eq.) was dissolved in toluene (5.0 mL), water (1 mL) was added, and then reacted at 110 °C for 3 hours under nitrogen protection. TLC (PE/EA=5/1) showed consumption of starting material and formation of desired product. The reaction was quenched by adding water (70 mL) and extracted with EA (80 mL×3). Combine the organic phases and wash with saturated brine (2×30 mL), anhydrous Na₂SO₄ Dry, filter and spin dry under reduced pressure. The residue was purified with a silica gel column and eluted with PE/EA (1/0-30:1, v/v) to obtain compound 3 (320 mg, 70%) as a colorless oil.

### 2. Synthesis of Compound 4

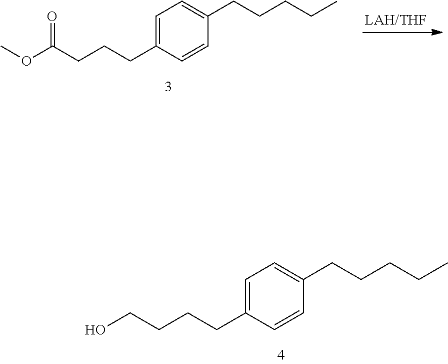 [SMILES: CCCCCc1ccc(CCCC(=O)OC)cc1 ; CCCCCc1ccc(CCCCO)cc1]

Compound 3 (320 mg, 1.29 mmol, 1.0 eq.) was dissolved in THF (3.0 mL), LAH (67 mg, 1.77 mmol, 2.0 eq.) was added at 0 °C and then reacted at room temperature under nitrogen protection for 2 hours. TLC (PE/EA=5/1) showed consumption of starting material and formation of desired product. The reaction uses HCl (1 M, 2 mL) solution and H₂ Quench with O (10 mL) and extract with EA (50 mL×3). The combined organic phases were washed with saturated brine (2×30 mL), anhydrous Na₂SO₄ Dry, filter and spin dry under reduced pressure. The residue was purified with a silica gel column and eluted with PE/EA (1/0-30:1, v/v) to obtain compound 4 (180 mg, 64%) as a colorless oil.

### 3. Synthesis of Compound 6

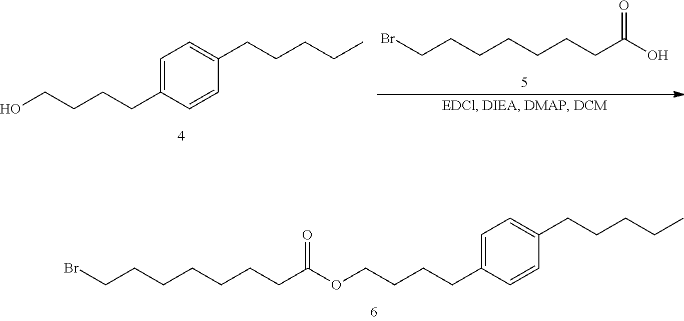

Compound 4 (180 mg, 0.82 mmol, 1.0 eq.) was dissolved in DCM (3.0 mL) and compound 5 (245 mg, 1.10 mmol, 1.2 eq.), EDCI (347 mg, 1.82 mmol, 2.0 eq.), DIEA (470 mg, 3.63 mmol, 4.0 eq.) and DMAP (45 mg, 0.36 mmol, 0.4 eq.). Then, the reaction was carried out overnight at room temperature under nitrogen protection. TLC (PE/EA=30/I) showed complete consumption of starting material and formation of the desired product. The reaction was quenched with HCl (1M) solution and adjusted to pH=5-6, and extracted with DCM (80 mL×3). The combined organic phases were washed with saturated brine (2×30 mL), anhydrous Na₂SO₄ Dry, filter and spin dry under reduced pressure. The residue was purified with a silica gel column and eluted with PE/EA (1/0-30:1, v/v) to obtain compound 6 (220 mg, 63.6%) as a colorless oil.

### 4. Synthesis of SW-II-138-2

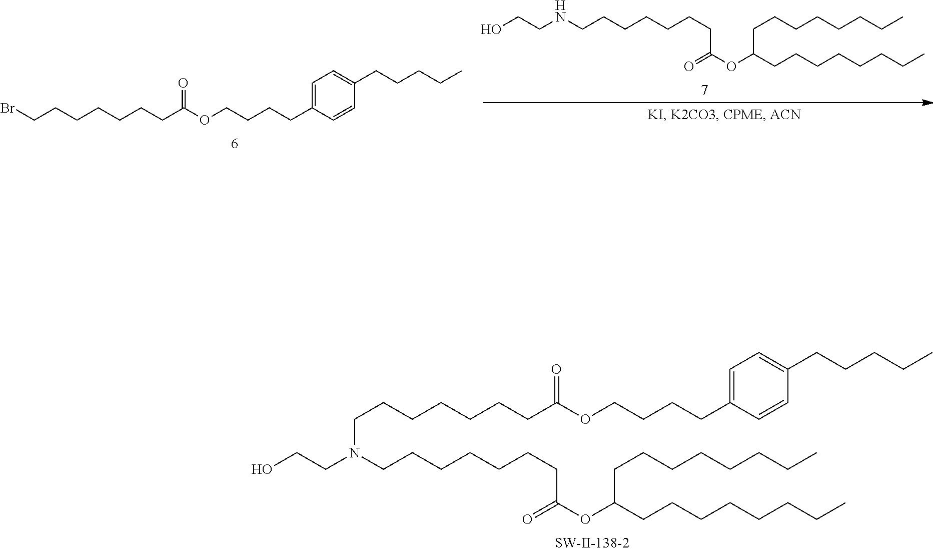 [SMILES: CCCCCCCCC(CCCCCCCC)OC(=O)CCCCCCCNCCO ; CCCCCc1ccc(CCCCOC(=O)CCCCCCCBr)cc1 ; CCCCCCCCC(CCCCCCCC)OC(=O)CCCCCCCN(CCO)CCCCCCCC(=O)OCCCCc1ccc(CCCCC)cc1]

Compound 6 (158 mg, 0.37 mmol, 1.5 eq.), compound 7 (110 mg, 0.25 mmol, 1.0 eq.), KI (83 mg, 0.50 mmol, 2.0 eq.) and CPME (2 mL) were dissolved in MeCN (2 mL), and K was added₂ CO₃ (172 mg, 1.25 mmol, 5.0 eq). Then, the reaction was carried out overnight at 90 °C under nitrogen protection. TLC (DCM/MeOH=10/1) showed consumption of starting material and formation of the desired product. The reaction was directly spun to dryness under reduced pressure, and the residue was purified with a silica gel column and eluted with DCM/MeOH (1/0-10:1, v/v) to obtain the target product as a colorless oil (100 mg, 51%, SW-II-138-2).

LCMS: Rt: 1.834 min; MS m/z (ELSD): 786.4[M+H]⁺;

HPLC: 99.20% purity, ELSD; RT=7.990 min.

¹H NMR (400 MHz, CDCl₃) δ 7.00 (s, 4H), 4.88-4.73 (m, 2H), 4.00 (t, J=5.6 Hz, 2H), 3.81-3.54 (m, 2H), 3.00-2.81 (m, 2H), 2.81-2.65 (m, 4H), 2.50 (dd, J=16.4, 8.4 Hz, 4H), 2.20 (td, J=7.6, 3.2 Hz, 4H), 1.56 (ddd, J=18.4, 10.4, 5.2 Hz, 13H), 1.43 (d, J=5.6 Hz, 4H), 1.34-1.07 (m, 40H), 0.8 I(dt, J=11.2, 5.6 Hz, 9H).

¹³C NMR (101 MHz, CDCl₃) δ 173.78 (s), 173.52 (s), 140.32 (s), 139.11 (s), 128.25 (d, J=11.6 Hz), 77.49 (s), 77.17 (s), 76.85 (s), 74.14 (s), 64.17 (s), 57.25 (s), 55.82 (s), 53.85 (s), 35.50 (s), 35.01 (s), 34.56 (s), 34.14 (d, J=7.2 Hz), 31.84 (s), 31.53 (s), 31.23 (s), 29.49 (d, J=2.8 Hz), 29.21 (s), 28.94 (dd, J=6.4, 4.4 Hz), 28.25 (s), 2 7.77 (s), 26.84 (d, J=4.4 Hz), 25.30 (s), 25.25-24.59 (m), 22.59 (d, J=11.2 Hz), 14.05 (d, J=7.6 Hz).

### R. SW-II-138-3

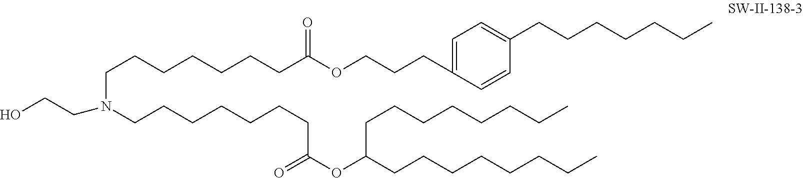 [SMILES: CCCCCCCCC(CCCCCCCC)OC(=O)CCCCCCCN(CCO)CCCCCCCC(=O)OCCCc1ccc(CCCCCCC)cc1]

### 1. Synthesis of Compound 3

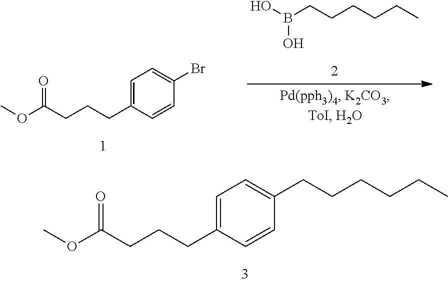

Compound 1 (500 mg, 1.95 mmol, 1.0 eq), Compound 2 (305 mg, 2.34 mmol, 1.2 eq.), Pd(PPh₃)₄ (225 mg, 0.20 mmol, 0.1 eq) and K₂CO₃ (809 g, 5.86 mmol, 3.0 eq.) was dissolved in toluene (5.0 mL) and water (1 mL) was added. Ten, the reaction was carried out at 110 °C for 3 hours under nitrogen protection. TLC (PE/EA=5/1) showed complete consumption of starting material and formation of the desired compound. The reaction was quenched by adding water (80 mL) and extracted with EA (80 mL×3). Combine the organic phases and wash with saturated brine (2×40 mL), anhydrous Na₂SO₄ Dry, filter and spin dry under reduced pressure. The residue was purified with a silica gel column and eluted with PE/EA (1/0-5:1, v/v) to obtain compound 3 (260 mg, 51.3%) as a colorless oil.

### 2. Synthesis of Compound 4

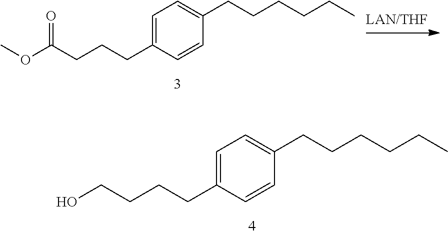 [SMILES: CCCCCCc1ccc(CCCC(=O)OC)cc1 ; CCCCCCc1ccc(CCCCO)cc1]

Compound 3 (260 mg, 0.99 mmol, 1.0 eq.) was dissolved in THF (4.0 mL), and LAH (75 mg, 1.98 mmol, 2.0 eq.) was added at 0 °C. Then, react at room temperature for 2 hours under nitrogen protection. TLC (PE/EA=5/1) showed that the starting material was completely reacted and the desired compound was formed. The reaction uses HCl (1M, 4 mL) solution and H₂ Quench with O (20 mL) and extract with EA (50 mL×3). The combined organic phases were washed with saturated brine (2×30 mL) and anhydrous Na₂SO₄ Dry, filter and spin dry under reduced pressure. The residue was purified with a silica gel column and eluted with PE/EA (1/0-5:1, v/v) to obtain compound 4 (230 mg, 98%) as a colorless oil.

### 3. Synthesis of Compound 6

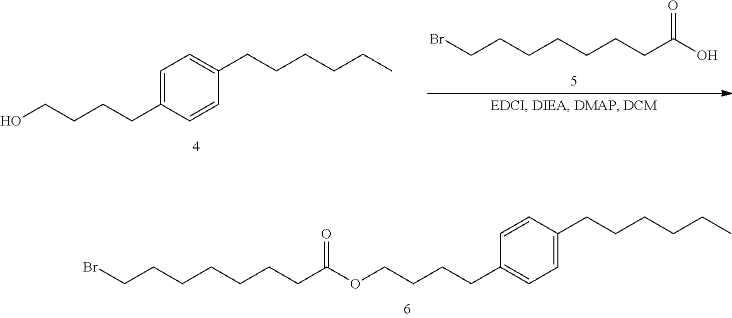 [SMILES: CCCCCCc1ccc(CCCCO)cc1 ; O=C(O)CCCCCCCBr ; CCCCCCc1ccc(CCCCOC(=O)CCCCCCCBr)cc1]

Compound 4 (240 mg, 1.03 mmol, 1.0 eq.) was dissolved in DCM (4.0 mL), and compound 5 (275 mg, 1.23 mmol, 1.2 eq.), EDCI (392 mg, 2.07 mmol, 2.0 eq.), DIEA (530 mg, 4.10 mmol, 4.0 eq.) and DMAP (50 mg, 0.41 mmol, 0.4 eq.). Then react overnight at room temperature under nitrogen protection. TLC (PE/EA=20/1) showed consumption of starting material and formation of the desired compound. The reaction was quenched with HCl (1M) and adjusted to pH=5~6, and extracted with DCM (80 mL×3). The combined organic phases were washed with saturated brine (2-30 mL), anhydrous Na₂SO₄ Dry, filter and spin dry under reduced pressure. The residue was purified with a silica gel column and eluted with PE/EA (1/0-20:1, v/v) to obtain compound 6 (180 mg, 40.9%) as a colorless oil.

### 4. Synthesis of SW-II-138-3

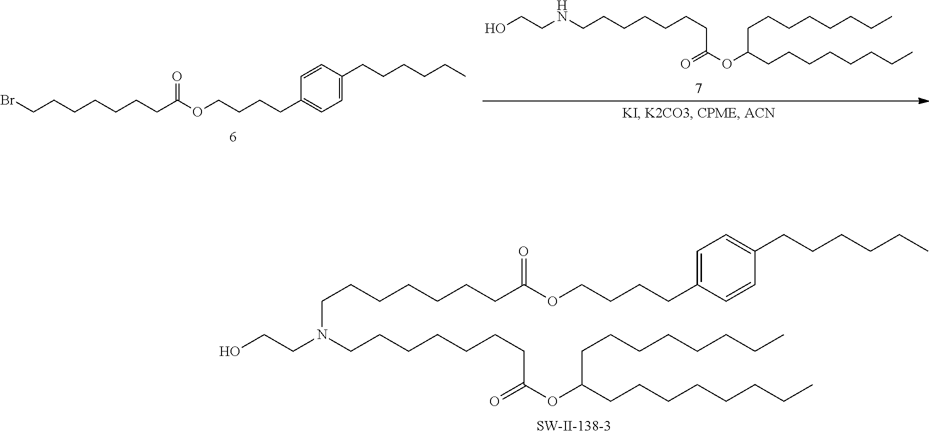 [SMILES: CCCCCCCCC(CCCCCCCC)OC(=O)CCCCCCCNCCO ; CCCCCCc1ccc(CCCCOC(=O)CCCCCCCBr)cc1 ; CCCCCCCCC(CCCCCCCC)OC(=O)CCCCCCCN(CCO)CCCCCCCC(=O)OCCCCc1ccc(CCCCCC)cc1]

Compound 6 (164 mg, 0.37 mmol, 1 eq.) and compound 7 (110 mg, 0.24 mmol, 1.0 eq.), KI (83 mg, 0.49 mmol, 2.0 eq.) and CPME (2 mL) were dissolved in MeCN (2 mL) and K was added₂ CO₃ (172 mg, 1.24 mmol, 5.0 eq). Then, the reaction was carried out at 90 °C overnight under nitrogen protection. TLC (DCM/MeOH=10/1) showed consumption of starting material and formation of desired product. The reaction was directly rotated to dryness under reduced pressure. The residue was purified with a silica gel column and eluted with DCM/MeOH (1/0-10:1, v/v) to obtain the target product (108 mg, 52.76%, SW-II-138-3) as a colorless oil.

LCMS: Rt: 2.007 min; MS m/z (ELSD): 800.4[M+H]⁺;

HPLC: 97.95% purity, ELSD; RT=9.455 min.

¹H NMR (400 MHz, CDCl₃) δ 7.08 (s, 4H), 4.86 (p, J=6.4 Hz, 1H), 4.08 (s, 2H), 3.60 (t, J=5.2 Hz, 3H), 2.76-2.42 (m, 10H), 2.28 (td, J=7.6, 2.8 Hz, 4H), 1.70-1.42 (m, 18H), 1.28 (d, J=20.0 Hz, 41H), 0.88 (t, J=6.8 Hz, 9H).

¹³C NMR (101 MHz, CDCl₃) δ 173.85 (s), 173.59 (s), 140.39 (s), 139.15 (s), 128.27 (d, J=12.0 Hz), 77.38 (s), 77.07 (s), 76.75 (s), 74.13 (s), 64.17 (s), 58.06 (s), 55.75 (s), 53.92 (s), 35.57 (s), 35.03 (s), 34.66 (s), 34.22 (d, J=13.2 Hz), 31.81 (d, J=12.4 Hz), 31.54 (s), 29.52 (d, J=2.9 Hz), 29.34-28.95 (m), 28.29 (s), 27.79 (s), 27.16 (d, J=3.6 Hz), 26.50 (s), 25.32 (s), 24.99 (d, J=17.6 Hz), 22.64 (d, J=5.6 Hz), 14.10 (s).

### S. Compound SW-II-139-1

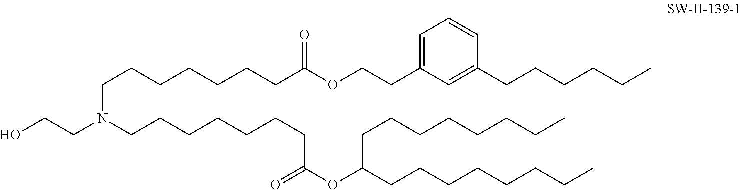 [SMILES: CCCCCCCCC(CCCCCCCC)OC(=O)CCCCCCCN(CCO)CCCCCCCC(=O)OCCc1cccc(CCCCCC)c1]

### 1. Synthesis of Compound 3

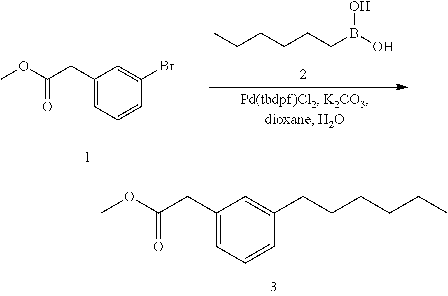

To a mixture of compound 1 (1 g, 4.37 mmol, 1 eq.) and compound 2 (852 g, 6.55 mmol, 1.5 eq.) in 1,4-dioxane/water (10 mL/1 mL) was added Pd(dtbpf)Cl₂ (286 mg, 0.437 mmol, 0.1 eq.) and potassium carbonate (1.8 g, 13.11 mmol, 3 eq.). The mixture was stirred at 100 °C overnight under nitrogen. TLC (PE/EA=20/1) showed the reaction was complete and new major spots were observed. The mixture was extracted with ethyl acetate and washed with water. The organic layer was dried over anhydrous sodium sulfate, filtered and concentrated in vacuo. The residue was purified by silica gel column chromatography, eluting with PE/EA (1/0-20/1), to obtain compound 3 (691 mg, 68%) as a colorless oil.

### 2. Synthesis of Compound 4

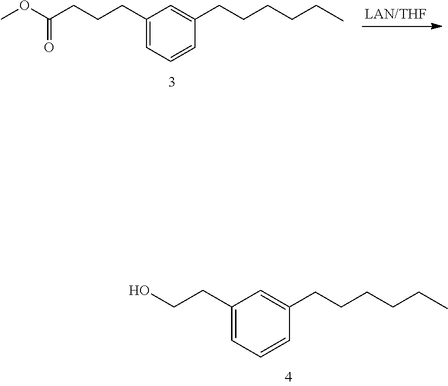 [SMILES: CCCCCCc1cccc(CCCC(=O)OC)c1 ; CCCCCCc1cccc(CCO)c1]

To a mixture of compound 3 (691 mg, 2.95 mmol, 1 eq.) in THF (7 mL) was added lithium aluminum hydride (3 mL, 2.95 mmol, 1 M, 1 eq. in THF) at 0 °C under nitrogen. The mixture was stirred at room temperature for 3 hours. TLC (PE/EA=5/1) indicated that the reaction was complete and new major spots were observed. The mixture was quenched with water (3 mL) and treated with 2N hydrochloric acid to adjust the pH between 6 and 7, extracted with ethyl acetate and washed with brine. The organic layer was dried over anhydrous sodium sulfate, filtered and concentrated in vacuo to afford compound 4 (547 mg, 90%) as a colorless oil without further purification.

### 3. Synthesis of Compound 6

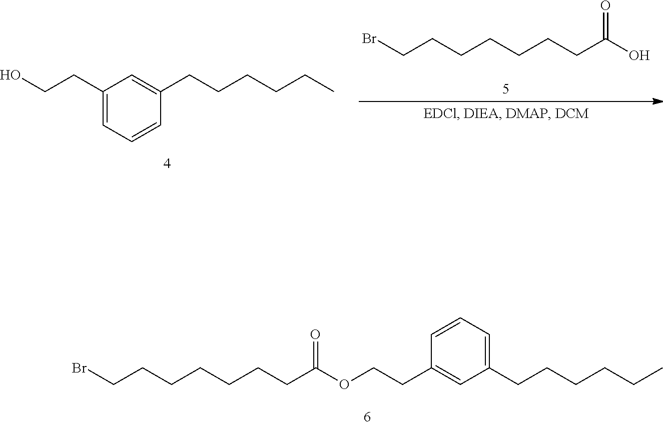 [SMILES: O=C(O)CCCCCCCBr ; CCCCCCc1cccc(CCO)c1 ; CCCCCCc1cccc(CCOC(=O)CCCCCCCBr)c1]

To compound 4 (447 mg, 2.17 mmol, 1 eq.) and compound 5 (581 mg, 2.6 mmol, 1.2 eq.) in DCM

To the mixture in (5 mL), EDCI (833 mg, 4.34 mmol, 2 eq.) and DMAP (106 mg, 0.87 mmol, 0.4 eq.) were added, followed by DIEA (1.12 g, 8.68 mmol, 4 eq.). The reaction mixture was stirred at room temperature under nitrogen for 16 hours. TLC (petroleum ether/ethyl acetate=15/1) showed the formation of the desired product. The reaction mixture was extracted with ethyl acetate and washed with water. The organic layer was dried over anhydrous sodium sulfate, filtered and concentrated in vacuo. The residue was purified by silica gel column chromatography, eluting with petroleum ether/ethyl acetate (1/0-20/1), to obtain compound 6 (455 mg, 51%) as a colorless oil.

### 4. Synthesis of SW-II-139-1

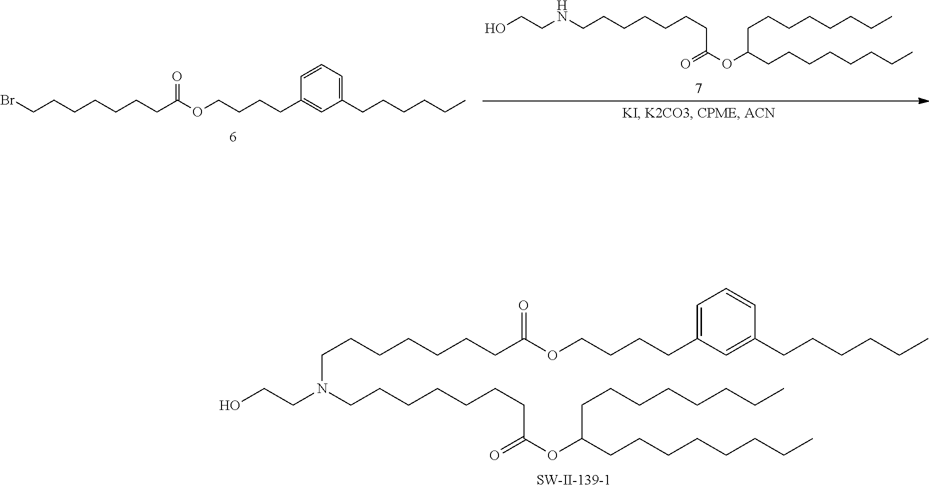 [SMILES: CCCCCCCCC(CCCCCCCC)OC(=O)CCCCCCCNCCO ; CCCCCCc1cccc(CCCCOC(=O)CCCCCCCBr)c1 ; CCCCCCCCC(CCCCCCCC)OC(=O)CCCCCCCN(CCO)CCCCCCCC(=O)OCCCCc1cccc(CCCCCC)c1]

To compound 6 (150 mg, 0.365 mmol, 1 eq.) and compound 7 (161 mg, 0.365 mmol, 1 eq.) in CPME/CH₃ To the mixture in CN (2 mL/2 mL) were added potassium carbonate (252 mg, 1.825 mmol, 6 eq.) and potassium iodide (121 mg, 0.73 mmol, 2 eq.). After addition, the mixture was stirred at 90 °C overnight under nitrogen. TLC (DCM/MeOH=10/1) showed the reaction was complete and new major spots were observed. The mixture was extracted with ethyl acetate and washed with water. The organic layer was dried over anhydrous sodium sulfate, filtered and concentrated under reduced pressure. The residue was purified by silica gel column chromatography, eluting with DCM/MeOH (1/0-10:1, v/v), to obtain compound SW-II-139-1 (54.53 mg, 19%) as a yellow oil.

LCMS: Rt: 1.521 min; MS m/z (ELSD): 772.4[M+H]⁺;

HPLC: 99.637% purity, ELSD: RT=12.347 min.

¹H NMR (400 MHz, CDCl₃) δ 7.20 (t, J=7.7 Hz, 1H), 7.03 (t, J=6.8 Hz, 3H), 4.94-4.78 (m, 1H), 4.27 (t, J=7.2 Hz, 2H), 3.65 (t, J=5.1 Hz, 2H), 2.90 (t, J=7.2 Hz, 2H), 2.73 (t, J=4.9 Hz, 2H), 2.67-2.41 (m, 6H), 2.28 (td, J=7.5, 2.7 Hz, 4H), 1.67-1.45 (m, 14H), 1.41-1.19 (m, 42H), 0.88 (dd, J=7.9, 5.7 Hz, 9H).

¹³C NMR (101 MHz, CDCl₃) δ 173.65 (d, J=11.3 Hz), 143.17 (s), 137.67 (s), 129.04 (s), 128.34 (s), 126.61 (s), 126.11 (s), 7 7.30 (d, J=11.6 Hz), 77.04 (s), 76.72 (s), 74.16 (s), 64.85 (s), 57.88 (s), 55.93 (s), 53.97 (s), 35.94 (s), 35.13 (s), 34.64 (s), 34.20 (d, J=10.5 Hz), 31.80 (d, J=13.7 Hz), 31.50 (s), 29.52 (d, J=2.9 Hz), 29.34-28.92 (m), 27.08 (d, J=3.9 Hz), 26.10 (s), 25.33 (s), 25.05 (s), 24.82 (s), 22.64 (d, J=6.5 Hz), 14.11 (s).

### T. Compound SW-II-139-2

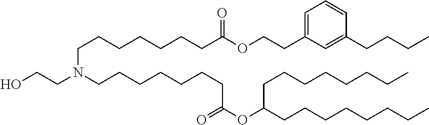 [SMILES: CCCCCCCCC(CCCCCCCC)OC(=O)CCCCCCCN(CCO)CCCCCCCC(=O)OCCc1cccc(CCCC)c1]

### 1. Synthesis of Compound 3

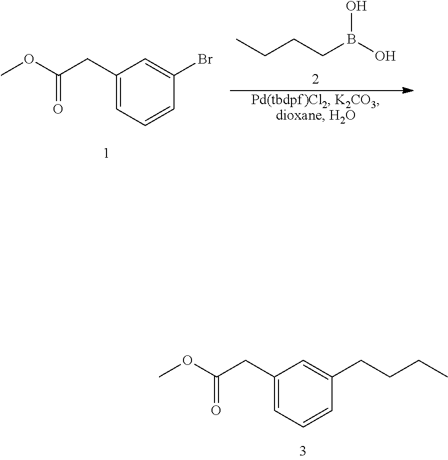

To a mixture of compound 1 (1 g, 4.37 mmol, 1 eq.) and compound 2 (668 g, 6.55 mmol, 1.5 eq.) in 1,4-dioxane/water (10 mL/1 mL) was added Pd(dtbpf)Cl₂ (286 mg, 0.437 mmol, 0.1 eq.) and potassium carbonate (1.8 g,

13.11 mmol, 3 eq). The mixture was stirred at 100 °C overnight under nitrogen. TLC (PE/EA=20/1) showed the reaction was complete and new major spots were observed. The mixture was extracted with ethyl acetate and washed with water. The organic layer was dried over anhydrous sodium sulfate, filtered and concentrated in vacuo. The residue was purified by silica gel column chromatography, eluting with PE/EA (1/0-20/1), to obtain compound 3 (605 mg, 67%) as a colorless oil.

### 2. Synthesis of Compound 4

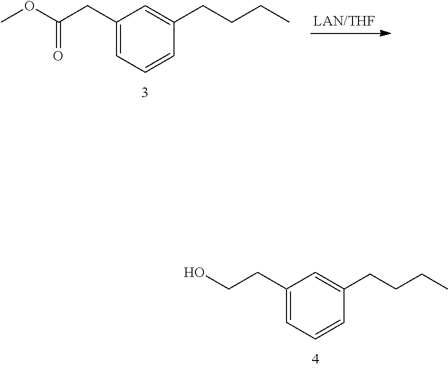

To a mixture of compound 3 (605 mg, 2.94 mmol, 1 eq.) in THF (7 mL) was added lithium aluminum hydride (3 mL, 2.94 mmol, 1 M, 1 eq. in THF) at 0 °C under nitrogen. The mixture was stirred at room temperature for 3 hours. TLC (PE/EA=5/1) indicated that the reaction was complete and new major spots were observed. The mixture was quenched with water (3 mL) and treated with 2N hydrochloric acid to adjust the pH between 6 and 7, extracted with ethyl acetate and washed with brine. The organic layer was dried over anhydrous sodium sulfate, filtered and concentrated in vacuo to afford compound 4 (534 mg, >100%) as a colorless oil without further purification.

### 3. Synthesis of Compound 6

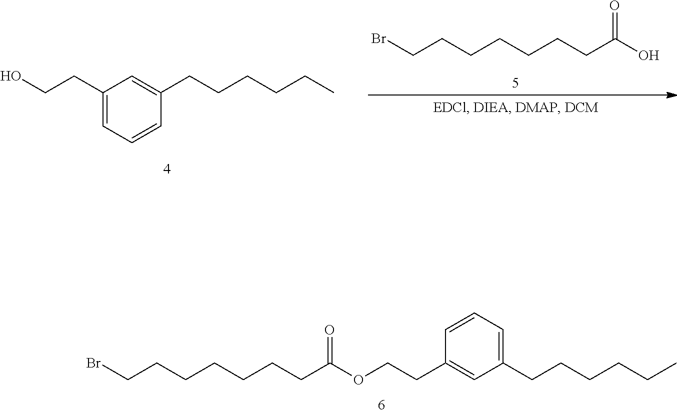

To a mixture of compound 4 (434 mg, 2.44 mmol, 1 eq.) and compound 5 (652 mg, 2.93 mmol, 1.2 eq.) in DCM (5 mL) was added EDCI (937 mg, 4.88 mmol, 2 eq.) and DMAP (119 mg, 0.976 mmol, 0.4 eq.), then DIEA (1.259 g, 9.76 mmol, 4 eq.) was added. The reaction mixture was stirred at room temperature under nitrogen for 16 hours. TLC (petroleum ether/ethyl acetate=15/1) showed the formation of the desired product. The reaction mixture was extracted with ethyl acetate and washed with water. The organic layer was dried over anhydrous sodium sulfate, filtered and concentrated in vacuo. The residue was purified by silica gel column chromatography, eluting with petroleum ether/ethyl acetate (1/0-20/1), to obtain compound 6 (355 mg, 38%) as a colorless oil.

### 4. Synthesis of SW-II-139-2

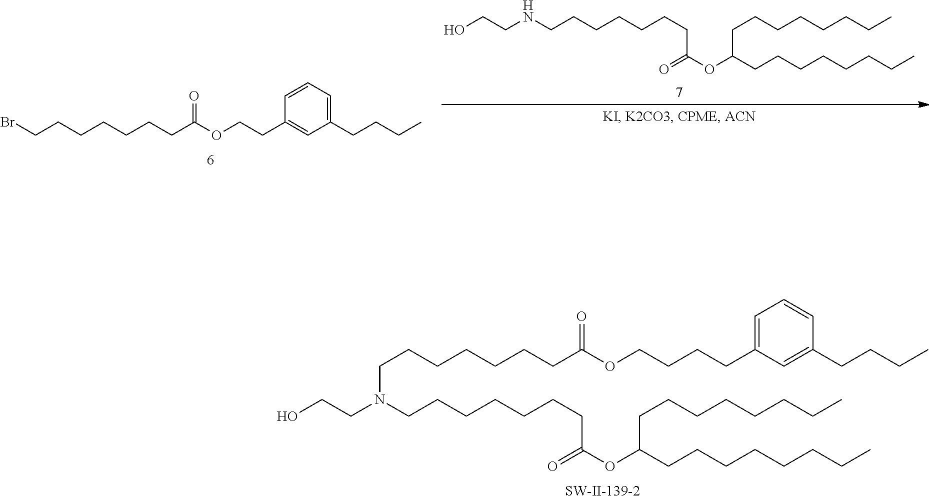 [SMILES: CCCCCCCCC(CCCCCCCC)OC(=O)CCCCCCCNCCO ; CCCCc1cccc(CCOC(=O)CCCCCCCBr)c1 ; CCCCCCCCC(CCCCCCCC)OC(=O)CCCCCCCN(CCO)CCCCCCCC(=O)OCCCCc1cccc(CCCC)c1]

To compound 6 (122 mg, 0.319 mmol, 1 eq.) and compound 7 (140 mg, 0.319 mmol, 1 eq.) in CPME/CH₃ Potassium carbonate (220 mg, 1.595 mmol, 5 eq.) and potassium iodide (106 mg, 0.638 mmol, 2 eq.) were added to the mixture in CN (2 mL/2 mL). After addition, the mixture was stirred at 90 °C overnight under nitrogen. TLC (DCM/MeOH=10/1) showed the reaction was complete and new major spots were observed. The mixture was extracted with ethyl acetate and washed with water. The organic layer was dried over anhydrous sodium sulfate, filtered and concentrated under reduced pressure. The residue was purified by silica gel column chromatography, eluting with DCM/MeOH (1/0-10:1, v/v), to obtain compound SW-II-139-2 (45.48 mg, 19%) as a yellow oil.

LCMS: Rt: 1.346 min; MS m/z (ELSD): 744.3[M+H]⁺;

HPLC: 97.994% purity, ELSD; RT=11.235 min.

¹H NMR (400 MHz, CDCl₃) δ 7.20 (t, J=7.8 Hz, 1H), 7.03 (t, J=7.6 Hz, 3H), 4.91-4.81 (m, 1H), 4.27 (t, J=7.2 Hz, 2H), 3.89-3.75 (m, 2H), 2.99-2.79 (m, 7H), 2.64-2.48 (m, 2H), 2.28 (td, J=7.5, 3.1 Hz, 4H), 1.74-1.08 (m, 53H), 0.90 (dt, J=13.6, 7.2 Hz, 9H).

¹³C NMR (101 MHz, CDCl₃) δ 173.60 (d, J=11.7 Hz), 143.13 (s), 137.65 (s), 129.06 (s), 128.34 (s), 126.64 (s), 126.11 (s), 7 7.30 (d, J=11.4 Hz), 77.04 (s), 76.72 (s), 74.22 (s), 64.88 (s), 57.28 (s), 56.55 (s), 54.11 (s), 35.60 (s), 35.12 (s), 34.56 (s), 34.15 (d, J=4.0 Hz), 33.68 (s), 31.86 (s), 29.52 (d, J=2.8 Hz), 29.24 (s), 28.91 (dd, J=7.0, 4.2 Hz), 26.81 (d, J=3.9 Hz), 25.33 (s), 25.12-24.98 (m), 24.83 (d, J=22.2 Hz), 22.67 (s), 22.40 (s), 14.04 (d, J=14.4 Hz).

### U. Compound SW-II-140-1

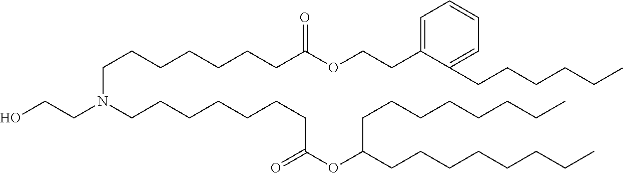 [SMILES: CCCCCCCCC(CCCCCCCC)OC(=O)CCCCCCCN(CCO)CCCCCCCC(=O)OCCc1ccccc1CCCCCC]

### 1. Synthesis of Compound 3

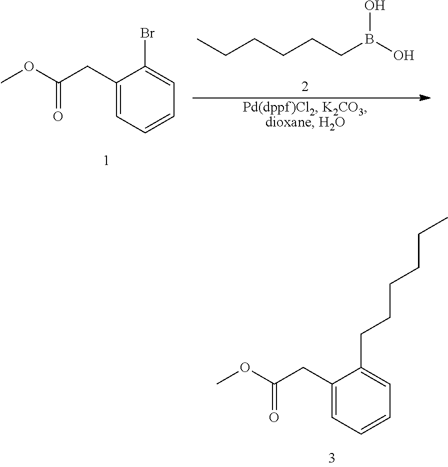

To a mixture of compound 1 (1 g, 4.37 mmol, 1 eq.) and compound 2 (852 g, 6.55 mmol, 1.5 eq.) in 1,4-dioxane/water (10 mL/1 mL) was added Pd(dppf)Cl₂ (286 mg, 0.437 mmol, 0.1 eq.) and potassium carbonate (1.8 g, 13.11 mmol, 3 eq.). The mixture was stirred at 100 °C overnight under nitrogen. TLC (PE/EA=20/1) showed the reaction was complete and new major spots were observed. The mixture was extracted with ethyl acetate and washed with water. The organic layer was dried over anhydrous sodium sulfate, filtered and concentrated in vacuo. The residue was purified by silica gel column chromatography, eluting with PE/EA (1/0-20/1), to obtain compound 3 (748 mg, 73%) as a colorless oil.

### 2. Synthesis of Compound 4

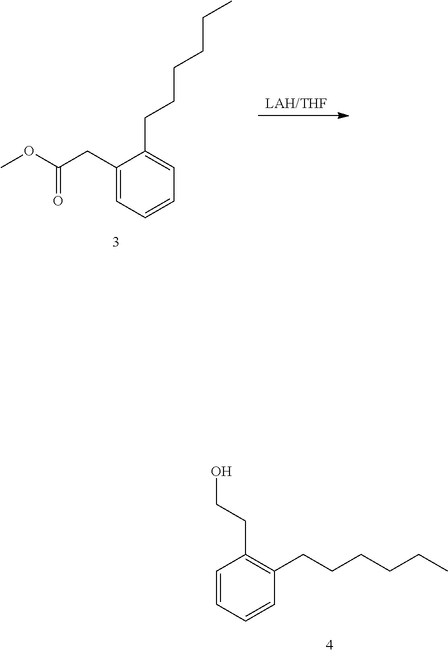 [SMILES: CCCCCCc1ccccc1CC(=O)OC ; CCCCCCc1ccccc1CCO]

To a mixture of compound 3 (748 mg, 3.2 mmol, 1 eq.) in THF (8 mL) was added lithium aluminum hydride (3.2 mL, 3.2 mmol, 1 M, 1 eq. in THF) at 0 °C under nitrogen. The mixture was stirred at room temperature for 3 hours. TLC (PE/EA=5/1) indicated that the reaction was complete and new major spots were observed. The mixture was quenched with water (3 mL) and treated with 2N hydrochloric acid to adjust the pH between 6 and 7, extracted with ethyl acetate and washed with brine. The organic layer was dried over anhydrous sodium sulfate, filtered and concentrated in vacuo to afford compound 4 (493 mg, 75%) as a colorless oil without further purification.

### 3. Synthesis of Compound 6

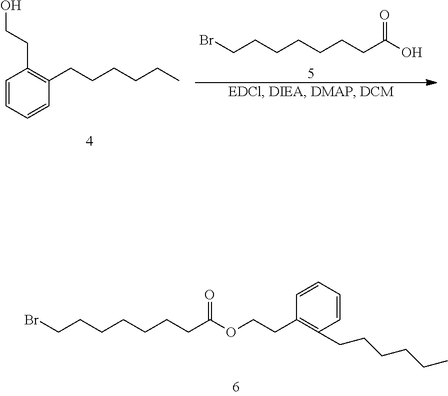

To a mixture of compound 4 (393 mg, 1.91 mmol, 1 eq.) and compound 5 (511 mg, 2.29 mmol, 1.2 eq.) in DCM (5 mL) was added EDCI (733 mg, 3.82 mmol, 2 eq.) and DMAP (93 mg, 0.76 mmol, 0.4 eq.), then DIEA (986 mg, 7.64 mmol, 4 eq.) was added. The reaction mixture was stirred at room temperature under nitrogen for 16 hours. TLC (petroleum ether/ethyl acetate=15/1) showed the formation of the desired product. The reaction mixture was extracted with ethyl acetate and washed with water. The organic layer was dried over anhydrous sodium sulfate, filtered and concentrated in vacuo. The residue was purified by silica gel column chromatography, eluting with petroleum ether/ethyl acetate (1/0-20/1), to give compound 6 (327 mg, 42%) as a colorless oil.

### 4. Synthesis of SW-II-140-1

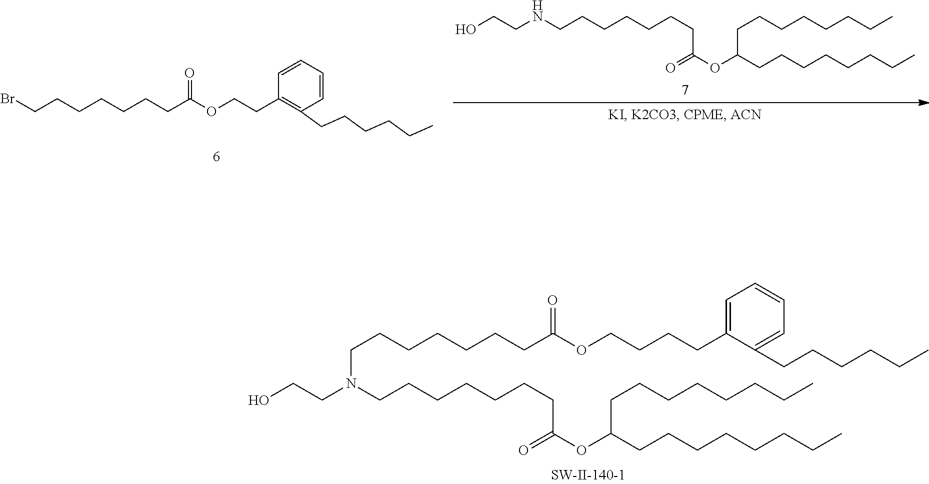 [SMILES: CCCCCCc1ccccc1CCOC(=O)CCCCCCCBr ; CCCCCCCCC(CCCCCCCC)OC(=O)CCCCCCCNCCO ; CCCCCCCCC(CCCCCCCC)OC(=O)CCCCCCCN(CCO)CCCCCCCC(=O)OCCCCc1ccccc1CCCCCC]

To compound 6 (150 mg, 0.365 mmol, 1 eq.) and compound 7 (161 mg, 0.365 mmol, 1 eq.) in CPME/CH₃Potassium carbonate (302 mg, 2.19 mmol, 6 eq.) and potassium iodide (121 mg, 0.73 mmol, 2 eq.) were added to the mixture in CN (2 mL/2 mL). After addition, the mixture was stirred at 90 °C overnight under nitrogen. TLC (DCM/MeOH=10/1) showed the reaction was complete and new major spots were observed. The mixture was extracted with ethyl acetate and washed with water. The organic layer was dried over anhydrous sodium sulfate, filtered and concentrated under reduced pressure. The residue was purified by silica gel column chromatography, eluting with DCM/MeOH (1/0-10:1, v/v), to obtain compound SW-II-140-1 (180 mg, 64%) as a yellow oil.

LCMS: Rt: 1.568 min; MS m/z (ELSD): 772.4[M+H]⁺;

HPLC: 98.053% purity, ELSD; RT=8.702 min.

¹H NMR (400 MHz, CDCl₃) δ 7.23-7.05 (m, 4H), 4.95-4.79 (m, 1H), 4.25 (t, J=7.4 Hz, 2H), 3.62 (t, J=4.8 Hz, 2H), 2.96 (dd, J=15.4, 8.0 Hz, 2H), 2.74-2.49 (m, 8H), 2.28 (dd, J=14.2, 7.2 Hz, 4H), 1.67-1.44 (m, 14H), 1.41-1.20 (m, 42H), 0.90 (dt, J=13.2, 7.1 Hz, 9H).

¹³C NMR (101 MHz, CDCl₃) δ 173.68 (d, J=10.2 Hz), 141.26 (s), 135.23 (s), 129.73 (s), 129.37 (s), 126.72 (s), 125.92 (s), 7 7.35 (s), 77.03 (s), 76.71 (s), 74.17 (s), 64.52 (s), 57.99 (s), 55.87 (s), 53.94 (s), 34.66 (s), 34.21 (d, J=11 0.5 Hz), 32.75 (s), 31.83 (d, J=9.8 Hz), 31.32 (s), 29.65-28.88 (m), 27.15 (d, J=3.7 Hz), 26.35 (s), 25.33 (s), 25.07 (s), 24.83 (s), 22.66 (d, J=3.4 Hz), 14.12 (s).

### V. SW-II-140-2

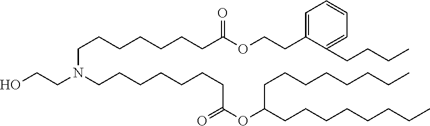 [SMILES: CCCCCCCCC(CCCCCCCC)OC(=O)CCCCCCCN(CCO)CCCCCCCC(=O)OCCc1ccccc1CCCC]

### 1. Synthesis of Compound 3

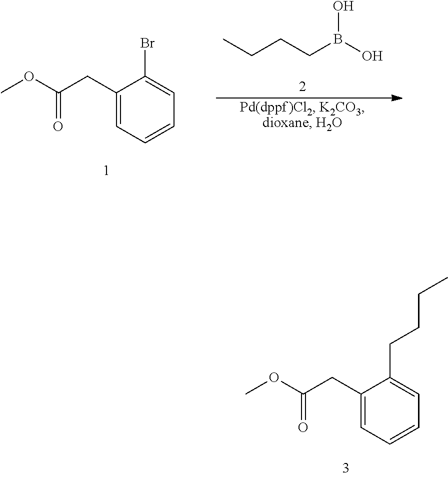

To a mixture of compound 1 (1 g, 4.37 mmol, 1 eq.) and compound 2 (668 g, 6.55 mmol, 1.5 eq.) in 1,4-dioxane/water (10 mL/1 mL) was added Pd(dppf)Cl₂ (286 mg, 0.437 mmol, 0.1 eq.) and potassium carbonate (1.8 g, 13.11 mmol, 3 eq.). The mixture was stirred at 100 °C overnight under nitrogen. TLC (PE/EA=20/1) showed the reaction was complete and new major spots were observed. The mixture was extracted with ethyl acetate and washed with water. The organic layer was dried over anhydrous sodium sulfate, filtered and concentrated in vacuo. The residue was purified by silica gel column chromatography, eluting with PE/EA (1/0-20/1), to obtain compound 3 (406 mg, 45%) as a colorless oil.

### 2. Synthesis of Compound 4

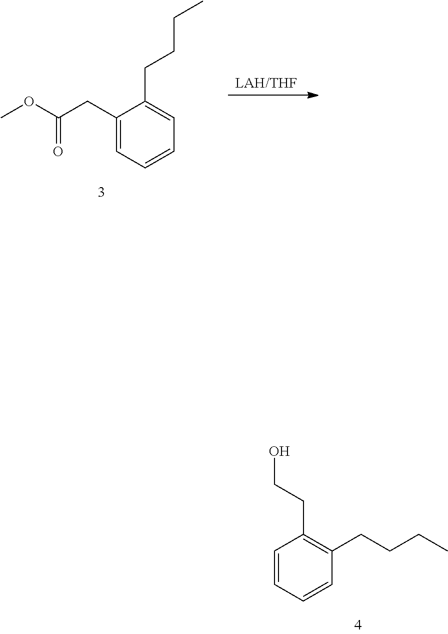

To a mixture of compound 3 (406 mg, 1.97 mmol, 1 eq.) in THF (5 mL) was added lithium aluminum hydride (2 mL, 1.97 mmol, 1 M, 1 eq. in THF) at 0 °C under nitrogen. The mixture was stirred at room temperature for 3 hours. TLC (PE/EA=5/1) indicated that the reaction was complete and new major spots were observed. The mixture was quenched with water (2 mL) and treated with 2N hydrochloric acid to adjust the pH between 6 and 7, extracted with ethyl acetate and washed with brine. The organic layer was dried over anhydrous sodium sulfate, filtered and concentrated in vacuo to afford compound 4 (341 mg, 97%) as a colorless oil without further purification.

### 3. Synthesis of Compound 6

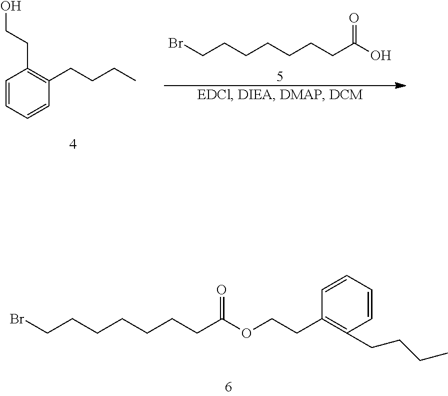

To a mixture of compound 4 (241 mg, 1.35 mmol, 1 eq.) and compound 5 (361 mg, 1.62 mmol, 1.2 eq.) in DCM (3 mL) was added EDCI (518 mg, 2.7 mmol, 2 eq.) and DMAP (66 mg, 0.54 mmol, 0.4 eq.), then DIEA (697 mg, 5.4 mmol, 4 eq.) was added. The reaction mixture was stirred at room temperature under nitrogen for 16 hours. TLC (petroleum ether/ethyl acetate=15/1) showed the formation of the desired product. The reaction mixture was extracted with ethyl acetate and washed with water. The organic layer was dried over anhydrous sodium sulfate, filtered and concentrated in vacuo. The residue was purified by silica gel column chromatography, eluting with petroleum ether/ethyl acetate (1/0-20/1), to obtain compound 6 (185 mg, 32%) as a colorless oil.

### 4. Synthesis of SW-II-140-2

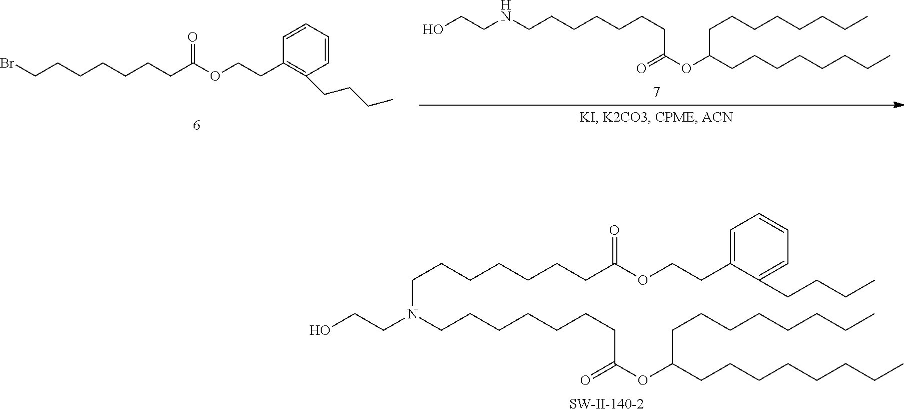 [SMILES: CCCCc1ccccc1CCOC(=O)CCCCCCCBr ; CCCCCCCCC(CCCCCCCC)OC(=O)CCCCCCCNCCO ; CCCCCCCCC(CCCCCCCC)OC(=O)CCCCCCCN(CCO)CCCCCCCC(=O)OCCc1ccccc1CCCC]

To compound 6 (185 mg, 0.483 mmol, 1 eq.) and compound 7 (213 mg, 0.483 mmol, 1 eq.) in CPME/CH₃ To the mixture in CN (2 mL/2 mL) were added potassium carbonate (400 mg, 2.898 mmol, 6 eq.) and potassium iodide (160 mg, 0.966 mmol, 2 eq.). After addition, the mixture was stirred at 90 °C overnight under nitrogen. TLC (DCM/MeOH=10/1) showed the reaction was complete and new major spots were observed. The mixture was extracted with ethyl acetate and washed with water. The organic layer was dried over anhydrous sodium sulfate, filtered and concentrated under reduced pressure. The residue was purified by silica gel column chromatography, eluting with DCM/MeOH (1/0-10:1, v/v), to obtain compound SW-II-140-2 (161 mg, 45%) as a yellow oil.

LCMS: Rt: 1.696 min; MS m/z (ELSD): 744.3[M+H]⁺;

HPLC: 94.658% purity, ELSD; RT=5.938 min.

¹H NMR (400 MHz, CDCl₃) δ 7.22-7.03 (m, 4H), 4.94-4.78 (m, 1H), 4.25 (t, J=7.3 Hz, 2H), 3.70-3.54 (m, 2H), 2.96 (t, J=7.4 Hz, 2H), 2.77-2.41 (m, 8H), 2.28 (dd, J=14.3, 7.1 Hz, 4H), 1.65-1.18 (m, 52H), 0.91 (dt, J=13.3, 7.1 Hz, 9H).

¹³C NMR (101 MHz, CDCl₃) δ 173.67 (d, J=10.8 Hz), 141.22 (s), 135.23 (s), 129.73 (s), 129.39 (s), 126.72 (s), 125.92 (s), 7 7.36 (s), 77.04 (s), 76.72 (s), 74.17 (s), 64.52 (s), 57.92 (s), 55.92 (s), 53.96 (s), 34.66 (s), 34.21 (d, J=II 0.2 Hz), 33.51 (s), 32.44 (s), 31.83 (d, J=9.3 Hz), 29.53 (d, J=2.9 Hz), 29.14 (dd, J=11.3, 8.5 Hz), 27.12 (d, J=4.1 Hz), 26.23 (s), 25.33 (s), 25.06 (s), 24.82 (s), 22.73 (d, J=9.9 Hz), 14.08 (d, J=8.8 Hz).

### Example 2 Comparison of the Physical and Chemical Properties and Expression Levels of LPP Preparations with Different Lipid-to-Lipid Ratios for Mucosal Administration

This example uses the prescriptions in Table 1, Table 2 and Table 3 to prepare a four-lipid LPP preparation containing luciferase (Luciferase) mRNA (the coding sequence is shown in SEQ ID NO: 1) for mucosal administration, and the The prepared LPP preparations were compared for three rounds of physical and chemical properties and in vivo expression levels to screen for lipid-to-lipid ratios suitable for mucosal administration. And the MC3 LNP preparation was used as a positive control.

### 2.1 Preparation of Lipid Nanoparticle (LNP-mRNA) Formulation

Preparation of mRNA aqueous solution: Dilute luciferase mRNA into 0.1 mg/mL mRNA aqueous solution with 10 mM sodium citrate (pH=4.0) buffer solution.

Preparation of lipid solution: Dissolve MC3:phospholipid:cholesterol:PEG in ethanol solution at a molar ratio of 50:10:38.5:1.5 to prepare a 6 mg/mL lipid solution.

Preparation of LNP: Using microfluidic technology (Maiana (Shanghai) Technology Co., Ltd., model: Inano D), the lipid solution and the mRNA aqueous solution were mixed under the following conditions: volume=4.0 mL: flow rate ratio=3 (mRNA aqueous solution): 1 (lipid solution), total flow rate=12 mL/min, to obtain LNP-mRNA solution.

Centrifugal ultrafiltration: Add the LNP-mRNA solution into the ultrafiltration tube for centrifugal ultrafiltration concentration (centrifugal force 3000 rpm, centrifugation time 60 min, temperature 4 °C), ultrafiltrate until the ethanol content is <0.5%, and adjust the LNP mRNA concentration to 0.2 mg/mL. The LNP-mRNA preparation numbered MC3 LNP was obtained.

### 2.2 Preparation of Lipid Polyplex (LPP-mRNA) Preparation

Preparation of lipid mixture: Dissolve M5, phospholipids, cholesterol and PEG lipids in ethanol solution according to the lipid and lipid ratios shown in Table 1, Table 2 and Table 3 to prepare a 6 mg/mL lipid mixture.

Preparation of the mRNA aqueous solution: Use 10 mM sodium citrate (pH=4.0) buffer solution to dilute the luciferase mRNA (the coding sequence is shown in SEQ ID NO: 1) into a 0.1 mg/mL mRNA aqueous solution.

Preparation of protamine sulfate solution: Dissolve protamine sulfate in nuclease-free water to prepare a protamine sulfate solution with a working concentration of 0.125 mg/mL.

Preparation of core nanoparticle solution: Use microfluidic technology to mix protamine sulfate solution and mRNA solution under the following conditions to obtain a core nanoparticle solution formed by protamine and mRNA: mass ratio=4 (mRNA Solution): 1 (protamine solution), flow rate ratio=5 (mRNA): 1 (protamine solution), total flow rate=12 mL/min, room temperature.

Preparation of LPP: Mix the nuclear nanoparticle solution and the lipid solution twice under the following conditions: mass ratio=20 (nuclear nanoparticle solution): 1 (lipid mixture), flow rate ratio=3 (nuclear nanoparticle solution)):1 (lipid solution), total flow rate=12 mL/min, room temperature, obtain LPP-mRNA solution.

Centrifugal ultrafiltration: Centrifuge the LPP-mRNA solution to remove ethanol through ultrafiltration (centrifugal force 3000 rpm, centrifugation time 60 min, temperature 4 °C), ultrafiltrate until the ethanol content is <0.5%, and adjust the LPP mRNA concentration to 0.2 mg/mL. The obtained numbers are IN-1-1, IN-1-2, IN-1-3, IN-1-4, IN-1-5, IN-1-6, IN-1-7, IN-1-8, IN-1-9, IN-1-10, IN-1-11, IN-1-12, IN-1-13, IN-1-14, IN-1-15, IN-1-16, IN-2-1, IN-2-2, IN-2-3, IN-2-4, IN-2-5, IN-2-6, IN-2-7, IN-2-8, IN—2-9, IN-2-10, IN-2-11, IN-2-12, IN-2-13, IN-2-14, IN-2-15, IN-2-16, IN-3-1. LPP preparations of IN-3-4, IN-3-5, IN-3-6, IN-3-7, IN-3-9, IN-3-11 and IN-3-12.

2.3 Detection of Physical and Chemical Properties of LPP Preparations with Different Lipid-to-Lipid Ratios

The physical and chemical properties of the prepared LPP preparations were tested to screen the lipid-to-lipid ratio suitable for mucosal administration. The specific detection methods are as follows:

Particle size detection: Take 50 μL of the LPP sample prepared in Example 2.2 and dilute it with 950 mL of purified water to obtain a diluted LPP sample. The sample is placed in a dynamic light scattering laser particle sizer (Malvern, ZS-90) for detection.

Encapsulation efficiency detection: Quant-iT RiboGreen RNA reagent (Thermo Scientific) was used to detect the encapsulation efficiency of mRNA in the LPP solution prepared in Example 2.2. First, detect the amount of free mRNA outside the LPP particles in the LPP solution, and dilute the LPP solution with nuclease-free water and 1×TE buffer (10 mM Tris-HCl, 1 mM EDTA, pH 7.5). Transfer 100 μL of each diluted sample to a 96-well plate, and then add 100 μL of 200-fold diluted RiboGreen RNA reagent to it. The 96-well plate was placed on a well plate mixer and mixed at room temperature for 10 minutes, and then the fluorescence value was read using a Bio-Tek Synergy I plate reader (BioTek). Standard samples were processed in the same manner. A calibration curve of fluorescence and mRNA concentration was drawn by linear regression, and the mRNA content of the sample free outside the LPP particles was calculated.

To detect the total mRNA content inside and outside the LPP sample particles, dilute the LPP solution with nuclease-free water, then vortex mix with an equal volume of 2% Triton X-100 and incubate at room temperature for 10 minutes to destroy the LPP structure and release the encapsulated in mRNA within LPP particles. Transfer 100 μL of each of the above samples to a 96-well plate, and then add 100 μL of 200-fold diluted RiboGreen RNA reagent to it. The %-well plate was placed on a well plate mixer and mixed at room temperature for 5 minutes, and then the fluorescence value was read using a Bio-Tek Synergy I plate reader (BioTek). Standard samples were processed in the same manner. A calibration curve of fluorescence and mRNA concentration was drawn by linear regression, from which the total mRNA content inside and outside the LPP sample particles was calculated.

The encapsulation efficiency of the LPP solution is defined as the percentage of mRNA encapsulated inside the LPP particles in the test sample to the total amount of mRNA.

Polydispersity index (PDI) detection: Zetasizer Nano ZS (Malvern Instruments Ltd, Malvern, Worcestershire, UK) was used to determine the polydispersity index (PDI) of the LPP solution.

Purity detection: Use Q-Analyzer and RNA Carisge Kit (RNA Low Marker (5×); Dilution Buffer (10×); Separation Buffer (10×); Mininer oil) to detect the purity of mRNA in the LPP solution prepared in Example 2.2. Take 50 μL sample, first dilute the LPP solution with an equal volume of 1× Dilution Buffer, and then add an equal volume of 2% Triton; add RNase inhibitor (Invitrogen) and 0.1 mg/mL heparin sodium solution in sequence, vortex to mix, and let stand. Leave for 5 minutes. After standing, place the sample in a 70 °C water bath for 20 minutes, then quickly transfer it to a 4 °C refrigerator to cool down for 5-10 minutes; take out 50 μL of the sample and add it to the eight-strip tube, and add 20 μL of Mininer oil above each. The purity of the sample was tested using capillary electrophoresis Qsep100 method.

The physical and chemical property test results are shown in Table 1, Table 2 and Table 3. Different lipids and lipid ratios will affect the physical and chemical properties of lipid compositions, such as polydispersity index, particle size, encapsulation efficiency, etc. In the first round of screening, the lipid ratio and lipids are shown in Table 1. The screening range is M5: 30-60%; phospholipids (DOPE, DSPC): 5-20%: PEG (DMG-PEG, DSPE-PEG, ACL-0159): 1.25-10%; cholesterol: 15-65%; in the second round of screening, the lipid ratio and lipids are shown in Table 2, and the screening range is M5: 35-50%: phospholipid (DOPE): 10-30%; PEG (DMG-PEG, DSPE-PEG): 1.25-10%: cholesterol: 15-55%: in the third round of screening, the lipid ratio and lipids are shown in Table 3, and the screening The range is M5: 37.542.5%: phospholipid (DOPE): 25-35%; PEG (DMG-PEG): 3.75-7.5%; cholesterol: 15-30%. It was observed that when M5: 37.5-42.5%; phospholipid (DOPE): 25-35%; PEG (DMG-PEG): 3.75-7.5%; cholesterol; 15-30%, it has a more excellent and stable particle size, more Dispersion index and encapsulation efficiency.

TABLE 1
First round of mucosal drug prescription screening

| | M5 | | lipid | | PEG | Chole [symbol] | size | | |
|---|---|---|---|---|---|---|---|---|---|
| NO. | % | lipid | % | PEG | % | % | (nm) | PDI | [symbol] |
| IN-1-1 | 30 | DOPE | 5 | DMG-PEG | 5 | 60 | 59.1 | [symbol] | 94. [symbol] |
| IN-1-2 | 30 | DOPE | 5 | DMG-PEG | 1.25 | 63. [symbol] 5 | 114.9 | 0.40 | 94. [symbol] |
| IN-1-3 | 45 | DOPE | 10 | DMG-PEG | 2.5 | 42.5 | 90.2 | 0.15 | 92.4 |
| IN-1-4 | 45 | DOPE | 20 | DMG-PEG | 10 | 25 | 57.4 | 0.12 | 59.4 |
| IN-1-5 | 30 | DOPE | 20 | DSPE-PEG | 1.25 | 48.75 | 103.5 | 0.08 | [symbol] 4.4 |
| IN-1-6 | 60 | DOPE | 5 | DSPE-PEG | 2.5 | [symbol] 2.5 | 102.5 | 0.06 | 83.9 |
| IN-1-7 | 30 | DOPE | 10 | ALC-0159 | 5 | 55 | 59. [symbol] | 0.34 | 94.2 |
| IN-1-8 | 60 | DOPE | 5 | ALC-0159 | 10 | 25 | 72.7 | 0.1 [symbol] | 31.8 |
| IN-1-9 | 30 | DSPC | 5 | DMG-PEG | 2.5 | 62.5 | 174.0 | 0.6 [symbol] | 88.5 |
| IN-1-10 | 30 | DSPC | 5 | DMG-PEG | 10 | 55 | 57.5 | 0.31 | 93.7 |
| IN-1-11 | 60 | DSPC | 20 | DMG-PEG | 5 | 1 [symbol] | 82. [symbol] | 0.17 | [symbol] 3.3 |
| IN-1-12 | 60 | DSPC | 10 | DMG-PEG | 1.25 | 28.75 | 118.1 | 0.08 | 83.5 |
| IN-1-13 | 30 | DSPC | 10 | DSPE-PEG | 10 | 50 | 64.9 | 0.34 | 86.5 |
| IN-1-14 | 45 | DSPC | 5 | DSPE-PEG | 5 | 45 | 74.5 | 0.33 | 90.9 |
| IN-1-15 | 30 | DSPC | 20 | ALC-0159 | 2.5 | 47.5 | 94.8 | 0.27 | 86.6 |
| IN-1-16 | 45 | DSPC | 5 | ALC-0159 | 1.25 | 48.75 | 103.6 | 0.20 | 92.9 |

[symbol]

indicates data missing or illegible when filed

TABLE 2
Second round of mucosal drug prescription screening

| | M5 | DOPE | | PEG | Chol [symbol] ol | particle | | [symbol] lation | purity |
|---|---|---|---|---|---|---|---|---|---|
| NO. | % | % | PEG | % | % | [symbol] | PDI | efficiency ( [symbol] ) | ( [symbol] ) |
| IN-2-1 | 35 | 10 | DMG-PEG | 1.25 | 53.75 | 108.5 | 0.0 [symbol] | 95.9 | 87.1 |
| IN-2-2 | 35 | 10 | DMG-PEG | 5 | 50 | 67.7 | 0.30 | [symbol] 0.7 | 86. [symbol] |
| IN-2-3 | 35 | 20 | DMG-PEG | 2.5 | 42.8 | 100.1 | 0.16 | 93.0 | 86.0 |
| IN-2-4 | 35 | 30 | DMG-PEG | 10 | 25 | 69. [symbol] | 0.20 | 39.3 | 82.7 |
| IN-2-5 | 42.5 | 10 | DMG-PEG | 2.5 | 45 | 102.8 | 0.12 | [symbol] 1.8 | 85.8 |
| IN-2-6 | 42.5 | 30 | DMG-PEG | 5 | 22.5 | 90.9 | 0.24 | 82.5 | 85.6 |
| IN-2-7 | 50 | 10 | DMG-PEG | 10 | 30 | 8 [symbol] . [symbol] | 0.14 | 13.8 | 82.1 |
| IN-2-8 | 50 | 20 | DMG-PEG | 1.25 | 28.75 | 146.1 | 0.14 | 93.4 | 83.8 |
| IN-2-9 | 35 | 10 | DSPE-PEG | 2.5 | 52.5 | 68.4 | 0.28 | 94.2 | 80.8 |
| IN-2-10 | 35 | 10 | DSPE-PEG | 10 | 45 | 60.5 | 0.33 | 82.6 | 81.7 |
| IN-2-11 | 35 | 20 | DSPE-PEG | 5 | 40 | 62.5 | 0.30 | 86. [symbol] | 80.9 |
| IN-2-12 | 35 | 30 | DSPE-PEG | 1.25 | 33.75 | 119.4 | 0.04 | [symbol] 4.6 | 82.3 |
| IN-2-13 | 42.5 | 10 | DSPE-PEG | 1.25 | 46.25 | 93.4 | 0.11 | 94.2 | 80.4 |
| IN-2-14 | 42.5 | 20 | DSPE-PEG | 10 | 27.5 | 48. [symbol] | 0. [symbol] 9 | 77.2 | 81.2 |
| IN-2-15 | 50 | 10 | DSPE-PEG | 5 | 35 | 62.1 | 0.15 | 87.8 | 80.8 |
| IN-2-16 | 50 | 30 | DSPE-PEG | 2.5 | 17.5 | 70.5 | 0.1 | 90.7 | 78. [symbol] |
| IN-1-4 | 45 | 20 | DMG-PEG | 10 | 25 | 62.8 | 0.18 | 51.4 | 80.8 |

[symbol]

indicates data missing or illegible when filed

TABLE 3
Third round of mucosal drug prescription screening

| | M5 | DOPE | DMG- | Cholesterol | size | | encapsulation |
|---|---|---|---|---|---|---|---|
| NO. | % | % | PEG % | % | (nm) | PDI | efficiency ( [symbol] ) |
| IN-3-1 | 37.5 | 25 | 7.5 | 30 | 66.5 | 0.24 | 83.4 |
| IN-3- [symbol] | 37.5 | 35 | 5 | 22.5 | 71. [symbol] | 0.26 | 90.6 |
| IN-3-5 | 40 | 25 | 5 | 30 | 88.7 | 0.19 | 89.1 |
| IN-3-6 | 40 | 30 | 3.75 | 26.25 | 92.0 | 0.21 | 93.0 |
| IN-3-7 | 40 | 30 | 5 | 25 | 89.6 | 0.25 | 89.6 |
| IN-3-9 | 42.5 | 25 | 5 | 27.5 | 88.1 | 0.21 | 86.9 |
| IN-3-11 | 42.5 | 35 | 3.75 | 18.75 | 75. [symbol] | 0.21 | 91.2 |
| IN-3-12 | 42.5 | 35 | 5 | 17.5 | 64.8 | 0.24 | 87.9 |
| IN-2-6 | 42.5 | 30 | 5 | 22.5 | 71.4 | 0.22 | 87.7 |

[symbol]

indicates data missing or illegible when filed

2.4 In Vivo Expression of LPP Preparations with Different Lipid-to-Lipid Ratios

While conducting three rounds of physical and chemical property tests on the prepared LPP preparations as in Example 2.3, the in vivo expression of the prepared LPP preparations was also tested to screen for lipids and lipid ratios suitable for mucosal administration. The specific detection methods are as follows:

During the three rounds of in vivo expression detection, 6-8 week old female BALB/c mice (Beijing Vitong Lever Experimental Animal Technology Co., Ltd.) were anesthetized with sodium pentobarbital (70 mg/kg), and then Prepared for administration by nasal instillation

LPP solution (3 mice per group), and during each round of screening, two groups of mice were administered nasal mucosa MC3 LNP solution and PBS solution (negative control) respectively, each LPP solution or LNP solution administered contained 2 μg of luciferase mRNA (10 μL). Mice were intraperitoneally injected with 3 mg of D-luciferin substrate (Maokang Biotechnology) 6 hours and 24 hours after drug administration, and 10 minutes after substrate injection, the mice were imaged in vivo using the Xenogen IVIS-200 imaging system., to detect the expression of luciferase in vivo.

Experimental results show that all LPP preparations are only expressed in the nasal cavity when M5: 37.5-42.5%; Phospholipid (DOPE): 25-35%; PEG (DMG-PEG): 3.75-5%; Cholesterol: 15-30%, with the highest luciferase expression. The AUC of the IN-3-1l LPP preparation that expressed the most luciferase in the third round was approximately 35 times that of the IN-1-4 that expressed the most luciferase in the first round and was approximately 100 times that of the MC3 LNP preparation.

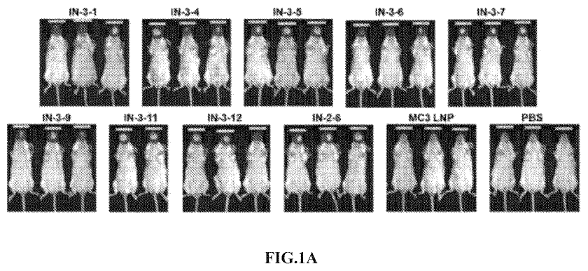

Among them, the luciferase expression in mice 6 hours after administration in the third round is shown in FIG. 1A and FIG. 1B. All LPP preparations are only expressed in the nasal cavity, indicating low systemic toxicity, and IN-3 The −11 LPP preparation had the highest luciferase expression.

According to the test results of physical and chemical properties in Example 2.3 and the results of in vivo expression in this example, the optimized lipid-to-lipid ratio range suitable for mucosal administration was confirmed, that is, cationic lipids (in this example M5): 37.5-42.5%; phospholipid (DOPE): 25-35%; PEG (DMG-PEG): 3.75-5%: cholesterol: 15-30%. And based on the above results, prescriptions using IN-2-6, IN-3-4, IN-3-5, IN-3-11 and IN-3-12 LPP preparations with higher luciferase expression levels in the nasal cavity were selected. Conduct further testing.

### Example 3 Application of LPP Preparations for Mucosal Administration in COVID-19 Vaccine Treatment

In this example, mice were immunized with different immunization regimens to test the application of the preferred LPP formulation in Example 2 in the treatment of COVID-19 vaccines.

### 3.1 Preparation of Lipid Polyplex (LPP-mRNA) Preparation

Preparation of lipid mixture: Dissolve M5, phospholipids, cholesterol and PEG lipids in ethanol solution according to the prescriptions of IN-2-6. IN-3-11 and IN-3-12 LPP preparations to prepare 6 mg/mL Lipid mixture.

Preparation of aqueous mRNA solution: Use 10 mM sodium citrate (pH=4.0) buffer solution to dilute COVID-19 mRNA (the coding sequence is shown in SEQ ID NO: 2) into a 0.1 mg/mL aqueous mRNA solution.

Preparation of protamine sulfate solution: Dissolve protamine sulfate in nuclease-free water to prepare a protamine sulfate solution with a working concentration of 0.125 mg/mL.

Preparation of core nanoparticle solution: Use microfluidic technology to mix protamine sulfate solution and mRNA solution under the following conditions to obtain a core nanoparticle solution formed by protamine and mRNA: mass ratio=4 (mRNA Solution): 1 (protamine solution), flow rate ratio=5 (mRNA): 1 (protamine solution), total flow rate=12 mL/min, room temperature.

Preparation of LPP: Mix the nuclear nanoparticle solution and the lipid solution twice under the following conditions: mass ratio=20 (nuclear nanoparticle solution): 1 (lipid mixture), flow rate ratio=3 (nuclear nanoparticle solution)): 1 (lipid solution), total flow rate=12 mL/min, room temperature, obtain LPP-mRNA solution.

Centrifugal ultrafiltration: Centrifuge the LPP-mRNA solution to remove ethanol through ultrafiltration (centrifugal force 3000 rpm, centrifugation time 60 min, temperature 4 °C), ultrafiltrate until the ethanol content is <0.5%, and adjust the LPP mRNA concentration to 1 mg/mL. LPP preparations numbered IN-2-6/Covid-19, IN-3-11/Covid-19 and IN-3-12/Covid-19 were prepared.

And prepare a B11/Covid-19 LPP preparation with a lipid ratio of M5: phospholipid: cholesterol: PEG with a molar ratio of 40:15:43.5:1.5 through the above method, and adjust the B11/Covid-19 LPP mRNA concentration to 0.1 mg/mL.

### 3.2 Immune Effects of Different Immunization Regimens

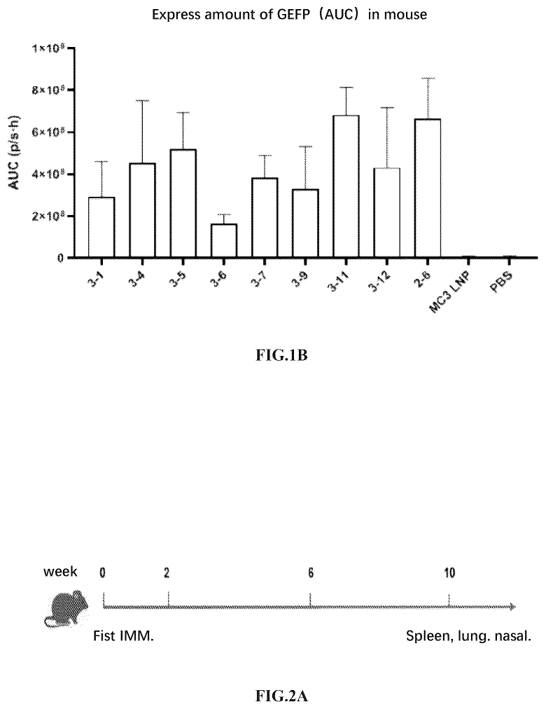

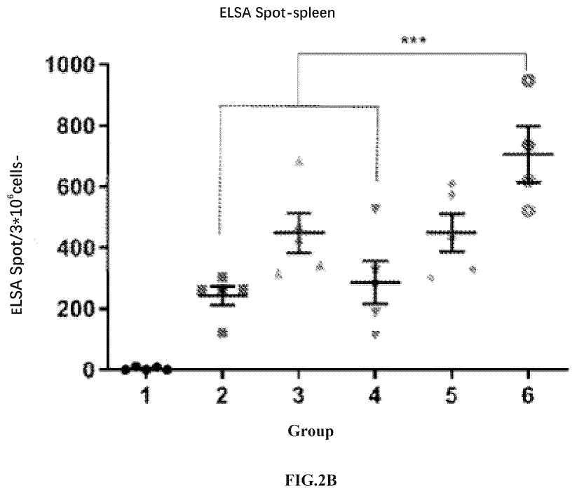

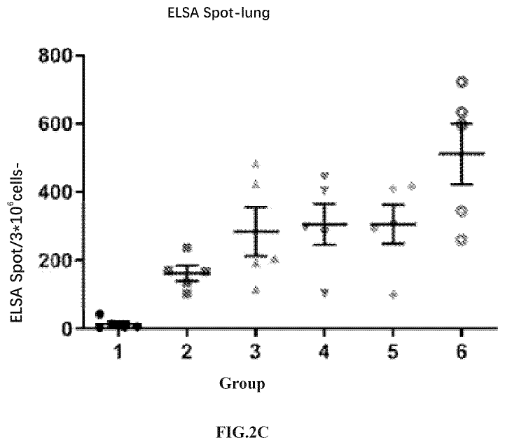

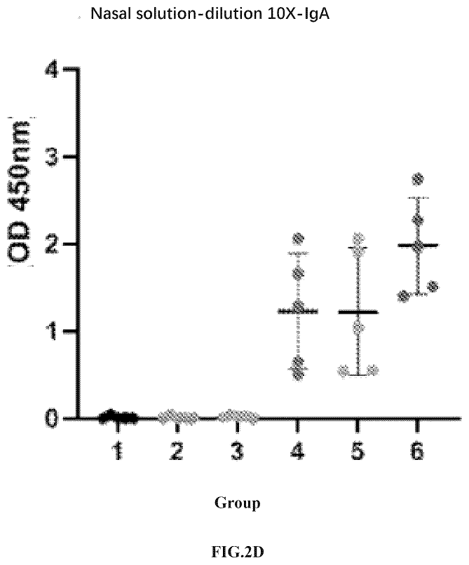

Mice were immunized by the immunization protocol shown in FIG. 2A and Table 4.

Female BALB/c mice (Beijing Weitonglihua Experimental Animal Technology Co., Ltd.) aged 6-8 weeks were divided into 6 groups (n=5). On days 0 and 14 (2 weeks after primary vaccination), mice in groups 2, 3, 4, 5, and 6 were immunized with a B11/Covid-19 LPP formulation containing 3 μg of mRNA via intramuscular injection. At the 6th week after the primary immunization, the third immunization was carried out, and mice in groups 2, 3, 4, 5 and 6 were administered PBS, B11/Covid-19 LPP preparation (intramuscular route), IN—2-6/Covid-19 LPP preparation (nasal drip administration), IN-3-11/Covid-19 LPP preparation (nasal drip administration) and IN-3-12/Covid-19 LPP preparation (nasal drip administration) Injection administration): the mice in the first group did not receive the first two immunizations, and were administered PBS as a control at the 6th week after the primary immunization. At 10 weeks after the primary immunization, the mice were euthanized, and the lung, spleen and nasal cavity lavage fluids (obtained by lavage with 0.7 mL PBS) were collected for subsequent testing.

Immune cell responses in splenocytes and lung cells were detected by ELISpot, and antibody levels in serum and nasal lavage fluid were detected by ELISA. The specific detection methods are as follows:

### Enzyme-Linked Immunospot (ELISpot) Assay

Mouse IFN-γ ELISpot assay was performed using the IFN-γ ELISpot PLUS kit (Mabtech, 3321-4APT-10) following the manufacturer's instructions. Briefly, the plate was blocked with 1640 medium containing 10% FBS, 200 μL/well, placed in a cell culture incubator, and left to stand for more than 1 h. Pulmonary cells or spleen cells were cultured at 3×10⁶ Cells/well were plated and stimulated in vitro with 100 μL/well S protein peptide, 100 μL medium was added to the negative control, and 100 μL PMA+Ionomycine was added to the positive control, and incubated at 37 °C, 5% CO2 for 20 hours. Afterwards, biotinylated IFN-γ-detection antibody and streptavidin-alkaline phosphatase (ALP) were used for detection, and BCIP/NBT-plus (5-bromo-4-chloro-3-indole-phosphate/nitro blue tetrazolium-plus) substrate for color development and counting using an ELISpot plate reader (ImmunoSpot S6 Core Analyzer (CTL)).

### Enzyme-Linked Immunosorbent Assay (ELISA)

When performing ELISA testing of animal serum/lavage fluid, use ELISA coating buffer to dilute the S protein antigen (SARS-CoV-2 (2019-nCoV) Spike Protein, purchased from Beijing Yiqiao Shenzhou Technology Co., Ltd.) to 2 μg./mL, use 100 μL per well, 200 ng/well antigen to coat a 96-well plate, and incubate overnight at 4 °C. After overnight incubation at 4 °C, the plates were washed three times with phosphate-buffered saline PBS-Tween (PBST) (0.5% tween-20, w/v) and blocked in 10% FCS-PBST for 2 h at 37 °C. The serum was then diluted in 10% FCS-PBST, and 100 μL of diluted serum or lavage fluid was added to the plate and incubated at 37 °C for 1 h. Then aspirate the liquid in the plate, wash the plate three times in PBST, use sample diluent to dilute the secondary antibody (Goat pAb to Mouse IgG-HRP) at a ratio of 1:10000, add 100 μL to each well and incubate at 37 °C. 0.5h. After incubation, the plate was washed three times with PBST, and 10 μL of 1×TMB substrate solution was added to each well to develop the color reaction for 3 minutes. After color development, 50 μL of TMB stop solution was added to terminate the color development, and a Synegry H1 microplate reader was used to record at 450 nm and 610 nm. Absorbance. To analyze the results, first subtract the OD610 value from the OD450 value of each well, and then subtract the average value of the blank wells to obtain the actual sample absorbance value. When the absorbance value corresponding to the highest dilution multiple is greater than 0.21, the highest dilution is used as the titer of the sample.

For IgA, add 100 μL of diluted perfusate to the plate and incubate at 37 °C for 1 h. Then aspirate the liquid in the plate, wash the plate three times in PBST, use sample diluent to dilute the secondary antibody (Goat pAb to Mouse IgA-biotin) at a ratio of 1:10000, add 100 μL to each well and incubate at 37 °C 1h. After incubation, plates were plated with

Wash three times with PBST, use sample diluent to dilute the antibody Streptavidin-HRP at a ratio of 1:5000, add 100 μL to each well, and incubate at 37 °C for 1 hour. After incubation, the plate was washed three times with PBST, and 100 μL of 1×TMB substrate solution was added to each well to develop the color reaction for 10 minutes. After color development, 100 μL of TMB stop solution was added to terminate the color development, and the absorbance was recorded at 450 nm using a Synegry H1 microplate reader. For result analysis, first subtract the average value of the blank wells from the OD450 of each well to obtain the actual sample absorbance value. When the absorbance value corresponding to the highest dilution multiple is greater than 0.21, the highest dilution is used as the titer of the sample.

The ELISpot test results are shown in Table 5 and FIG. 2B and FIG. 2C. The booster immunization of the third immunization will have a better immune effect. In the third immunization, IN-3—was administered through nasal drip. The cellular immune response after 11/Covid-19 LPP formulation and IN-3-12/Covid-19 LPP formulation was comparable or higher than that after intramuscular administration of B11/Covid-19 LPP formulation.

The ELISA test results are shown in Table 5 and FIG. 2D. After the third booster immunization, there will be higher antibody levels in the serum and nasal lavage fluid, and a better immune effect. Compared with the B11/Covid-19 LPP formulation administered by intramuscular injection in the third immunization, the IN-2-6/Covid-19 LPP formulation, IN-3 was administered by intranasal instillation in the third immunization The −11/Covid-19 LPP formulation or the IN-3-12/Covid-19 LPP formulation produced comparable or higher cellular immune responses and activated mucosal immunity, resulting in mice with significantly higher IgA antibody levels.

The above results show that the “primary immunity+boost” method (systemic immunity+mucosal immunity) can combine the advantages of the two immune methods and induce a higher level of immune effect.

TABLE 4
Immunization scheme

| | | | Third | dose | volume | place of |
|---|---|---|---|---|---|---|
| G | NO | two [symbol] | [symbol] | (mg/kg) | (μL) | third |
| 1 | 5 | — | PBS | — | 30 | Muscle |
| 2 | 5 | B [symbol] /Covid-19 | PBS | — | 30 | Muscle |
| | | 3 μg [symbol] im | | | | |
| 3 | 5 | B [symbol] /Covid-19 | B [symbol] / | 0.15 ( [symbol] μg) | 30 | Muscle |
| | | 3 μg [symbol] im | Covid-19 | | | |
| 4 | 5 | B [symbol] /Covid-19 | IN-2-6/ | 0.5 (10 μg) | 10 | nasal |
| | | 3 μg [symbol] im | Covid-19 | | | |
| 5 | 5 | B [symbol] /Covid-19 | IN-3-11/ | 0.5 (10 μg) | 10 | nasal |
| | | 3 μg [symbol] im | Covid-19 | | | |
| 6 | 5 | B [symbol] /Covid-19 | IN-3-12/ | 0.5 (10 μg) | 10 | nasal |
| | | 3 μg [symbol] im | Covid-19 | | | |

[symbol]

indicates data missing or illegible when filed

TABLE 5
Immune effects of different immunization regimens

| | | | ELISpot- | ELISpot- |
|---|---|---|---|---|
| G | LgG serum | nasal IgA | Spleen(p [symbol] / [symbol] cells | Lung(p [symbol] / [symbol] cells) |
| 1 | 1 | 1 | 4 | 15 |
| 2 | 73517 | 1 | 243 | 162 |
| 3 | 222861 | 1 | 449 | 285 |
| 4 | 128000 | 40 | 286 | 306 |
| 5 | 97006 | 40 | 443 | 307 |
| 6 | 90510 | 70 | 7 [symbol] 6 | 512 |

[symbol]

indicates data missing or illegible when filed

### Example 4 Application of LPP Formulation for Mucosal Administration in Influenza Vaccine Treatment

In this example, mice were immunized with different immunization regimens to test the application of the preferred LPP formulation in Example 2 in influenza vaccine treatment.

### 4.1 Preparation of Lipid Polyplex (LPP-mRNA) Preparation

Preparation of lipid mixture: Mix M5, phospholipids, cholesterol and PEG lipids according to the prescriptions of IN-2-6, IN-3-4, IN-3-5, IN-3-11 and IN-3-12 LPP preparations. Dissolve the lipids in ethanol solution to prepare a lipid mixture of 6 mg/mL.

Preparation of the mRNA aqueous solution: Dilute the mRNA encoding the influenza virus antigen (the coding sequence is shown in SEQ ID NO: 3) with 10 mM sodium citrate (pH=4.0) buffer solution into a 0.1 mg/mL mRNA aqueous solution.

Preparation of protamine sulfate solution: Dissolve protamine sulfate in nuclease-free water to prepare a protamine sulfate solution with a working concentration of 0.125 mg/mL.

Preparation of core nanoparticle solution: Use microfluidic technology to mix protamine sulfate solution and mRNA solution under the following conditions to obtain a core nanoparticle solution formed by protamine and mRNA: mass ratio=4 (mRNA Solution): 1 (protamine solution), flow rate ratio=5 (mRNA): 1 (protamine solution), total flow rate=12 mL/min, room temperature.

Preparation of LPP: Mix the nuclear nanoparticle solution and the lipid solution twice under the following conditions: mass ratio=20 (nuclear nanoparticle solution): 1 (lipid mixture), flow rate ratio=3 (nuclear nanoparticle solution)): 1 (lipid solution), total flow rate=12 mL/min, room temperature, obtain LPP-mRNA solution.

Centrifugal ultrafiltration: Centrifuge the LPP-mRNA solution to remove ethanol through ultrafiltration (centrifugal force 3000 rpm, centrifugation time 60 min, temperature 4 °C), ultrafiltrate until the ethanol content is <0.5%, and adjust the LPP mRNA concentration to 1 mg/mL. LPP formulations numbered IN-2-6/Flu, IN-3-4/Flu, IN-3-5/Flu, IN-3-11/Flu and IN-3-12/Flu were prepared.

And a B11/Flu LPP preparation with a lipid ratio of M5:phospholipid:cholesterol:PEG of 40:15:43.5:1.5 was prepared by the above method, and the B11/Flu LPP mRNA concentration was adjusted to 0.1 mg/mL

### 4.2 Immune Effects of Different Immunization Regimens

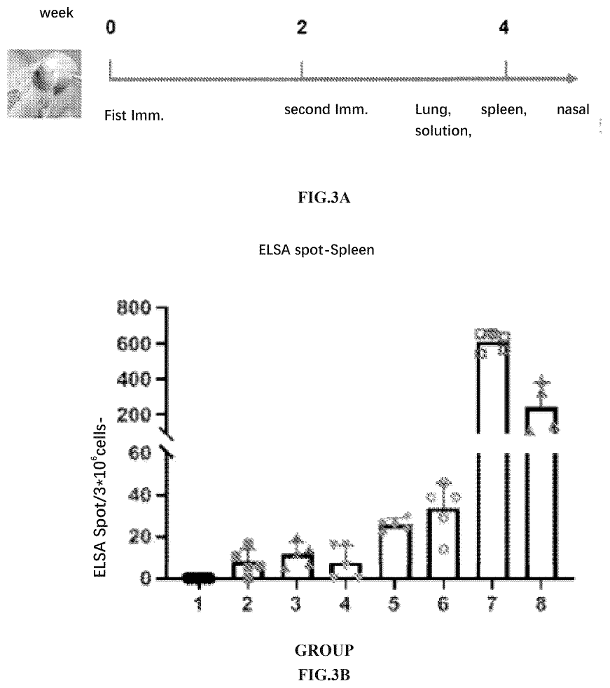

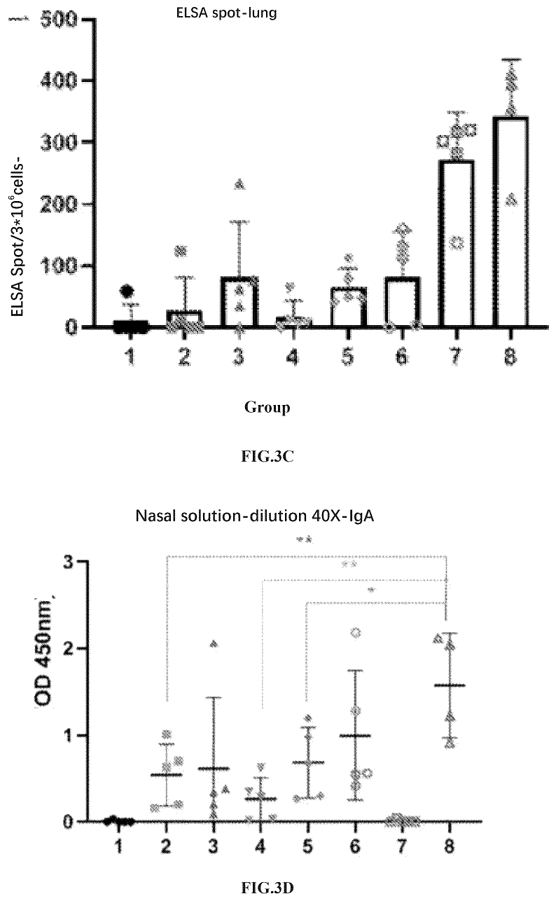

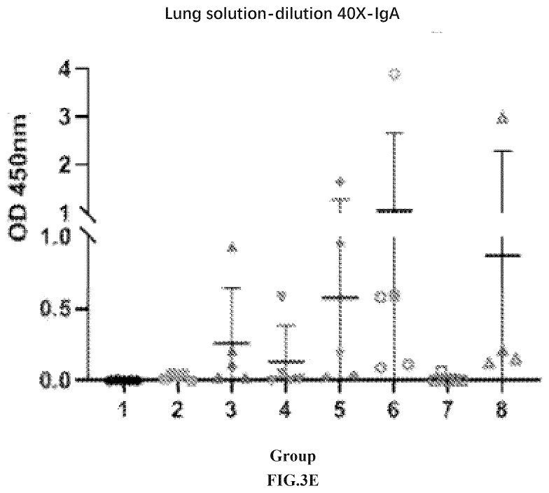

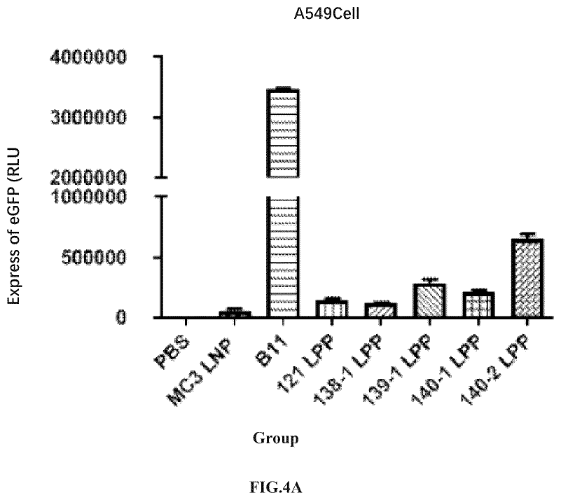

Mice were immunized by the immunization protocol shown in FIG. 3A and Table 6. Female BALB/c mice (Beijing Weitonglihua Experimental Animal Technology Co., Ltd.) aged 6-8 weeks were divided into 8 groups (n=5). On days 0 and 14 (2 weeks after primary vaccination) Mice were immunized twice. In two immunizations, mice in group 1 were administered 10% sucrose solution by nasal instillation; mice in groups 2, 3, 4, 5 and 6 were administered IN-2-6/Flu LPP formulation, IN-3-4/Flu LPP formulation, IN-3-5/Flu LPP formulation, IN-3-11/Flu LPP formulation, and IN-3-12/Flu LPP formulation: via intramuscular route Group 7 mice were administered B11/Flu LPP preparation. During the primary immunization, mice in Group 8 were administered the B11/Flu LPP formulation via intramuscular route, and during the second immunization, mice in Group 8 were administered IN-3-11/Flu LPP via nasal instillation, preparation. At the fourth week after the primary immunization, the mice were sacrificed, and the lung, spleen and lung/nasal cavity lavage fluids (obtained by lavage with 0.7 mL PBS) were collected for subsequent testing.

ELiSpot was used to detect immune cell responses in splenocytes and lung cells, and ELISA was used to detect antibody levels in serum, nasal lavage fluid, and lung lavage fluid. For specific detection methods of ELISpot, please refer to Example 3.2. The specific detection methods of ELISA are as follows:

When performing ELISA detection of animal serum, use ELISA coating buffer to dilute influenza virus antigen (NP protein, purchased from Beijing Yiqiao Shenzhou Technology Co., Ltd.) to 1 μg/mL, and coat with 100 μL per well and 100 ng/well antigen, 96-well plate and incubate overnight at 4 °C. After overnight incubation at 4 °C, the plates were washed three times with phosphate-buffered saline PBS-Tween (PBST) (0.5% tween-20, w/v) and blocked in 10% FCS-PBST for 2 h at 37 °C. The plate was washed 3 times in PBST, and then the serum was diluted in 10% FCS-PBST. For IgG detection, the sample was diluted 2-fold, and 100 μL of diluted serum was added to the plate and incubated at 37 °C for 1 h. Then suck up and discard the board

Wash the plate three times in PBST, use sample diluent to dilute the secondary antibody (Goat pAb to Mouse IgG-HRP) at a ratio of 1:50000, add 100 μL to each well, and incubate at 37 °C for 1 hour. After incubation, the plate was washed three times with PBST, and 100 μL of 1×TMB substrate solution was added to each well to develop the color reaction for 10 minutes. After color development, 50 μL of TMB stop solution was added to terminate the color development, and the absorbance was recorded at 450 nm using a Synegry H1 microplate reader. For result analysis, first subtract the average value of the blank wells from the OD450 value of each well to obtain the actual sample absorbance value. When the absorbance value corresponding to the highest dilution multiple is greater than 0.21, the highest dilution is used as the titer of the sample.

For IgA, when performing ELISA detection of animal serum/lavage fluid, use ELISA coating buffer to dilute the influenza virus antigen to 2 μg/mL, coat a 96-well plate with 100 μL per well, 200 ng/well antigen, and incubate at 4 °C. Incubate overnight. The sample was diluted in 10% FCS-PBST, 100 μL of the diluted sample was added to each well, and incubated at 37 °C for 1 h. After incubation, the plate was washed three times with PBST, and the secondary antibody (Goat pAb to Mouse IgA-biotin) was diluted with sample diluent at a ratio of 1:10000. 100 μL was added to each well and incubated at 37 °C for 1 hour. After incubation, the plate was washed three times with PBST, and the antibody Streptavidin-HRP was diluted with sample diluent at a ratio of 1:5000. 100 μL was added to each well and incubated at 37 °C for 1 h. After incubation, the plate was washed three times with PBST, and 100 μL of 1×TMB substrate solution was added to each well to develop the color reaction for 10 minutes. After color development, 100 μL of TMB stop solution was added to terminate the color development, and the absorbance was recorded at 450 nm using a Synegry H1 microplate reader. For result analysis, first subtract the average value of the blank wells from the OD450 of each well to obtain the actual sample absorbance value. When the absorbance value corresponding to the highest dilution multiple is greater than 0.21, the highest dilution is used as the titer of the sample.

The ELISpot test results and ELISA test results are shown in Table 7 and FIG. 3B, FIG. 3C, and FIG. 3D. During the primary immunization, the B11/Flu LPP preparation was administered intramuscularly, and during the second immunization, it was administered through nasal instillation. Mice administered the IN-3-11/Flu LPP formulation had excellent cellular immune responses and higher antibody levels in their serum, nasal lavage fluid, and alveolar lavage fluid.

The above results once again demonstrate that the “primary immunity+boost” approach (systemic immunity+mucosal immunity) can combine the advantages of the two immune methods, induce a higher level of humoral immunity and cellular immunity, and at the same time induce a mucosal immune response.

TABLE 6
Immunization scheme

| G | sample | place | does (mg/kg) |
|---|---|---|---|
| 1 | 10% sucrose | nasal | — |
| 2 | IN-2-6/Flu | nasal | 0.5 (10 μg) |
| 3 | IN-3-4/Flu | nasal | 0.5 (10 μg) |
| 4 | IN-3-5/Flu | nasal | 0.5 (10 μg) |
| 5 | IN-3-11/Flu | nasal | 0.5 (10 μg) |
| 6 | IN-3-12/Flu | nasal | 0.5 (10 μg) |
| 7 | B [symbol] /Flu | Muscle | 0.5 (10 μg) |
| 8 | B [symbol] /Flu + | Muscle | 0.5 (10 μg) |
| | IN-3-11/Flu | nasal | |

[symbol]

indicates data missing or illegible when filed

TABLE 7
Immune effects of different immunization regimens

| | ELISpot- | ELISpot- | LgG | nasal | |
|---|---|---|---|---|---|
| G | Spleen(p [symbol] / [symbol] cells) | Lung(p [symbol] / [symbol] cells) | serum | IgA | [symbol] |
| 1 | 0 | 12 | 1 | 1 | 1 |
| 2 | 8 | 27 | 8455 | 53 | 3 |
| 3 | 12 | 82 | 2 [symbol] 600 | [symbol] | 2 [symbol] |
| 4 | 8 | 18 | 8455 | 11 | [symbol] |
| 5 | 26 | 66 | 2 [symbol] 600 | 106 | 3 [symbol] |
| 6 | 33 | 82 | 51200 | 106 | 53 |
| 7 | 612 | 272 | 819200 | 1 | 1 |
| 8 | 244 | 343 | [symbol] 79262 | 226 | [symbol] 7 |

[symbol]

indicates data missing or illegible when filed

### Example 5 Effects of Different Cationic Lipids on the Physical and Chemical Properties and Expression Levels of LPP Preparations for Mucosal Administration

In this example, the prescription in Table 8 is used to prepare a tetralipid LPP preparation containing luciferase (Luciferase) mRNA (the coding sequence is shown in SEQ ID NO: 1) for mucosal administration, and the prepared LPP preparation is Comparison of physical and chemical properties and in vivo expression levels.

### 5.1 Preparation of Lipid Nanoparticle (LNP-mRNA) Formulation

Preparation of mRNA aqueous solution; Dilute luciferase mRNA into 0.1 mg/mL mRNA aqueous solution with 10 mM sodium citrate (pH=4.0) buffer solution.

Preparation of lipid solution: Dissolve MC3:phospholipid:cholesterol:PEG in ethanol solution at a molar ratio of 50:10:38.5:1.5 to prepare a 6 mg/mL lipid solution.

Preparation of LNP: Using microfluidic technology (Maiana (Shanghai) Technology Co. Ltd., model; Inano D), the lipid solution and the mRNA aqueous solution were mixed under the following conditions: volume=4.0 mL; flow rate ratio=3 (mRNA aqueous solution): 1 (lipid solution), total flow rate=12 mL/min, to obtain LNP-mRNA solution.

Centrifugal ultrafiltration: Add the LNP-mRNA solution into the ultrafiltration tube for centrifugal ultrafiltration concentration (centrifugal force 3000 rpm, centrifugation time 60 min, temperature 4 °C), ultrafiltrate until the ethanol content is <0.5%, and adjust the LNP mRNA concentration to 0.2 mg/mL. The LNP-mRNA preparation numbered MC3 LNP was obtained.

### 5.2 Preparation of Lipid Polyplex (LPP-mRNA) Preparation

Preparation of lipid mixture: Dissolve cationic lipids, phospholipids, cholesterol and PEG lipids in ethanol solution according to the lipid ratio in the recipe of IN-3-11 and the cationic lipids shown in Table 8 to prepare 6 mg/mL of lipid mixture.

Preparation of mRNA aqueous solution: Dilute luciferase mRNA into 0.1 mg/mL mRNA aqueous solution with 10 mM sodium citrate (pH=4.0) buffer solution.

Preparation of protamine sulfate solution: Dissolve protamine sulfate in nuclease-free water to prepare a protamine sulfate solution with a working concentration of 0.125 mg/mL.

Preparation of core nanoparticle solution: Use microfluidic technology to mix protamine sulfate solution and mRNA solution under the following conditions to obtain a core nanoparticle solution formed by protamine and mRNA: mass ratio=4 (mRNA Solution): 1 (protamine solution), flow rate ratio=5 (mRNA): 1 (protamine solution), total flow rate=12 mL/min, room temperature.

Preparation of LPP: Mix the nuclear nanoparticle solution and the lipid solution twice under the following conditions: mass ratio=20 (nuclear nanoparticle solution): 1 (lipid mixture), flow rate ratio=3 (nuclear nanoparticle solution)): 1 (lipid solution), total flow rate=12 mL/min, room temperature, obtain LPP-mRNA solution.

Centrifugal ultrafiltration: Centrifuge the LPP-mRNA solution to remove ethanol through ultrafiltration (centrifugal force 3000 rpm, centrifugation time 60 min, temperature 4 °C), ultrafiltrate until the ethanol content is <0.5%, and adjust the LPP mRNA concentration to 0.2 mg/mL.

LPP formulations numbered 121 LPP, 138-1 LPP, 139-1 LPP, 140-1 LPP and 140-2 LPP were prepared.

And prepare a B11 LPP preparation with a lipid ratio of M5: phospholipid: cholesterol: PEG of a molar ratio of 40:15:43.5:1.5 through the above method, and adjust the LPP mRNA concentration to 0.2 mg/mL.

5.3 Detection of Physical and Chemical Properties of LPP Preparations with Different Cationic Lipids

The physical and chemical properties of the prepared LPP preparations and LNP preparations were tested. For specific detection methods of particle size detection, encapsulation efficiency detection, polydispersity index (PDI) detection and purity detection, please refer to Example 2.3.

The physical and chemical property test results are shown in Table 8. LPP preparations prepared with different cationic lipids have higher encapsulation efficiency and purity and lower polydispersity index.

TABLE 8
Formulations and physical and chemical properties of LPP formulations with different cationic lipids

| | | Cationic | DOPE | lipid | DMG- | cholesterol | size | | | purity |
|---|---|---|---|---|---|---|---|---|---|---|
| Nu | Cationic lipid | lipid (%) | (%) | (%) | PEG (%) | % | (nm) | PDI | [symbol] | ( [symbol] ) |
| MC3 LNP | MC3 | 50 | DSPC | 10 | 1.5 | 38.5 | 97.0 | 0.17 | 95.8 | 79.2 |
| B [symbol] LPP | M5 | 40 | DOPE | 15 | 1.5 | 43.5 | 110. [symbol] | 0.09 | 97.9 | 84.0 |
| 121 LPP | SW- [symbol] -121 | 42.5 | DOPE | 35 | 3.75 | 18.75 | 69.2 | 0.18 | 93.4 | 82. [symbol] |
| 138-1 LPP | SW- [symbol] -138-1 | 42.5 | DOPE | 35 | 3.75 | 18.75 | 70.0 | 0.24 | 92.5 | 8 [symbol] .8 |
| 139-1 LPP | SW- [symbol] -139-1 | 42.5 | DOPE | 35 | 3.75 | 18.75 | 78.9 | 0.20 | 93.1 | 84.0 |
| 140-1 LPP | SW- [symbol] -140-1 | 42.5 | DOPE | 35 | 3.75 | 18.75 | 73. [symbol] | 0.22 | 94.0 | 79.4 |
| 140-2 LPP | SW- [symbol] -140-2 | 42.5 | DOPE | 35 | 3.75 | 18.75 | 98.1 | 0.22 | 93.4 | 8 [symbol] . [symbol] |

[symbol]

indicates data missing or illegible when filed

5.4 In Vitro Expression of LPP Preparations with Different Cationic Lipids

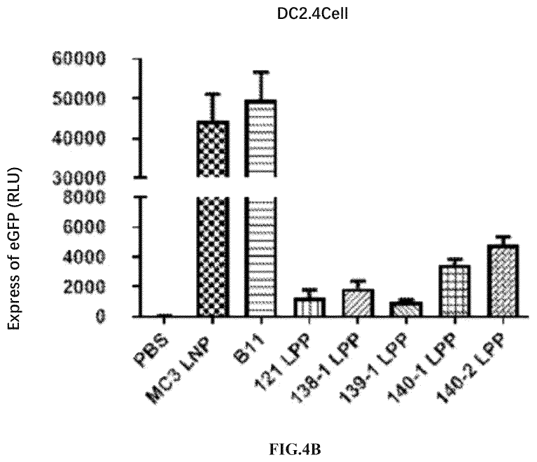

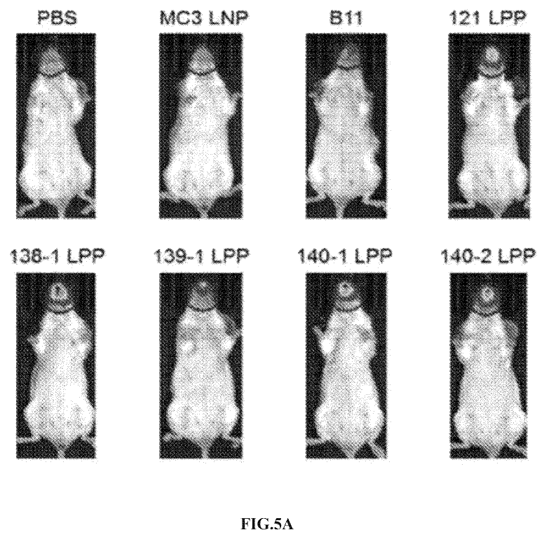

Non-small cell lung cancer cells (A549 cells) were cultured in Dulbecco's modified Eagle medium (DMEM, GIBCO, 10566-016), cultured at 37 °C and 5% CO2. Dendritic cells (DC2.4 cells) were grown in Gibco RPMI 1640 medium supplemented with 10% FBS (Hyclone, 35-081-CV) and 1% penicillin-streptomycin (GIBCO, 15140-122) at 37 °C and cultured under 5% CO2 conditions. Take the LPP solutions of B11, 121 LPP, 138-1 LPP, 139-1 LPP, 140-1 LPP and 140-2 LPP and the LNP solution of MC3 LNP containing 100 ng of luciferase mRNA respectively, and use PBS as a control. A549 cells and dendritic cells DC2.4 cells were transfected. The cells were lysed 24 hours after administration and the expression of luciferase protein was detected. The results are shown in FIG. 4A and FIG. 4B, in which B11 LPP and 140-2 LPP both showed good in vitro transfection effects and had high expression levels.

5.5 In Vivo Expression of LPP Preparations with Different Cationic Lipids

The in vivo expression of the LPP preparation prepared in Example 5.2 was detected.

The specific detection methods are as follows: Female BALB/c mice (Beijing Vitonglihua Experimental Animal Technology Co., Ltd.) aged 6 to 8 weeks were anesthetized with sodium pentobarbital (70 mg/kg), and then the prepared LPP was administered via nasal instillation, solution (3 mice per group), and 2 groups of mice were administered nasal mucosa MC3 LNP solution and PBS solution (negative control) respectively, each LPP solution or LNP solution administered contained 2 μg of luciferase mRNA (10 μL). Six hours after administration, mice were intraperitoneally injected with 3 mg of D-luciferin substrate (Maokang Biotechnology), and 10 minutes after substrate injection, the mice were treated with Xenogen IVIS-200.

The imaging system performs in vivo imaging of mice to detect luciferase expression in vivo.

The experimental results are shown in FIG. 5A and FIG. 5B. All LPP preparations are only expressed in the nasal cavity, with high local expression and low systemic toxicity. Among them, the luciferase expression levels of LPP preparations containing different cationic lipids prepared at the lipid ratio in the IN-3-11 formula were similar, but they had higher luciferase expression in the nasal cavity than the B11 LPP preparation.

The above results show that the lipid ratio screened by the present invention is particularly suitable for intranasal administration. LPP preparations containing homemade new cationic lipids have better expression in the nasal cavity at the same lipid ratio. Among them, SW-II-140-2 cationic lipid is particularly preferred.

### Example 6 Effect of Atomization on the Physical and Chemical Properties and Transfection Efficiency of LPP Preparations

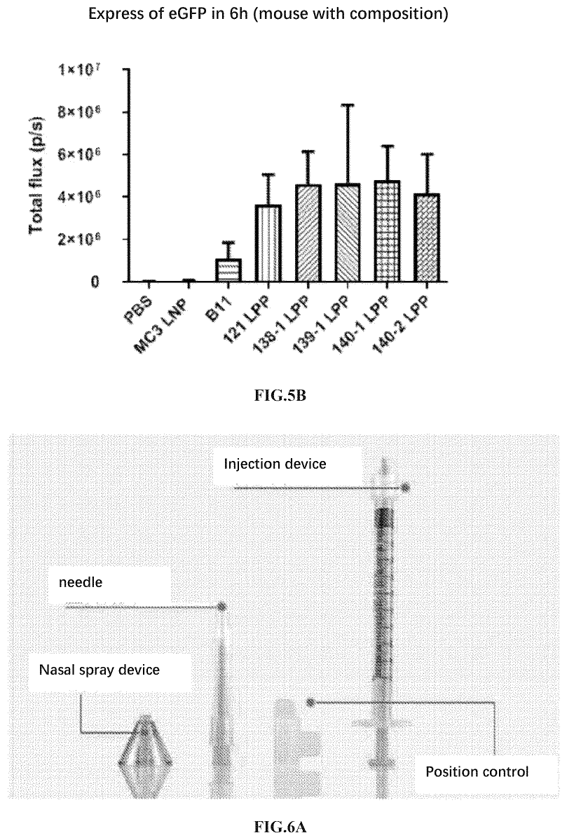

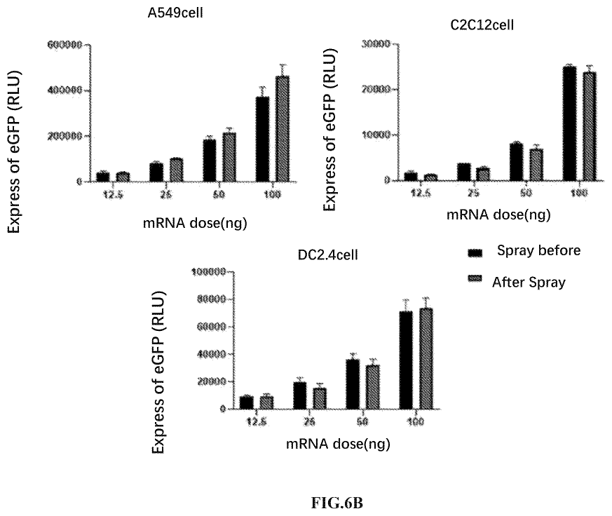

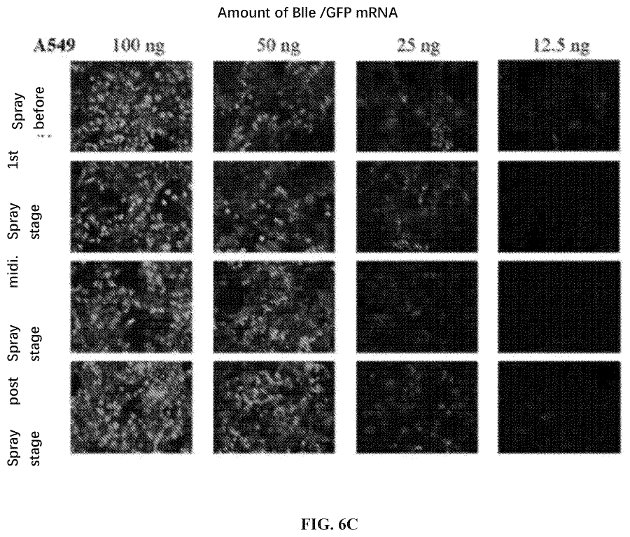

In this example, the prescription of B11 is used to prepare a four-lipid LPP preparation containing luciferase (Luciferase) mRNA (coding sequence is shown in SEQ ID NO: 1) or eGFP protein mRNA (coding sequence is shown in SEQ ID NO: 4), and atomized by an atomizing drug delivery device as shown in FIG. 6A. The physical and chemical properties and transfection efficiency of the LPP preparation before atomization and after atomization were measured to determine the impact of atomization on it.

### 6.1 Preparation of Lipid Polyplex (LPP-mRNA) Preparation

Preparation of lipid mixture: Dissolve M5, phospholipids, cholesterol and PEG lipids in ethanol solution according to the lipid ratio in the B11 prescription to prepare a 6 mg/mL lipid mixture.

Preparation of mRNA aqueous solution: Dilute luciferase mRNA or eGFP mRNA into 0.1 mg/mL mRNA aqueous solution with 10 mM sodium citrate (pH=4.0) buffer solution.

Preparation of protamine sulfate solution: Dissolve protamine sulfate in nuclease-free water to prepare a protamine sulfate solution with a working concentration of 0.125 mg/mL.

Preparation of core nanoparticle solution: Use microfluidic technology to mix protamine sulfate solution and mRNA solution under the following conditions to obtain a core nanoparticle solution formed by protamine and mRNA: mass ratio=4 (mRNA Solution): 1 (protamine solution), flow rate ratio=5 (mRNA): 1 (protamine solution), total flow rate=12 mL/min, room temperature.

Preparation of LPP: Mix the nuclear nanoparticle solution and the lipid solution twice under the following conditions: mass ratio=20 (nuclear nanoparticle solution): 1 (lipid mixture), flow rate ratio=3 (nuclear nanoparticle solution)): 1 (lipid solution), total flow rate=12 mL/min, room temperature, obtain LPP-mRNA solution.

Centrifugal ultrafiltration: Centrifuge the LPP-mRNA solution to remove ethanol through ultrafiltration (centrifugal force 3000 rpm, centrifugation time 60 min, temperature 4 °C), ultrafiltrate until the ethanol content is <0.5%, and adjust the LPP mRNA concentration to 0.1 mg/mL. LPP preparations numbered B11/Luc and B11/eGFP were prepared.

### 6.2 Test the Physical and Chemical Properties of LPP Preparations Before and After Atomization

The LPP preparations of B11/Luc and B11/eGFP were atomized using the atomizing administration device shown in FIG. 6A, and the physical and chemical properties of the preparations before and after atomization were tested. For specific detection methods of particle size detection, encapsulation efficiency detection, polydispersity index (PDI) detection and purity detection, please refer to Example 2.3.

The physical and chemical property test results are shown in Table 9. After the nasal spray device atomized the LPP sample, there was no significant change in the physical and chemical properties of the sample before and after atomization.

TABLE 9
Physical and chemical properties before and after atomization

| Name of sample | size/nm | PDI | Zeta/mV |
|---|---|---|---|
| [symbol] /L [symbol] Spray | 128. [symbol] | 0.13 [symbol] 00 | 28.9 [symbol] 4 |
| [symbol] /L [symbol] after sprayed | 130.1 [symbol] 9 | 0.12 [symbol] 04 | 28.0 [symbol] 0.5 |

[symbol]

indicates data missing or illegible when filed

### 6.3 Detect the Transfection Efficiency of LPP Preparations Before and After Atomization

Mouse myoblasts (C2C12 cells) and non-small cell lung cancer cells (A549 cells) were cultured in the presence of 10% FBS (Hyclone, 35-081-CV) and 1% penicillin-streptomycin (GIBCO, 15140-122). Culture in Dulbecco's modified Eagle medium (DMEM, GIBCO, 10566-016) at 37 °C and 5% CO2. Dendritic cells (DC2.4 cells) were grown in Gibco RPMI 1640 medium supplemented with 10% FBS (Hyclone, 35-081-CV) and 1% penicillin-streptomycin (GIBCO, 15140-122) at 37 °C and cultured under 5% CO2 conditions.

Take the B11/Luc LPP preparations containing 12.5, 25, 50, and 100 ng of luciferase mRNA before and after atomization, respectively, and transfect C2C12 cells, A549 cells, and dendritic cells DC2.4 cells, 24 hours after treatment, the cells were lysed and the expression of luciferase protein was detected.

Take the B11/eGFP LPP preparations containing 12.5, 25, 50, and 100 ng of luciferase mRNA and eGFP mRNA before atomization, before atomization, during atomization, and after atomization, respectively, and transfect A549 cells. 24 hours after taking the drug, observe the expression of green fluorescent protein under a fluorescence microscope, take pictures and compare

The test results are shown in FIG. 6B and FIG. 6C respectively. After the LPP sample was atomized by the nasal spray device, there was no significant change in cell transfection efficiency and in vitro expression between the atomized sample and the sample before atomization.

Although the present invention has been disclosed above in terms of preferred embodiments, they are not intended to limit the present invention. Anyone familiar with this technology can make various changes and modifications without departing from the spirit and scope of the present invention. Therefore, the protection scope of the present invention should be defined by the claims.

Sequence list

| SEQ ID | | |
|---|---|---|
| NO: | decription | SEQUENCE |
| 1 | eGFP | AUGGAGGACGCCAAGAACAUCAAGAAGGGCCCCGCCCCCUUCUACCCCCUGGAGGAC |
| | | GGCACCGCCGGCGAGCAGCUGCACAAGGCCAUGAAGCGGUACGCCCUGGUGCCCGGC |
| | | ACCAUCGCCUUCACCGACGCCCACAUCGAGGUGGACAUCACCUACGCCGAGUACUUC |
| | | GAGAUGAGCGUGCGGCUGGCCGAGGCCAUGAAGCGGUACGGCCUGAACACCAACCAC |
| | | CGGAUCGUGGUGUGCAGCGAGAACAGCCUGCAGUUCUUCAUGCCCGUGCUGGGCGCC |
| | | CUGUUCAUCGGCGUGGCCGUGGCCCCGCCAACGACAUCUAUCAACGAGCGGGAGCUG |
| | | CUGAACAGCAUGGGCAUCAGCCAGCCCACCGUGGUGUUCGGUGAGCAAGAAGGGCUG |
| | | CAGAAGAUCCUGAACGUGCAGAAGAAGCUGCCCAUCAUCCAGAAGAUCAUCAUCAUG |
| | | GACAGCAAGACCGACUACCAGGGCUUCCAGAGCAUGUACACCUUCGUGACCAGCCAC |
| | | CUGCCCCCCGGCUUCAACGAGUACGACUUCGUGCCCUAGAGCUUCGACCGGGACAAG |
| | | ACCAUCGCCCCGAUCAUGAACAGCAGCGGCAGCCCGGCCUGGCCCAAGGGCGUGGCC |
| | | CUGCCCCACCGGACCGCCUGCGUGCGGUUCAGCCACGGCCGGGACCCCAUCUUCGGC |
| | | AACCAGAUCAUCCCCGACACCGCCAUCCUGAGCGUGGUGCCCUUCCACCACGGCUUC |
| | | GGCAUGUUCACCACCCUGGGCUACCUGAUCUGCGGCUUCCGGGUGGUGCUGAUGUAC |
| | | CGGUUCGAGGAGGAGCUGUUCCUGCGGAGCCUGCAGGACUACAAGAUCCAGAGCGCC |
| | | CUGCUGGUGCCCACCCUGUUCAGCUCUUCGCCAAGAGCAACCCUGAUCGACAAGUAC |
| | | GACCUGAGCAACCUGCACGAGAUCGCCAGCGGCGGCGCCCCCCUGAGCAAGGAGGUG |
| | | GGCGAGGCCGUGGCCAAGCGGUUCCACCUGCCCGGCAUCCGGCAGGGCUACGGCCUG |
| | | ACCGAGACCACCAGCGCCAGCCUGAUCACCCCCGAGGGCGACGACAAGCCCGGCGCC |
| | | GUGGGCAAGGUGGUGCCCUUCUUCGAGGCCAAGGUGGUGCGACCUGGACACCUUCAA |
| | | GACCCUGGCCGUGAACCAGCGGGGCGAGCUGUCCGUGCGGGGCCCCAUGAUCAUGAG |
| | | CGGCUACGCGAACAACCCCGAGGCCACCAACGCCCUGAUCGACAAGGACGGCUGGCU |
| | | GCACAGCGGCGACAUCGCCUACUGGGACGAGGACGAGCACUUCUUCAUCGUGGACCG |
| | | GCUGAFGAGGCUGAUCAACUACAAGGGCUACCAGGUGGCCCCCGCCGAGGUGGAGAG |
| | | CAUCCUGCUGCAGCACCCAAACAUCUUCGACGCCGGCGUGGCCGGCCUGCCCGACGA |
| | | CGACGCCGGCGAGCUGCCCGCCGCCGUGGUGGUGCUGGAGCACGGCAAGACCAUGAC |
| | | CGAGAAGGAGAUCGUGGACUACGGUGGCCAGCCAGGUGACACCGCCAAGAAGCUGCG |
| | | GGGCGGCGUGGUGUUCGUGGACGAGGUGCCCAAGGGCCUGACCGGCAAGCUGGACGC |
| | | CCGGAAGAUCCGGGAGAUCCUGAUCAAGGCCAAGAAGGGCAGGCAAGAUCGCCGUGU |
| | | GA |
| 2 | COVID- | AUGUUCGUAUUCCUAGUGCUGCUGCCCCUGGUGAGCAGCCAGUGCGUGAACCUGACC |
| | 19 | ACACGCACUCAGCUGCCACCAGCCUACACCAACUCAUUCACCAGGGGUGUGUACUAU |
| | | CCUGACAAGGUGUUCAGGAGCAGCGUGCUGCACUCCACCCAGGAUCUGUUCCUGCCC |
| | | UUCUUCAGCAACGUGACAUGGUUCCACGCCAUCCACGUGAGCGGGACAAACGGCACC |
| | | AAGCGGUUUGACAAUCCCGUUCUGCCAUUCAACGAUGGCGUGUACUUCGCCAGCACC |
| | | GAGAAAAGCAACAUCAUCCGGGGCUGGAUCUUCGGCACCACACUGGACAGCAAGACC |
| | | CAGAGUCUGCUGAUCGUGAACAACGCCACCAACGUGGUGAUCAAGGUGUGUGUAGUU |
| | | CCAGUUCUGUAAUGACCCCUUCCUGGGGGUCUAUUACCAGAAGAACAAUAAAAGCUG |
| | | GAUGGAGAGCGAGUUUCGGGUGUACAGUUAGUGCCAACAACUGUACAUUCGAGUACG |
| | | UGUCCCAGCCAUUCCUGAUGGACCUGGAGGGGAAGCAGGGGAACUUCAAGAACCUGC |
| | | GGGAGUUCGUGUUUAAGAAUAUCGACGGUAUAUUUUAAGAUCUACUCCAAGCAGACC |
| | | CCCAUCAACCUGGUGCGGGAUCUGCCCCAGGGCUUCAGCGCCCUGGAGCCCCUGGUG |
| | | GACCUGCCCAUGGGAUCAACAUCACCCCGGUUCCAGACCCUGCUGGCCCUGCAUCGG |
| | | AGUUACCUFACCCCAGGUGACUCCAGCAGCGGCUGGACAGCCGGGGCUGCAGCCUAC |
| | | UACGUGGGCUACCUGCAGCCCCGGACCUUCCUCCUGAAGUACAAUGAGAACGGCACC |
| | | AUCACCGAUGCCGUGGAUUGUGCUGUGGACCCCCUGUCUUAGACCAAGUGCACCCUG |
| | | AAAAGCUUCACCGUGGAGAAGGGGAUCUACCAGACAAGCAACUUUCGCGUGCAGCCC |
| | | ACCGAGAGUAUCGUGAGGUUCCCCAACAUCACCAACCUGUUGCCCAUUCGGGAGGUG |
| | | UUCAACGCCACCCGGUUCGCAAGCGUGUACGCUUGGAACCCGGAACGGAUCAGCAAU |
| | | UGCGUGGCUGAUUACAGCGUGCUGUACAACAGCGCCUCCUUCAGCACAUUCAAGUGC |
| | | UACGGCGUCUCCCCCACAAAGCUGAACGAUCUGUGUUUCACCAACGUGUACGCAGAC |
| | | AGCUUUGUGAUCCGGGGAGACGAGGUGCGGCAGAUCGCACCCGGGCAGACCGGCAAG |
| | | AUUGCUGACUACAAUUACAAGCUGCCUCGAUGACUUCAUAGGCUGCGUGAUUGCCUG |
| | | GAACAGCAAUAACCUFFACAGCAAGGUCAGGAGGUAAUUACAAUUACCGCUACCGGC |
| | | UGUUCAGGAAGUCCAAUCUGAAGCCCUUCGAGAGGGACAUCUCCACCGAGAUCUACC |
| | | AGGCUGGGUCCACCCCAUGCAAUGGGGUGGAGGGCUUCAACUGCUACUUCCCCCUGC |
| | | AGUCCUACGGCUUCCAGCCCACCAAUGGGGUGGGCUACCAGCCCUACCGGGUGGUGG |
| | | UGCUGAGCUUCGAGCUCCUGCACGCCCCCGCCACUGUGUGCGGCCCCAAGAAGUCCA |
| | | CCAACCUGGUGAAGAACAACUGCGUGAACUUCAACUUCAACGGGUUUGACCGGGACU |
| | | GGGGUGCUCACAGAGAGCAACAAGAAGUCCUGCCCUUCCAGGAGUUGCGGAAGGGAC |
| | | AUCGCCGACACCACGGAUGCCGUGAGGGAUCCCCAGACCCUCGAGAUCCUGGACAUC |
| | | ACGCCAUGCUCCUUCGGGGGCGUGAGCGUGAUCACUCCUGGCACAAACACCUCCAAC |
| | | CAGGUGGCUGUGCUGUACCAGGGCCUGAACUGCACCGAGGUGCCUGUGGCCAUCCAU |
| | | GCUGACCAGCUGACCCCCACCUGGCGGGUCUACAGCACCGGCAGCAACGUGUUCCAG |
| | | ACCCGGGCCGGCUGCCUGAUCGGGGCCGAGCACGUGAACAACAGCUACGAGUGCGAU |
| | | AUCCCCAUCGGCGCCGGCAUCUGCGCUAGCUACCAGACACAGACCAAUAGCCCCCAA |
| | | UCCGCUCAAUCUGUGGCUAGCCAGAGCAUCAUCGCCUACACUAUGUCUCUGGGCGCA |
| | | GAGAAUAGUGUGGCUUACAGCAACAACAGCAUCGCCAUUCCCACUAAUUUCACAAUU |
| | | AGUGUGACCACCGAGAUCCUGCCUGUGAGCAUGACCAAGACCAGCGUGGACUGCACC |
| | | GUGUACAUCUGCGGGGAUAGCACCCACUGCAGCAACCUGGUGUUGCAGUACGGCAGC |
| | | UUCUGCACACAGCUGAACCGGGCCCUGACCGGUAUCGCUGUGGAGCAGGACAAGAAC |
| | | ACCCAGGAGGUGUUUGCGCAGGUGAAGCAGAUCUACAAGACCCCUCCUAUCAAGGAC |
| | | UUCGGGGGCUUUAACUUCAGCCAGAUCCUGCCAGACCCCCUCCAAGCCUCCAAGAGG |
| | | AGCUUCAUCGAGGAUCUCCUGUUCAACAAGGUGACCCUGGCCGAUGCCGGCUUCAUC |
| | | AAGCAGUACGGAGAUUGCCUGGGCGACAUCGCCGCCAGGGAUCUCAUCUGCGCCCAG |
| | | AAGUUCAACGGCCYGACCGUGCUGCCCCCCCUGCUGACCGAUGAGAUGAUCGCUCAG |
| | | UACACCAGCGCCCUGCUGGCUGGGACCAUCACCCAGCGGGUGGACCUUCGGGCCGGC |
| | | GCCGCCCUGCAGAUCCCCUUCGCCAUGCAGAUGGCCUACCGGUUCAACGGUAUUGGG |
| | | GUGACCCAGAAUGUGCUGUAUGAGAACCAGAAGCUGAUCGCCAACCAGUUCAACUCU |
| | | GCCAUCGGCAAGAUCCAGGACUCCCUGACCAGCACUGCCAGCGCCCUGGGACCGCUG |
| | | CAGGACGUGGUGAACCAGAACGCCCAGGCCCUGAAUACCCUGGUGAAGCAGCUGUCC |
| | | AGCAACUUCGGGGCCAUCAGCAGCGUGCUGAACGACAUCCUGUCCCGCCUGGACCCA |
| | | CCAGAGGCAGAGGUGCAGAUUGAUCCGGCUGAUCACAGGUAGGCUGCAGAGCCUGCA |
| | | GACCUACGUGACCCAGCAGCUGAUCCGCGCCGCUGAGAUUAGGGCCAGCGCCAACCU |
| | | GGCCGCCACUAAGAUGAGCGAGUGCCGUGCUGGGCCAGAGCAAGCGGGUGGACUUCU |
| | | GCGGCAAGGGGUAUCACCUGAUGACCUUCCCUCCAGUCCGCCCCCACGGGGUGGUGU |
| | | UCCUGCACGUGACAUACGUGCCCGCCCAGGAGAAGAACUUCACCACCGCCCCUGCCA |
| | | UCUGCCACGAUGGCAAGGCCCACUUUCCCCGGGAGGGCGUGUUCGUGUCCAAUGGCA |
| | | CACAUUGGUUCGUGACCCAGCGGAACUUCUACGAGCCACAGAUCAUUACCACUGACA |
| | | AUACCUUUGUCAGUGGUAACUGUGAACGUGGUGAUCGGCAUUGUGAACAACACAGUG |
| | | UACGAUCCUCUGCAGCCAGAGCUGGACAGCUUCAAGGAGGAGCUGGACAAGUACUUC |
| | | AAGAACAGAGGAGCCCCCGACGUGGAUCUUGGAGACAUCUCUGGCAUCAACGCCAGC |
| | | GUGGUCAACAUCCAGAAGGAGAUUCGACCGCCUGAACGAGGUUGCCAAGAACCUGAA |
| | | CGAGAGUCUGAUCGACCUCCAGGAGCUUGGCAAGUACGAGCAGUACAUCAAGUGGCC |
| | | AUGGUACAUCUGGCUGGGCUUCAUCGCCGGCCUGAUCGCCAUCGUGAUGGUGACCAU |
| | | CAUGCUGUGCUGCAUGACCUCUUGUUGCAGCUGCCUGAAGGGGUGCUGCAGCUGCGG |
| | | UAGUUGCUGUAAGUUCGACGAGGACCGACAGCGAGCCCGUGCUGAAGGGCGUGAAGC |
| | | UGCACUACACGUAAUAG |
| 3 | antigen | AUGGCGUCCCAAGGCACCAAACGGUCUUAUGAACAGAUGGAAACUGAUGGGGAACG |
| | of | CCAGAAUGCAACUGAGAUUAGGGCAUCCGUCGGGAAGAUGAUUGAUGGAAUUGGGC |
| | Flu | GAUUCUACAUCCAAAUGUGCACUGAACUUAAACUCAGUGAUUAUGAAGGGCGGUUG |
| | | AUCCAGAACAGCUUGACAAUAGAGAGAAUGGUGCUCUCUGCUUUUGAUGAGAGAAG |
| | | GAAUAGAUAUCUGGAAGAACACCCCAGCGCGGGGAAAGAUCCUAAGAAAACUGGAG |
| | | GGCCCAUAUACAAGAGAGUAGAUGGAAGAUGGAUGAGGGAACUCGUCCUUUAUGAC |
| | | AAAGAAGAAAUAAGGCGAAUCUGGCGCCAAGCCAACAAUGGUGAUGAUGCGACAGC |
| | | UGGUCUAACUCACAUGAUGAUCUGGCAUUCCAAUUUGAAUGAUACAACAUACCAGA |
| | | GGACAAGAGCUCUUGUUCGCACCGGAAUGGACCCAGGAUGUGCUCUCUGAUGCAGG |
| | | GUUCGACUCUCCCUAGAAGGUCCGGAGCUGCAGGCGCUGCAGUCAAGGAAUCGGGA |
| | | CAAUGGUGAUGGAGCUGAUCAGAAUGAUCAAACGGGGGAUCAACGAUCGAAAUUUC |
| | | UGGAGAGGUGAGAAUGGGCGGAAAACAAGGAGUGCUUAUGAGAGAAUGUGCAACAU |
| | | UCUUAAAGGAAAAUUUCAAACAGCUGCACAAAGAGCAAUGAUGGAUCAAGUGAGAG |
| | | AAAGCCGGAACCCAGGAAAUGUGAGAUCCGAAGAUCUCAUAUUUUCGGCAAGAUCU |
| | | GCACUAAUAUUGAGAGGGUCAGUUGCUCACAAAUCUUGCCUACCUGCCUGUGUGUA |
| | | UGGACCUGCAGUAUCCAGUGGGGACAACUUCGAAAAAGAGGGAUAUUCCUUAGUGG |
| | | GAAUAGACCCUUCAAACUACUUCAAAAUAGCCAAGUAUACAGCCUAAUCAGACCGA |
| | | ACGAGAAUCCAGCAGCGCCGAGUCAGUUGGUGUGGAUGGCAUGCCAUUCUGCUGCA |
| | | UUUGAAGAUUUAAGAUUGUUAAGCUUCAUCAGAGGGACCAAAGUAUCUCCGCGGGG |
| | | GAAACUUUCAACUAGAGGAGUACAAAUUGCUUCAAAUFAFAACAUFFAUAAUAUGG |
| | | AAUCAAGUACUCUUGAACUGAGAAGCAGGUACUGGGCCAUAAGGACCAGGAGUGGA |
| | | GGAACACUAAUCAACAGAGGGCCUCUGCAGGCCAAAUCAGUGUGCAACCUACGUUU |
| | | UCUGUACAAAGAAACCUCCCAUUUGAAAAUCAACCGUCAUGGCAGCAUUCACUGGA |
| | | AAUACGGAGGGAAGAACCUCAGACAUGAGGGCAGAAAUCAUAAGGAUGAUGGAAGG |
| | | UACAAAACCAGAAGAAGUGUCCUUCCGUGGGCGGGGAGUUUUCGAGCUCUCAGACG |
| | | AAAAGGCAACGAACCGAUCGUCGGGUCUUUUUGACAUGAGUAAUGAAGGAUCUUAU |
| | | UUCUUCGGAGACAAUGCAGAAGAGUACGACAAUAGCCUUCUAACCGAGGUCGAAAC |
| | | GCCUAUCAGAAACGAAUGGGGGUGCAGAUGCAACGAUUCAAGUGAC |
| 4 | eGFP | AUGGUGAGCAAGGGCGAGGAGCUGUUCACCGGGGUGGUGCCCAUCCUGGUCGAGCU |
| | | GGACGGCGACGUAACGGCCACAAGUUCAGCGUGUCCGGCGAGGGCGAGGGCGAUGC |
| | | CACCUACGGCAAGCUGACCCUGAAGUUCAUCUGCACACCGGCAAGCUGCCCGUGCC |
| | | CUGGCCCACCCUCGUGACCACCCUGCCUACGGCGUGCAGUGCUUCAGCCGCUACCC |
| | | CGACCACAUGAAGCAGCACGACUUCUUCAAGUCCGCCAUGCCCGAAGGCUACGCCA |
| | | GGAGCGCACCAUCUUCUUCAAGACGACGGCAACUACAAGACCCGCGCCGAGGUGAA |
| | | GUUCGAGGGCGACACCCUGGUGAACCGCAUCGAGCUGAAGGCAUCGACUUCAAGGA |
| | | GGACGGCAACAUCCUGGGCACAAGCUGGAGUACAACUACAACAGCCACAACGUCUA |
| | | UAUCAUGGCCGACAAGCAGAGAACGGCAUCAAGGUGAACUUCAAGAUCCGCCACAA |
| | | CAUCGAGGACGGCAGCGUGCAGCUCGCCGACCACUACCAGCAGAACACCCCAUCGG |
| | | CGACGGCCCCGUGCUGCUGCCCGACACCACUACCUGAGCACCCAGUCCGCCCUGAG |
| | | CAAAGACCCCAACGAGAAGCGCGAUCACAUGGUCUGCUGGAGUUCGUGACCGCCGC |
| | | CGGGAUCACUCUCGGCAUGGACGAGCUGUACAAGUAA |

1-35. (canceled)

36. A lipid composition for mucosal administration, comprising a therapeutic agent or a prophylactic agent and a lipid encapsulating the therapeutic agent or the prophylactic agent, wherein the lipid encapsulating the therapeutic agent or the prophylactic agent comprises a cationic lipid lipids, phospholipids, steroids and polyethylene glycol modified lipids; the lipid composition also includes a cationic polymer, wherein the cationic polymer and the therapeutic or preventive agent are associated into a complex and co-encapsulated Lipid multimeric complexes are formed in the lipid; the lipid composition contains 2.5-20 mole % of polyethylene glycol-modified lipids, based on the total amount of all lipids in the lipid composition.

37. The lipid composition of claim 36, wherein the therapeutic or prophylactic agent is a nucleic acid, such as RNA, in particular mRNA.

38. The lipid composition of claim 36, wherein the cationic lipid comprises a compound of formula (I), or a pharmaceutically acceptable salt thereof 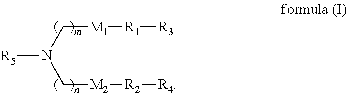
wherein,
R₁ and R₂ each independently selected from key, C₁-C₁₂ Alkyl and C₂-C₁₂ Alkenyl;
R₃ and R₄ each is independently selected from C₁-C₁₂ Alkyl, C₂-C₁₂ Alkenyl, C₆-C₁₀ Aryl and 5-10 membered heteroaryl; and R₃ and R₄ each independently selects t R₆ Instead, t is an integer selected from 1-5;
R₆ each is independently selected from C₁-C₁₂ Alkyl and C₂-C₁₂ Alkenyl;
M₁ and M₂ each independently selected from bond, H, —O—, —S—, —C(O)—, —OC(O)—, —C(O)O—, —OC(O)O—, —SC(S)—, —C(S)S—, 3-10 membered heterocycle, —NR₇—, or
R₅ with M₁ and M₂ One of them together with the connected N atom forms a 3-10 membered heterocyclic ring, and the corresponding R₁/R₃ or R₂/R₄ is not present, the heterocycle is optionally replaced by R₇ replace;
R₅ selected from C₃₋₈ Carbocyclic ring, —C₁₋₁₂ Alkylene-Q, Q is selected from H, —OR₇, —SR₇, —OC(O)R₇, —C(O)OR₇, —N(R₇)C(O)R₇, —N(R₇)S(O)₂R₇, —N(R₇)C(S)R₇, —N(R₇)₂, cyano group, C₃ Carbocyclic ring, 3-10 membered heterocyclic ring, C₆-C₁₀ Aryl, each of the above groups is optionally substituted by one or more C₁-C₁₂ Alkyl, C₂-C₁₂ Alkenyl, C₁-C₁₂ Alkoxy, C₆-C₁₀ Aryl, 5-10 membered heteroaryl, 3-10 membered heterocycle, halogen, hydroxyl, oxo (═O) substitution;
m and n are each independently an integer selected from 0-12;
the alkyl, alkenyl and alkylene groups are each optionally and independently interrupted by one or more groups selected from: —O—, —S—, —NR₇—, —C(O)—, —OC(O)—, —C(O)O—, —SC(S)—, —C(S)S—, C₃₋₈ carbocyclic ring, and each of the alkyl, alkenyl and alkylene groups is optionally replaced by one or more R₇ replace;
R₇ each is independently selected from H, C₁-C₁₂ Alkyl, C₂-C₁₂ Alkenyl, C₁-C₁₂ Alkoxy, carboxylic acid, sulfinic acid, sulfonic acid, sulfonyl, nitro, cyano, amino, carbamoyl, sulfonamide, C₆-C₁₀ Aryl, 5-10 membered heteroaryl, 3-10 membered heterocycle, halogen, C₃ Carbocyclic ring, each of the above groups is optionally replaced by one or more C₁-C₁₂ Alkyl, C₂-C₁₂ base, C₁-C₁₂ Alkoxy, C₆-C₁₀ Aryl, 5-10 membered heteroaryl, 3-10 membered heterocycle, halogen, hydroxyl, oxo (═O) substitution.

39. The lipid composition of claim 38, wherein, R₁ and R₂ each is independently selected from C₁-C₁₂ Alkyl and C₂-C₁₂ Alkenyl; wherein R₃ and R₄ each is independently selected from C₁-C₁₂ Alkyl and C₂-C₁₂ Alkenyl; and R₃ and R₄ each independently selects t R₆ Replace, t is an integer selected from 1-5; R₆ each is independently selected from C₁-C₁₂ Alkyl and C₂-C₁₂ alkenyl; M₁ and M₂ each is independently selected from —OC(O)—, —C(O)O—, —OC(O)O—, —SC(S)— and —C(S)S—; R₅ selected from —C₁₋₁₂ Alkylene-Q, Q is selected from —OR₇ and —SR₇, R₇ Independently selected from H, C₁-C₁₂ Alkyl, C₂-C₁₂ Alkenyl, C₁-C₁₂ Alkoxy, carboxylic acid, sulfinic acid, sulfonic acid, sulfonyl, nitro, cyano, amino, carbamoyl, sulfonamide, C₆-C₁₀ Aryl and 5-10 membered heteroaryl; m and n are each independently an integer selected from 1-12.

40. The lipid composition of claim 38, wherein the cationic lipid comprises a lipid compound having the structure shown below or a pharmaceutically acceptable salt thereof:

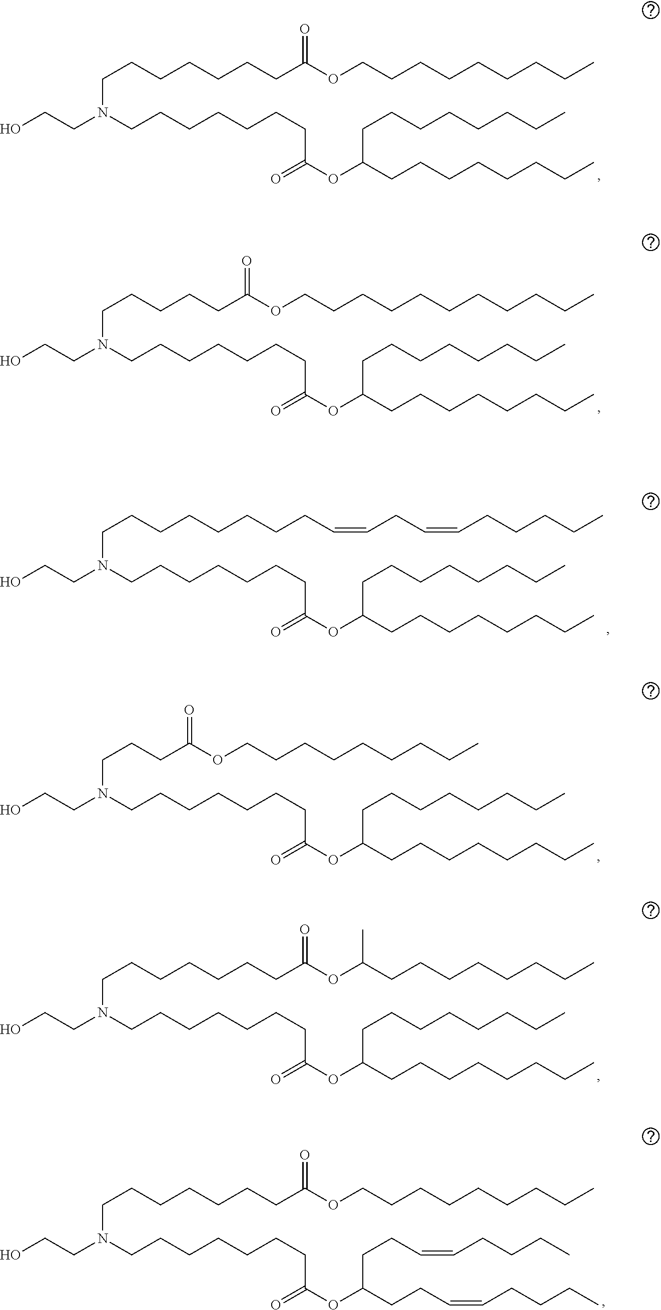 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC ; CCCCC/C=C\C/C=C\CCCCCCCCN(CCO)CCCCCCCC(=O)OC(CCCCCCCC)CCCCCCCC ; CCCCCCCCCOC(=O)CCCN(CCO)CCCCCCCC(=O)OC(CCCCCCCC)CCCCCCCC ; CCCCCCCCC(C)OC(=O)CCCCCCCN(CCO)CCCCCCCC(=O)OC(CCCCCCCC)CCCCCCCC ; CCCC/C=C\CCC(CC/C=C\CCCC)OC(=O)CCCCCCCN(CCO)CCCCCCCC(=O)OCCCCCCCCC]

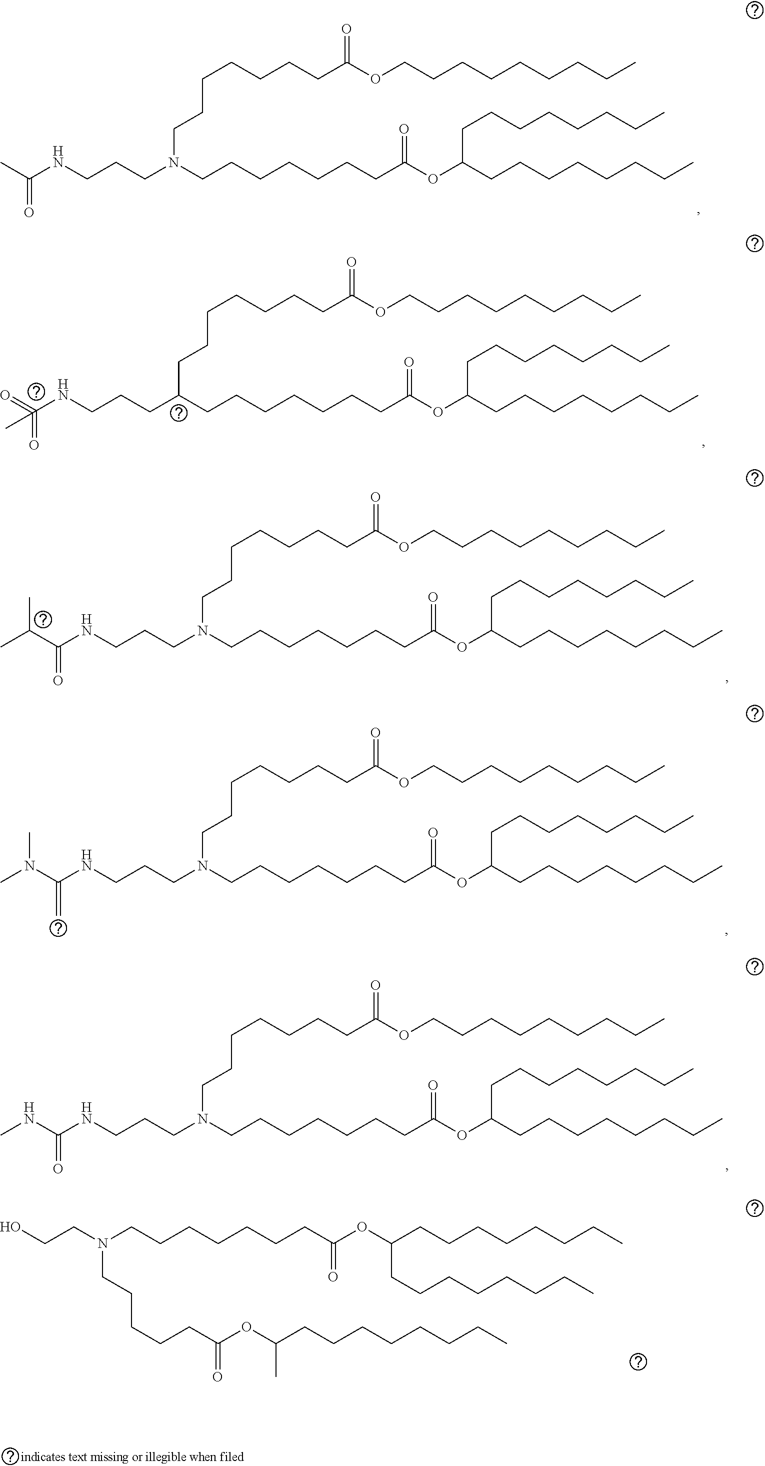

41. The lipid composition of claim 38, wherein the cationic lipid comprises a lipid compound having the structure shown below or a pharmaceutically acceptable salt thereof:

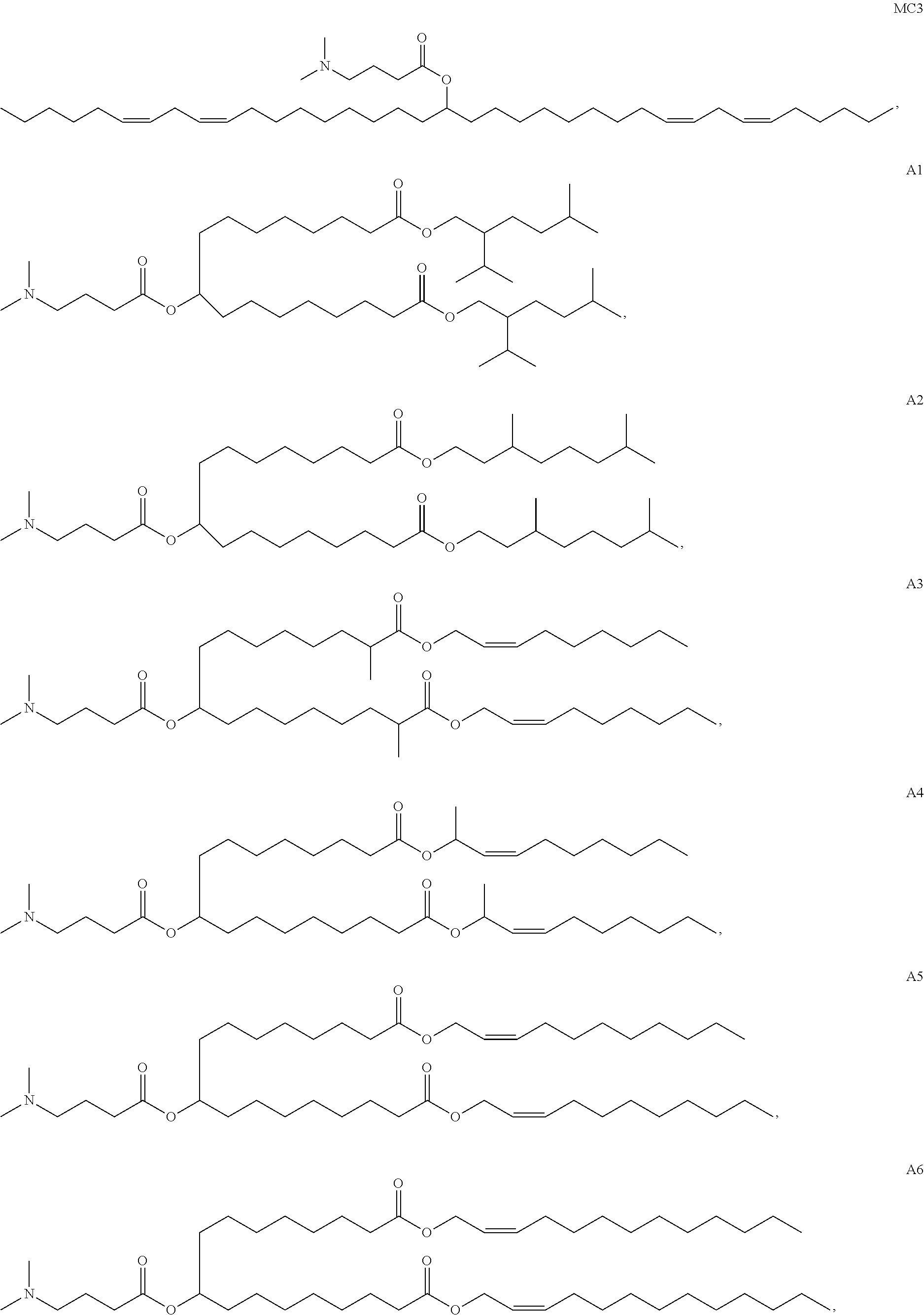

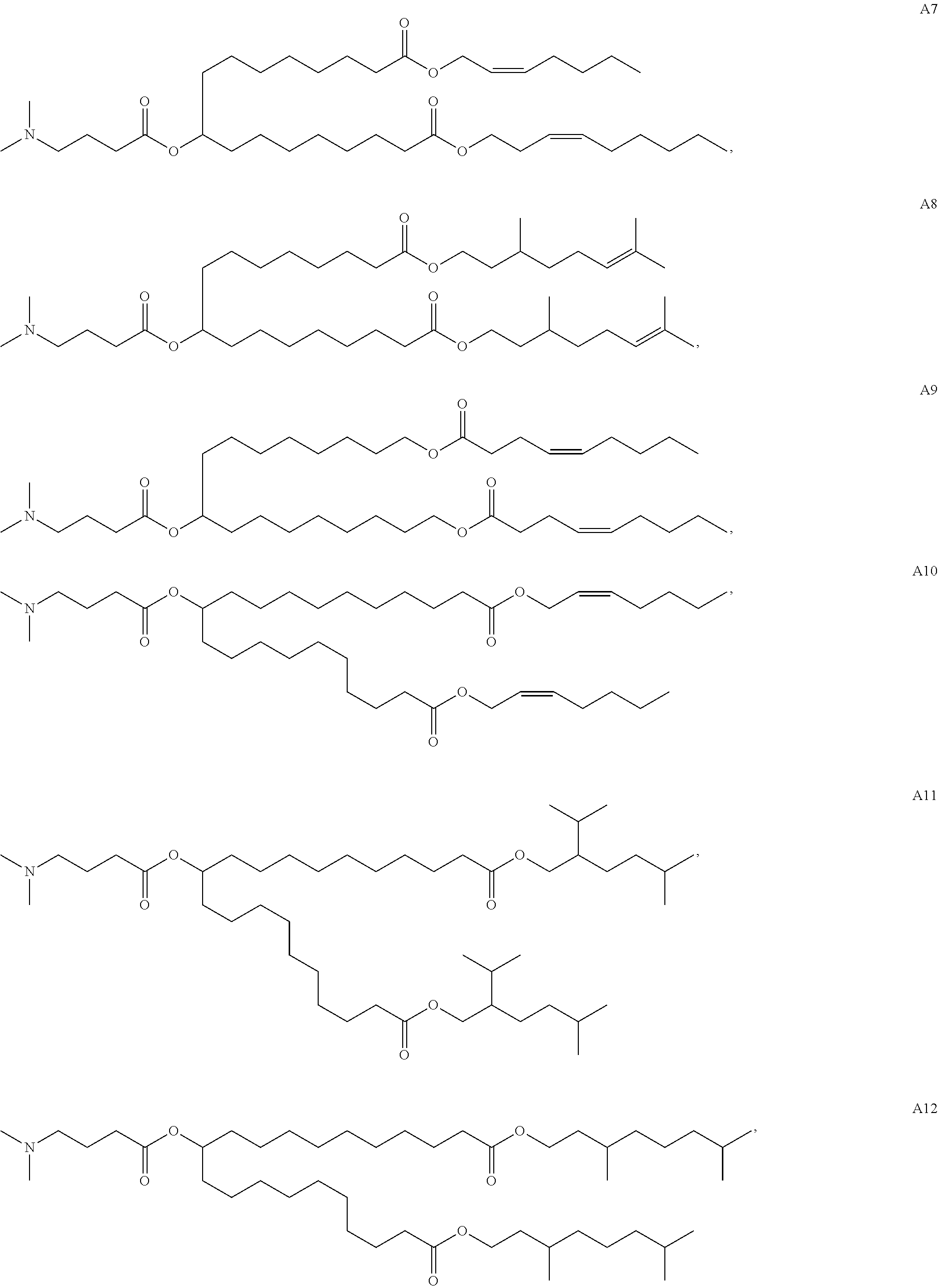

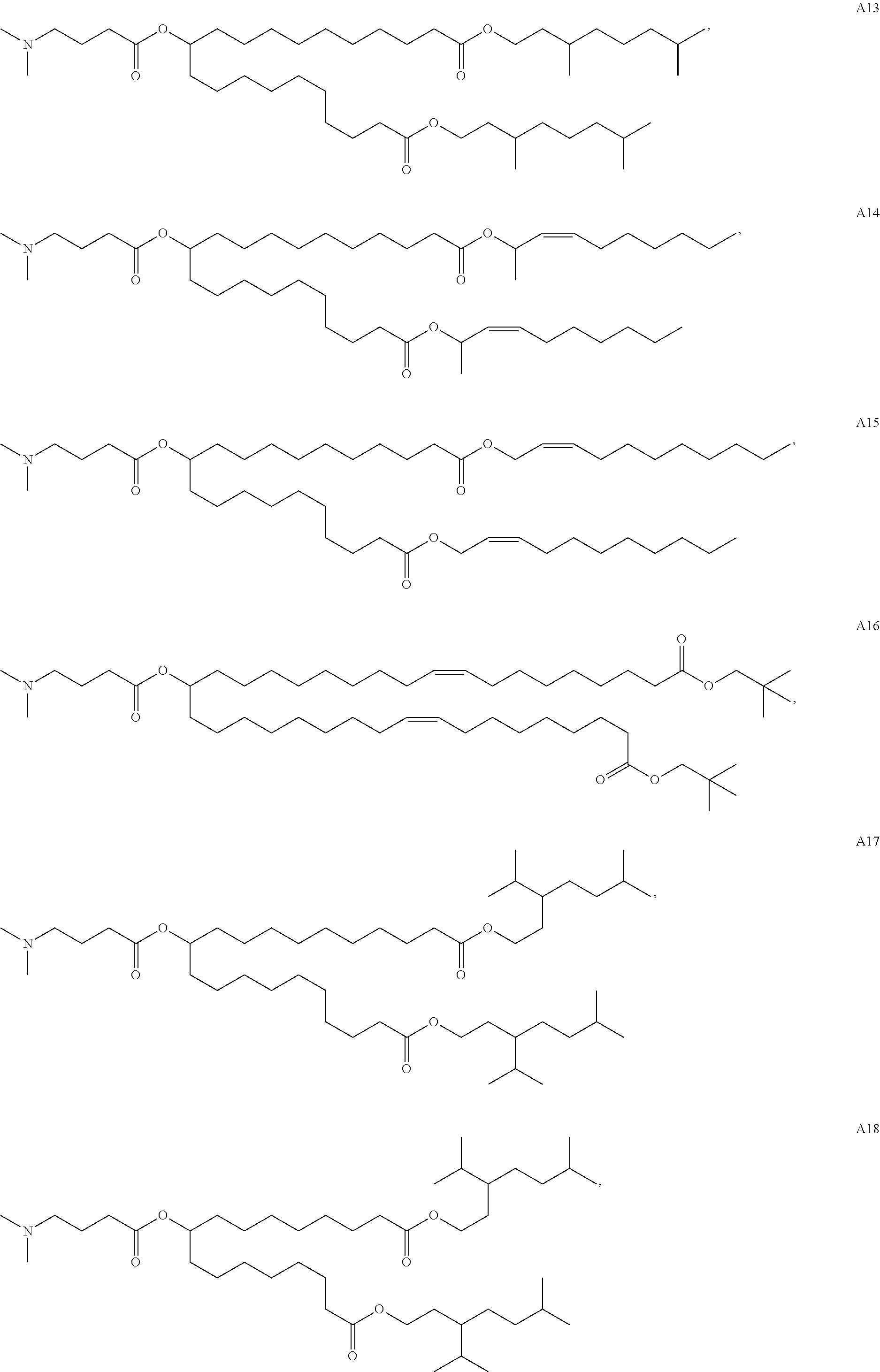

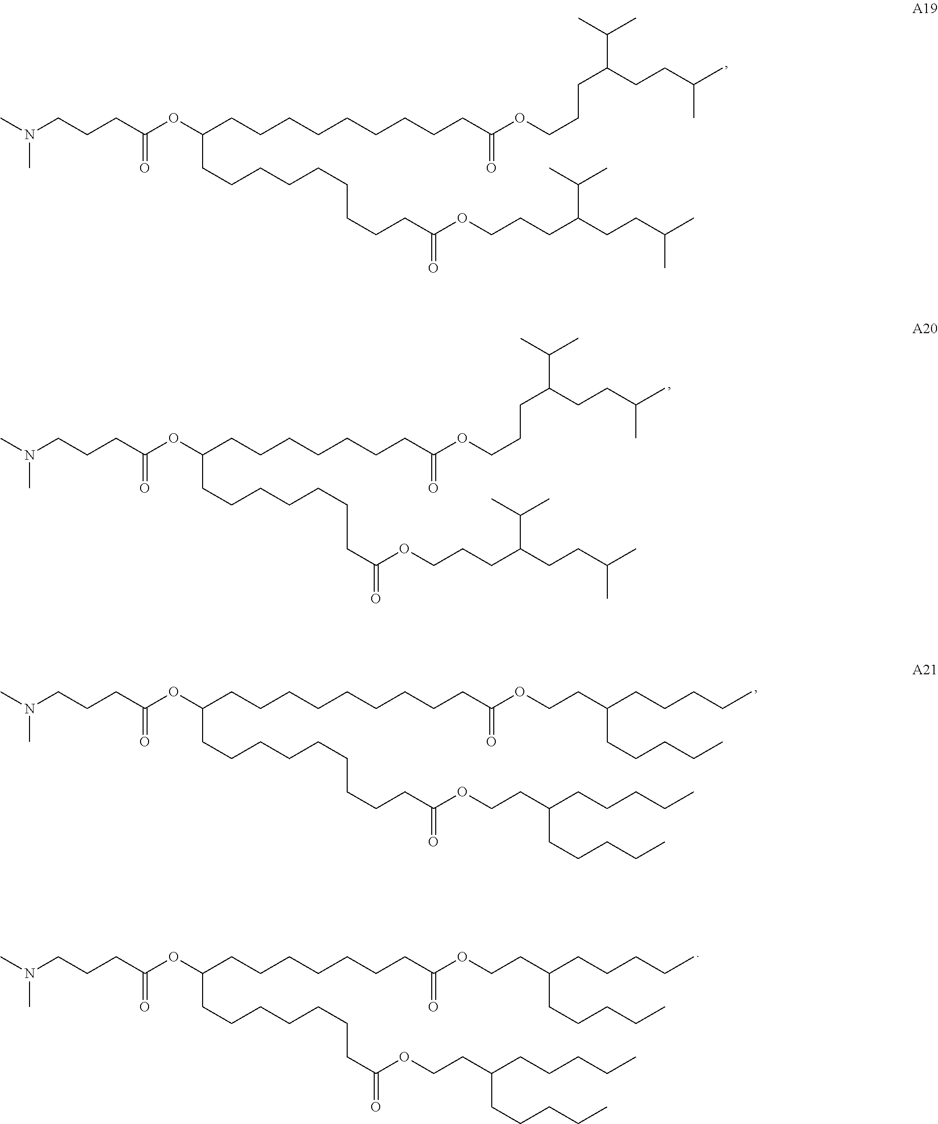

42. The lipid composition of claim 38, wherein the cationic lipid comprises a lipid compound having the structure shown below or a pharmaceutically acceptable salt thereof

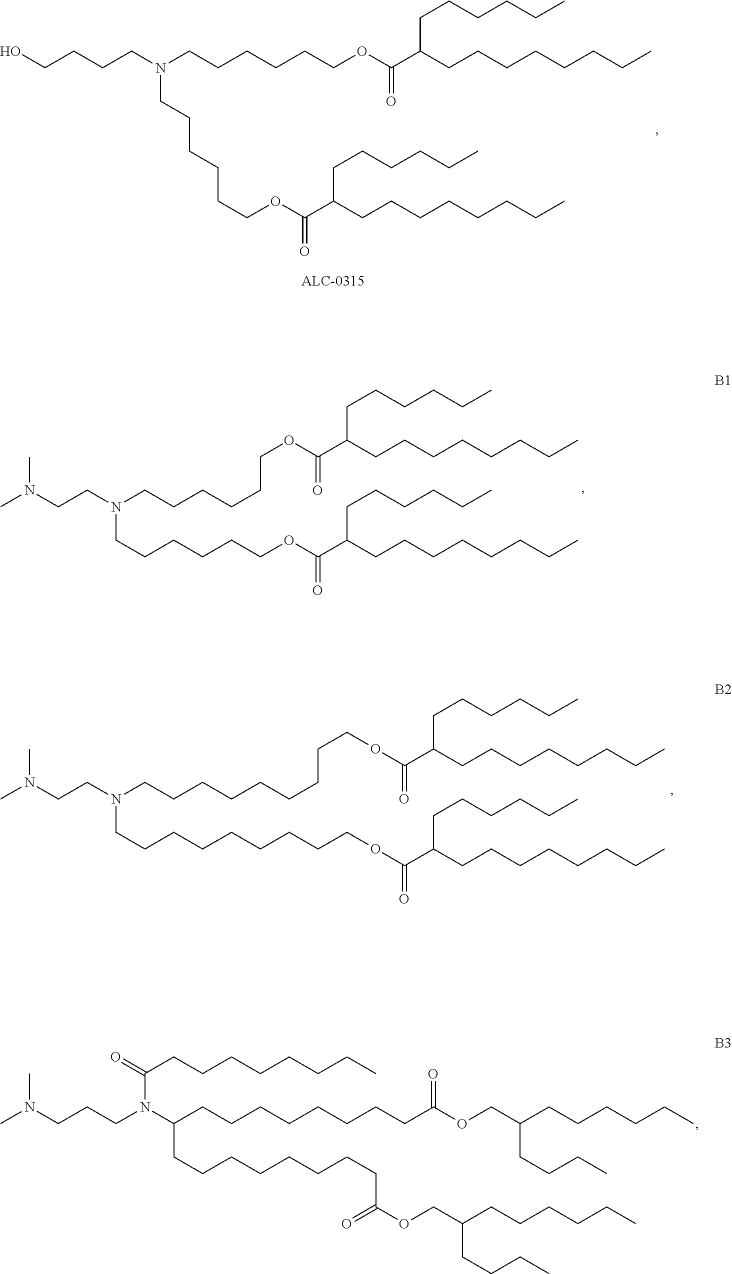

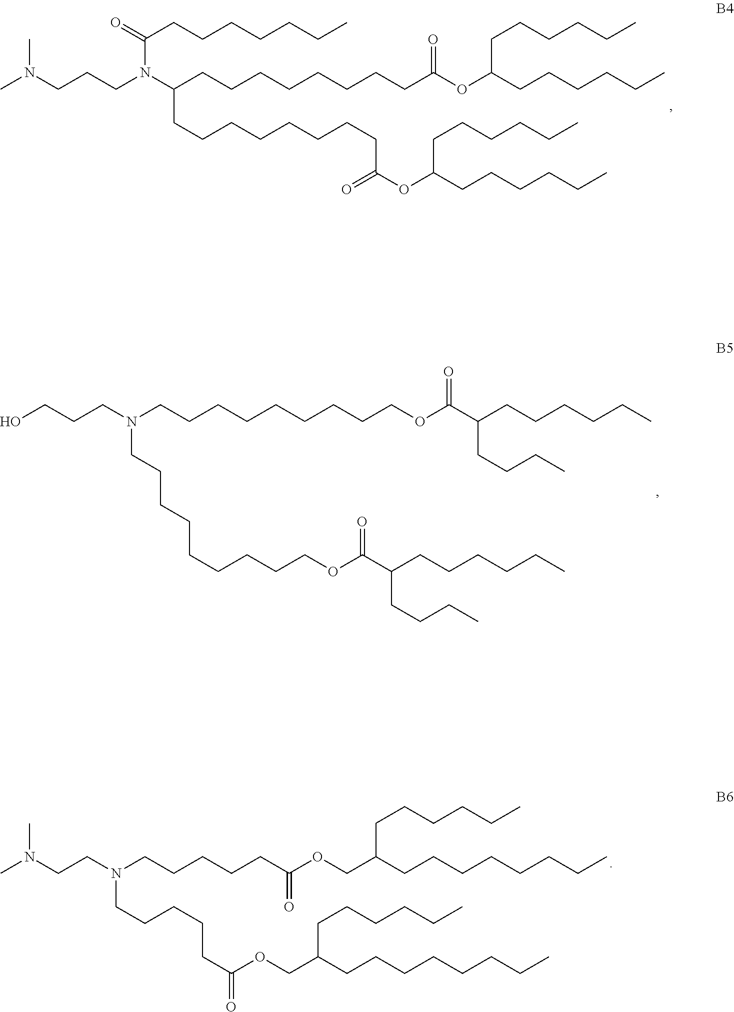

43. The lipid composition of claim 38, wherein, R₁ and R₂ each is independently selected from C₁-C₁₂ Alkyl and C₂-C₁₂ Alkenyl; R₃ and R₄ each is independently selected from C₁-C₁₂ Alkyl, C₂-C₁₂ Alkenyl, C₆-C₁₀ Aryl and 5-10 membered heteroaryl; The condition is R₃ and R₄ At least one of them is C₆-C₁₀ to Aryl or 5-10 membered heteroaryl, and R₃ and R₄ each independently selects t R₆ Replace, t is an integer selected from 1-5; R₆ each is independently selected from C₁-C₁₂ Alkyl and C₂-C₁₂ Alkenyl;
M₁ and M₂ each is independently selected from —OC(O)—, —C(O)O—, —OC(O)O—, —SC(S)— and —C(S)S—; R₅ selected from —C₁₋₁₂ Alkylene-Q, Q is selected from —OR₇ and —SR₇, R₇ Independently selected from H, C₁-C₁₂ Alkyl, C₂-C₁₂ Alkenyl, C₁-C₁₂ Alkoxy, carboxylic acid, sulfinic acid, sulfonic acid, sulfonyl, nitro, cyano, amino, carbamoyl, sulfonamide, C₆-C₁₀ Aryl and 5-10 membered heteroaryl; m and n are each independently an integer selected from 1-12.

44. The lipid composition of claim 43, wherein, R₁ and R₂ each is independently selected from C₁-C₁₂ alkyl,

45. The lipid composition of claim 43, wherein, R₃ and R₄ each is independently selected from C₁-C₁₂ Alkyl and C₆-C₁₀ Aryl; The condition is R₃ and R₄ One is C₆-C₁₀ Aryl, the other is C₁-C₁₂ alkyl; R₃ and R₄ each independently by t R₆ Instead, t is an integer selected from 1-3; R₆ each is independently selected from C₁-C₁₂ alkyl.

46. The lipid composition according to claim 43, wherein, M₁ and M₂ each is independently selected from: —OC(O)—, —C(O)O— and —OC(O)O—.

47. The lipid composition according to claim 43, in, R₅ selected from —C₁₋₅ Alkylene-Q, Q is —OH.

48. The lipid composition according to claim 43, wherein, m and n are each independently an integer selected from 2-7.

49. The lipid composition according to claim 43, wherein, R₄ Replaced by R₂ The 1st or last digit; and/or R₃ Replaced by R₁ The 1st or last digit.

50. The lipid composition according to claim 43, wherein, t is 1 or 2, R₆ Substituted on the benzene ring relative to R₁ or R₂ of metaposition and/or counterposition.

51. The lipid composition according to claim 43, wherein, t is 1 or 2, R₆ each is independently selected from C₁-C₁₀ alkyl.

52. The lipid composition of claim 43, wherein the cationic lipid comprises a compound of formula (II), or a pharmaceutically acceptable salt thereof 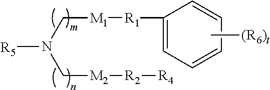
wherein:
R₁, R₂, R₄, R₅, R₆, M₁, M₂, t, m and n are as defined as:
R₁ Selected from C₁-C₆ alkyl;
R₂ Selected from C₁-C₁₀ alkyl;
R₄ Selected from C₁-C₁₀ to alkyl;
M₁ and M₂ each is independently selected from: —OC(O)—, —C(O)O— and —OC(O)O—;
R₅ selected from —C₁₋₅ Alkylene-Q, Q is selected from —OR₇ and —SR₇, R₇ Independently selected from H, C₁-C₁₂ Alkyl and C₂-C₁₂ Alkenyl;
m and n are each independently an integer selected from 2-9;
t is an integer selected from 1-3;
R₆ each is independently selected from C₁-C₁₂ Alkyl and C₂-C₁₂ alkenyl.

53. The lipid composition of claim 43, wherein the cationic lipid comprises a compound of formula (III), or a pharmaceutically acceptable salt thereof: 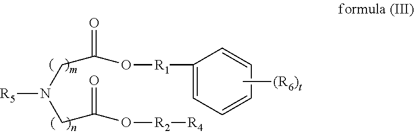
wherein
R₁, R₂, R₄, R₅, R₆, t, m and n are defined as:
R₁ Selected from C₁-C₆ alkyl;
R₂ Selected from C₁-C₁₀ to alkyl;
R₄ Selected from C₁—C to alkyl;
R₅ selected from —C₁₋₃ Alkylene -Q, Q is selected from —OH and —SH;
t is 1 or 2;
R₆ Selected from C₁-C₁₂ Alkyl and C₂-C₁₂ Alkenyl;
m and n are each independently an integer selected from 2-7.

54. The lipid composition of claim 43, wherein the cationic lipid comprises a compound of formula (IV), or a pharmaceutically acceptable salt thereof: 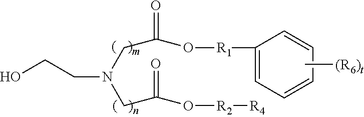
wherein
R₁, R₂, R₄, R₆, t, m and n are defined as:
R₁ Selected from C₁-C₆ alkyl;
R₂ Selected from C₁-C₁₀ alkyl;
R₄ Selected from C₁-C₁₀ to alkyl;
t is 1 or 2;
R₆ each is independently selected from C₁-C₁₂ Alkyl and C₂-C₁₂ Alkenyl;
m and n are each independently an integer selected from 2-7.

55. The lipid composition of claim 43, wherein the cationic lipid comprises a lipid compound having the structure shown below or a pharmaceutically acceptable salt thereof: 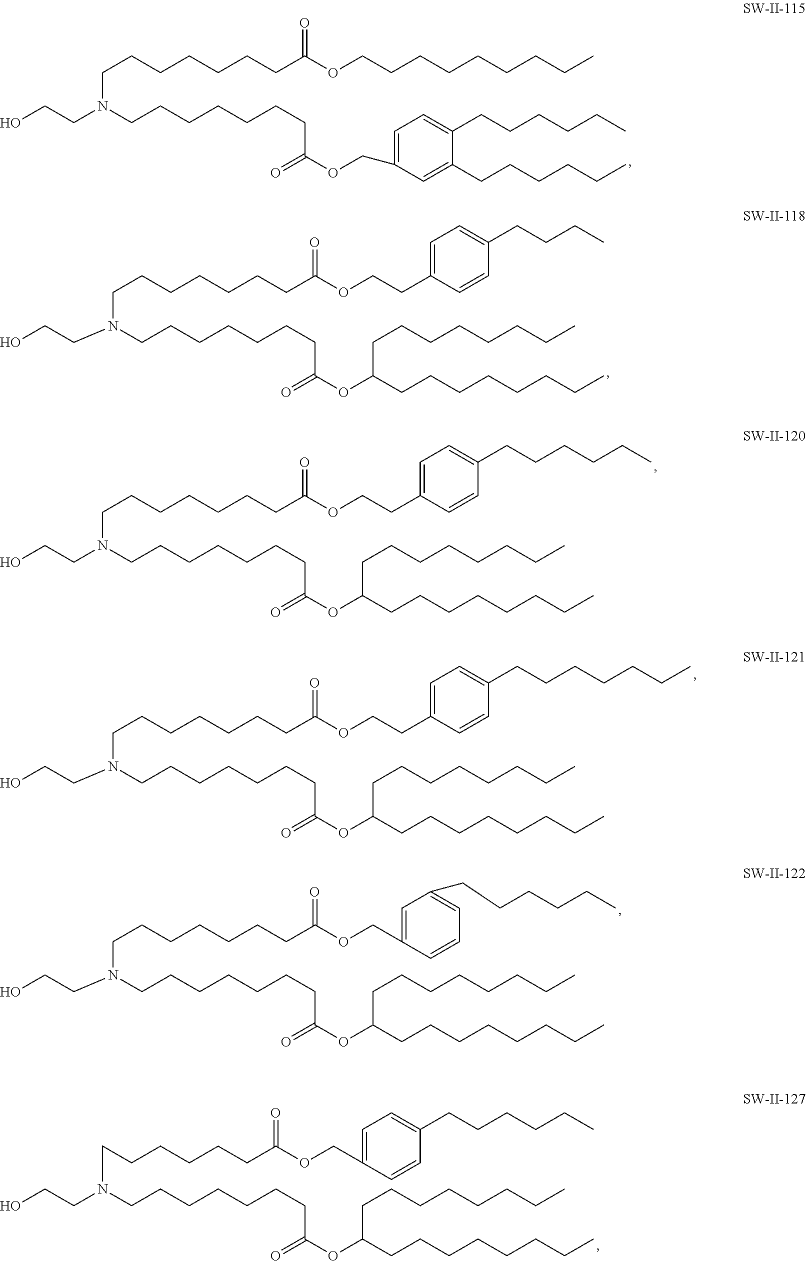 [SMILES: CCCCCCCCCOC(=O)CCCCCCCN(CCO)CCCCCCCC(=O)OCc1ccc(CCCCCC)c(CCCCCC)c1 ; CCCCCCCCC(CCCCCCCC)OC(=O)CCCCCCCN(CCO)CCCCCCCC(=O)OCCc1ccc(CCCC)cc1 ; CCCCCCCCC(CCCCCCCC)OC(=O)CCCCCCCN(CCO)CCCCCCCC(=O)OCCc1ccc(CCCCCC)cc1 ; CCCCCCCCC(CCCCCCCC)OC(=O)CCCCCCCN(CCO)CCCCCCCC(=O)OCCc1ccc(CCCCCCC)cc1 ; CCCCCCCCC(CCCCCCCC)OC(=O)CCCCCCCN(CCO)CCCCCCCC(=O)OCc1cccc(CCCCCC)c1 ; CCCCCCCCC(CCCCCCCC)OC(=O)CCCCCCCN(CCO)CCCCCCC(=O)OCc1ccc(CCCCCC)cc1] 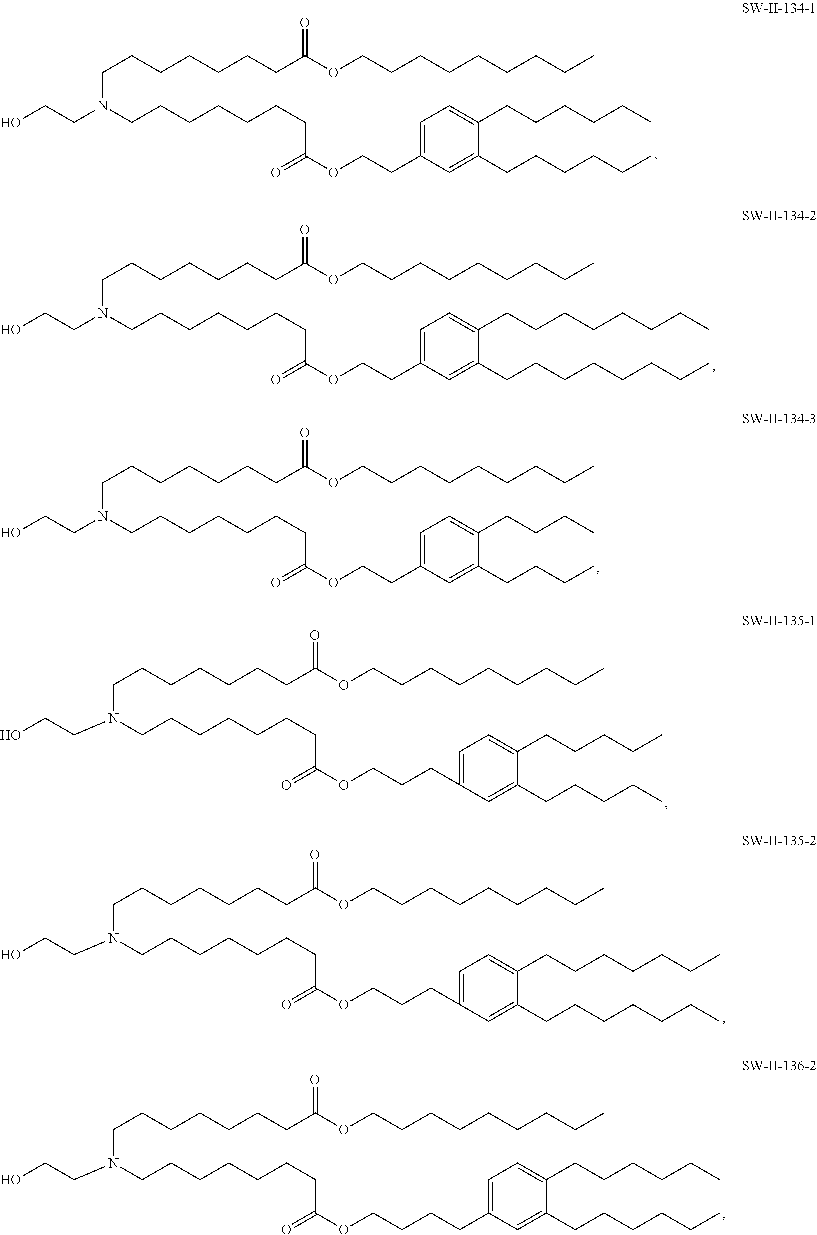 [SMILES: CCCCCCCCCOC(=O)CCCCCCCN(CCO)CCCCCCCC(=O)OCCc1ccc(CCCCCC)c(CCCCCC)c1 ; CCCCCCCCCOC(=O)CCCCCCCN(CCO)CCCCCCCC(=O)OCCc1ccc(CCCCCCCC)c(CCCCCCCC)c1 ; CCCCCCCCCOC(=O)CCCCCCCN(CCO)CCCCCCCC(=O)OCCc1ccc(CCCC)c(CCCC)c1 ; CCCCCCCCCOC(=O)CCCCCCCN(CCO)CCCCCCCC(=O)OCCCc1ccc(CCCCC)c(CCCCC)c1 ; CCCCCCCCCOC(=O)CCCCCCCN(CCO)CCCCCCCC(=O)OCCCc1ccc(CCCCCCC)c(CCCCCCC)c1 ; CCCCCCCCCOC(=O)CCCCCCCN(CCO)CCCCCCCC(=O)OCCCCc1ccc(CCCCCC)c(CCCCCC)c1] 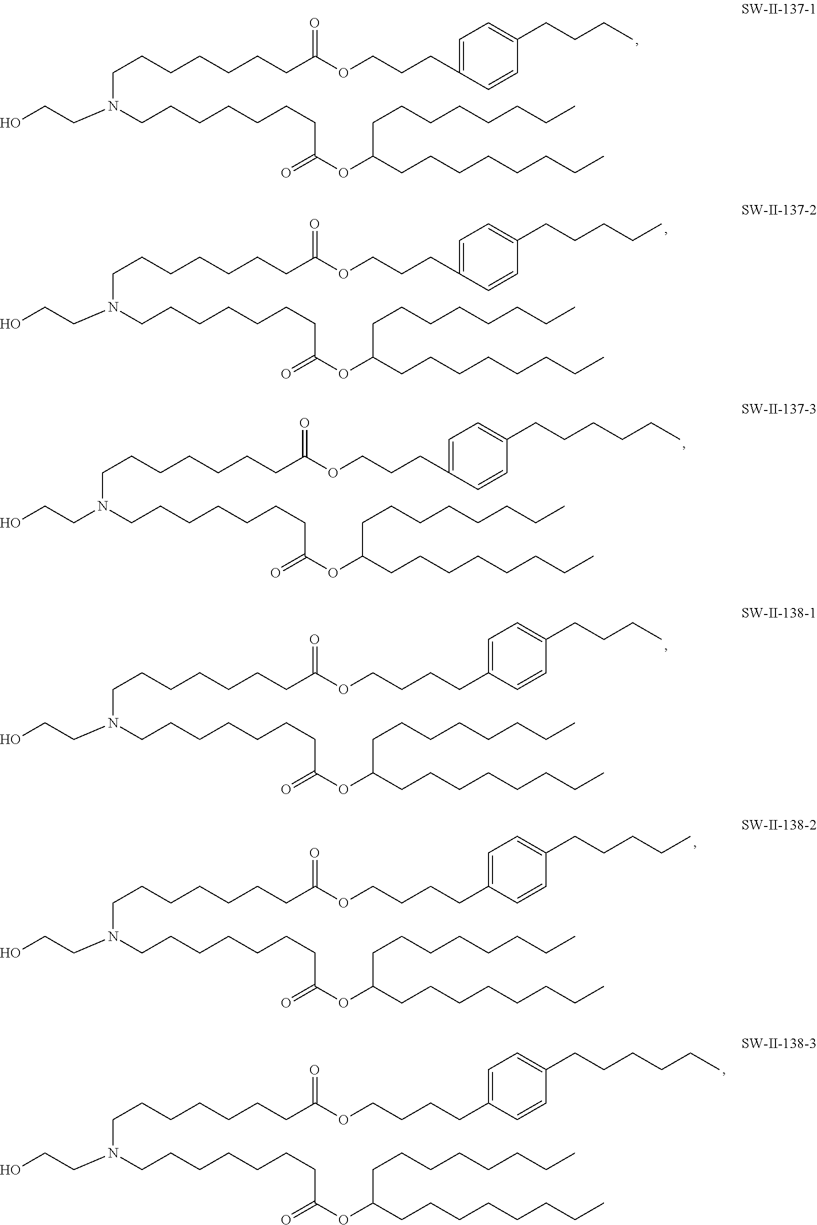 [SMILES: CCCCCCCCC(CCCCCCCC)OC(=O)CCCCCCCN(CCO)CCCCCCCC(=O)OCCCc1ccc(CCCC)cc1 ; CCCCCCCCC(CCCCCCCC)OC(=O)CCCCCCCN(CCO)CCCCCCCC(=O)OCCCc1ccc(CCCCC)cc1 ; CCCCCCCCC(CCCCCCCC)OC(=O)CCCCCCCN(CCO)CCCCCCCC(=O)OCCCc1ccc(CCCCCC)cc1 ; CCCCCCCCC(CCCCCCCC)OC(=O)CCCCCCCN(CCO)CCCCCCCC(=O)OCCCCc1ccc(CCCC)cc1 ; CCCCCCCCC(CCCCCCCC)OC(=O)CCCCCCCN(CCO)CCCCCCCC(=O)OCCCCc1ccc(CCCCC)cc1 ; CCCCCCCCC(CCCCCCCC)OC(=O)CCCCCCCN(CCO)CCCCCCCC(=O)OCCCCc1ccc(CCCCCC)cc1] [chemical structure]


SEQUENCE LISTING (4 sequences)

SEQ ID NO: 1 - RNA, 1653 nt, synthetic construct
  source 1..1653: mol_type = other RNA
atggaggacgccaagaacatcaagaagggccccgcccccttctaccccctggaggacggcaccgccggcgagcagctgcacaaggccatgaagcggtacgccctggtgcccggcaccatcgccttcaccgacgcccacatcgaggtggacatcacctacgccgagtacttcgagatgagcgtgcggctggccgaggccatgaagcggtacggcctgaacaccaaccaccggatcgtggtgtgcagcgagaacagcctgcagttcttcatgcccgtgctgggcgccctgttcatcggcgtggccgtggcccccgccaacgacatctacaacgagcgggagctgctgaacagcatgggcatcagccagcccaccgtggtgttcgtgagcaagaagggcctgcagaagatcctgaacgtgcagaagaagctgcccatcatccagaagatcatcatcatggacagcaagaccgactaccagggcttccagagcatgtacaccttcgtgaccagccacctgccccccggcttcaacgagtacgacttcgtgcccgagagcttcgaccgggacaagaccatcgccctgatcatgaacagcagcggcagcaccggcctgcccaagggcgtggccctgccccaccggaccgcctgcgtgcggttcagccacgcccgggaccccatcttcggcaaccagatcatccccgacaccgccatcctgagcgtggtgcccttccaccacggcttcggcatgttcaccaccctgggctacctgatctgcggcttccgggtggtgctgatgtaccggttcgaggaggagctgttcctgcggagcctgcaggactacaagatccagagcgccctgctggtgcccaccctgttcagcttcttcgccaagagcaccctgatcgacaagtacgacctgagcaacctgcacgagatcgccagcggcggcgcccccctgagcaaggaggtgggcgaggccgtggccaagcggttccacctgcccggcatccggcagggctacggcctgaccgagaccaccagcgccatcctgatcacccccgagggcgacgacaagcccggcgccgtgggcaaggtggtgcccttcttcgaggccaaggtggtggacctggacaccggcaagaccctgggcgtgaaccagcggggcgagctgtgcgtgcggggccccatgatcatgagcggctacgtgaacaaccccgaggccaccaacgccctgatcgacaaggacggctggctgcacagcggcgacatcgcctactgggacgaggacgagcacttcttcatcgtggaccggctgaagagcctgatcaagtacaagggctaccaggtggcccccgccgagctggagagcatcctgctgcagcaccccaacatcttcgacgccggcgtggccggcctgcccgacgacgacgccggcgagctgcccgccgccgtggtggtgctggagcacggcaagaccatgaccgagaaggagatcgtggactacgtggccagccaggtgaccaccgccaagaagctgcggggcggcgtggtgttcgtggacgaggtgcccaagggcctgaccggcaagctggacgcccggaagatccgggagatcctgatcaaggccaagaagggcggcaagatcgccgtgtga

SEQ ID NO: 2 - RNA, 3825 nt, synthetic construct
  source 1..3825: mol_type = other RNA
atgttcgtattcctagtgctgctgcccctggtgagcagccagtgcgtgaacctgaccacacgcactcagctgccaccagcctacaccaactcattcaccaggggcgtgtactatcctgacaaggtgttcaggagcagcgtgctgcactccacccaggatctgttcctgcccttcttcagcaacgtgacatggttccacgccatccacgtgagcgggacaaacggcaccaagcggtttgacaatcccgttctgccattcaacgatggcgtgtacttcgccagcaccgagaaaagcaacatcatccggggctggatcttcggcaccacactggacagcaagacccagagtctgctgatcgtgaacaacgccaccaacgtggtgatcaaggtgtgtgagttccagttctgtaatgaccccttcctgggggtctattaccacaagaacaataaaagctggatggagagcgagtttcgggtgtacagtagtgccaacaactgtacattcgagtacgtgtcccagccattcctgatggacctggaggggaagcaggggaacttcaagaacctgcgggagttcgtgtttaagaatatcgacggatattttaagatctactccaagcacacccccatcaacctggtgcgggatctgccccagggcttcagcgccctggagcccctggtggacctgcccatcgggatcaacatcacccggttccagaccctgctggccctgcaccggagttacctgaccccaggtgactccagcagcggctggacagccggggctgcagcctactacgtgggctacctgcagccccggaccttcctcctgaagtacaatgagaacggcaccatcaccgatgccgtggattgtgctctggaccccctgtctgagaccaagtgcaccctgaaaagcttcaccgtggagaaggggatctaccagacaagcaactttcgggtgcagcccaccgagagtatcgtgaggttccccaacatcaccaacctgtgtccattcggggaggtgttcaacgccacccggttcgcaagcgtgtacgcttggaaccggaagcgcatcagcaattgcgtggctgattacagcgtgctgtacaacagcgcctccttcagcacattcaagtgctacggcgtctcccccacaaagctgaacgatctgtgtttcaccaacgtgtacgcagacagctttgtgatccggggagacgaggtgcggcagatcgcacccgggcagaccggcaagattgctgactacaattacaagctgcctgatgacttcacaggctgcgtgattgcctggaacagcaataacctggacagcaaggtgggaggtaattacaattacctctaccggctgttcaggaagtccaatctgaagcccttcgagagggacatctccaccgagatctaccaggctgggtccaccccatgcaatggggtggagggcttcaactgctacttccccctgcagtcctacggcttccagcccaccaatggggtgggctaccagccctaccgggtggtggtgctgagcttcgagctcctgcacgcccccgccactgtgtgcggccccaagaagtccaccaacctggtgaagaacaagtgcgtgaacttcaacttcaacgggttgaccgggactggggtgctcacagagagcaacaagaagttcctgcccttccagcagttcggaagggacatcgccgacaccacggatgccgtgagggatccccagaccctcgagatcctggacatcacgccatgctccttcgggggcgtgagcgtgatcactcctggcacaaacacctccaaccaggtggctgtgctgtaccagggcgtgaactgcaccgaggtgcctgtggccatccatgctgaccagctgacccccacctggcgggtctacagcaccggcagcaacgtgttccagacccgggccggctgcctgatcggggccgagcacgtgaacaacagctacgagtgcgatatccccatcggcgccggcatctgcgctagctaccagacacagaccaatagcccccaatccgctcaatctgtggctagccagagcatcatcgcctacactatgtctctgggcgcagagaatagtgtggcttacagcaacaacagcatcgccattcccactaatttcacaattagtgtgaccaccgagatcctgcctgtgagcatgaccaagaccagcgtggactgcaccatgtacatctgcggggatagcaccgagtgcagcaacctgctgctgcagtacggcagcttctgcacacagctgaaccgggccctgaccggtatcgctgtggagcaggacaagaacacccaggaggtgtttgcgcaggtgaagcagatctacaagacccctcctatcaaggacttcgggggctttaacttcagccagatcctgccagacccctccaagccctccaagaggagcttcatcgaggatctcctgttcaacaaggtgaccctggccgatgccggcttcatcaagcagtacggagattgcctgggcgacatcgccgccagggatctcatctgcgcccagaagttcaacggcctgaccgtgctgccccccctgctgaccgatgagatgatcgctcagtacaccagcgccctgctggctgggaccatcaccagcgggtggaccttcggggccggcgccgccctgcagatccccttcgccatgcagatggcctaccggttcaacggtattggggtgacccagaatgtgctgtatgagaaccagaagctgatcgccaaccagttcaactctgccatcggcaagatccaggactccctgagcagcactgccagcgccctgggcaagctgcaggacgtggtgaaccagaacgcccaggccctgaataccctggtgaagcagctgtccagcaacttcggggccatcagcagcgtgctgaacgacatcctgtcccgcctggacccaccagaggcagaggtgcagatcgatcggctgatcacaggtaggctgcagagcctgcagacctacgtgacccagcagctgatccgcgccgctgagattagggccagcgccaacctggccgccactaagatgagcgagtgcgtgctgggccagagcaagcgggtggacttctgcggcaaggggtatcacctgatgagcttccctcagtccgccccccacggggtggtgttcctgcacgtgacatacgtgcccgcccaggagaagaacttcaccaccgcccctgccatctgccacgatggcaaggcccactttccccgggagggcgtgttcgtgtccaatggcacacattggttcgtgacccagcggaacttctacgagccacagatcattaccactgacaatacctttgtcagtggtaactgtgacgtggtgatcggcattgtgaacaacacagtgtacgatcctctgcagccagagctggacagcttcaaggaggagctggacaagtacttcaagaaccacaccagccccgacgtggatcttggagacatctctggcatcaacgccagcgtggtcaacatccagaaggagattgaccgcctgaacgaggttgccaagaacctgaacgagagtctgatcgacctccaggagcttggcaagtacgagcagtacatcaagtggccatggtacatctggctgggcttcatcgccggcctgatcgccatcgtgatggtgaccatcatgctgtgctgcatgacctcttgttgcagctgcctgaaggggtgctgcagctgcggtagttgctgtaagttcgacgaggacgacagcgagcccgtgctgaagggcgtgaagctgcactacacgtaatag

SEQ ID NO: 3 - RNA, 1563 nt, synthetic construct
  source 1..1563: mol_type = other RNA
atggcgtcccaaggcaccaaacggtcttatgaacagatggaaactgatggggaacgccagaatgcaactgagattagggcatccgtcgggaagatgattgatggaattgggcgattctacatccaaatgtgcactgaacttaaactcagtgattatgaagggcggttgatccagaacagcttgacaatagagagaatggtgctctctgcttttgatgagagaaggaatagatatctggaagaacaccccagcgcggggaaagatcctaagaaaactggagggcccatatacaagagagtagatggaagatggatgagggaactcgtcctttatgacaaagaagaaataaggcgaatctggcgccaagccaacaatggtgatgatgcgacagctggtctaactcacatgatgatctggcattccaatttgaatgatacaacataccagaggacaagagctcttgttcgcaccggaatggaccccaggatgtgctctctgatgcagggttcgactctccctagaaggtccggagctgcaggcgctgcagtcaaaggaatcgggacaatggtgatggagctgatcagaatgatcaaacgggggatcaacgatcgaaatttctggagaggtgagaatgggcggaaaacaaggagtgcttatgagagaatgtgcaacattcttaaaggaaaatttcaaacagctgcacaaagagcaatgatggatcaagtgagagaaagccggaacccaggaaatgctgagatcgaagatctcatattttcggcaagatctgcactaatattgagagggtcagttgctcacaaatcttgcctacctgcctgtgtgtatggacctgcagtatccagtgggtacaacttcgaaaaagagggatattccttagtgggaatagaccctttcaaactacttcaaaatagccaagtatacagcctaatcagaccgaacgagaatccagcacacaagagtcagctggtgtggatggcatgccattctgctgcatttgaagatttaagattgttaagcttcatcagagggaccaaagtatctccgcgggggaaactttcaactagaggagtacaaattgcttcaaatgagaacatggataatatggaatcaagtactcttgaactgagaagcaggtactgggccataaggaccaggagtggaggaaacactaatcaacagagggcctctgcaggccaaatcagtgtgcaacctacgttttctgtacaaagaaacctcccatttgaaaaatcaaccgtcatggcagcattcactggaaatacggagggaagaacctcagacatgagggcagaaatcataaggatgatggaaggtacaaaaccagaagaagtgtccttccgtgggcggggagttttcgagctctcagacgaaaaggcaacgaacccgatcgtgccctcttttgacatgagtaatgaaggatcttatttcttcggagacaatgcagaagagtacgacaatagccttctaaccgaggtcgaaacgcctatcagaaacgaatgggggtgcagatgcaacgattcaagtgac

SEQ ID NO: 4 - RNA, 720 nt, synthetic construct
  source 1..720: mol_type = other RNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa